(19) United States
(12) Patent Application Publication
(10) Pub. No.: US 2020/0329685 A1
(43) Pub. Date: Oct. 22, 2020

(54) TRANSGENIC EUKARYOTIC ORGANISMS AND METHODS FOR GENDER SELECTION

(71) Applicant: RAMOT AT TEL-AVIV UNIVERSITY LTD., Tel Aviv (IL)

(72) Inventors: Ehud QIMRON, Tel Aviv (IL); Ido YOSEF, Moshav Netiv Ha'Asara (IL); Mordechay GERLIC, Meitar (IL)

(21) Appl. No.: 16/959,878

(22) PCT Filed: Jan. 3, 2019
(86) PCT No.: PCT/IL2019/050015

§ 371 Date: Jul. 2, 2020

Related U.S. Application Data
(60) Provisional application No. 62/613,531, filed on Jan. 4, 2018.

(51) Int. Cl. A01K 67/027; A01K 67/033; C12N 15/82; C12N 15/11; C12N 9/22

(52) U.S. Cl. CPC A01K 67/0275; A01K 67/02; A01K 67/0339; C12N 15/8216; C12N 15/11; C12N 9/22; A01K 67/0334; A01K 67/0335; A01K 2227/105; A01K 2227/30; A01K 2227/40; A01K 2227/70; A01K 2227/706; A01K 2217/05; C12N 2310/20; C12N 2800/80; A01K 67/0331; A01K 67/0338

(57) ABSTRACT

The present invention provides systems and non-invasive methods for gender selection of eukaryotic organisms More specifically, the invention applies the CRISPR-Cas system as well as any derivatives and fusion proteins thereof for creation of transgenic eukaryotic organisms and for selecting the desired gender of the resulting progeny.

### FIELD OF THE INVENTION

The present invention relates to transgenic eukaryotic organisms, systems and non-invasive methods for gender selection of eukaryotic organisms. More specifically, the invention applies the CRISPR-Cas system for creation of transgenic eukaryotic organisms and for selecting the desired gender of the resulting progeny.

### BACKGROUND ART

References considered to be relevant as background to the presently disclosed subject matter are listed below:
[1] Levy, T. et al. A Single Injection of Hypertrophied Androgenic Gland Cells Produces All-Female Aquaculture. Mar Biotechnol (NY) 18, 554-563 (2016);
[2] Liu, H. et al. Genetic manipulation of sex ratio for the large-scale breeding of YY super-male and XY all-male yellow catfish (Pelteobagrus fulvidraco (Richardson)). Mar Biotechnol (NY) 15, 321-328 (2013);
[3] Yamamoto, T. O. A YY male goldfish from mating estrone-induced XY female and normal male. J Hered 66, 2-4 (1975);
[4] Chevassus, B., Devaux, A., Chourrout, D. & Jalabert, B. Production of YY rainbow trout males by self-fertilization of induced hermaphrodites. J Hered 79, 89-92 (1988);
[5] Hickey, W. A. & Craig, G. B., Jr. Genetic distortion of sex ratio in a mosquito, Aedes aegypti. Genetics 53, 1177-1196 (1966);
[6] Novitski, E. & Hanks, G. D. Analysis of Irradiated Drosophila Populations for Meiotic Drive. Nature 190, 989 (1961);
[7] Hall, A. B. et al. SEX DETERMINATION. A male-determining factor in the mosquito Aedes aegypti. Science 348, 1268-1270 (2015);
[8] Galizi, R. et al. A CRISPR-Cas9 sex-ratio distortion system for genetic control. Sci Rep 6, 31139 (2016);
[9] Galizi, R. et al. A synthetic sex ratio distortion system for the control of the human malaria mosquito. Nat Commm 5, 3977 (2014);
[10] Kyrou, K. et al. A CRISPR-Cas9 gene drive targeting doublesex causes complete population suppression in caged Anopheles gambiae mosquitoes. Nat Biotechnol (2018);
[11] Hammond, A. et al. A CRISPR-Cas9 gene drive system targeting female reproduction in the malaria mosquito vector Anopheles gambiae. Nat Biotechnol 34, 78-83 (2016);
[12] WO2015105928;
[13] CN105861554;
[14] U.S. Pat. No. 5,596,089;
[15] Zuo Q, et al. J Cell Biochem., 118(8): 2380-2386 (2017);
[16] Hirst C E et al. Methods Mol Biol. 1650:177-190 (2017); Acknowledgement of the above references herein is not to be inferred as meaning that these are in any way relevant to the patentability of the presently disclosed subject matter.

### BACKGROUND OF THE INVENTION

Gender selection of plants and farm animals confers economic advantages and significantly reduces cruelty to the animals. In the poultry industry, for example, almost all male progeny are brutally killed shortly after hatching. The selection process of females from males, as well as the massive killing of the males, require time and intensive labor, and thus constitute a huge economic burden. Some aquatic organisms as well as plants that benefit from single-sex cultivation have been produced mostly by hormonal feminization of males or by masculinization of females and the subsequent production of a single-sex progeny. This was demonstrated in crustaceans [1], fish [2-4], and is also common in growing Cannabis sativa, where feminized seeds are desired. However, these practices are not feasible for terrestrial livestock. The sex ratio in a population of mosquitoes and flies was shifted by manipulating specific genes that distort the sex ratio [5-7]. In recent breakthrough studies, researchers have even completely distorted the sex ratio, accompanied by the sterility of females, thus resulting in a collapsed population [8-11]. Such an outcome is desirable for disease-transferring insects in the wild, but not for domesticated livestock. For domesticated livestock, a different approach should be adopted, which produces a desired sex, while retaining a reservoir of males and females to maintain such a set-up. Manipulated animals that produce only one sex are impossible to sustain by self-crossing, because either the male or female is absent. WO2015105928 [12], disclose methods for manipulating population of animals, by “releasing” in a wild type population a genetically engineered transgenic animal expressing the CRISPR-Cas system. One example for such manipulation relates to biasing the sex-ratio of a population by creating a transgenic animal that encode on one or more gender chromosome/s thereof (a) RNA guided nuclease (e.g., Cas9); and (b) express gRNAs that target the nuclease to cleave sequences uniquely found on the other chromosome (e.g., “X-derived” that targets the Y chromosome or alternatively, “Y-derived” that targets the X chromosome. However, the safer separation of both elements of the CRISPR-Cas system and creation of the CRISPR-Cas system in two transgenic animals each expressing only one of the elements of this system is not disclosed or even hinted by this document. CN105861554 [13], relates to the use of CRISPR system to target the Rbmy gene, that is required for spermatogenesis and display multiple copies on Y chromosome. The method involves injection of CRISPR-Cas9 and specific gRNAs targeting sequences within the Rbmy gene, into a fertilized egg. This document relates thus to invasive methods and does not suggest or even hints the creation of two strains of transgenic animals separating the two elements of the CRISPR-Cas system. U.S. Pat. No. 5,596,089 [14] is based on the Sex-Determining Region Y, or SRY gene that is represented uniquely on the Y chromosome, with no X-chromosome homologous sequence and uses thereof as a target for gene-based methods for sex determination. More specifically, this publication describes methods for manipulation of sex phenotype using control elements of the SRY, linked to a toxin, e.g., diphtheria toxin subunit A, etc. The method involves creation of two strains of animals (e.g., pigs) based on the Cre-loxP system for selective production of the toxin protein in males. The present invention is however advantageous as no toxic compounds are involved, and no temporal expression regulated by specific promoters are required. Zuo Q, et al. [15] describes CRISPR-Cas9 mediated knockout of the C1EIS gene in chicken embryonic stem cells, thereby inhibiting differentiation thereof into spermatogonadial stem cells (SSCs). In the present study, the CRISPR-Cas9 is directed against essential genes for embryonic development. Hirst et al. [16] describe functional analysis of genes involved in avian sex determination. CRISPR-Cas9 technology is used herein for targeted genome editing in the avian urogenital system. This document does not suggest the creation of two strains of transgenic animals separating the two elements of the CRISPR-Cas system.

Thus, the provision of safe, efficient and non-invasive methods for selecting a desired gender of an eukaryotic organism and optionally for manipulating properties of the selected gender, are needs unmet by the methods of the art. Another cardinal unmet need is the creation of genetically stable transgenic animals that can be propagated without adverse genetic alterations. These needs are clearly addressed by the present invention that provides stable systems and non-invasive methods for gender selection in the embryonic stage, i.e. prior to birth, saving labor, costs, and animal misery.

### SUMMARY OF THE INVENTION

In a first aspect, the invention relates to a system comprising at least one transgenic homogametic organism and at least one transgenic heterogametic organism.

More specifically, the system of the invention comprising:

(A) a transgenic eukaryotic heterogametic organism comprising one of (a) or (b):

In some embodiments (a), the heterogametic organism of the invention may comprise at least one nucleic acid sequence that may be (i), a sequence encoding or forming at least one target recognition element for at least one nucleic acids modifier protein, specifically, at least one nuclease; or at least (ii), a sequence encoding or forming the at least one target recognition element and a nucleic acid sequence encoding a first fragment, domain or subunit of at least one said nucleic acids modifier protein (nuclease).

In another option (b), the heterogametic organism of the system of the invention may comprise at least one nucleic acid sequence encoding: in some embodiments (i), at least one nucleic acids modifier protein; or alternatively, in some other embodiments (ii), a second fragment, domain or subunit of such at least one nucleic acids modifier protein (nuclease).

Still further, in some embodiments, the nucleic acid sequence of (a) or (b) comprised within the heterogametic transgenic organism may be integrated into one of the gender-chromosomes of the transgenic heterogametic organism.

(B) a transgenic eukaryotic homogametic organism comprising one of (a) or (b):

In one option (a), at least one nucleic acid sequence encoding:

In some embodiments (i), at least one nucleic acids modifier protein; or alternatively, (ii), a second fragment, domain or subunit of the at least one nucleic acids modifier protein (e.g., nuclease); or In yet an alternative option (b), the homogametic organism may comprise at least one nucleic acid sequence that may comprise in some embodiments (i), a sequence encoding or forming at least one target recognition element for at least one nucleic acids modifier protein (e.g., nuclease). In yet some alternative embodiments, such sequence may comprise (ii), a sequence encoding or forming the at least one target recognition element; and in addition, a nucleic acid sequence encoding a first fragment, domain or subunit of at least one such nucleic acids modifier protein (e.g., nuclease).

In a second aspect, the invention relates to a method for selecting a desired gender of an eukaryotic organism. More specifically, the method may comprise the steps of:

In a first step (A), providing a transgenic eukaryotic heterogametic organism comprising one of (a) or (b): In one option (a), the transgenic eukaryotic heterogametic organism comprises at least one nucleic acid sequence: (i) said sequence encoding or forming at least one target recognition element for at least one nucleic acids modifier protein (e.g., nuclease); or (ii) said sequence encoding or forming said at least one target recognition element and a nucleic acid sequence encoding a first fragment, domain or subunit of at least one said nucleic acids modifier protein (e.g., nuclease). In another option (b), the transgenic eukaryotic heterogametic organism comprises at least one nucleic acid sequence encoding: either (i), at least one nucleic acids modifier protein (e.g., nuclease); or (ii), a second fragment, domain or subunit of said at least one nucleic acids modifier protein (e.g., nuclease). The nucleic acid sequence may be integrated into one of the gender-chromosomes of the transgenic heterogametic organism.

The next step (B), involves providing a transgenic eukaryotic homogametic organism comprising one of (a) or (b). More specifically, in one option (a), the transgenic eukaryotic homogametic organism comprises at least one nucleic acid sequence encoding: either (i), at least one nucleic acids modifier protein (e.g., nuclease); or (ii), a first fragment, domain or subunit of said at least one nucleic acids modifier protein (e.g., nuclease). In the second option (b), the transgenic eukaryotic homogametic organism comprises at least one nucleic acid sequence: (i) said sequence encoding or forming at least one target recognition element for at least one nucleic acids modifier protein (e.g., nuclease); or (ii) said sequence encoding or forming said at least one target recognition element and a nucleic acid sequence encoding a first fragment, domain or subunit of at least one said nucleic acids modifier protein (e.g., nuclease).

The nucleic acid sequence may be integrated into at least one allele of any chromosomal or mitochondrial DNA of said transgenic homogametic organism.

The next step (C), involves breeding said transgenic heterogametic organism provided in step (A) with said transgenic homogametic organism provided in step (B), thereby obtaining a progeny predominantly composed of said one desired gender.

In yet another aspect, the invention relates to a method for selecting a desired gender of an eukaryotic organism and for modifying at least one undesired trait in the selected organism. More specifically, the method comprising the steps of:

In a first step (a), providing a transgenic eukaryotic heterogametic organism comprising:

(i) at least one nucleic acid sequence encoding at least one guide RNA directed against at least one target sequence within at least one of coding and non-coding sequences or any product/s thereof, of at least one chromosome of the organism. It should be noted that the said nucleic acid sequence may be integrated into one of the gender-chromosomes of said transgenic heterogametic organism; and (ii) at least one nucleic acid sequence encoding at least one guide RNA directed against at least one gene or any product/s thereof encoding a product determining or a product essential for an undesired trait. It should be noted that in certain embodiments, these nucleic acid sequence may be integrated into the other gender-chromosome of the transgenic heterogametic organism.

In the next step (b), providing a transgenic eukaryotic homogametic organism comprising at least one nucleic acid sequence encoding at least one RNA guided DNA binding protein nucleic acids modifier protein (e.g., nuclease), or any fragments, domains, or subunits thereof or any non-active variant or mutant thereof and any fusion protein comprising the same. It should be noted that in certain embodiments, the nucleic acid sequence may be integrated into at least one allele of any chromosome of said transgenic homogametic organism.

The next step (c), involves breeding the transgenic heterogametic organism provided in step (a) with the transgenic homogametic organism provided in step (b), thereby obtaining a progeny predominantly composed of said one desired gender having at least one modified undesired trait. In a further aspect, the invention provides a method for reducing the population of an eukaryotic species. In some embodiments the method may comprise the steps of:

In a first step (a), providing a transgenic heterogametic organism of said species comprising:

(i) least one nucleic acid sequence encoding at least one guide RNA directed against at least one target sequence within at least one of coding and non-coding sequences or any product/s thereof of at least one chromosome of said organism. It should be noted that such nucleic acid sequence may be integrated into one of the gender-chromosomes of the transgenic heterogametic organism; and (ii) at least one nucleic acid sequence encoding at least one guide RNA directed against at least one gene encoding a product essential for fertility. In some embodiments, the nucleic acid sequence may be integrated into the other gender-chromosomes of the transgenic heterogametic organism.

In the next step (b), providing a transgenic eukaryotic homogametic organism comprising at least one nucleic acid sequence encoding at least one RNA guided DNA binding protein nucleic acids modifier protein (e.g., nuclease). It should be appreciated that the nucleic acid sequence may be integrated into at least one allele of any chromosome of the transgenic homogametic organism. The next step (c), involves breeding the transgenic heterogametic organism provided in step (a) with the transgenic homogametic organism provided in step (b), thereby obtaining sterile progenies predominantly composed of the one desired gender; and

The next step (d), involves releasing the sterile progeny obtained in step (c) into the wild, thereby reducing the population of said species.

The invention further provides in other aspects thereof the transgenic eukaryotic homogametic organisms and heterogametic organisms as defined by the invention. The invention further provides any progeny of the transgenic heterogametic and/or homogametic organisms discussed herein, any cells thereof or any component or product thereof, as well as any uses of said transgenic organisms, progeny, cell, component or products thereof. These and other aspects of the invention will become apparent by the hand of the following figures.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1A-1B. Schematic illustration of the expected progeny of two different mice breeding

FIG. 1A represents the breeding of female WT mice (unmodified wild type mice) (light grey) with male from the Y-line males (dark gray) (encoding self-destructing sgRNAs on the Y chromosome) providing a non-affected progeny.

FIG. 1B shows the breeding of Cas9-line females (light gray) (encoding the Cas9 nuclease) with the Y-line males (dark gray) resulting in lethality of all males. Specifically, the crossed mice are the males carrying both Cas9 and Y-encoded gRNAs, and are expected to be inviable (because of the presence of both a functional Cas9 nuclease and self-destructing sgRNAs on the Y chromosome). Numbers at the bottom indicate the number of mice from each cross surviving at least 7 days. * P value of 0.09 indicating that the results are statistically insignificant from the expected 1:1 ratio between males and females. ** P<0.001 indicating that the results are statistically significant from the expected 1:1 ratio. Comparisons were performed using Binomial test (two-tailed, assuming normal 50:50% distribution).

FIG. 2. Cloning strategy of the gRNAs: the KI sequence

The figure presents the sequence used for knocking in (KI) the spacers encoding the gRNAs into the Uty gene located at mouse chromosome Y. The U6 promoter (a RNA polymerase III promoter) is underlined and bold (as denoted by SEQ ID NO: 5, the specific guides are appearing in bold capitals (i.e. gRNA1, gRNA2 and gRNA3 as denoted by SEQ ID NO: 1, SEQ ID NO: 2 and SEQ ID NO: 3 respectively) and the sgRNA core is underlined (as denoted by SEQ ID NO: 4). The remaining sequences are the vector backbone and connecting sequences.

FIG. 3A-3B. Schematic representation of the targeting strategy of the KI sequence into the Y chromosome of mice

FIG. 3A. Linearized targeting vector, showing restriction enzyme sites.

FIG. 3B. The targeted gene is the Uty gene located on mouse chromosome Y that has 27 exons. The KI sequence is inserted into intron 2 in the reverse orientation. The targeting vector also contains a Neo (neomycin) cassette flanked by loxP sites. DTA (diphtheria toxin A) is used for negative selection.

FIG. 4A-4C. Pictures of sporadic males exhibiting late onset of lethal phenotype.

FIG. 4A-4B. show pictures of the deformed male born from a cross between the Y-line males and the Cas9-line females. This male was either born dead or died soon after birth.

FIG. 4C. shows a picture of another male that was born smaller and paler (arrowed) from a cross between the Y-line males and the Cas9-line females. This male survived less than 36 hours.

### DETAILED DESCRIPTION OF THE INVENTION

Genetically stable transgenic animals and plants enabling safe and non-invasive selection of a desired gender of an eukaryotic organism are of great significance in farm industry, aquaculture, agriculture as well as in research.

The ability to predetermine the sex of livestock is economically beneficial and significantly increases the welfare and proper use of animals. In the poultry industry, for example, almost all males are brutally and unnecessarily killed shortly after hatching. The labor and associated costs of separation of females from males, as well as the massive killing of males, could be eliminated by using an effective sex-determination system. The present invention provides a proof of concept for a sex determination system in an eukaryotic organism (i.e., mice) by crossing two genetically engineered lines. In the exemplified non-limiting embodiments of the invention, the maternal line encodes a functional Cas9 protein, whereas the paternal line encodes guide RNAs on the Y chromosome that target vital mouse genes. After fertilization, the presence of both the Y-encoded guide RNAs from the paternal sperm and the Cas9 protein from the maternal egg target these vital genes in males. The present invention clearly shows that this breeding consequently self-destructs solely the males, but not the females. These results pave the way for sex determination of livestock, thus saving labor, costs, and eliminating significant animal suffering.

Therefore, in a first aspect, the invention relates to a system comprising at least one heterogametic transgenic organism (A) and at least one homogametic transgenic organism (B). More specifically, in some embodiments, the systems provided by the invention may comprise:

(A), a transgenic eukaryotic heterogametic organism comprising one of:

In one option (a), the heterogametic organism of the invention may comprise at least one nucleic acid sequence:

In some embodiments, such nucleic acid sequence may comprise (i) a sequence encoding or forming at least one target recognition element for at least one nucleic acids modifier protein; or

In other embodiments (ii), such sequence may comprise a sequence encoding or forming the at least one target recognition element, and a nucleic acid sequence encoding a first fragment, domain or subunit of at least one of said nucleic acids modifier protein.

In yet another option (b), the heterogametic organism of the system of the invention may comprise at least one nucleic acid sequence encoding:

In some embodiments (i), at least one nucleic acids modifier protein; or alternatively, in some other embodiments (ii), a second fragment, domain or subunit of such at least one nucleic acids modifier protein.

It should be noted that in some embodiments the nucleic acids modifier protein may be active only in the presence of the first and second fragments, domains or subunits thereof.

In some embodiments, “the nucleic acid modifier” protein may be any protein or polypeptide that upon direct or indirect interaction with a nucleic acid sequence modify or modulate the structure or function thereof. Such modification may include the modification of at least one functional group, addition or deletion of at least one chemical group by modifying an existing functional group or introducing a new one such as methyl. The modifications may include cleavage, methylation, demethylation, deamination and the like. Specific modifier proteins applicable in the present invention may include but are not limited to a nuclease, a methyltransferases, a methylated DNA binding factor, a transcription factor, a chromatin remodeling factor, a polymerase, a demethylase, an acetylase, a deacetylase, a kinase, a phosphatase, an integrase, a recombinase, a ligase, a topoisomerase, a girase, a helicase, any combinations thereof or any fusion proteins comprising at least one of the modifier proteins. It should be noted that in some embodiments, the nucleic acids modifier protein may be at least one active or non-active nuclease or any fusion protein thereof.

As will be elaborated herein below, “activity” of the nucleic acids modifier protein referred to herein may relate in some embodiments to any modification performed in any nucleic acid molecule or sequence, for example, any sequence encoding a product, or alternatively any non-coding sequences. Such modification in some embodiments may result (specifically in case performed on a coding sequence), in modulation of the expression, stability or activity of the encoded product. Non-limiting examples for such modification may be nucleolytic distraction, methylation, demethylation, acetylation and the like. In some specific embodiments, such nucleic acid modifier protein may be a nuclease, and the activity referred to herein may be the nucleolytic activity of the nuclease. However, in some alternative embodiments, the invention further encompasses other activities that do not relate to nucleolytic activity. More specifically, as the invention may further encompass the use of an inactive nuclease or any fusion proteins thereof, “activity” may refer to any additional non-nucleolytic activity that may in some embodiments include repression or alternatively, activation of gene expression. More specifically, in case a non-active nuclease is used as part of a fusion protein, such modulation of gene expression may be achieved by including proteins having methylation or de-methylation activity in such fusion protein or alternatively, by recruiting either transcription factors or transcription suppressors to the non-active nuclease. In more specific embodiments, demethylation and/or recruitment of transcription factors may increase the expression of the encoded product, whereas methylation or recruitment of transcription repressors may inhibit or reduce the expression of the encode product.

Still further, in some embodiments, the nucleic acid sequence of (a) or (b) comprised within the heterogametic transgenic organism may be integrated into one of the gender-chromosomes of the transgenic heterogametic organism.

As noted above, the system of the invention may comprise as the homogametic organism:

(B), a transgenic eukaryotic homogametic organism comprising one of:

In one option (a), at least one nucleic acid sequence encoding:

In some embodiments (i), at least one nucleic acids modifier protein; or alternatively, (ii), a second fragment, domain or subunit of the at least one nucleic acids modifier protein.

In yet another option (b), the homogametic organism may comprise at least one nucleic acid sequence that may comprise in some embodiments (i) a sequence encoding or forming at least one target recognition element for at least one nucleic acids modifier protein. In yet some alternative embodiments, such sequence may comprise (ii), a sequence encoding or forming said at least one target recognition element; and in addition, at least one a nucleic acid sequence encoding a first fragment, domain or subunit of at least one said nucleic acids modifier protein.

It should be noted that in some embodiments, the nucleic acids modifier protein may be active only in the presence of the first and second fragments or subunits thereof. As indicated above, in some embodiments, the nucleic acids modifier protein may be any of the proteins disclosed above. In yet some further embodiments, the nucleic acids modifier protein may be at least one nuclease. In this connection, an “active” nuclease refer in some embodiments to the nucleolytic activity of the nuclease. However, in cases when an inactive nuclease is used or any fusion proteins thereof, “activity” may refer to other functions of the fusion protein that do not relate to nucleolytic activity. Still further, in some embodiments, the nucleic acid sequence may be integrated into at least one allele of any chromosome or into mitochondrial DNA of the transgenic homogametic organism. As noted above, the invention provides a system comprising a heterogametic and a homogametic transgenic organisms. A “transgenic organism” generally refers to an organism that encodes a heterologous DNA sequence, or one or more additional DNA sequences that are not normally endogenous to the organism (collectively referred to herein as “transgenes”) chromosomally integrated into the germ cells of the organism. As a result of such transfer and integration, the transferred sequence may be transmitted through germ cells to the offspring of a transgenic organism. The transgenic organism (including its progeny) also have the transgene integrated into the gender chromosomes of somatic cells. Germ cells are embryonic cells that undergo meiosis, followed by cellular differentiation into a mature gamete. A gamete is a haploid cell that fuses with another haploid cell during fertilization (conception) in organisms that sexually reproduce. In species that produce two morphologically distinct types of gametes, and in which each individual produces only one type, a female is any individual that produces the larger type of gamete, called an ovum (or egg), and a male produces the smaller tadpole-like type, called a sperm. In such organisms, the gender i.e. male or female is dictated by a specific sex-determination system.

A sex-determination system is a biological system that determines the development of sexual characteristics in an organism. The two main sex-determination systems in eukaryotic species are the XY sex-determination system and the ZW sex-determination system (e.g., XY or ZW).

The term “gender-chromosome” or “sex chromosome”, refers to a chromosome that when paired with another gender chromosome, determine the gender/sex of an organism (e.g., XX, XY or ZZ, WZ). In some organisms (insects such as flies), the number of a specific gender chromosome may also determine the gender of the organism. More specifically, in such cases one gender (e.g., females) have at least one more gender chromosome than the other gender (e.g., males). For example, one gender carry two X chromosomes (XX) and the other gender carry only one (XO) gender chromosome. Thus, it should be understood that the invention further encompasses such options for gender determination.

The systems of the invention comprise at least one transgenic homogametic organism and at least one transgenic heterogametic organism.

The term “heterogametic” refers to the sex/gender of a species in which the gender-chromosomes are not the same. For example, an organism containing the X and Y gender chromosomes, or alternatively, the Z and W chromosomes. As indicated above, heterogametic organism may also carry only one gender chromosome, or at least one less gender chromosome as compared to the other gender (e.g., XO vs. XX).

The term “homogametic” refers to the sex/gender of a species in which the gender-chromosomes are the same. More specifically, an organism having at least two copies of one sex chromosome, for example, two copies of the X chromosome, or two copies of the Z chromosome. Similarly, in some embodiments, the homogametic gender may carry at least two copies of one gender chromosome (e.g., XX vs., XO).

Thus, a heterogametic organism is an organism having two different sex chromosomes (W and Z or X and Y), or alternatively, only one copy of one gender chromosome) and a homogametic organism carry at least two copies of the same sex chromosome.

In the XY sex-determination system, the male is the heterogametic organism and the gender-chromosome specific for the heterogametic gender is the Y chromosome while the gender-chromosome specific for the homogametic gender is the X chromosome. Therefore, in the XY sex-determination system concerning the gender-chromosomes of the male (heterogametic organism), the gender-chromosome determining for a male progeny is the Y chromosome and the gender-chromosome determining for a female progeny is the X chromosome.

In the WZ sex-determination system, the female is the heterogametic organism and the gender-chromosome specific for the heterogametic gender is the W chromosome while the gender-chromosome specific for the homogametic gender is the Z chromosome. Therefore, in the WZ sex-determination system concerning the gender-chromosomes of the female (heterogametic organism), the gender-chromosome determining for a male progeny is the Z chromosome and the gender-chromosome determining for a female progeny is the W chromosome.

As indicated above, in the systems of the invention, nucleic acid sequences encoding at least one nucleic acids-modifier protein may be incorporated into the homogametic or heterogametic transgenic organism of the invention. In some embodiments, nucleic acids-modifier protein may include any protein or protein complex that modifies an encoding or non-encoding nucleic acid sequence. In yet some further embodiments, the modification caused by said modifier protein may modulate the expression of a protein product encoded or alternatively, controlled by the target nucleic acid sequence. Alternatively, the modifier may modulate the stability or the activity of such product. Such modification include nucleolytic cleavage (e.g., by a nuclease), methylation, demethylation, (of either coding or non-coding sequences, such as control elements) activation or repression of protein expression and the like. Thus, in some embodiments, the nucleic acid modifier may be at least one of a nuclease, methylase, demethylase, transcription factor, transcription repressor, any fusion proteins thereof, and any complex comprising at least one of said modifier and any combinations thereof.

In some embodiments, the modifier protein of the invention may be at least one nuclease. In the systems of the invention and as detailed above, at least one nuclease (either partly or entirely, either active or inactive, either as a fragment, a mutant and/or any fusion protein thereof) may be incorporated either into the heterogametic or the homogametic organism. It should be appreciated that the term “nuclease” as used herein relates in some embodiments to an active nuclease having a nucleolytic activity. However, it should be appreciated that in some embodiments, the term “nuclease” as used herein further encompasses a molecule having the structural features of a nuclease but display reduced, defective or no nucleolytic activity on any nucleic acid molecule, specifically, DNA or RNA (referred to herein as an inactive nuclease). The inactive-nuclease as used herein further encompasses any fragment, mutant or fusion protein of an inactive nuclease. More specifically, a fusion protein of an inactive nuclease with any other protein e.g., transcription factor or repressor, methylase, demethylase, as will be elaborated herein after.

Thus, in some specific embodiments, the nuclease encoded by the transgenic heterogametic or homogametic organisms of the systems of the invention may be at least one of: (i) a nuclease having a nucleolytic activity; (ii) a non-active nuclease and/or a fusion protein thereof, or alternatively (iii) any fragment, domain or subunit of the nuclease of (i) or the inactive nuclease of (ii) or of any fusion protein thereof.

More specifically, as used herein, the term “nuclease” is an enzyme that in some embodiments display a nucleolytic activity, specifically, capable of cleaving the phosphodiester bonds between monomers of nucleic acids (e.g., DNA and/or RNA). Nucleases variously effect single and double stranded breaks in their target molecules. There are two primary classifications based on the locus of activity. Exonucleases digest nucleic acids from the ends. Endonucleases act on regions in the middle of target molecules. They are further subcategorized as deoxyribonucleases and ribonucleases. The former acts on DNA, the latter on RNA. A nuclease must associate with a nucleic acid before it can cleave the molecule, providing a degree of recognition. The nucleases belong just like phosphodiesterase, lipase and phosphatase to the esterases, a subgroup of the hydrolases. This subgroup includes the Exonucleases are enzymes that work by cleaving nucleotides one at a time from the end (exo) of a polynucleotide chain. A hydrolyzing reaction that breaks phosphodiester bonds at either the 3′ or the 5′ end occurs. Eukaryotes and prokaryotes have three types of exonucleases involved in the normal turnover of mRNA: 5′ to 3′ exonuclease (Xrnl), which is a dependent decapping protein; 3′ to 5′ exonuclease, an independent protein; and poly(A)-specific 3′ to 5′ exonuclease. Members of this family include Exodeoxyribonucleases producing 5′-phosphomonoesters, Exoribonucleases producing 5′-phosphomonoesters, Exoribonucleases producing 3′-phosphomonoesters and Exonucleases active with either ribo- or deoxy-. Members of this family include exonuclease, II, III, IV, V, VI, VII, and VIII. As noted above, Endonucleases are enzymes that cleave the phosphodiester bond within a polynucleotide chain. Some endonucleases, such as deoxyribonuclease I, cut DNA relatively nonspecifically (without regard to sequence), while many, typically called restriction endonucleases or restriction enzymes, cleave only at very specific nucleotide sequences.

In some embodiment, the nuclease may be an active enzyme having a nucleolytic activity as specified above. In some alternative embodiments, the nuclease may be a defective enzyme. A defective enzyme (e.g., a defective mutant, variant or fragment) may relate to an enzyme that display an activity reduced in about 1%, 2%, 3%, 4%, 5% to about 100%, specifically, about 5% to about 10%, about 10% to about 15%, about 15% to about 20%, about 20% to about 25%, about 25% to about 30%, about 35% to about 40%, about 40% to about 45%, about 45% to about 50%, about 50% to about 55%, about 55% to about 60%, about 65% to about 70%, about 75% to about 80%, about 80% to about 85%, about 85% to about 90%, about 90% to about 95%, about 95% to about 99.9%, more specifically, reduced activity of about 98% to about 100% as compared to the active nuclease. In yet some further embodiments, the system of the invention may comprise one organism (either the heterogametic or the homogametic transgenic organism) that comprise at least one nucleic acid sequence encoding, or alternatively, forming a target recognition element. As used herein a “target recognition element” is a nucleic acid sequence (either RNA or DNA) that will direct the nucleic acid-modifier protein, for example, the nuclease to a specific target position within a nucleic acid sequence. The recognition of the target by the target recognition element is facilitated in some embodiments by base-pairing interactions. These target recognition elements are specifically relevant in case of guided nucleases. In yet some further alternative embodiments, the target recognition element itself may be a sequence within the target site that is recognized by the nuclease (e.g., a restriction site). In some embodiments, for nucleases displaying a nucleolytic activity, directing the nuclease to a specific site may result in cleaving the phosphodiester bonds between monomers of nucleic acids (e.g., DNA and/or RNA) that may lead in some embodiments to specific destruction thereof. In yet some alternative embodiments, where a non-active nuclease is used, and specifically, a fusion protein thereof, directing such defective nuclease to, or alternatively, by a target recognition element, may result in targeted modulation (e.g., activation or repression, methylation or demethylation) of the target nucleic acid sequence that comprises, or is targeted by the target recognition element. It should be noted that a target recognition element may comprise between about 10 nucleotides to 70 nucleotides or more. In yet some further embodiments, the target recognition element may comprise a number of nucleotides as specified for the spacers herein after. In certain embodiments, the nuclease encoded by the transgenic heterogametic or homogametic organism of the systems of the invention may be either a guided or a non-guided nuclease.

In some embodiments, the systems of the invention may comprise a transgenic organism (either the heterogametic or the homogametic organisms) that comprise a nucleic acid sequence encoding at least one non-guided nuclease. In some specific embodiments, such non-guided nuclease may be at least one restriction enzyme or any fusion protein thereof. Thus, in some specific and non-limiting embodiments, the nuclease may be at least one restriction enzyme. In yet some further embodiments, the transgenic organisms of the systems of the invention may comprise at least one nucleic acid sequence forming a target recognition element. Such target recognition element may be in some embodiments, a restriction site of the restriction enzyme. Such target recognition element, specifically, at least one restriction site, may be incorporated within at least one of coding and non-coding sequences of at least one chromosomal or mitochondrial DNA of the organism. In some embodiments, such organism is any organism of the same species of the transgenic organism, and in more specific embodiments, such organism is the embryo of the transgenic organism of the invention.

A restriction enzyme is an endonuclease that cleaves DNA into fragments at or near its specific recognition sites within the molecule. To cut DNA, most restriction enzymes make two incisions, through each sugar-phosphate backbone (i.e. each strand) of the DNA double helix. Restriction enzymes with long recognition sites (recognition site of at least 10 nucleotides) may be in some embodiments, suitable nuclease for the system of the invention (mega nucleases/homing endonucleases). Homing endonucleases constitute a family of very rare-cutting endonucleases. They have recognition sequences that span 12-40 bp of DNA, whereas “classical” restriction enzymes recognize much shorter stretches of DNA, in the 3-8 bp range (up to 12 bp for rare-cutter). In such embodiments, the restriction site may be incorporated (e.g., as a target recognition element) either into the gender chromosome of the heterogametic transgenic organism, or alternatively, into a chromosomal or mitochondrial DNA of the homogametic transgenic organism of the system of the invention. Non-limiting examples of such restriction enzymes may include, but are not limited to I-Sce I, I-Chu I, I-Dmo I, I-Cre I, I-Csm I, Pl-Sce I, PI-Tli I, PI-Mtu I, I-Ceu I, I-Sce II, I-Sce III, HO, Pi-Civ I, Pl-Ctr I, PI-Aae I, PI-Bsu I, PI-Dha I, PI-Dra I, PI-Mav I, PI-Mch I, PI-Mfu I, PI-Mfl I, PI-Mga I, PI-Mgo I, PI-Min I, PI-Mka I, PI-Mle I, PI-Mma I, PI-Msh I, PI-Msm I, PI-Mth I, PI-Mtu I, PI-Mxe I, PI-Npu I, Pl-Pfu I, PI-Rma I, Pl-Spb I, PI-Ssp I, PI-Fac I. In yet some further embodiments “nucleases” as referred to herein, also relates to nucleases that cut ribonucleic acids, specifically, RNA molecules. In some specific embodiments, PNAzymes that specifically cut RNAs or any artificial restriction systems such as argonautes with guides may serve as non-limiting examples for such nucleases. More specifically, in some embodiments, Argonaute protein taken from Pyrococcus furiosus (PfAgo) along with guide DNA, may be used as artificial restriction enzyme. A PNA-based system, called PNAzymes, has a Cu(II)-2,9-dimethylphenanthroline group that mimics ribonucleases for specific RNA sequence and cleaves at a non-base-paired region (RNA bulge) of the targeted RNA formed when the enzyme binds the RNA. This enzyme shows selectivity by cleaving only at one site that either does not have a mismatch or is kinetically preferred out of two possible cleavage sites. As indicated above, in some further embodiments, the nuclease used by the systems of the invention may be either a guided or non-guided nuclease. A guided nuclease is according to some embodiments a nuclease targeted to its specific target site by a nucleic acid sequence that specifically interacts with the target site by base-pairing interactions between nucleotides of the guide nucleic acid sequence and the nucleotide sequence of the target site. A non-guided nuclease is a nuclease that achieves sequence specificity without the use of guiding nucleic acid sequences, for example, using protein-nucleic acid sequence interactions. In more specific embodiments, the non-guided nuclease may be a classical restriction enzyme (e.g., having a restriction site of up to 10 bp), or any derivative or fusion protein thereof, and the guided nuclease may be a TALEN or a ZFN. In such embodiments, the target recognition element may be an element endogenously comprised within the chromosomal or mitochondrial DNA of both, the heterogametic and the homogametic organisms of the invention. In such particular embodiments, the nuclease (or any inactive mutants and/or any fusion protein thereof), is split between the homogametic and heterogametic organisms of the systems of the invention (e.g., one of the organisms may comprise nucleic acid sequence encoding a first fragment, subunit or domain of such guided nuclease and the other organism may comprise nucleic acid sequence encoding a second fragment, subunit or domain of such guided nuclease).

More specifically, as the target recognition element exists in both transgenic organisms, the heterogametic organism for example, may comprise nucleic acid sequence encoding a first fragment, subunit or domain of such guided nuclease and the homogametic organism may comprise nucleic acid sequence encoding a second fragment, subunit or domain of such guided nuclease. In the presence of both, the nuclease displays the required activity (either nucleolytic activity or any other activity as discussed above).

In some other alternative embodiments, for example in case of TALEN, ZFN or a restriction enzyme having a restriction site comprising 10 nucleotides or more, or homing endonucleases such as I-SceI with longer restriction sites (18 bp), the target recognition element may be inserted into the heterogametic or homogametic organisms of the systems of the invention.

In some embodiments, the nuclease used by the systems of the invention may be a guided nuclease. In yet some specific embodiments, the nuclease may be at least one Transcription activator-like effector nucleases (TALEN). TALEN are restriction enzymes that can be engineered to cut specific sequences of DNA. They are made by fusing a TAL effector DNA-binding domain to a DNA cleavage domain of a nuclease. More specifically, TALENs are artificial endonucleases designed by fusing the DNA-binding domain (multiples of nearly identical repeats each comprised of ˜34 amino acids) obtained from TAL (transcription activator-like) effector (TALE) protein to the cleavage domain of the FokI endonuclease. Each TALE repeat independently recognizes its corresponding nucleotide (nt) base with two variable residues [termed the repeat variable di-residues (RVDs)] such that the repeats linearly represent the nucleotide sequence of the binding site.

In such case, in some particular and non-limiting embodiments, at least one fragment, subunit or domain thereof may be TALE DNA-binding domain thereof that may be a first domain and a DNA cleavage domain, that may form a second domain thereof. Together, these first and second domains form a functional TALEN. In yet some further embodiments, where TALEN is used in the systems of the invention as a nuclease, the target recognition element may be a TALEN recognition sequence within at least one of coding and non-coding sequences of at least one chromosomal or mitochondrial DNA of said organism.

In yet some further alternative embodiments, the guided nuclease that may be used by the systems of the invention may be at least one Zinc-finger nucleases (ZFNs).

ZFNs are artificial restriction enzymes generated by fusing a zinc finger DNA-binding domain to a DNA-cleavage domain. Zinc finger domains can be engineered to target specific desired DNA sequences and this enables zinc-finger nucleases to target unique sequences within complex genomes. More specifically, the ZFNs are artificial endonucleases that have been generated by combining a small zinc finger (ZF; about 30 amino acids) DNA-binding/recognition domain (Cys₂His₂) to a type IIS nonspecific DNA-cleavage domain from the FokI restriction enzyme. However, the cleavage activity of the FokI endonuclease demands dimerization. As a ZF module recognizes a 3 bp sequence, there is a requirement for multiple fingers in each ZFN monomer for recognizing and binding to longer DNA target sequences.

In some particular and non-limiting embodiments, the at least one fragment, subunit or domain thereof may be any one of the DNA-binding domain and DNA-cleavage domain of said ZFN. In some specific embodiments, together, these first and second domains form a functional ZFN. In yet some further embodiment, where ZFN is used in the systems of the invention as a nuclease, the target recognition element may be a ZFN recognition sequence within at least one of coding and non-coding sequences of at least one chromosomal or mitochondrial DNA of said organism. In yet some further alternative embodiments, the artificial zinc-finger protein (AZP)-staphylococcal nuclease (SNase) hybrid (AZP-SNase) may be also used in the same manner. Still further, it should be noted that in some embodiments, any nuclease (e.g., FokI) or any chimera or fusion protein thereof may be used by the systems and transgenic organisms of the invention. In yet some further embodiments, as will be elaborated in more detail herein after, the guided nuclease used by the systems of the invention may be an RNA guided nuclease

Still further, the transgenic heterogametic or homogametic organisms of the systems of the invention may comprise nucleic acid sequence encoding or forming a target recognition element. In some embodiments, wherein said that the nucleic acid sequence of the organism form the target recognition element, it means that the transgenic organism comprise a particular sequence recognized by the nuclease. Alternatively, the nucleic acid sequence inserted to the transgenic organism of the invention encodes a target recognition element, that is an element that facilitates recognition of a desired target sequence by the nuclease, for example, by guiding the nuclease to the specific target site via base pairing interactions. It should be noted that the target recognition element as well as the target sequence itself, may be within a specific gene but also could be a repetitive coding or non-coding region. It should be noted however that a “target sequence” for a specific nuclease employed by the invention may be within a DNA sequence as discussed above but however, may be in some embodiments within any product of such DNA, e.g., an RNA sequence. Such target sequence may be relevant for nucleases (either active or non-active) that cleave RNA.

“Coding region” as used herein refer to nucleic acid sequences, specifically, DNA that are transcribed to RNA and translated into a protein product. “Non-coding region” as used herein, refers to components of an organism's DNA that do not encode protein sequences. Some noncoding DNA region is transcribed into functional non-coding RNA molecules, other functions of noncoding DNA regions include the transcriptional and translational regulation of protein-coding sequences, scaffold attachment regions, origins of DNA replication, centromeres and telomeres. The hypothesized non-functional portion (or DNA of unknown function) has often been referred to as “junk DNA”.

In more specific embodiments, the at least one target recognition element of the system of the invention may be at least one ribonucleic acid guide, specifically, guide RNA directed against at least one target sequence within at least one of coding and non-coding sequences of at least one chromosomal or mitochondrial DNA of the organism. Alternatively, such targeting sequence may be any sequence within a product of said coding or non-coding sequences, for example, RNA molecules.

In yet some further embodiments, the guided nuclease may be at least one RNA guided DNA binding protein nuclease. As used herein, an RNA guided DNA binding protein nuclease is a nuclease which is guided to its cleavage site (or alternatively, a site for any other alternative activity), by an RNA molecule. This RNA molecule is referred as a guide RNA, or gRNA.

In some further embodiments, the systems of the invention may comprise a transgenic heterogametic organism and a transgenic homogametic organism. More specifically:

(A) a transgenic eukaryotic heterogametic organism comprising one of:

In some optional embodiments (a), at least one nucleic acid sequence encoding: (i) at least one guide RNA directed against at least one target sequence within at least one of coding and non-coding sequences or any product/s thereof of at least one chromosome of the organism; or alternatively, (ii) at least one said guide RNA and a nucleic acid sequence encoding a first fragment, domain or subunit of at least one RNA guided DNA binding protein nuclease.

In yet some other optional embodiments, the transgenic heterogametic organism (A) may comprise (b), at least one nucleic acid sequence encoding: (i) at least one RNA guided DNA binding protein nuclease; or alternatively, (ii), a second fragment, domain or subunit of at least one RNA guided DNA binding protein nuclease, or any non-active mutant thereof or any fusion protein comprising said non-active nuclease.

It should be noted that in case a domain, fragment or subunit of the nuclease (either an active or a non-active nuclease) is used, the RNA guided DNA binding protein nuclease may be active only in the presence of the first and second fragments or subunits thereof. It should be appreciated that “activity” as referred to herein relates to activity as defined herein before.

In yet some further embodiments, the nucleic acid sequence may be integrated into one of the gender-chromosomes of the transgenic heterogametic organism of (A).

The system of the invention further comprises a homogametic transgenic organism. More specifically, (B) a transgenic eukaryotic homogametic organism comprising one of:

In one option (a), the transgenic homogametic organism of (B) may comprise at least one nucleic acid sequence encoding: in some embodiments (i), at least one RNA guided DNA binding protein nuclease; or alternatively, in (ii), a second fragment, domain or subunit of at least one RNA guided DNA binding protein nuclease.

In yet some other optional embodiments, the transgenic organism of (B) may comprise (b) at least one nucleic acid sequence encoding: either (i), at least one guide RNA directed against at least one target sequence within at least one of coding and non-coding sequences or any product/s thereof of at least one chromosome of said organism; or alternatively, (ii), at least one said guide RNA and a nucleic acid sequence encoding a first fragment, domain or subunit of at least one RNA guided DNA binding protein nuclease.

In case a fragment, domain or subunits of the nuclease are used, it should be noted that the RNA guided DNA binding protein nuclease may be active only in the presence of the first and second fragments or subunits thereof (activity refers to herein to a nucleolytic activity or a non-nucleolytic activity as will be elaborated herein after).

Still further, it should be noted that the nucleic acid sequence of (a) or (b) may be integrated into at least one allele of any chromosome or into mitochondrial DNA of the transgenic homogametic organism of the system of the invention as specified in (B).

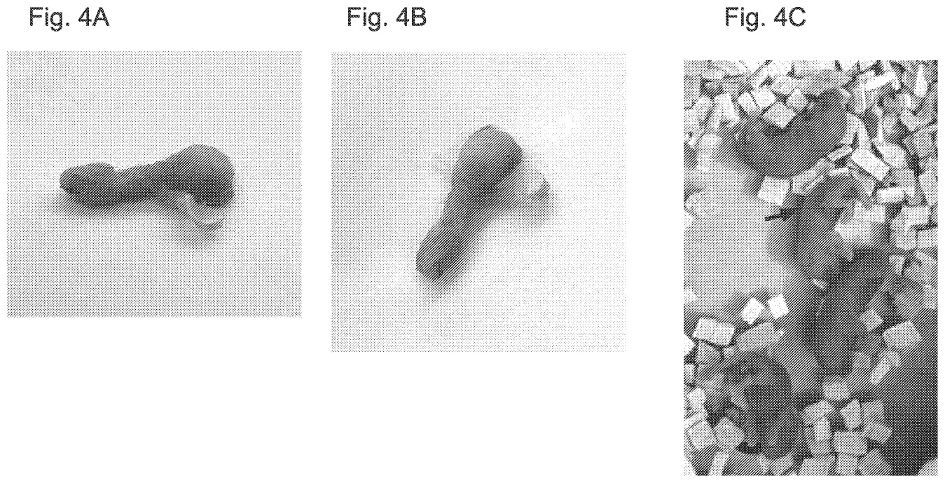

It should be noted that the phrase “target sequence within at least one of coding and non-coding sequences or any product/s thereof of at least one chromosome of the organism”, is meant in some embodiments, that an organism of the same species of the transgenic organism carry the sequence. In yet some further embodiments, the guide RNA will target a target sequence within the coding and non-coding sequences or any product/s thereof of at least one chromosome or non-chromosomal sequences of an embryo, or vital or non-vital progeny of the transgenic organism discussed herein. Embryo as used herein refers to embryo of any embryonic stage, including the fertilized gamete or zygote of the transgenic organism of the invention. Still further, it must be understood that also born progenies, either vital or non-vital (as shown by the following examples, specifically, FIG. 4), are also encompassed herein.

In more specific embodiments of the systems of the invention, when the heterogametic organism of (A) of the system of the invention comprises at least one nucleic acid sequence encoding at least one of the guide RNA; or at least one said guide RNA and a nucleic acid sequence encoding a first fragment, domain or subunit of at least one RNA guided DNA binding protein nuclease or any non-active mutant or any fusion protein thereof, then the homogametic organism of (B) comprises at least one nucleic acid sequence encoding at least one RNA guided DNA binding protein nuclease or any non-active mutant or any fusion protein thereof; or a second fragment, domain or subunit of at least one RNA guided DNA binding protein nuclease or any non-active mutant or any fusion protein thereof.

In yet some further alternative embodiments, when the heterogametic organism of (A) comprises at least one nucleic acid sequence encoding at least one RNA guided DNA binding protein nuclease or any non-active mutant or any fusion protein thereof, or a alternatively, second fragment, domain or subunit of at least one RNA guided DNA binding protein nuclease, then the homogametic organism of (B) comprises at least one nucleic acid sequence encoding at least one of said guide RNA; or alternatively, at least one said guide RNA and a nucleic acid sequence encoding a first fragment, domain or subunit of at least one RNA guided DNA binding protein nuclease or any non-active mutant or any fusion protein thereof.

In some embodiments, the target sequence targeted by the targeting element of the invention (e.g., gRNA), may be within the coding or non-coding regions of at least one of autosomal or gender-chromosomes and in mitochondrial DNA of the organism. As noted above, in some embodiments, the target sequence within at least one of coding and non-coding sequences or any product/s thereof of at least one chromosome of the organism, is meant that an organism of the same species of the transgenic organism carry the sequence, and specifically, an embryo or progeny of the transgenic organism.

In yet some further embodiments, the target sequence may be only within at least one autosomal chromosome of the organism. In yet some further embodiments, the target sequence may be within a DNA molecule or any product/s thereof, for example, an RNA molecule. Still further, in some embodiments, target sequences may be any nucleic acid sequence essential for survival or embryonic development of the organism. Essential genes are those genes of an organism that are critical for its development and survival. These essential genes encode proteins to maintain a central metabolism, replicate DNA, translate genes into proteins, maintain a basic cellular structure, mediate transport processes into and out of the cell, and the core machineries of all eukaryotic cells, the ribosome, RNA polymerase, and central metabolic enzymes, translation, transcription, and metabolism. The essential genes tend to be highly expressed; involved in fundamental biological processes, including DNA replication, RNA transcription, and translation of messenger RNA. It should be noted that essential genes according to the invention may be any gene that involved in cell cycle, and cell cycle checkpoints.

In other specific embodiments, the system of the invention may comprise at least one heterogametic organism and at least one homogametic transgenic organisms:

More specifically (a), a transgenic eukaryotic heterogametic organism comprising at least one nucleic acid sequence encoding at least one guide RNA directed against at least one target sequence within at least one of coding and non-coding sequence or any product/s thereof, of at least one chromosome of said organism. It should be noted that the nucleic acid sequence may be integrated into one of the gender-chromosomes of the transgenic heterogametic organism.

The system of the invention further comprises (b), a transgenic eukaryotic homogametic organism comprising at least one nucleic acid sequence encoding at least one RNA guided DNA binding protein nuclease. Such nucleic acid sequence may be integrated into at least one allele of any chromosome of the transgenic homogametic organism.

It should be noted that in some embodiments, the transgenic heterogametic organism of (a) and the transgenic homogametic organism of (b) of the system of the invention are of the same species. In some alternative embodiments, the transgenic heterogametic organism of the system of the invention may further comprise a nucleic acid sequence encoding at least one guide RNA directed against at least one gene or any product/s thereof encoding a product determining, or a product essential for, an undesired trait. In such case, the nucleic acid sequence may be integrated into the other gender-chromosomes of the transgenic heterogametic organism. In more specific embodiments, the undesired trait may be related to fertility.

In some more specific embodiments, the RNA guided DNA binding protein nuclease of the system of the invention may be any one of a clustered regularly interspaced short palindromic repeat (CRISPR) Class 2 or Class 1 system. The Clustered Regularly Interspaced Short Palindromic Repeats (CRISPR) system is a bacterial immune system that has been modified for genome engineering. CRISPR-Cas systems fall into two classes. Class 1 systems use a complex of multiple Cas proteins to degrade foreign nucleic acids. Class 2 systems use a single large Cas protein for the same purpose. More specifically, Class 1 may be divided into types I, III, and IV and class 2 may be divided into types II, V, and VI.

As used herein, CRISPR arrays also known as SPIDRs (Spacer Interspersed Direct Repeats) constitute a family of DNA loci that are usually specific to a particular bacterial species. The CRISPR array is a distinct class of interspersed short sequence repeats (SSRs) that were first recognized in E. coli. In subsequent years, similar CRISPR arrays were found in Mycobacterium tuberculosis, Haloferax mediterranei, Methanocaldococcus jannaschii, Thermotoga maritima and other bacteria and archaea. It should be understood that the invention contemplates the use of any of the known CRISPR systems, particularly and of the CRISPR systems disclosed herein. The CRISPR-Cas system has evolved in prokaryotes to protect against phage attack and undesired plasmid replication by targeting foreign DNA or RNA. The CRISPR-Cas system, targets DNA molecules based on short homologous DNA sequences, called spacers that exist between repeats. These spacers guide CRISPR-associated (Cas) proteins to matching (and/or complementary) sequences within the foreign DNA, called proto-spacers, which are subsequently cleaved. The spacers can be rationally designed to target any DNA sequence. Moreover, this recognition element may be designed separately to recognize and target any desired target.

In some specific embodiment, the RNA guided DNA binding protein nuclease of the system of the invention may be a CRISPR Class 2 system. In yet some further particular embodiments, such class 2 system may be a CRISPR type II system. In a more specific embodiment, the RNA guided DNA binding protein nuclease may be CRISPR-associated endonuclease 9 (Cas9) system. The type II CRISPR-Cas systems include the ‘HNH’-type system (Streptococcus-like; also known as the Nmeni subtype, for Neisseria meningitidis serogroup A str. Z2491, or CASS4), in which Cas9, a single, very large protein, seems to be sufficient for generating crRNA and cleaving the target DNA, in addition to the ubiquitous Cas1 and Cas2. Cas9 contains at least two nuclease domains, a RuvC-like nuclease domain near the amino terminus and the HNH (or McrA-like) nuclease domain in the middle of the protein, but the function of these domains remains to be elucidated. However, as the HNH nuclease domain is abundant in restriction enzymes and possesses endonuclease activity responsible for target cleavage. Still further, it should be noted that type II system comprise at least one of cas9, cas1, cas2 csn2, and cas4 genes. It should be appreciated that any type II CRISPR-Cas systems may be applicable in the present invention, specifically, any one of type II-A or B. Thus, in yet some further and alternative embodiments, at least one cas gene used in the methods and systems of the invention may be at least one cas gene of type II CRISPR system (either typeII-A or typeII-B). In more particular embodiments, at least one cas gene of type II CRISPR system used by the methods and systems of the invention may be the cas9 gene. It should be appreciated that such system may further comprise at least one of cas1, cas1, csn2 and cas4 genes. Double-stranded DNA (dsDNA) cleavage by Cas9 is a hallmark of “type II CRISPR-Cas” immune systems. The CRISPR-associated protein Cas9 is an RNA-guided DNA endonuclease that uses RNA:DNA complementarity to a target site (proto-spacer). After recognition between Cas9 and the target sequence double stranded DNA (dsDNA) cleavage occur, creating the double strand brakes (DSBs).

CRISPR type II system as used herein requires the inclusion of two essential components: a “guide” RNA (gRNA) and a non-specific CRISPR-associated endonuclease (Cas9). The gRNA is a short synthetic RNA composed of a “scaffold” sequence necessary for Cas9-binding (also named tracrRNA) and about 20 nucleotide long “spacer” or “targeting” sequence, which defines the genomic target to be modified. Guide RNA (gRNA), as used herein refers to a synthetic fusion of the endogenous tracrRNA with a targeting sequence (also named crRNA), providing both scaffolding/binding ability for Cas9 nuclease and targeting specificity. Also referred to as “single guide RNA” or “sgRNA”.

CRISPR was originally employed to “knock-out” target genes in various cell types and organisms, but modifications to the Cas9 enzyme have extended the application of CRISPR to “knock-in” target genes, selectively activate or repress target genes, purify specific regions of DNA, and even image DNA in live cells using fluorescence microscopy. Furthermore, the ease of generating gRNAs makes CRISPR one of the most scalable genome editing technologies and has been recently utilized for genome-wide screens.

In most of CRISPR systems, the target sequence within the genome to be edited, should be present immediately upstream of a Protospacer Adjacent Motif (PAM). In other systems, such as type III, there is no PAM. In CRISPR systems based on PAM sequence recognition like CRISPR Type II, the PAM is absolutely necessary for target binding and the exact sequence is dependent upon the species of Cas9 (5′ NGG 3′ for Streptococcus pyogenes Cas9). In certain embodiments, Cas9 from S. pyogenes may be used in the methods, transgenic organisms and systems of the invention. Nevertheless, it should be appreciated that any known Cas9 may be applicable. Non-limiting examples for Cas9 useful in the present disclosure include but are not limited to Streptococcus pyogenes (SP), also indicated herein as SpCas9, Staphylococcus aureus (SA), also indicated herein as SaCas9, Neisseria meningitidis (NM), also indicated herein as NmCas9, Streptococcus thermophilus (ST), also indicated herein as StCas9 and Treponema denticola (TD), also indicated herein as TdCas9. In some specific embodiments, the Cas9 of Streptococcus pyogenes M1 GAS, specifically, the Cas9 of protein id: AAK33936.1, may be applicable in the methods and systems of the invention. In some embodiments, the Cas9 protein may be encoded by the nucleic acid sequence as denoted by SEQ ID NO. 11. In further specific embodiments, the Cas9 protein may comprise the amino acid sequence as denoted by SEQ ID NO. 12, or any derivatives, mutants, variants or any fusion proteins thereof. In yet some further embodiments, Cas9 adapted for mammalian use, may be also applicable in the present invention. A non-limiting embodiments for such Cas9 is disclosed by SEQ ID NO. 41 and the encoding nucleic acid sequence of said adapted Cas9 is denoted by SEQ ID NO. 40. In yet some further embodiments, additional variants of Cas9, for example, Cas9-P2a peptide attached to green fluorescent protein (GFP), may be also used (specifically for cloning purpose). Such variant is denoted by amino acid sequence as denoted by SEQ ID NO. 43, and encoded by the nucleic acids sequence as denoted by SEQ ID NO. 42. Once expressed, the Cas9 protein provided by one transgenic organism, and the gRNA provided by a second transgenic organism, form a riboprotein complex through interactions between the gRNA “scaffold” domain and surface-exposed positively-charged grooves on Cas9. Cas9 undergoes a conformational change upon gRNA binding that shifts the molecule from an inactive, non-DNA binding conformation, into an active DNA-binding conformation. Importantly, the “spacer” sequence of the gRNA remains free to interact with target DNA. The Cas9-gRNA complex binds any target genomic sequence with a PAM, but the extent to which the gRNA spacer matches the target DNA determines whether Cas9 will cut, or alternatively, perform any other manipulation in case a fusion protein comprising a catalytically inactive cas9 is used. Once the Cas9-gRNA complex binds a putative DNA target, a “seed” sequence at the 3′ end of the gRNA targeting sequence begins to anneal to the target DNA. If the seed and target DNA sequences match, the gRNA continues to anneal to the target DNA in a 3′ to 5′ direction. Cas9 will only cleave the target if sufficient homology exists between the gRNA spacer and target sequences. Sufficient homology is meant between about 10% to about 99.9% homology or identity between the target site and the gRNA, that is complementary to the complementary strand. Still further, the Cas9 nuclease has two functional endonuclease domains: RuvC and HNH. Cas9 undergoes a second conformational change upon target binding that positions the nuclease domains to cleave opposite strands of the target DNA. The end result of Cas9-mediated DNA cleavage is a double strand break (DSB) within the target DNA that occurs about 3 to 4 nucleotides upstream of the PAM sequence. The resulting DSB may be then repaired by one of two general repair pathways, the efficient but error-prone Non-Homologous End Joining (NHEJ) pathway and the less efficient but high-fidelity Homology Directed Repair (HDR) pathway.

Programmable engineered nucleases (PEN) strategies for genome editing, may be based either on cell activation of the HDR mechanism following specific double stranded DNA cleavage (knock-in system) or on NHEJ mechanism (knock-out system). In some specific embodiments, the targeted genes to be knockout, are repaired through the NHEJ pathway, resulting in most cases in dysfunction of the target genes (deletions/insertions/non-sense mutations etc.). As discussed previously, Cas9 generates double strand breaks (DSBs) through the combined activity of two nuclease domains, RuvC and HNH. The exact amino acid residues within each nuclease domain that are critical for endonuclease activity are known (D10A for HNH and H840A for RuvC in S. pyogenes Cas9) and modified versions of the Cas9 enzyme containing only one active catalytic domain (called “Cas9 nickase”) have been generated. Cas9 nickases still bind DNA based on gRNA specificity, but nickases are only capable of cutting one of the DNA strands, resulting in a “nick”, or single strand break, instead of a DSB. DNA nicks are rapidly repaired by HDR (homology directed repair) using the intact complementary DNA strand as the template. Thus, two nickases targeting opposite strands are required to generate a DSB within the target DNA (often referred to as a “double nick” or “dual nickase” CRISPR system). This requirement dramatically increases target specificity, since it is unlikely that two off-target nicks will be generated within close enough proximity to cause a DSB. It should be therefore understood, that the invention further encompasses the use of the dual nickase approach to create a double nick-induced DSB for increasing specificity and reducing off-target effects, in the systems, methods and transgenic organisms of the invention. Additional examples of increasing specificity is the use of a nuclease such as FokI fused to dCas9 that serves as a linker to the targeting gRNA.

In yet another embodiment, the invention further encompasses the option of providing a pre-crRNA that can be processed to several final gRNA products that may target identical or different targets, or plurality of targets. In yet some more specific embodiments, the crRNA comprised within the gRNA of the invention may be a single-stranded ribonucleic acid (ssRNA) sequence complementary to a target genomic DNA sequence. In some specific embodiments, the target genomic DNA sequence (e.g., gene essential for embryogenesis) may be located immediately upstream of a protospacer adjacent motif (PAM) sequence and further. Specific targets applicable in the present invention will be discussed herein after. As indicated herein, the gRNA transcribed by the transgene of the invention may be complementary, at least in part, to the target genomic DNA. More specifically, the gRNA encoded by the transgene, may comprising sequences identical, at least in part, to the target sequence, that are complementary to the complementary strand. In certain embodiments, “Complementarity” refers to a relationship between two structures each following the lock-and-key principle. In nature complementarity is the base principle of DNA replication and transcription as it is a property shared between two DNA or RNA sequences, such that when they are aligned antiparallel to each other, the nucleotide bases at each position in the sequences will be complementary (e.g., A and T or U, C and G). As indicated above in some particular embodiments, the genomic DNA sequence targeted by the gRNA of the system of the invention may be located immediately upstream to a PAM sequence.

According to one embodiment, the polynucleotide encoding the gRNA of the invention integrated in the transgenic organism of the invention may comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more, specifically, 110, 120, 130, 140, 150, 160, 170, 180, 190, 200 or more spacers, and as such, may encode at least 1 to 200 or more gRNAs. It should be further understood that the spacers of the nucleic acid sequence encoding the gRNA of the invention may be either identical or different spacers. In more embodiments, these spacers may target either an identical or different target genomic DNA. In yet some other embodiments, such spacer, and thereby the encoded gRNAs, may target at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more target genomic DNA sequence. As shown by Example 1, although successful in most cases, in some rare cases, even when three different target genes essential for survival were targeted, the target escaped targeting, possibly due to non-functional guides in certain settings. Thus, in some embodiments, three or more different targets may be targeted by at least three different gRNAs used by the systems, transgenic organisms and methods of the invention. In yet some further embodiments, 3 to 100, 150, 200, 250 or more targets may be targeted by different gRNAs used by the invention. It should be appreciated that the length of the spacers as discussed herein is also relevant in some embodiments for any target recognition element used by the invention. These target sequences may be derived from a single locus or alternatively, from several target loci.

As used herein, the term “spacer” refers to either a non-repetitive or repetitive spacer sequence that is designed to target a specific sequence. In some specific embodiments, spacers may comprise between about 15 to about 50 nucleotides, specifically, about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more nucleotides. More specifically, spacers may comprise between about 20-35 nucleotides. The guide or targeting RNA encoded by the CRISPR system of the invention may comprise a CRISPR RNA (crRNA) and a trans activating RNA (tracrRNA). However, it should be noted that in some specific CRISPR system, the guide RNA does not include a tracrRNA, such as CPF1 based CRISPR-Cas systems and CRISPR type I-E. The sequence of the targeting RNA encoded by the CRISPR spacers is not particularly limited, other than by the requirement for it to be directed to (i.e., having a segment that is the same as or complementarity to) a target sequence in genomic DNA that is also referred to herein as a “proto-spacer”. Such proto-spacers comprise nucleic acid sequence having sufficient identity to a targeting RNA encoded by the CRISPR spacers comprised within the nucleic acid sequence encoding the gRNA of the methods and systems of the invention. In some embodiments, a crRNA comprises or consists of 20, 21, 22, 23, 24, 25, 26, 27, 28, 29, 30, 31, 32, 33, 34, 35, 36, 37, 38, 39 or 40 nt of the spacer (targeting) sequence. In specific and non-limiting embodiments, the targeting spacer may comprise or consist of a segment that targets any one of the genomic DNA target sequence for which representative spacer sequences are indicated herein.

In some other embodiment, the RNA guided DNA binding protein nuclease of the system of the invention may be any one of a clustered regularly interspaced short palindromic repeat (CRISPR) of a newly identified system. In addition, a recent study have demonstrated that Cas9 can be split into two distinct fragments, which will reconstitute a functional full-length Cas9 nuclease when brought back together. Examples of efficient N and C terminal fragments of Cas9 being able to perform auto-assembly are described in Zetche B et al. (2015) Nat Biotechnol; 33(2): 139-142. As indicated above, the nucleases, and specifically, the guided nucleases such as cas9 used by the systems, transgenic organism and methods of the invention may be in some embodiments, catalytically inactive nucleases. In such cases, only the targeting properties of these guided nucleases are used (e.g., targeting a target nucleic acid sequence using gRNAs as targeting guides), for targeted manipulation of a target sequence, and the nucleolytic activity in such cases is undesired. Thus, in some embodiments, a guided nuclease with no nucleolytic activity may be used. In some particular and non-limiting embodiments, the Cas9 enzyme used for the systems of the invention may be a cas9 devoid of any nucleolytic activity, for example, a defective enzyme such as dCas9. dCas9 is a mutant Cas9 that lacks endonucleolytic activity. A non-limiting example for such mutant may be a mutant that carries a point mutation in at least one of D10A (aspartic acid to alanine in position 10) and H840A (histidine to alanine in position 840). Such mutant can be used as a modular RNA-guided platform to recruit different protein effectors to DNA in a highly specific manner in cells (Qi et al., Cell 152: 1173-1183 (2013)). Both repressive and activating effectors can be fused to dCas9 to repress or alternatively, activate gene expression, respectively (e.g., using methylases (methyl transferases), demethylases, transcription factors or transcription repressors etc.). Thus, in some embodiments, a fusion protein of dCas9 and activating effectors (e.g., transcription factors or enzymes that perform de methylation) or a fusion of dCas9 with repressors, may be used by the systems of the invention. More specifically, the activation or repression of a specific target sequence may be determined by the sgRNAs that recognize the target DNA sequences based only on homologous base pairing. The use of dCas9 fusion protein to repress genes that are essential for embryo development, or to activate genes that are detrimental for embryo development, can be harnessed to determine viability of the fertilized zygote. In fact, such use may be safer than the use of the natural Cas9 as the changes made do not alter the DNA, but only the transcription level. Thus, regulatory-wise, it may be more acceptable.

Repression by dCas9 is achieved when the naked mutated protein is guided to the target. This repression is more efficient when the guide targets dCas9 to the promoter region of the desired gene and when the guide sequence is complementary to the non-template strand of the gene, and therefore is identical, at least in part to the template strand, e.g., that includes the protospacer. However, this is not absolutely essential and in some instances guides to different regions in the gene or the opposite template can also repress efficiently.

Repression by dCas9 can be enhanced by fusing the dCas9 to known repressors. A non-limiting example for such repressor may be the Krüppel associated box (KRAB) domain, which enhances repression of the targets (Gilbert et al., Cell 154:442-451 (2013)). Activation by dCas9 is achieved when a transcriptional activator is fused to it. A non-limiting example for such activator may be the Herpes simplex virus protein vmw65, also known as VP16 (Gilbert et al., Cell 154:442-451 (2013)). Alternatively, the guide RNAs themselves can be engineered (instead or in addition to dCas9) to recruit either activators or repressors, and thus recruit naked dCas9 and dictate the outcome (Zalatan et al., Cell 160: 339-350 (2015)). For example, the guides can encode an RNA-domain that recruits a specific RNA-binding protein. This RNA-binding protein may be fused to an activator (e.g., VP16) or a repressor (e.g., Krab) and thus the entire recruitment of dCas9 along with the repressor or activator results in a desired outcome, specifically, the manipulation of the target sequence.

As indicated above, the systems and methods of the invention may further encompass the use of nucleases that cut RNA. Thus, in some embodiments, guided RNA nucleases that may be used by the invention may be CRISPR-Cas systems that target RNA, and can be advantageous (e.g., CRISPR-Cas Type III and type VI). It should be therefore appreciated that any of the nucleases described herein before or after or any other modifiers or any combinations, complexes and fusion proteins thereof are applicable for this aspect and for any of the aspects of the invention. It should be understood that the systems as well as the methods of the invention are suitable to any eukaryotic species possessing a heterogametic and homogametic organisms. Such organisms are present both in the Animalia and Plantae biological kingdoms.

In some further embodiments, the eukaryotic heterogametic organism and homogametic organism of the system of the invention may be of the biological kingdom Animalia.

In other embodiments, the eukaryotic heterogametic organism and homogametic organism of the system of the invention may be any one of a vertebrate or an invertebrate.

Invertebrates are animals that neither possess nor develop a vertebral column (commonly known as a backbone or spine), derived from the notochord. This includes all animals apart from the subphylum Vertebrata. Familiar examples of invertebrates include insects; crabs, lobsters and their kin; snails, clams, octopuses and their kin; starfish, sea-urchins and their kin; jellyfish and worms. Vertebrates comprise all species of animals within the subphylum Vertebrata (chordates with backbones). Vertebrates represent the overwhelming majority of the phylum Chordata, with currently about 66,000 species described. Vertebrates include the jawless fish and the jawed vertebrates, which include the cartilaginous fish (sharks, rays, and ratfish) and the bony fish. More specifically, in certain embodiments, the transgenic organism of the systems and methods of the invention may be any one of a mammal, an avian, an insect, a fish, an amphibian, a reptile, a crustacean, a crab, a lobster, a snail, a clam, an octopus, a starfish, a sea urchin, jellyfish, and worms.

In some embodiments, the system of the invention may be relevant for mammalian organisms. In yet some further embodiments, such mammalian organisms may include any member of the mammalian nineteen orders, specifically, Order Artiodactyla (even-toed hoofed animals), Order Carnivora (meat-eaters), Order Cetacea (whales and purpoises), Order Chiroptera (bats), Order Dermoptera (colugos or flying lemurs), Order Edentata (toothless mammals), Order Hyracoidae (hyraxes, dassies), Order Insectivora (insect-eaters), Order Lagomorpha (pikas, hares, and rabbits), Order Marsupialia (pouched animals), Order Monotremata (egg-laying mammals), Order Perissodactyla (odd-toed hoofed animals), Order Pholidata, Order Pinnipedia (seals and walruses), Order Primates (primates), Order Proboscidea (elephants), Order Rodentia (gnawing mammals), Order Sirenia (dugongs and manatees), Order Tubulidentata (aardvarks).

In yet some further embodiments, the systems of the invention are of particular relevance to rodent since it represents the most popular and commonly accepted animal model in research. Thus, in some further embodiment, the mammal of the system of the invention may be a rodent. Rodents are mammals of the order Rodentia, which are characterized by a single pair of continuously growing incisors in each of the upper and lower jaws. Rodents are the largest group of mammals. In some embodiments, the transgenic organisms of the systems and methods of the invention may be any rodent. Non-limiting examples for such rodents that are applicable in the present invention, appear in the following list of rodents, arranged alphabetically by suborder and family. Suborder Anomaluromorpha includes the anomalure family (Anomaluridae) [anomalure (genera Anomalurus, Idiurus, and Zenkerella)], the spring hare family (Pedetidae) [spring hare (Pedetes capensis)]. The suborder Castorimorpha includes the beaver family (Castoridae) [beaver (genus Castor), giant beaver (genus Castoroides; extinct)], the kangaroo mice and rats (family Heteromyidae) [kangaroo mouse (genus Microdipodops), kangaroo rat (genus Dipodornys), pocket mouse (several genera)], the pocket gopher family (Geomyidae) [pocket gopher (multiple genera)]. Suborder Hystricomorpha, includes the agouti family (Dasyproctidae), acouchy (genus Myoprocta) [agouti (genus Dasyprocta)], the American spiny rat family (Echimyidae), the American spiny rat (multiple genera), the blesmol family (Bathyergidae) [blesmol (multiple genera)], the cane rat family (Thryonomyidae) [cane rat (genus Thryonomys)], the cavy family (Caviidae) [capybara (Hydrochoerus hydrochaeris), guinea pig (Cavia porcellus) mara (genus Dolichotis)], the chinchilla family (Chinchillidae) [chinchilla (genus Chinchilla), viscacha (genera Lagidium and Lagostomus)], the chinchilla rat family (Abrocomidae) [chinchilla rat (genera Cuscomys and Abrocoma)], the dassie rat family (Petromuridae) [dassie rat (Petromus typicus)], the degu family (Octodontidae) [degu (genus Octodon)], the diatomyid family (Diatomyidae), the giant hutia family (Heptaxodontidae), the gundi family (Ctenodactylidae) [gundi (multiple genera)], the hutia family (Capromyidae) [hutia (multiple genera)], the New World porcupine family (Erethizontidae) [New World porcupine (multiple genera)], the nutria family (Myocastoridae) [nutria (Myocastor coypus)], the Old World porcupine family (Hystricidae) [Old World porcupine (genera Atherurus, Hystrix, and Trichys)], the paca family (Cuniculidae) [paca (genus Cuniculus)], the pacarana family (Dinomyidae) [pacarana (Dinomys branickii)], the tuco-tuco family (Ctenomyidae) [tuco-tuco (genus Ctenomys)]. The suborder Myomorpha that includes the cricetid family (Cricetidae) [American harvest mouse (genus Reithrodontomys), cotton rat (genus Sigmodon), deer mouse (genus Peromyscus), grasshopper mouse (genus Onychomys), hamster (various genera), golden hamster (Mesocricetus auratus), lemming (various genera) maned rat (Lophiomys imhausi), muskrat (genera Neofiber and Ondatra), rice rat (genus Oryzomys), vole (various genera), meadow vole (genus Microtus), woodland vole (Microtus pinetorum), water rat (various genera), woodrat (genus Neotoma), dipodid family (Dipodidae), birch mouse (genus Sicista), jerboa (various genera), jumping mouse (genera Eozapus, Napaeozapus, and Zapus)], the mouselike hamster family (Calomyscidae), the murid family (Muridae) [African spiny mouse (genus Acomys), bandicoot rat (genera Bandicota and Nesokia), cloud rat (genera Phloeomys and Crateromys), gerbil (multiple genera), sand rat (genus Psammomys), mouse (genus Mus), house mouse (Mus musculus), Old World harvest mouse (genus Micromys), Old World rat (genus Rattus), shrew rat (various genera), water rat (genera Hydromys, Crossomys, and Colomys), wood mouse (genus Apodemus)], thenesomyid family (Nesomyidae), African pouched rat (genera Beamys, Cricetomys, and Saccostomus)], the Oriental dormouse family (Platacanthomyidae)[Asian tree mouse (genera Platacanthomys and Typhlornys)], the spalacid family (Spalacidae) [bamboo rat (genera Rhizomys and Cannomys), blind mole rat (genera Nannospalax and Spalax), zokor (genus Myospalax), suborder Sciuromorpha], the dormouse family (Gliridae) [dormouse (various genera), desert dormouse (Selevinia betpakdalaensis)], the mountain beaver family (Aplodontiidae) [mountain beaver (Aplodontia rufa)], the squirrel family (Sciuridae) [chipmunk (genus Taenias), flying squirrel (multiple genera), ground squirrel (multiple genera), suslik (genus Spermophilus), marmot (genus Marmota), groundhog (Marmota monax), prairie dog (genus Cynomys), tree squirrel (multiple genera)]. In yet some further embodiments, the system of the invention may be applicable for mice. A mouse, plural mice, is a small rodent characteristically having a pointed snout, small rounded ears, a body-length scaly tail and a high breeding rate. The best known mouse species is the common house mouse (Mus musculus). Species of mice are mostly found in Rodentia, and are present throughout the order. Typical mice are found in the genus Mus.

In some embodiments, the rodent may be a mouse and the at least one nucleic acid sequence encoding or forming at least one guide RNA directed against at least one target sequence may be incorporated in the female (the homogametic subject, XX) and the nuclease (e.g., Cas9 or any mutant, derivative or fusion protein thereof) may be integrated by the male subject (the heterogametic organism, XY). In yet some other alternative embodiments, the at least one nucleic acid sequence encoding or forming at least one guide RNA directed against at least one target sequence may be incorporated in the male (the heterogametic subject) and the nuclease (e.g., dCas9) may be integrated in the female subject (the homogametic organism).

In a more specific embodiment, the rodent may be a mouse and the at least one nucleic acid sequence encoding at least one guide RNA directed against at least one target sequence may be integrated into the X chromosome of said mouse, specifically, heterogametic mouse. Alternatively, the least one nucleic acid sequence encoding at least one guide RNA directed against at least one target sequence may be integrated into the Y chromosome of the mouse.

In some further embodiments, the rodent of the system of the invention may be a mouse and the at least one nucleic acid sequence encoding at least one guide RNA directed against at least one target sequence may be integrated into the X chromosome of the mouse, specifically, the heterogametic mouse. Such system may be used for selecting for male progeny. In yet some alternative embodiments, the at least one nucleic acid sequence encoding at least one guide RNA directed against at least one target sequence may be integrated into the Y chromosome of the mouse. Such system may be used in some embodiments to select for female progeny.

In some specific and non-limiting embodiments, the at least one target sequence may be any sequence encoding or controlling the expression of a product essential for embryogenesis, survival or development, as specified above. In more specific embodiments, at least one gene essential for embryogenesis may be any one of Atp5b (ATP synthase subunit beta, mitochondrial), Cdc20 (cell-division cycle protein 20) and Casp8 (Caspase-8).

More specifically, ATP5B (ATP Synthase, H+ Transporting, Mitochondrial Fl Complex, Beta Polypeptide) is a Protein Coding gene. Among its related pathways are Metabolism and Respiratory electron transport, ATP synthesis by chemiosmotic coupling, and heat production by uncoupling proteins. GO annotations related to this gene include transporter activity and transmembrane transporter activity. atp5b deficiency in mice results in embryonic lethality prior to organogenesis. In some specific embodiments, Atp5b used by the invention may comprise the nucleic acid sequence as denoted by SEQ ID NO: 13.

The cell-division cycle protein 20 is an essential regulator of cell division that is encoded by the CDC20 gene in humans. The gene product activates the anaphase promoting complex (APC/C), a large 11-13 subunit complex that initiates chromatid separation and entrance into anaphase. The APC/CCdc20 protein complex has two main downstream targets. Firstly, it targets securin for destruction, enabling the eventual destruction of cohesin and thus sister chromatid separation. It also targets S and M-phase (S/M) cyclins for destruction, which inactivates S/M cyclin-dependent kinases (Cdks) and allows the cell to exit from mitosis. A closely related protein, Cdc20homologue-1 (Cdhl) plays a complementary role in the cell cycle. CDC20 appears to act as a regulatory protein interacting with many other proteins at multiple points in the cell cycle. It is required for two microtubule-dependent processes: nuclear movement prior to anaphase, and chromosome separation. cdc20 deficiency in mice results in metaphase arrest in two-cell stage embryos and consequently in early embryonic death (M. Li, et al., (2007) Mol Cell Biol 27, 3481-3488). In some specific embodiments, CDC20 used by the invention may comprise the nucleic acid sequence as denoted by SEQ ID NO: 14.

Caspase-8 is a cysteine-aspartic acid protease (caspase) protein, encoded by the Casp8 gene. This protein is involved in the programmed cell death by various apoptotic stimuli. On the other hand, Caspase-8 activity was shown to be essential in early mouse embryonic development as mutants lacking Caspase-8 catalytic activity all die at embryonic day (E) 10.5-12.5 (Varfolomeev, E. E., et al., (1998) Immunity. 9(2): 267-76; Sakamaki, K., S., et al., (1998), Eur J Biochem. 253(2): 399-405). This was later found to be due its regulatory role during necroptosis. In some specific embodiments, Caspase-8 used by the invention may comprise the nucleic acid sequence as denoted by SEQ ID NO: 15.

In yet some further specific and non-limiting embodiments, at least one nucleic acid sequence (spacer) encoding said guide RNAs directed against at least one essential gene for embryogenesis may comprise the nucleic acid sequence as denoted by SEQ ID NO:1, SEQ ID NO:2 or SEQ ID NO:3.

Thus, in certain embodiments, the heterogametic mouse (male) of the invention, used by the systems and methods described herein, may comprise at least one gRNA specific for ATPSB, at least one gRNA specific for Cdc20, at least one gRNA specific for Casp8. In yet some further embodiments, these gRNA sequences may be incorporated either to the X or to the Y gender chromosomes of the male mouse.

As indicated above, any gene essential for survival, embryogenesis and cell cycle, may be targeted by the gRNA transgenes of the invention. Some non-limiting embodiments for mouse essential genes may include at least one of Abca1, Acat2, Acbd3, Ackr3, Actr8, Acvr1, Acvr1b, Adam23, Adar, Ads1, Ahcy, Aim1, A1g10b, A1g2, Anapc15, Anapc4, Ankrd11, Anks6, Arid3a, Ascc2, Asf1a, Atg3, Atp1a3, Atp2a1, Atp2a2, Atp5b, Atp5e, Atp51, Atp6v0a1, Atp6v0a4, Atp6v1b2, Atp6v1d, Atr, Atrip, Atxn10, Atxn713, Avp, Bag3, Bbs10, BC055324, Bclaf1, Bloc1s2, Bmp10, Bms1, Cacna1s, Carm1, Casc3, Casq2, Casz1, Cbx2, Cbx4, Ccdc94, Ccnb1, Cdc123, Cdc20, Cdc26, Cdca5, Cdipt, Cdk8, Cdk9, Cenpe, Cenph, Cenpo, Cep164, Cgn, Chd1, Chd4, Chmp3, Chmp6, Chrna1, Chtop, Cinp, Clcf1, Cldn1, Clspn, Coa5, Cog3, Commd9, Coq2, Coq6, Cox19, Cox4i1, Cox5b, Cox7c, Cr11, Crip1, Crispld2, Cs, Ctla2b, Ctr9, Daam1, Dach1, Dcc, Dcp2, Dcps, Ddost, Ddx41, Dennd4c, Denr, Depdc5, Der12, Dhfr, Dhodh, Dhx30, Dhx33, Diaph3, Dicer1, Dis3, Dmap1, Dnaaf2, Dnajc17, Dnajc8, Dnajc9, Dnml1, Dpm1, Dpm2, Dpp9, Dppa1, Dt1, Eaf1, Ect2, Eif2b3, Eif2b4, Eif4g2, Elof1, Eomes, Epas1, Epc2, Eprs, Ercc3, Ewsr1, Exoc312, Exoc8, Exosc8, Exosc9, Eya4, F10, Fadd, Fam20c, Fam210a, Fam46c, Fbxo16, Fdx1, Fgf10, Fgf8, Fgf9, Fgfr1, Fgfr1op, Flii, Flnb, Fntb, Foxj3, Frs2, Furin, Fus, Gabarapl2, Gart, Gdi2, Gfi1b, Gfpt1, Ggps1, Gnao1, Gnpda1, Gosr2, Gps1, Gpx3, Gtf2b, Gyg, Hectd1, Hells, Hspa5, Htr2b, Ift88, Ino80b, Inpp5e, Ints12, Ivd, Jag2, Jmjd6, Jup, Kars, Katnb1, Kctd15, Kdm5c, Kdm8, Kif1b, Kif20a, Kif26b, Kif3b, Klf7, Klhdc2, Krit1, Krt8, Lama5, Lamtor5, Lifr, Lmbrd1, Lmna, Lrp1, Lss, Maea, Map3k7, Mapkap1, Mat2a, Mbd6, Mcrs1, Mcu, Med11, Med25, Med28, Memo1, Mett116, Mir141, Mir143, Mir145a, Mir200a, Mir203, Mir320, Mir429, Mllt3, Mocs2, Mogs, Mrp151, Mrps25, Mrps5, Msx1, Mtch2, Mtf1, Mybbp1a, Myo18a, Nabp2, Nael, Nampt, Ndufa8, Ndufs1, Ndufs7, Nemf, Nepro, Nfatc3, Nme6, No18, Nr6a1, Nsf, Ntrk1, Nubp1, Nutf2, Nxn, Orc1, Otub1, Palb2, Pam16, Pard3, Patzl, Pax4, Pbx3, Pcgf2, Pcsk5, Pcx, Pdcd2, Pde12, Pdgfrb, Pdia6, Pex26, Pgs1, Phf11a, Phf6, Pibf1, Pigh, Pig1, Pigu, Pik3c2a, Pitx2, Plk1, Plpp3, Pnn, Pold3, Polr2h, Pparg, Ppp1r35, Ppp2r4, Ppp4r2, Ppp6c, Prdm10, Prep, Prpf31, Prpsl, Psenl, Psmd14, Psmf1, Psph, Ptma, Ptpmt1, Ptpn12, Ptpn23, Pyroxd1, Rab23, Rab34, Rab35, Rabggta, Raf1, Ranbp2, Rapsn, Rasa3, Rbms1, Recq14, Rest, Rexo2, Rfc1, Rgp1, Ric8b, Rint1, Ripk1, Rnf20, Rnmtl1, Rpa1, Rptor, Rrm2, Rsbn1, Rsf1, Rufy3, Rwdd3, Ryr2, Sacml1, Satb1, Scn4a, Scnn1b, Scrib, Sdha, Sdhb, Sdhc, Sgpl1, Shh, Ska2, Slc25a3, Slc2a2, Slc39a7, Slc39a8, Slc40a1, Slc52a2, Slc6a5, Smc5, Smdt1, Smo, Snapin, Snrnp200, Snrnp27, Socs1, Spata5, Speg, Spop, Sptssa, Srd5a3, Srp9, Strn3, Stx3, Sufu, Supt4a, Svep1, Synj1, Synpo2, Tango6, Tbx4, Teadl, Tfeb, Tgif2, Timm22, Timm50, Tmed10, Tmem100, Tmem132a, Tmem63b, Trpc3, Tulp3, Ube2c, Ube2h, Ube2m, Uch15, Unc45a, Uqcrb, Usp10, Usp14, Usp16, Usp22, Usp36, Usp39, Usp50, Vezt, Vps13d, Vps25, Vps37d, Vps4a, Wars, Wars2, Wdr12, Wdr59, Weel, Wnt6, Wrap53, Xbp1, Xrcc3, Yaeldl, Yars, Yipf5, Zbtb24, Zfp148, Zfp207, Zfp36, Zfp536, Zkscan17, Zmiz2. It should be appreciated that the invention further encompasses any derivatives, homologs and any orthologs of any of the above essential mouse genes, as targets for the gRNAs of any of the transgenic organisms of the invention, and specifically for any mammalian organism discussed herein (e.g., cattle).

It should be appreciated that the present invention provides in additional aspects thereof any of the transgenic organisms described herein before, and in connection with other aspects of the invention. In yet some further specific embodiments, the invention provides transgenic male mice comprising at least one sequence forming or encoding at least one gRNA directed against at least one essential gene. Such transgenic sequences may be incorporated in some embodiments into the X chromosome of said male mice. In yet some further embodiments, such sequences may be incorporated into the Y chromosome thereof. Example for such mice are presented by the following examples (designated herein as the “Y-line”). These transgenic male mice comprise nucleic acid sequences encoding at least one gRNA specific for Atp5b, at least one gRNA specific for Cdc20 and at least one gRNA specific for Casp8. It should be noted that any cell, specifically, zygote cell (e.g., sperm or semen) of said transgenic mice or any progeny thereof, are also encompassed by the invention. It should be also understood that the invention further pertains to any use of the transgenic organisms disclosed by the invention. The invention further encompasses any cell, any progeny, any product or use thereof. Still further, in some embodiments, the invention provides transgenic male mice comprising at least one sequence encoding at least one nuclease. Such transgenic sequences may be incorporated in some embodiments into the X chromosome of said male mice. In yet some further embodiments, such sequences may be incorporated into the Y chromosome thereof. In yet some further specific embodiments, the invention provides transgenic female mice comprising at least one sequence forming or encoding at least one gRNA directed against at least one essential gene. Such transgenic sequences may be incorporated in some embodiments into any chromosome of such female mice. Still further, in some embodiments, the invention provides transgenic female mice comprising at least one sequence encoding at least one nuclease. Such transgenic sequences may be incorporated in some embodiments into any chromosome of such female mice.

In yet some further embodiments, the system of the invention may be applicable for avian organisms. In yet some further specific embodiments, the system of the invention may use birds as the transgenic organisms. More specifically, domesticated and an undomesticated birds are also suitable organisms for the systems of the invention.

Therefore in certain embodiments, the avian organism of the system of the invention may be any one of a domesticated and an undomesticated bird. In more specific embodiment, the avian organism may be any one of a poultry or a game bird. In some specific embodiments, the avian organism may be of the order Galliformes which comprise without limitation, chicken, quail, turkey, duck, Gallinacea sp, goose, pheasant and other fowl. The term “avian” relates to any species derived from birds characterized by feathers, toothless beaked jaws, the laying of hard-shelled eggs, a high metabolic rate, a four-chambered heart, and a lightweight but strong skeleton. The term “hen” includes all females of the avian species. In yet some specific embodiments, the systems of the invention may be also suitable for chicken.

Chimeric avian are generated which are derived in part from the modified embryonic stem cells or zygote cells, capable of transmitting the genetic modifications through the germ line. Mating avian strains containing exogenous sequences should result in progenies displaying the desired gender. Still further, transgenic avian can be produced by different methods, some of which are discussed below and in the examples section. Among the avian cells suitable for transformation for generating transgenic animals are primordial germ cells (PGC), sperm cells and zygote cells (including embryonic stem cells). Sperm cells can be transformed with DNA constructs by any suitable method, including electroporation, microparticle bombardment, lipofection and the like. The sperm can be used for artificial insemination of avian. Progeny of the inseminated avian can be examined for the exogenous sequence as described above. The developmental stage of chicken is as detailed in the following: the chicken germ-line develops from a small population of primordial germ cells (PGCs), migrating to the genital ridge from an extragonadal site, while undergoing phases of active migration, as well as passive circulation in the bloodstream. PGCs are located in the center of the epiblast of freshly laid, un-incubated egg, a developmental stage referred as stage X. During incubation PGCs migrate anteriorly and accumulate at the germinal crescent of stage 10HH embryo (approximately 33-38 hours of incubation), considered the main site for intravasation. Later, PGCs can be found in the circulation from stage 12HH to 17HH (approximately from 45 to 64 hours of incubation), reaching a peak concentration in stage 14HH (approximately 50-53 hours of incubation). PGCs leave the circulation at a site adjacent to the gonad anlage at the intermediate mesoderm, where they can be found as early as stage 15HH (55-55 hours of incubation). PGCs reach the genital ridge by active migration along the dorsal mesentery, and colonize both gonads, where they later differentiate into spermatogonia or oogonia. “Primordial germ cells (PGCs)”, as used herein relates to germline stem cells that serve as progenitors of the gametes and give rise to pluripotent embryonic stem cells. The cells in the gastrulating embryo that are signaled to become PGCs during embryogenesis, migrate into the genital ridges which becomes the gonads, and differentiate into mature gametes.

Newly hatched avian can be tested for the presence of the target construct sequences, for example by examining a biological sample thereof, e.g., a blood sample. After the avian have reached maturity, they are bred and their progeny may be examined to determine whether the exogenous integrated sequences are transmitted through the germ line.

In some embodiments, the avian transgenic organism may be a chicken and the at least one nucleic acid sequence encoding or forming at least one guide RNA directed against at least one target sequence may be incorporated in the male (the homogametic subject ZZ), and the nuclease (e.g., Cas9 or any mutant, derivative or fusion protein thereof) may be integrated in the female subject (the heterogametic organism, ZW). In yet some other alternative embodiments, the at least one nucleic acid sequence encoding or forming at least one guide RNA directed against at least one target sequence may be incorporated in the female (the heterogametic subject), and the nuclease (e.g., Cas9 or any mutant, derivative or fusion protein thereof) may be integrated in the male subject (the homogametic organism).

In more specific embodiments, the domesticated bird may be a chicken and the at least one nucleic acid sequence encoding at least one guide RNA directed against at least one target sequence may be integrated into the W chromosome of the chicken, specifically, the heterogametic chicken. Alternatively, the at least one nucleic acid sequence encoding at least one guide RNA directed against at least one target sequence may be integrated into the Z chromosome of the chicken, specifically, the heterogametic chicken. In more specific embodiments, the domesticated bird may be a chicken and the at least one nucleic acid sequence encoding at least one guide RNA directed against at least one target sequence may be integrated into the W chromosome of the chicken, specifically, the heterogametic chicken. In some embodiments, such system may be used for selecting for a male progeny. In yet some other alternative embodiments, the at least one nucleic acid sequence encoding at least one guide RNA directed against at least one target sequence may be integrated into the Z chromosome of the chicken, specifically, the heterogametic chicken. In some embodiments, such system may be used to select for a female progeny.

In some specific and non-limiting embodiments, the at least one target sequence may be a gene or any product/s thereof essential for embryogenesis, survival or development. In yet some specific embodiments, a gene essential for avian embryogenesis may be at least one of Casp8, Atp5b (ATP synthase subunit beta, mitochondrial) and Cdc20 (cell-division cycle protein 20), as denoted by SEQ ID NO: 28, 26 and 27, respectively. IHH (Indian Hedgehog)

In yet some further specific embodiments at least one nucleic acid sequence (spacer) encoding said guide RNAs directed against at least one essential gene for embryogenesis may comprise the nucleic acid sequence as denoted by at least one of SEQ ID NO:8, SEQ ID NO:9 or SEQ ID NO:10, or any combinations thereof. In some embodiments, the transgenic avian organism of the invention may encode at least one gRNA targeting the Casp8, Atp5b (ATP synthase subunit beta, mitochondrial) and Cdc20 (cell-division cycle protein 20 as denoted by SEQ ID NO: 28, 26 and 27. It should be noted that the Indian hedgehog (IHH), may be also used as a target in the present invention. More specifically, gene was suggested to be essential for embryonic chick development, as its deficiency was only found in the whole-genome sequencing of lethal embryos and Creeper chickens (Jin, S. et al. (2016). Sci. Rep. 6, 30172). It encodes for a protein involved in intercellular signals essential for a variety of patterning events during development. It should be noted that the avian Atp5b and Cdc20 are orthologs of the rodent genes discussed above.

Thus, in certain embodiments, the heterogametic avian subject, specifically the chicken (female) of the invention, used by the systems and methods described herein, may comprise nucleic acid sequences encoding or forming at least one gRNA specific for Ihh, at least one gRNA specific for Cdc20, at least one gRNA specific for Atp5b. In yet some further embodiments, these gRNA encoding sequences may be incorporated either to the Z or to the W gender chromosomes of the female avian. In yet some further alternative or additional embodiments, the target sequence may be sequences appearing in non-coding regions of at least one chromosomes.

Still further, in some embodiments, the transgenic avian organism of the invention may express at least one gRNA specific for any avian gene essential for survival and/or embryonic development. It should be noted that any avian ortholog of any of the mice essential genes listed above may be also applicable for avian organisms of the invention.

It should be appreciated that the present invention provides in additional aspects thereof any of the transgenic avian organisms described herein before, and in connection with other aspects of the invention. The invention further encompasses any progeny of the transgenic avian organism disclosed herein, any cell thereof or any product or uses thereof. In yet some further specific embodiments, the invention provides transgenic female avian subject (chicken) comprising at least one sequence forming or encoding at least one gRNA directed against at least one essential gene, as discussed above. Such transgenic sequences may be incorporated in some embodiments into the W chromosome of said female avian. In yet some further embodiments, such sequences may be incorporated into the Z chromosome thereof. It should be noted that any cell, specifically, zygote cell (e.g., ovum) of said transgenic hen or any progeny thereof, are also encompassed by the invention. It should be also understood that the invention further pertains to any use of the transgenic organisms disclosed by the invention. Still further, in some embodiments, the invention provides transgenic female avian organism comprising at least one sequence encoding at least one nuclease. Such transgenic sequences may be incorporated in some embodiments into the Z chromosome of said female avian subject. In yet some further embodiments, such sequences may be incorporated into the W chromosome thereof. In yet some further specific embodiments, the invention provides transgenic male avian subjects comprising at least one sequence forming or encoding at least one gRNA directed against at least one essential gene, e.g., as discussed above. Such transgenic sequences may be incorporated in some embodiments into any chromosome of such male avian. Still further, in some embodiments, the invention provides transgenic male avian subject comprising at least one sequence encoding at least one nuclease (e.g., Cas9 or any mutant, derivative or fusion protein thereof). Such transgenic sequences may be incorporated in some embodiments into any chromosome of such male avian subject.

In yet some further embodiments, the systems of the invention are may be also applicable to the aquaculture industry. Aquaculture, also known as aquafarming, is the farming of fish, crustaceans, molluscs, aquatic plants, algae, and other organisms. Aquaculture involves cultivating freshwater and saltwater populations under controlled conditions, and can be contrasted with commercial fishing, which is the harvesting of wild fish. It should be noted that the present invention further pertains to Mariculture that refers to aquaculture practiced in marine environments and in underwater habitats. Unisex populations are often preferred by the aquaculture industry. More specifically, male fingerlings are preferred by tilapia growers and females by salmonid fish arms. Sex chromosomes were identified in both salmonid and in tilapia. All salmonid have the XX/XY mode of sex determination while different tilapine species may have the XX/XY or ZZ/ZW mode (reviewed in Cnaani et al. 2008 Sex Dev; 2:43-54).

More specifically, in the Nile tilapia O. niloticus, the male is heterogametic (XX/XY system), while in O. aureus from Israel, O. karongae and Tilapia mariae, the female is heterogametic (WZ/ZZ system). Thus, the present invention in some embodiments thereof also encompasses transgenic fish, as well as systems and methods using these fish. Fish, as used herein refer to gill-bearing aquatic craniate animals that lack limbs with digits. They form a sister group to the tunicates, together forming the olfactores. Included in this definition are the living hagfish, lampreys, and cartilaginous and bony fish as well as various related groups. It should be noted that the present invention relates to any group, class, subclass or any family of fish. Specifically, any fish of the following families, specifically, Cyprinidae, Gobiidae, Cichlidae, Characidae, Loricariidae, Balitoridae, Serranidae, Labridae, and Scorpaenidae.

In some specific embodiments, the fish of the system of the invention may be of the genus tilapia. Tilapia, as used herein is the common name for nearly a hundred species of cichlid fish from the tilapiine cichlid tribe. Tilapia are mainly freshwater fish inhabiting shallow streams, ponds, rivers, and lakes, and less commonly found living inbrackish water. It should be noted that the invention relates to any species of tilapia.

In more specific embodiments, the tilapia fish may be of the Oreochromis niloticus species. Still further, the at least one nucleic acid sequence encoding at least one guide RNA directed against at least one target sequence may be integrated into the X chromosome of the fish, specifically, the heterogametic fish. Alternatively, the at least one nucleic acid sequence encoding at least one guide RNA directed against at least one target sequence may be integrated into the Y chromosome of the fish, specifically, the heterogametic fish.

In some further embodiments, the tilapia fish may be of the Oreochromis niloticus species, and the at least one nucleic acid sequence encoding at least one guide RNA directed against at least one target sequence may be integrated into the X chromosome of the fish, specifically, of the heterogametic fish. In some embodiments, such system may be used to select for a male progeny. In yet some alternative embodiments, the at least one nucleic acid sequence encoding at least one guide RNA directed against at least one target sequence may be integrated into the Y chromosome of the fish. In some embodiments, such system may be used to select for a female progeny. In yet other embodiment, the tilapia fish may be of any one of the species Oreochromis aureus, Oreochromis karongae or Pelmatolapia mariae, and the at least one nucleic acid sequence encoding at least one guide RNA directed against at least one target sequence may be integrated into the W chromosome of the fish. In yet some other alternative embodiments, the at least one nucleic acid sequence encoding at least one guide RNA directed against at least one target sequence may be integrated into the Z chromosome of the fish, specifically, the heterogametic fish. It should be noted that the invention further encompasses any of the transgenic fish described herein, as well as any progeny, cell or embryo thereof, any product, component or uses of any of the above. In more specific embodiments, the tilapia fish used as the transgenic organisms of the systems of the invention may be of any one of the species Oreochromis aureus, Oreochromis karongae or Pelmatolapia mariae. In such case, the at least one nucleic acid sequence encoding at least one guide RNA directed against at least one target sequence may be integrated into the W chromosome of the fish. In some embodiments, such system may be used for selecting for a male progeny. In yet some alternative embodiments, the at least one nucleic acid sequence encoding at least one guide RNA directed against at least one target sequence may be integrated into the Z chromosome of the heterogametic fish. Such system may be used for selecting for a female progeny.

Still further, the systems of the invention may be useful for crustaceans organisms. Crustaceans, as used herein, form a large, diverse arthropod taxon which includes crabs, lobsters, crayfish, shrimp, hill, woodlice, and barnacles, that are all encompassed by the present invention. The crustacean group is usually considered as a paraphyletic group, and comprises all animals in the Pancrustacea Glade other than hexapods. Some crustaceans are more closely related to insects and other hexapods than they are to certain other crustaceans. In some embodiments, such crustaceans may be shrimp. The term shrimp is used to refer to decapod crustaceans, and covers any of the groups with elongated bodies and a primarily swimming mode of locomotion i.e. Caridea and Dendrobranchiata. In shrimps, the female heterogamety of the ZZ/ZW type enables sex determination. The female shrimps are preferred since they are brighter in color and are larger compared to the males.

Thus, in some embodiments, the crustaceans of the system of the invention may be shrimp. In some embodiments, the at least one nucleic acid sequence encoding at least one guide RNA directed against at least one target sequence may be integrated into the W chromosome of the shrimp. In some alternative embodiments, the at least one nucleic acid sequence encoding at least one guide RNA directed against at least one target sequence may be integrated into the Z chromosome of the shrimp, specifically, the heterogametic shrimp.

In more specific embodiments, the crustaceans may be shrimp, and the at least one nucleic acid sequence encoding at least one guide RNA directed against at least one target sequence may be integrated into the W chromosome of the shrimp. Such system may be used for selecting for a male progeny. In yet some further embodiments, the at least one nucleic acid sequence encoding at least one guide RNA directed against at least one target sequence may be integrated into the Z chromosome of the shrimp, specifically, the heterogametic shrimp. Such system may be used for selecting for a female progeny.

Still further, in some embodiments, the systems provided by the invention may be applicable for insects and thus, may provide and use transgenic insects. Insects or Insecta are hexapod invertebrates and the largest group within the arthropod phylum. Definitions and circumscriptions vary; usually, insects comprise a class within the Arthropoda. As used here, the term Insecta is synonymous with Ectognatha. Insects have a chitinous exoskeleton, a three-part body (head, thorax and abdomen), three pairs of jointed legs, compound eyes and one pair of antennae. Insects are the most diverse group of animals; they include more than a million described species and represent more than half of all known living organisms. Insects can be divided into two groups historically treated as subclasses: wingless insects, known as Apterygota, and winged insects, known as Pterygota. The Apterygota consist of the primitively wingless order of the silverfish (Zygentoma). Archaeognatha make up the Monocondylia based on the shape of their mandibles, while Zygentoma and Pterygota are grouped together as Dicondylia. The Zygentoma themselves possibly are not monophyletic, with the family Lepidotrichidae being a sister group to the Dicondylia (Pterygota and the remaining Zygentoma). Paleoptera and Neoptera are the winged orders of insects differentiated by the presence of hardened body parts called sclerites, and in the Neoptera, muscles that allow their wings to fold flatly over the abdomen. Neoptera can further be divided into incomplete metamorphosis-based (Polyneoptera and Paraneoptera) and complete metamorphosis-based groups. It should be noted that the present invention is applicable for any of the insects of any of the groups and species disclosed herein.

Still further, many insects are considered pests by humans. Insects commonly regarded as pests include those that are parasitic (e.g. lice, bed bugs), transmit diseases (mosquitoes, flies), damage structures (termites), or destroy agricultural goods (locusts, weevils). Insects considered pests of some sort occur among all major living orders with the exception of Ephemeroptera (mayflies), Odonata, Plecoptera (stoneflies), Embioptera (webspinners), Trichoptera (caddisflies), Neuroptera (in the broad sense), and Mecoptera (also, the tiny groups Zoraptera, Grylloblattodea, and Mantophasmatodea). Of particular interest of this group is the Mosquito. More specifically, in some embodiments, the systems of the invention may be suitable for insects such as mosquito for example. Mosquitoes are a group of about 3500 species of small insects that are a type of fly (order Diptera). Within that order they constitute the family Culicidae. Superficially, mosquitoes resemble crane flies (family Tipulidae) and chironomid flies (family Chironomidae). It should be appreciated that in some embodiments, the term mosquito, as used herein includes all genera encompassed by the subfamilies Anophelinae and Culicinae. In yet some further embodiments, mosquito as used herein include, but is not limited to any mosquito of the following genera, Aedeomyia, Aedes, Anopheles, Armigeres, Ayurakitia, Borachinda, Coquillettidia, Culex, Culiseta, Deinocerites, Eretmapodites, Ficalbia, Galindomyia, Haemagogus, Heizmannia, Hodgesia, Isostomyia, Johnbelkinia, Kimia, Limatus, Lutzia, Malaya, Mansonia, Maorigoeldia, Mimomyia, Onirion, Opifex, Orthopodomyia, Psorophora, Runchomyia, Sabethes, Shannoniana, Topomyia, Toxorhynchites, Trichoprosopon, Tripteroides, Udaya, Uranotaenia, Verrallina, and Wyeomyia. Females of most species are ectoparasites, whose tube-like mouthparts (called a proboscis) pierce the hosts' skin to consume blood. Though the loss of blood is seldom of any importance to the victim, the saliva of the mosquito often causes an irritating rash that is a serious nuisance. Much more serious though, are the roles of many species of mosquitoes as vectors of diseases. In passing from host to host, some transmit extremely harmful infections such as malaria, yellow fever, Chikungunya, West Nile virus, dengue fever, filariasis, Zika virus and other arboviruses, rendering it the deadliest animal family in the world. Therefore, reducing the population of mosquitoes and particularly of female mosquitoes is of great relevance. Sex is determined in most mosquito by heterogamety, males being XY and females being XX.

Thus, in some further embodiments, the insects of the system of the invention may be mosquitoes. In some embodiments, the at least one nucleic acid sequence encoding or forming at least one guide RNA directed against at least one target sequence may be incorporated in the female (the homogametic subject, XX) and the nuclease (e.g., Cas9 or any mutant, derivative or fusion protein thereof) may be integrated by the male subject (the heterogametic organism, XY). In yet some other alternative embodiments, the at least one nucleic acid sequence encoding or forming at least one guide RNA directed against at least one target sequence may be incorporated in the male (the heterogametic subject) and the nuclease (e.g., Cas9 or any mutant, derivative or fusion protein thereof) may be integrated in the female subject (the homogametic organism). In some specific embodiments, at least one nucleic acid sequence encoding at least one guide RNA directed against at least one target sequence may be integrated into the X chromosome of the mosquito, specifically, the heterogametic transgenic mosquito. Alternatively, the at least one nucleic acid sequence encoding at least one guide RNA directed against at least one target sequence may be integrated into the Y chromosome of the transgenic mosquito.

In more specific embodiments, at least one nucleic acid sequence encoding at least one guide RNA directed against at least one target sequence may be integrated into the X chromosome of the heterogametic mosquito. Such system may be used in some embodiments, for selecting a male progeny. In yet some other alternative embodiments, the at least one nucleic acid sequence encoding at least one guide RNA directed against at least one target sequence may be integrated into the Y chromosome of the transgenic mosquito. Such system may be used in some embodiments to select for a female progeny. In some specific and non-limiting embodiments, the at least one target sequence may be a gene that may be essential for embryogenesis, survival or development of the mosquito. Some specific embodiments for such at least one gene essential for embryogenesis may be any one of the mosquito Cyclin A, Iap1 (Inhibitor of apoptosis 1) and Gpdh (Glycerol-3-phosphate dehydrogenase).

More specifically, the Cyclin A gene is a protein-coding gene for a member of the cyclin family, a group of proteins that function in regulating progression through the cell cycle. Since the successful division and replication of a cell is essential for its survival, the cell cycle is tightly regulated by several components to ensure the efficient and error-free progression through the cell cycle. In some specific embodiments, a gene encoding said Cyclin A may comprise the nucleic acid sequence as denoted by SEQ ID NO: 16.

Still further, the Iap1 (Inhibitor of apoptosis 1) gene is encoding for a protein that regulates apoptosis through its interaction with downstream TNF receptor effectors by binding to and inhibiting certain caspases, and by controlling the levels of specific proapoptotic stimuli (e.g., Smac/DIABLO) within the cell. In some specific embodiments, a gene encoding said IAP1 may comprise the nucleic acid sequence as denoted by SEQ ID NO: 17.

In yet some further embodiments, the Gpdh (Glycerol-3-phosphate dehydrogenase) encodes for an enzyme that catalyzes the reversible redox conversion of dihydroxyacetone phosphate to sn-glycerol 3-phosphate. Glycerol-3-phosphate dehydrogenase serves as a major link between carbohydrate metabolism and lipid metabolism. It is also a major contributor of electrons to the electron transport chain in the mitochondria. In some specific embodiments, a gene encoding said GPDH may comprise the nucleic acid sequence as denoted by SEQ ID NO: 18.

Thus, in certain embodiments, the heterogametic mosquito (male) of the invention, used by the systems and methods described herein, may comprise at least one gRNA specific for Cyclin A, at least one gRNA specific for Iap1, at least one gRNA specific for Gpdh. In yet some further embodiments, these gRNA sequences may be incorporated either to the X or to the Y gender chromosomes of the male mosquito.

It should be understood that any gene essential for the survival of the insect of the invention may be used as a target for the gRNAs encoded by the transgenic mosquitos of the invention. In some specific embodiments such essential genes may include the mosquito orthologs of any of the following Drosophila genes. More specifically, at least one of CG4238, CG4502, CG10473, CG1512(cul-2), CG12397(debcl), CG1782(Uba1), CG13343, CG8428(spin), CG6829(Ark), CG4943(lack), CG5370(Dcp-1), CG12284(th), CG7238(sip1), CG11326(Tsp), CG7123(LanB1), CG15288(wb), CG7527(DN-cad2), CG18464(sns), CG2040(hig), CG17579(sca), CG10145(mspo), CG8095(scb), CG3722(shg), CG5372(aPS5), CG3938(Cyc E), CG11397(glu), CG1772(dap), CG6191, CG5072(Cdk4), CG3510(Cyc B), CG5940(Cyc A), CG7682, CG3660, CG9884(oaf), CG2937(mRpS2), CG7380, CG5838(Dref), CG5722(NPC1), CG10718(neb), CG9342, CG17436, CG14592, CG1616(dpa), CG2368(psq), CG10897(tou), CG8964, CG12372(spt4), CG3905(Su(z)2), CG3886(Psc), CG4088(lat), CG8787(Asx), CG8153(mus210), CG8171(dup), CG5785(thr), CG5170(Dp1), CG9183(plu), CG9193(mus209), CG11716, CG4817(Ssrp), CG1070(Alhambra), Z50152(trx), CG7467, CG5499(His2Av), CG5061(capt), CG16858(vkg), CG4145(Cg25C), CG9553(chic), CG5972, CG8902, CG5958, CG4636, CG10846(dynactin-subunit-p25), CG13279(Cyt-b5-r), CG10954(Arc-p34), CG1768(dia), CG3265(Eb1), CG9446(coro), CG1708(cos), CG3451(rexin), CG18076(shot), CG18250(Dg), CG7765(Khc), CG9325(hts), CG9277(bTub56D), CG11312(insc), CG4254(tsr), CG15792(zip), CG2718(Gs1), CG3018(1wr), CG4749, CG7254(GlyP), CG7263, CG31690, CG3123, CG3523, CG3488, CG3326, CG3714, CG8890, CG7269(He125E), CG9171, CG9078(ifc), CG9535, CG9542, CG9547, CG31637, CG9261(Nrv2), CG10354, CG5261, CG8668, CG3881, CG4008(und), CG4600(yip2), CG4747, CG5395(nmd), CG5029(SamDC), CG5355, CG5353(Aats-thr), CG4501(bgm), CG3481(Adh), CG3688, CG3903, CG4993(PRL-1), CG4455, CG10621, CG17323, CG16784(pr), CG9247, CG9249, CG9244(Acon), CG31619, CG5922, CG3161(Vha16), CG1851, CG2064, CG12055(Gapdh1), CG18495(Prosa6), CG8723, CG15483, CG8732, CG8251(Pgi), CG8213, CG8181, CG8029, CG1827, CG1794(Mmp2), CG1519(Prosalpha7), CG2331(TER94), CG7712, CG7741, CG9006, CG8983(ERp60), CG8545, CG8776, CG3821(Aats-asp), CG4016, CG6016, CG13334, CG6543, CG12366(O-fut1), CG10117(ttv), CG8210(Vha14), CG8256, CG8392, CG8322(ATPCL), CG8421(Asph), CG8446, CG8048(Vha44), CG8938(Gst2), CG4827, CG5164(Gst3), CG17524, CG17527, CG17725(Pepck), CG15100, CG11007, CG30394, CG15669(MESK2), CG9858(c1t), CG6393, CG3612(b1w), CG3495(Gmer), CG2952(Dox-A3), CG3082, CG10330(bgcn), CG3725(Ca-P60A), CG3209, CG3333(Nop60B), CG4634(Nurf-38), CG4692, CG9047, CG10142(Ance-5), CG1004(rho), CG1009(psa), CG6661, CG6778, CG14637(abs), CG10279(Rm62), CG10272, CG4264(Hsc70-4), CG4261(He189B), CG31973, CG11376, CG3345, CG11555, CG11604, CG3943(kraken), CG5118, CG4764, CG4775, CG31666, CG31938, CG10908, CG15362, CG7291, CG15361, CG10874, CG9866, CG9867, CG4272, CG9641, CG3558, CG9660(toc), CG15414, CG17593, CG3921, CG3920, CG18013, CG12787, CG8886, CG31917, CG8895, CG4230, CG31651, CG14021, CG11030, CG31644, CG9016, CG9088(lid), CG9159(Kr-h2), CG9162, CG31641(stai), CG9536, CG9537(DLP), CG11050, CG11324, CG11188, CG13773, CG3430, CG18304, CG31907, CG4495, cuc, CG7224, CG7233, CG7392, CG31605, CG31756, CG17834, CG31886, CG9584, CG4036, CG5924, CG4539(Bka), CG31720, CG5385, CG13143, CG7456, CG6144, CG6700, CG4621, CG4738, CG4713, CG4751, bft, CG16969, CG31764, CG12292, CG9933, CG6043, CG7110, CG9239, BG:DS00929.16, CG16863, CG8954, CG3506 (vas), CG4180(1(2)35Bg), CG3975, CG18109, CG4930, CG13258, CG5968, CG5953, CG31817, CG17912, CG31782, CG7200, CG12750, CG10372(Faf), CG17321, CG10336, CG15168, CG10563, CG10528(fs(2)1toPP43), CG31692, CG10746(fok), CG10949, CG9318, CG9333, CG2614, CG9339, CG9340, CG9246, CG9252, CG8671, CG9243, CG8677, CG31617, CG3305, CG3278, CG1832, CG17706, Y13272(idr), CG14471, CG8276(bin3), CG8325, CG9397, CG30443, CG3268, CG30444, 1(2)01289, CG15242, CG12165(Incenp), CG1845, CG1624(dpld), CG1600, CG30491, CG30496, CG30497, CG12769, CG12770, CG8715, CG14757, CG8635, CG8740, CG30349, CG8229, CG30346, CG8069, CG30342, CG8055, CG1968, CG13954, CG1888, CG1814, CG12928, CG1623, CG30007, CG12128, CG2292, CG12912, CG12340, CG7699, CG18353, 1(2)k15826, CG13211, CG9035, CG9003, CG8998, CG13189, CG13178, CG13162, CG30055, CG8632, CG17574, CG4646, CG6061, CG17716(fas), CG6315(fl(2)d), CG18368, CG6209, CG6315(fl(2)d), CG10808(synaptogyrin), CG6329, CG8323, CG8531, CG8547, CG8561, CG8603, CG10155, CG10209, CG10220, CG11798, CG8092, CG8253, CG8370, CG8414, CG12711, CG10731, CG15707, CG15709, CG8306, CG5065, CG5859, CG5935, CG6426, CG15610, CG6657(veg), CG8963, CG30457, CG14478, CG6424, CG5109(Pc1), CG5733, CG16859, CG30118, CG12263, CG17533(Gst3-2), CG30332, CG5469, CG15092, CG30131, CG30132, CG18367, CG8920, CG13867, CG13432, CG13433, CG30149, CG30152, CG15666, CG15678, CG30403, CG30404, CG11296, CG10955, CG3624, CG4071, CG30193, CG3831, CG9896, CG5360, CG3941, CG11183, CG3735, CG3060, CG11390(PebIII), CG30173, CG13563, CG13589, CG13583, CG18510, CG3776, CG12851, CG2765, CG9358, CG14954, CG15001, CG8634, CG17667, CG7372, CG10712, CG2503, CG11745, CG12402, CG8927, CG4936, CG5315, CG6954, CG10365, CG6204, CG11839, CG17370, CG14066(larp), CG2245, CG18497(spen), CG4260, CG4114(ex), CG5574(lea), CG7074(mio), CG8814, CG17259, CG2774, CG8885, CG6944(Lam), CG9093(Tsp26A), CG11527(Tig), CG9543, CG3423(SA), CG13777, CG4494, CG6717, CG7851(Scga), CG18405(Sema-1a), CG3779(numb), CG5885, CG4535(FKBP59), CG4758(Trp1), CG4799(Pen), CG4995, CG6094, CG6743, CG4579, CG9828, CG5813(chif), CG17905, CG6605(BicD), CG5803(Fas3), CG10449(Catsup), CG2637(Fs(2)Ket), CG1099(Dap160), CG12792, CG8390(v1c), CG7843, CG12845(Tsp42Ef), CG11084(pk), CG17800(Dscam), CG1363(blow), CG30372, CG8639(Cirl), CG8739(cmp44E), CG14745(PGRP-SC2), CG8026, CG17870, CG12894, tRNA:T3:47F, CG7776(E(Pc), CG13180(jeb), CG16747(guf), CG6692(Cp1), CG10119(LamC), CG10149(Rpn6), CG10246(CYP6a9), CG10941(mm), CG6556(cnk), CG15077, CG5580(sbb), CG9847, CG3413(windpipe), CG5504(1(2)tid), CG4354(slbo), CG3416, CG11282(caps), CG3971(Baldspot), CG4289, CG7437(mub), SD9251, CG10328(NonA-1), CG10578(DnaJ-1), CG6383(crb), CG4963, CG2216(Fer1HCH), CG3727(dock), CG4385(S), CG3664(Rab5), CG10033(for), CG14026(tkv), CG13995, CG9493(Pez), CG4889(wg), CG4698(Wnt4), CG7109(mts), CG14472(poe), CG8222(Pvr), CG13383(Pp2A-29B), CG13388(Akap200), CG4379(Pka-C1), CG4904(Pros35), CG12314, CG7147(kuz), CG7393(p38b), CG7793(Sos), CG31811(cenG1A), CG4711(grp), CG7157(Acp36DE), CG17492, CG10641, CG17348(dr1), CG10334(spi), CG10628, CG10538(CdGAPr), CG10043(rtGEF), CG1864(Hr38), CG8681(clumsy), CG9242, CG11628, CG12110(p1d), CG7873(Src42A), CG3427, CG3572(vimar), CG1854(0r43a), CG1341, CG11546, CG2411(ptc), CG8261(Gg1), CG8224(babo), CG8068[Su(var)2-10], CG2049, CG2078(Myd88), CG8804(wun), CG8805(wun2), CG1916(Wnt2), CG11823(Hr46), CG2204(G-oalpha47A), CG13219(skf), CG8472(Cam), CG8581(fra), CG6033(drk), CG8118(mam), CG8553(Se1D), CG8166(unc-5), CG8212, CG8416(Rhol), CG6805, CG15609, CG4798, CG6530, CG5729(Dgp-1), CG10917(fj), CG5576(imd), CG15072, CG7097, CG11960(par-1), CG8896(18w), CG9985(sktl), CG10497(Sdc), CG10079(Egfr), CG9856(PTP-ER), CG9820(0r59a), CG3957, CG2835(G-sa60A), CG3204(Rap21), CG4012(gek), CG4589, CG8114(pb1), CG6827(Nrx), CG6235(tws), CG11502(svp), CG31196(14-3-3 e), CG6027(cdi), CG4733, CG4257(Stat92E), CG17077(pnt), CG1658(Doa), CG1395(stg), CG4033(RpI135), CG4427, CG2851(Gsc), CG3166(aop), CG8817(lilli), CG12399(Mad), CG14029(vri), CG18783(Kr-h1), CG11199(Liprin-a), CG7562(Trf), CG3998(zf30C), CG5102(da), CG4807(ab), CG14938(crol), CG5461(bun), CG7885(RpII33), CG4491(Noc), CG3497(Su(H)), CG3758(esg), CG7664(crp), CG5848(cact), CG6667(d1), CG10699(Lim 3), CG10719(brat), CG1071(E2f2), CG18362(Mio), CG1374(tsh), CG31612, CG1765(EcR), CG9403, CG15845(Adf1), CG8704(dpn), CG8643(rgr), CG12052(lola), CG6751, CG7734(shn), CG8815(Sin3A), CG3644(bic), CG3991(Tppii), CG4037(seq), CG4654(Dp), CG8367(cg), CG8151(Tfb1), CG10122(RpI1), CG5033, CG5058(grh), CG12767(Dip3), CG5738(lolal), CG9291(Elongin-C), CG10438, CG9415(Xbp1), CG9433(Xpd), CG9696(dom), CG10318(NC2a), CG5799(dve), CG5393(apt), CG5575(ken), CG4882, CG6854, CG7757, CG7405(Cyc H), CG7688(fru), CG7361(RfeSP), CG3057(colt), CG9663, CG3036, CG9539(Sec61a), CG3811, CG6647(porin), CG5304, CG12455, CG13281(Cas), CG12548, CG30437, CG3409, CG2140(Cyt-b5), CG10844(Rya-r44F), CG7777, CG30035, CG8996(wal), CG6119, CG10130(Sec61b), CG8291, CG8389, CG10939(Sipl), CG15095, CG4797, CG4324, CG11779, CG5670(Atpa), CG3696(kis), CG3582(U2af38), CG2807, CG4291, CG4258(dbe), CG3151(Rbp9), CG3605, CG3542, CG8846(Thor), CG15442(RpL27A), CG9124(e1F-3p40), CG9075(e1F-4a), CG10203(x16), CG10377(Hrb27C), CG4567, CG7424, CG13096, CG13109(tai), CG3949(hoip), CG5920(sop), CG4602, CG5352(SmB), CG31762(aret), CG6382(E1f), 1(2)10333, CG12396, CG4152(1(2)35Df), CG10302(bsf), CG10305(RpS26), CG10652(RpL30), CG10922(La), CG9253, CG2163(Pabp2), CG1821, CG12131, CG12921(mRpS32), CG8280(Efla48D), CG8427(SmD3), CG3845, CG6050(EfTuM), CG6671(AG01), CG8338(mRpS16), CG10228, CG8443, CG4878(eIF3-s9), CG4954(eIF3-S8), CG5119(pAbp), CG9854(hrg), CG9143, CG13425(b1), CG4266, CG9450(tud), CG3633(mRpS29), CG3751, CG4207(bonsai), CG3661(RpL17A), CG3186(e1F-5A), CG4806, CG2746(RpL19), CG4035(eIF-4E), CG10753(snRNP69D), CG16941, Z14974(cpo), CG17838, or any derivatives, orthologs and homologs thereof and any combinations thereof.

In yet some further embodiments, the system of the invention may be applicable for Aedes aegypti mosquito. More specifically, the dengue, yellow fever, chikungunya and zika vector, Aedes aegypti, has a dominant male-determining sex locus (M) on chromosome 1, for which males are heterozygous (Mm). This locus is primarily responsible for sex determination, however male and female chromosomes are also cytologically distinct. The male-determining factor (M factor) nix, an M-linked myosin heavy chain gene, myo-sex, and two sex determination transcription factors have been characterized but little else is known about the specific genes contributing dimorphic phenotypes in aedine mosquitoes. Thus, in some specific embodiments, since the male is the heterogametic organism, and the M gene may be equivalent to the heterogametic chromosome, where the mm homozygotes are equivalent to the homogametic organism (female). Thus, in some embodiments, at least one nucleic acid sequence encoding at least one guide RNA directed against at least one target sequence may be integrated into the m gene of the heterogametic mosquito (male). Such system may be used in some embodiments, for selecting a male progeny. In yet some other alternative embodiments, the at least one nucleic acid sequence encoding at least one guide RNA directed against at least one target sequence may be integrated into the M chromosome of the transgenic mosquito. Such system may be used in some embodiments to select for a female progeny. It should be appreciated that the present invention provides in additional aspects thereof any of the transgenic mosquitos described herein before, and in connection with other aspects of the invention.

Still further, some insects, like wasps, bees, butterflies and ants, pollinate flowering plants. Pollination is a mutualistic relationship between plants and insects. This greatly increases diversity in plants and significantly affects agriculture. The present invention provides systems and methods for determining insect gender and therefore may be of a high economic value, when applied for insects useful in agriculture. Of particular interest in some embodiments of the present invention is the bee. Bees are flying insects closely related to wasps and ants, known for their role in pollination and, in the case of the best-known bee species, the western honey bee, for producing honey and beeswax. Bees are a monophyletic lineage within the superfamily Apoidea and are presently considered a Glade, called Anthophila. There are nearly 20,000 known species of bees in seven recognized biological families, specifically, Andrenidae, Apidae, Colletidae, Halictidae, Megachilidae, Melittidae, Stenotritidae. Some species including honey bees, bumblebees, and stingless bees live socially in colonies. It should be understood that the present invention encompasses any of the bee species of any of the bee families indicated herein.

It should be understood that the present invention further provides any of the transgenic mosquitos, bees, or insects of the invention, any cells, progenies and embryos thereof, an any use or product thereof.

The present systems of the invention as well as the methods disclosed herein above offer great economic advantage for any industrial or agricultural use of animals, specifically, livestock. Thus, in some specific embodiments, the invention (systems, transgenic organisms and methods thereof) may be applicable for mammalian livestock, specifically those used for meat, milk and leather industries. Livestock are domesticated animals raised in an agricultural setting to produce labor and commodities such as meat, eggs, milk, fur, leather, and wool. The term includes but is not limited to Cattle, sheep, domestic pig (swine, hog), horse, goat, alpaca, lama and Camels. Of particular interest are cattle applicable in the meat and milk industry, as well as in the leather industry. More specifically, in certain embodiments, the transgenic animals of the invention, as well as the animals used by the systems and methods of the invention may be Cattle, colloquially cows, that are the most common type of large domesticated ungulates, that belong to the Bovidae family. The Bovidae are the biological family of cloven-hoofed, ruminant mammals that includes bison, African buffalo, water buffalo, antelopes, wildebeest, impala, gazelles, sheep, goats, muskoxen. The biological subfamily Bovinae includes a diverse group of ten genera of medium to large-sized ungulates, including domestic cattle, bison, African buffalo, the water buffalo, the yak, and the four-horned and spiral-horned antelopes. Of particular interest in the present invention may be the domestic cattle are the most widespread species of the genus Bos, and are most commonly classified collectively as Bos taurus. More specifically, Bos is the genus of wild and domestic cattle. Bos can be divided into four subgenera: Bos, Bibos, Novibos, and Poephagus. Subgenus Bos includes Bos primigenius (cattle, including aurochs), Bos primigenius primigenius (aurochs), Bos primigenius taurus (taurine cattle, domesticated) and Bos primigenius indicus (zebu, domesticated).

In some embodiments, the cattle may be cows, specifically, domestic cows and the at least one nucleic acid sequence encoding or forming at least one guide RNA directed against at least one target sequence may be incorporated in the female (the homogametic subject, XX) and the nuclease (e.g., Cas9, or any derivative, mutant or fusion protein thereof) may be integrated by the male subject (the heterogametic organism, XY). In yet some other alternative embodiments, the at least one nucleic acid sequence encoding or forming at least one guide RNA directed against at least one target sequence may be incorporated in the male (the heterogametic subject) and the nuclease (e.g., Cas9, or any derivative, mutant or fusion protein thereof) may be integrated in the female subject (the homogametic organism).

In a more specific embodiment, the cattle may be a cow and the at least one nucleic acid sequence encoding at least one guide RNA directed against at least one target sequence may be integrated into the X chromosome of said cow, specifically, heterogametic cow. Alternatively, the least one nucleic acid sequence encoding at least one guide RNA directed against at least one target sequence may be integrated into the Y chromosome of the cow. In some further embodiments, the at least one nucleic acid sequence encoding at least one guide RNA directed against at least one target sequence may be integrated into the X chromosome of the cow, specifically, the heterogametic cow. Such system may be used for selecting for male progeny. In yet some alternative embodiments, the at least one nucleic acid sequence encoding at least one guide RNA directed against at least one target sequence may be integrated into the Y chromosome of the cow. Such system may be use in some embodiments to select for female progeny.

In some specific and non-limiting embodiments, the at least one target sequence may be any sequence encoding or controlling the expression of a product essential for embryogenesis, survival or development of cows. Non-limiting examples for such product include but are not limited to any cow ortholog of any of the mouse essential genes specified herein above in connection with the rodent transgenic organisms of the invention. It should be appreciated that the present invention provides in additional aspects thereof any of the transgenic mammalian livestock described herein before, and in connection with other aspects of the invention. In yet some further specific embodiments, the invention provides transgenic male cow comprising at least one sequence forming or encoding at least one gRNA directed against at least one essential gene. Such transgenic sequences may be incorporated in some embodiments into the X chromosome of said male cow. In yet some further embodiments, such sequences may be incorporated into the Y chromosome thereof. It should be noted that any cell, specifically, zygote cell (e.g., sperm or semen) of said transgenic cow or any progeny or product thereof, are also encompassed by the invention. It should be also understood that the invention further pertains to any use of the transgenic livestock disclosed by the invention or any product thereof. Still further, in some embodiments, the invention provides transgenic male cow comprising at least one sequence encoding at least one nuclease (e.g., Cas9, or any derivative, mutant or fusion protein thereof). Such transgenic sequences may be incorporated in some embodiments into the X chromosome of said male cow. In yet some further embodiments, such sequences may be incorporated into the Y chromosome thereof. In yet some further specific embodiments, the invention provides transgenic female cow comprising at least one sequence forming or encoding at least one gRNA directed against at least one essential gene. Such transgenic sequences may be incorporated in some embodiments into any chromosome of such female cow. Still further, in some embodiments, the invention provides transgenic female cow comprising at least one sequence encoding at least one nuclease (e.g., Cas9, or any derivative, mutant or fusion protein thereof). Such transgenic sequences may be incorporated in some embodiments into any chromosome of such female cow.

As mentioned above, the systems of the invention concerns any eukaryotic organism and as such may be also applicable for members of the biological kingdom Plantae. Particularly, in certain plants that display heterogametic gender determination. Thus, in some further embodiments, the eukaryotic heterogametic organism and homogametic organism of the system of the invention may be of the biological kingdom Plantae. In more specific embodiments, the organisms may be a dioecious plant. More specifically, plants presenting biparental reproduction. In dioecious plants the male and female reproductive systems occur on separate plants. While both plants produce flowers, one plant has the male reproductive parts and the other plant has the female parts. This is unlike a monoecious plant, which has both male and female flowers.

In more specific embodiments, the dioecious plant may be of the family Cannabaceae. In some specific embodiments, the plant of the family Cannabaceae may be any one of Cannabis (hemp, marijuana) and Humulus (hops). and the at least one nucleic acid sequence encoding at least one guide RNA directed against at least one target sequence may be integrated into the X chromosome of the heterogametic plant or the at least one nucleic acid sequence encoding at least one guide RNA directed against at least one target sequence may be integrated into the Y chromosome of said plant. In more specific embodiments, the plant of the family Cannabaceae may be Cannabis (hemp, marijuana). Thus, in some embodiments, the invention provides a system comprising at least one heterogametic transgenic plant and at least one homogametic transgenic Cannabis plant. In some specific embodiments, at least one nucleic acid sequence encoding at least one guide RNA directed against at least one target sequence may be integrated into the X chromosome of the heterogametic Cannabis plant. In certain embodiments, such system may be used for selection of a male progeny. In yet some alternative embodiments, at least one nucleic acid sequence encoding at least one guide RNA directed against at least one target sequence may be integrated into the Y chromosome of the heterogametic Cannabis plant. Such system may be useful in some embodiments for selecting a female progeny.

More specifically, Cannabis is a dioecious plant producing either male or female reproductive organs i.e., cannabis grows as either a male or female plant. Removing male plants from crop allows female plants to grow large, an unfertilized flower named sensimilla. Sensimilla is a highly concentrated type of cannabis and does not contains seeds. Sensimilla refers to many strains of marijuana where the female plant is allowed to only produce flowers, but is left unfertilized so does not progress on to produce seeds. Its unfertilized state contributes to the plant's ability to produce higher levels of tetrahydrocannabinol (THC) and other cannabinoids.

Currently, one way to obtain only female plant is by using feminized cannabis seeds. These seeds are produced by causing the monoecious, or hermaphrodite condition in a female cannabis plant. This is achieved through several methods such as spraying the plant with a solution of colloidal silver, or with gibberellic acid. Feminized seeds produce plants that are nearly identical to this self-pollinated female parent plant, as only one set of genes is present and will not produce any male plants. Another way is by collecting pollens may for fertilizing other females. In both cases, only feminized seeds will be produced because only the X chromosome is found in the pollen and the female gamete. However, most feminized seeds end up being hermaphrodites, which results in flowers possessing seeds and reduces yields. Genetically, the cannabis plant gender is regulated by two chromosomes the X and Y chromosomes. A plant with two X chromosomes becomes female. A plant with an X and Y chromosome turns into a male.

In yet some further embodiments, the plant of the family Cannabaceae may be Humulus (hops). Thus, in some embodiments, the invention provides a system comprising at least one heterogametic transgenic plant and at least one homogametic transgenic Humulus plant. In some specific embodiments, at least one nucleic acid sequence encoding at least one guide RNA directed against at least one target sequence may be integrated into the X chromosome of the heterogametic Humulus plant. In certain embodiments, such system may be used for selection of a male progeny. In yet some alternative embodiments, at least one nucleic acid sequence encoding at least one guide RNA directed against at least one target sequence may be integrated into the Y chromosome of the heterogametic Humulus plant. Such system may be useful in some embodiments for selecting a female progeny. More specifically, Hops are the flowers (also called seed cones or strobiles) of the hop plant Humulus lupulus. They are used primarily as a flavoring and stability agent in beer, to which they impart bitter, zesty, or citric flavors; though they are also used for various purposes in other beverages and herbal medicine. Male and female flowers of the hop plant usually develop on separate plants (dioecious plant), although fertile monoecious individuals appear occasionally. Because viable seeds are undesirable for brewing beer, only female plants are grown in hop fields, thus preventing pollination. Female plants are propagated vegetatively, and male plants are culled if plants are grown from seeds. The plant Humulus lupulus has heteromorphic sex chromosomes is hop. The genotypes carrying XX or XY chromosomes correspond to female and male plants, respectively.

It should be understood that the invention provides in further aspects thereof any of the transgenic organisms described herein, specifically, any animal or plant organism described herein, any cell thereof, specifically, zygote cell (e.g., ovum or sperm), any progeny thereof (either viable or non-viable), any product or components thereof, as well as any use of said organism, cell or progeny or any parts or components thereof.

The systems of the invention as detailed above enable to perform methods for selection of gender which are of crucial relevance in the agriculture, as well as in the aquaculture and farm industry. In addition, these methods further enable the control of particular population of undesired/problematic or alternatively, desired organisms that display a desired trait. Thus, in a second aspect, the invention relates to a method for selecting a desired gender of an eukaryotic organism. More specifically, the method may comprise the steps of: In a first step (A), providing a transgenic eukaryotic heterogametic organism comprising either at least one nucleic acids modifier protein, specifically, a nuclease (or a fragment thereof) or alternatively, a target recognition for such nucleic acids modifier protein, specifically, nuclease (or optionally, in addition to such target recognition element, the transgenic organism may comprise a fragment or domain of the respective nuclease). More specifically, the heterogametic transgenic organism provided by the method of the invention may comprise in some embodiments (a), at least one nucleic acid sequence. Such sequence may be in some embodiments (i), at least one sequence encoding or forming at least one target recognition element for at least one nucleic acids modifier protein (e.g., nuclease). In yet some further embodiments, the nucleic acid sequence may be (ii), at least one sequence encoding or forming the at least one target recognition element and in addition, a nucleic acid sequence encoding a first fragment, domain or subunit of at least one said nucleic acids modifier protein (e.g., nuclease).

In yet some further alternative embodiments, the transgenic heterogametic organism provided in step (A) may comprise (b), at least one nucleic acid sequence encoding: either at least one nuclease; or alternatively, (ii) a second fragment, domain or subunit of said at least one nucleic acids modifier protein (e.g., nuclease).

It should be noted that the nuclease used by the methods of the invention may be active only in the presence of the first and second fragments or subunits thereof. As defined herein before in connection with the systems of the invention “activity” of the nucleic acids modifier protein, specifically, nuclease used by the invention may include in some embodiments, a nucleolytic activity, or any of the activities specified herein before in connection with other aspects of the invention, however, it should be understood that in some embodiments, specifically when an inactive nucleic acids modifier protein, for example, an inactive nuclease is used, or any fusion proteins thereof, “activity” may further refer to any targeted manipulation of the expression of a target gene (e.g., either repression or enhancement).

Still further, it should be noted that the nucleic acid sequence may be integrated into one of the gender-chromosomes of the transgenic heterogametic organism provided in step (A).

The next step (B), involves providing a transgenic eukaryotic homogametic organism comprising one of a nucleic acids modifier protein, specifically, nuclease (or a fragment thereof) or alternatively, a target recognition for such nucleic acids modifier protein, e.g., nuclease, (or optionally, in addition to such target recognition element, the transgenic organism may comprise a fragment or domain of the respective nucleic acids modifier protein, e.g., nuclease). Thus, in some specific embodiments (a), the homogametic transgenic organism provided by the method of the invention may comprise at least one nucleic acid sequence encoding either (i) at least one nucleic acids modifier protein (e.g., nuclease), or (ii), a first fragment, domain or subunit of said at least one nucleic acids modifier protein (e.g., nuclease).

Alternatively, in some embodiments, the homogametic transgenic organism provided by the invention may comprise (b), at least one nucleic acid sequence. Such sequence may be either (i) a sequence encoding or forming at least one target recognition element for at least one nucleic acids modifier protein (e.g., nuclease); or (ii) a sequence encoding or forming the at least one target recognition element and a nucleic acid sequence encoding a first fragment, domain or subunit of at least one the nucleic acids modifier protein (nuclease).

It should be noted that also here, when fragments, subunits or domains of the nucleic acids modifier protein (e.g., nuclease) are used, in some embodiments, the nucleic acids modifier protein may be active only in the presence of the first and second fragments or subunits thereof. It should be appreciated that in case of nuclease that may be in some embodiments the modifier protein used by the methods of the invention, “activity” as used herein encompasses either a nucleolytic activity or any other non-nucleolytic activity of the nuclease or any fusion protein thereof. Still further, in some embodiments, the nucleic acid sequence may be integrated into at least one allele of any chromosomal or mitochondrial DNA of the transgenic homogametic organism provided in step (B). The next step (C), involves breeding the transgenic heterogametic organism provided in step (A) with the transgenic homogametic organism provided in step (B), thereby obtaining a progeny that is predominantly composed of one desired gender.

It should be noted that the nucleic acids modifier protein may be any protein, polypeptide or any complex or fusion protein thereof that affects or modify a nucleic acid sequence. In certain embodiments, such modifier may affect the expression, stability or activity of a product encoded by or regulated by the modified nucleic acid sequence. In some embodiments, such nucleic acids modifier protein may be at least one of; a nuclease, a methylase, a methylated DNA binding factor, a transcription factor, a chromatin remodeling factor, a polymerase, a demethylase, an acetylase, a deacetylase, a kinase, a phosphatase, an integrase, a recombinase, a ligase, a topoisomerase, a girase and a helicase. In yet some further embodiments, such nucleic acids modifier protein may be a nuclease. As indicated in connection with the systems of the invention, the “nuclease” as used herein encompasses a protein that display a nucleolytic activity, as well as any mutant or variant that display a reduced or no nucleolytic activity (e.g., defective or catalytically dead nuclease) and any fusion protein thereof. It should be further understood that all fusion proteins discussed above in connection with the systems of the invention may be also applicable for this aspect as well, specifically for a method for selection for a desired gender. It should be noted that the following embodiments refer to nuclease as the nucleic acids modifier protein, however, may be also applicable for any of the nucleic acids modifier proteins discussed above.

Thus, in some specific embodiments, the nuclease encoded by the transgenic heterogametic or homogametic organisms used by the methods of the invention may be at least one of: (i) a nuclease having a nucleolytic activity; (ii) a non-active nuclease and/or a fusion protein thereof, or alternatively (iii) any fragment, domain or subunit of the nuclease of (i) or the inactive nuclease of (ii) or of any fusion protein thereof. Still further, it should be understood that all guided and non-guided nucleases discussed in connection with the systems of the invention, specifically, classical or non-classical restriction enzymes, TALEN, ZFN or any fragments, domains, subunits or any fusion proteins thereof or any protein complex comprising the same, may be also applicable for the methods of the invention that is some embodiments, use any of the systems of the invention described above.

More specifically, in some further embodiments and as detailed for the systems of the invention, the nuclease may be at least one restriction enzyme. In yet some further embodiments, the target recognition element may be a restriction site of the enzyme within at least one of coding and non-coding sequences of at least one chromosomal or mitochondrial DNA of the organism. As noted above, the invention further encompasses the option of using nucleases that target and cut RNA molecules, for example, RNAzyme or any artificial or natural ribonuclease. In some embodiments, the target recognition element may be located (either endogenously or exogenously) within a specific gene but also could be within a repetitive coding/none coding region.

In some specific embodiments, the nuclease may be any one of a classical restriction enzyme (restriction site of up to 10 bp), homing endonucleases such as I-SceI with longer restriction sites (18 bp), a TALEN, a ZFN or any combinations thereof. In some embodiments, the target recognition element may be an endogenous sequence present in both the heterogametic and homogametic organism. In such case, the nuclease is split between the homogametic and heterogametic organism (e.g., a first fragment, subunit or domain in one organism and a second fragment, subunit or domain in the other organism).

In more specific embodiments for such case, the nuclease may be any one of a classical restriction enzyme, a TALEN or a ZFN, the nucleic acid sequence encoding or forming the target recognition element may be the restriction site, TALEN or ZFN recognition site that may be present in both the heterogametic and homogametic organism. In such case, the homogametic organism for example, may comprise a nucleic acid sequence encoding a first fragment, domain or subunit of the nuclease whereas the heterogametic organism may comprise a nucleic acid sequence encoding a second fragment, domain or subunit of the nuclease and vice versa.

In yet some other alternative embodiments, the nuclease may be at least one guided nuclease, for example, TALEN or ZFN, or alternatively, a restriction enzyme having a restriction site of 10 nucleotides or more. In such case, in some embodiments the target recognition element (with or without a fragment, subunit or domain of the respective nuclease or any mutant or fusion protein thereof) may be inserted, incorporated or integrated into one of the transgenic organisms used and provided by the methods of the invention (either the heterogametic or homogametic organisms), and the other transgenic organism (either the homogametic or the heterogametic) may comprise nucleic acid sequence encoding the respective nuclease or any non-active mutant or fusion protein thereof or any fragment, domain or subunits thereof.

In yet some further embodiments, the target recognition element may be incorporated into the chromosomal or mitochondrial DNA of either heterogametic/homogametic organism according to the methods of the invention.

In some embodiments, the at least one target recognition element of the method of the invention may be at least one nucleic acid sequence encoding at least one guide RNA directed against at least one target sequence within at least one of coding and non-coding sequences or any product/s thereof of at least one chromosome of the organism. In such embodiments, the nuclease may be at least one RNA guided DNA binding protein nuclease. It should be understood that “organism” as indicated herein, refer in some embodiments to an organism of the same species. In yet some further embodiments, “organism” as indicated herein means the embryonic progeny of the transgenic organism used by the invention. Such embryo may be an embryo at any embryonic stage. It should be noted that also born progeny, either vital or non-vital, are also encompassed herein as an organism. Thus, in the presence of nuclease and guide RNAs directed to essential coding or non-coding nucleic acid sequences in the embryo (of the undesired gender, if containing both elements, the nuclease and the gRNAs), the destruction of the target sequences by the nuclease will be lethal to the embryo at any stage and even after birth.

In some specific embodiments, the methods of the invention may use RNA guided nucleases. Thus, in more specific embodiments, the method of the invention may comprise the steps of:

In a first step (a), providing a transgenic eukaryotic heterogametic organism comprising at least one nucleic acid sequence encoding at least one guide RNA directed against at least one target sequence. Alternatively, the heterogametic organism may comprise at least one nucleic acid sequence encoding at least one RNA guided DNA binding protein nuclease, or any fragments, domains, or subunits thereof or any non-active variant or mutant thereof and any fusion protein comprising the same. It should be noted that in some embodiments, the nucleic acid sequence may be integrated into one of the gender-chromosomes of the transgenic heterogametic organism.

In the next step (b), providing a transgenic eukaryotic homogametic organism comprising at least one nucleic acid sequence encoding at least one RNA guided DNA binding protein nuclease or any fragments, domains, or subunits thereof or any non-active variant or mutant thereof and any fusion protein comprising the same. Alternatively, the homogametic organism may comprise at least one nucleic acid sequence encoding at least one guide RNA directed against at least one target sequence. It should be noted that the nucleic acid sequence may be integrated into at least one allele of any chromosomal or mitochondrial DNA of the transgenic homogametic organism.

The next step (c), involves breeding said transgenic heterogametic organism provided in step (a) with said transgenic homogametic organism provided in step (b), thereby obtaining a progeny predominantly composed of the one desired gender.

In some embodiments, when the heterogametic organism of (a) comprises at least one nucleic acid sequence encoding at least one guide RNA directed against at least one target sequence, the homogametic organism of (b) comprises at least one nucleic acid sequence encoding at least one RNA guided DNA binding protein nuclease. In yet another alternative embodiment, when the heterogametic organism of (a) comprises at least one nucleic acid sequence encoding at least one RNA guided DNA binding protein nuclease, the homogametic organism of (b) comprises at least one nucleic acid sequence encoding at least one guide RNA directed against at least one target sequence.

In certain and particular embodiments, the invention provides a method comprising the steps of: First step (a) involves providing a transgenic eukaryotic heterogametic organism comprising at least one nucleic acid sequence encoding at least one guide RNA directed against at least one target sequence (the target sequence may be either DNA or any product/s thereof, for example, RNA). It should be noted that such nucleic acid sequence may be integrated into one of the gender-chromosomes of said transgenic heterogametic organism.

In a second step (b), providing a transgenic eukaryotic homogametic organism comprising at least one nucleic acid sequence encoding at least one RNA guided DNA binding protein nuclease. It should be noted that the nucleic acid sequence may be integrated into at least one allele of any chromosomal or mitochondrial DNA of said transgenic homogametic organism.

In the next step (c), breeding said transgenic heterogametic organism provided in step (a) with said transgenic homogametic organism provided in step (b), thereby obtaining a progeny predominantly composed of said one desired gender.

As used herein the term “predominantly” refers to a progeny wherein is the selected gender represents 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, or 100% of the total progeny. In one embodiment, the transgenic heterogametic organism of (a) and the transgenic homogametic organism of (b) are of the same species.

In other embodiments, the method of the invention may be useful for:

In some embodiments (a), for selection towards the homogametic gender. In such case, the at least one nucleic acid sequence encoding at least one guide RNA directed against at least one target sequence (either DNA or RNA), may be integrated into the gender-chromosome specific for the heterogametic gender of the transgenic heterogametic organism.

In yet some further embodiments (b), the method may allow selection towards the heterogametic gender. In such case, the at least one nucleic acid sequence encoding at least one guide RNA directed against at least one target sequence may be integrated into the gender-chromosome specific for the homogametic gender of the transgenic heterogametic organism.

In some embodiments, the at least one nucleic acid sequence encoding at least one RNA guided DNA binding protein nuclease may be integrated into the two alleles of any chromosome of the homogametic organism provided in step (b), thereby obtaining a progeny exclusively composed of one gender. As used herein the term “exclusively” refers to a progeny wherein is the selected gender represents 100% of the total progeny.

It should be noted that in some embodiments, the transgenic heterogametic organism of (a) and the transgenic homogametic organism of (b) used and provided by the methods of the invention may be of the same species. Simply put, in some embodiments of the method of the invention, where a heterogametic organism that carry at one of its gender chromosomes (either the chromosome specific for the heterogametic gender, if this is the undesired gender, or gender chromosome of the homogametic gender, if the homogametic gender is the undesired gender), a nucleic acid sequence encoding at least one guide RNA directed against essential genes or products as discussed herein before, and a homogametic organism that carry nucleic acid sequence encoding at least one nuclease, are provided by the method of the invention, breeding of both transgenic organisms (as in step C), will result viable progenies of only the desired gender. More specifically, an embryo of the undesired gender will comprise gRNAs from the heterogametic parent and nuclease from the homogametic gender that together will direct destruction of the target essential genes that results in lethality of such embryo. It should be noted that lethality may occur at any stage of the embryonic development, and in some cases even several days after birth. An embryo of the desired gender will carry only the nuclease provided by the homogametic parent, and the gender chromosome of the desired gender from the heterogametic parent that does not carry any nucleic acid sequence encoding gRNA, and therefore will develop normally into a viable offspring. It should be understood that in other alternative embodiments the method may be performed at the same manner also in case nucleic acid sequence encoding the nuclease are provided by the heterogametic organism, and nucleic acid sequences providing the gRNAs are provided by the homogametic parent.

In further specific embodiments, the transgenic heterogametic organism provided in step (a) of the method of the invention, may further comprise a nucleic acid sequence encoding at least one guide RNA directed against at least one gene or any product/s thereof, encoding a product determining, or product essential for, an undesired trait, or alternatively, a desired trait. Such nucleic acid sequence may be integrated in some embodiments into the other gender-chromosomes of the transgenic heterogametic organism.

The step of further selection of the desired gender that carry a desired trait, or alternatively do not carry an undesired trait, will be described in more detail in connection with other aspects of the invention. It should be understood, that such description is also applicable in the present aspect. In more specific embodiment, the undesired trait may be related to fertility. Thus, according to such embodiments, the invention may provide methods for gender selection and in some embodiments methods for selecting organisms of a particular gender with a specific modulated trait. Selection for a specific gender with or without a further modification may be particularly suitable for controlling animal populations, live-stock as well as animal-based research studies. In more specific embodiments, the RNA guided DNA binding protein nuclease of the method of the invention may be any one of CRISPR Class 2 or Class 1. In some more specific embodiments, the RNA guided DNA binding protein nuclease may be of a CRISPR Class 2 system. More specifically, such CRISPR Class 2, may be a CRISPR type II system. In some further more specific embodiments, the RNA guided DNA binding protein nuclease used by the methods of the invention may be Cas9, or any fragments, domains, or subunits thereof or any non-active variant or mutant thereof and any fusion protein comprising the same. As indicated above, the systems and methods of the invention may further encompasses the use of nucleases that cut RNA. Thus, in some embodiments, guided RNA nucleases that may be used by the invention may be CRISPR-Cas systems that target RNA, and can be advantageous (e.g., CRISPR-Cas Type III and VI).

As indicated above, methods for gender selection may be useful in farm animals conferring huge economic advantages. Thus, in certain embodiments, the method of the invention may be applicable for gender selection of eukaryotic organisms of the biological kingdom Animalia. In yet some further embodiments, the eukaryotic heterogametic organism and homogametic organism of the method of the invention may be of the biological kingdom Animalia. In more specific embodiments, the organisms of the method of the invention may be any one of a vertebrate or an invertebrate.

In more specific embodiments, the organism of the method of the invention may be any one of a mammal, an avian, an insect, a fish, an amphibian, a reptile, a crustacean, a crab, a lobster, a snail, a clam, an octopus, a starfish, a sea-urchin, jellyfish, and worms. In yet some further specific embodiments, the eukaryotic organisms may be mammalian organisms of any of the mammalian orders disclosed above in connection with other aspects of the invention. As mentioned previously concerning the systems of the invention, rodents are the most popular animal model in research. Therefore, in some particular fields of research males or females may be preferred and methods for gender selection enables to produce only the relevant gender (cost effective as well as reducing animal misery). In more specific embodiments, the mammal may be a rodent. In some specific embodiments, the rodent may be a mouse. In yet some further specific embodiments (a) for selecting for males, the at least one nucleic acid sequence encoding at least one guide RNA directed against at least one target sequence may be integrated into the X chromosome of the heterogametic mouse (that may be in some embodiments, male). In yet some further embodiments (b), for selecting for females, the at least one nucleic acid sequence encoding at least one guide RNA directed against at least one target sequence may be integrated into the Y chromosome of the heterogametic mouse (male).

In some specific and non-limiting embodiments, the at least one target may be a gene essential for embryogenesis, survival or development. In yet some specific embodiments, a gene essential for embryogenesis may be any one of Atp5b (ATP synthase subunit beta, mitochondrial), Cdc20 (cell-division cycle protein 20), and Casp8 (Caspase-8). It should be noted that any essential gene as disclosed above in connection with other aspects of the invention are also applicable in connection with the present aspect.

In yet some further specific embodiments at least one nucleic acid sequence (spacer) encoding the guide RNAs directed against at least one gene or any product/s thereof (e.g., RNA) essential for embryogenesis may comprise the nucleic acid sequence as denoted by SEQ ID NO:1, SEQ ID NO:2 or SEQ ID NO:3. In yet some further alternative or additional embodiments, the target sequence may be sequences appearing in non-coding regions of at least one chromosomes. Cleavage of such sequences by the targeted Cas9, leads to inability of repair and/or destruction of the chromosome, thereby leading to death of the embryo. In yet some further embodiments, such targeted cleavage leads to specific knock out of a target gene essential for embryogenesis, survival or viability of the embryo. In yet some further embodiments, where a non-active nuclease, specifically, catalytically dead Cas9 (dCas9) is used, or any fusion protein thereof with activator or repressor, activation or alternatively, repression of essential genes may result in defective or impaired development of an embryo of the undesired gender.

In yet some additional embodiments, the methods for gender selection in accordance with the invention may be applicable for avian organisms. In yet some more specific embodiments, methods for gender selection in birds are also contemplates by the methods of the invention.

Thus, in some embodiments, the avian organism of the method of the invention may be any one of a domesticated and an undomesticated bird. In some specific embodiments, such avian organism may be any one of a poultry or a game bird. In some specific embodiments, the avian organism may be of the order Galliformes which comprises chickens, quails and turkeys).

More particularly, in the poultry industry, methods for gender selection are of particular relevance both in an economical and ethical point of view. Currently, culling male chicks post-hatch creates a major dilemma. Thus, in some specific embodiments, the methods of the invention may be applicable for domesticated bird that may be in some embodiments a chicken. More specifically, in some embodiments (a), for selecting for males, the at least one nucleic acid sequence encoding at least one guide RNA directed against at least one target sequence may be integrated into the W chromosome of the heterogametic chicken (female) provided by the methods of the invention. In yet some further embodiments (b), for selecting for females, the at least one nucleic acid sequence encoding at least one guide RNA directed against at least one target sequence may be integrated into the Z chromosome of the heterogametic chicken (female), provided by the method of the invention.

In some specific and non-limiting embodiments, the at least one target may be a gene essential for embryogenesis, survival or development. In yet some specific embodiments, a gene essential for embryogenesis may be any one of Casp8, Atp5b (ATP synthase subunit beta, mitochondrial) and Cdc20 (cell-division cycle protein 20). It should be noted that also IHH (Indian Hedgehog) may be used as a target.

In yet some further specific embodiments at least one nucleic acid sequence (spacer) encoding said guide RNAs directed against at least one essential gene for embryogenesis (or any product/s thereof) may comprise the nucleic acid sequence as denoted by SEQ ID NO:8, SEQ ID NO:9 or SEQ ID NO:10. In yet some further alternative or additional embodiments, the target sequence may be sequences appearing in non-coding regions of at least one chromosomes. Cleavage of such sequences by the targeted Cas9, leads to inability of repair and/or destruction of the chromosome, thereby leading to death of the embryo. The alternative of targeted repression or activation of target genes, achieved by the use of a fusion protein of a defective, catalytically inactive nuclease, such as dCas9, with a repressor or activator is also applicable in the disclosed methods for gender selection in chickens.

It should be understood, that in case of avian gender selection, breading the transgenic male and female avian organisms of the invention results in a fertilized egg laid by the transgenic avian female that will be in some embodiments, developed into a viable embryo that upon completion of its embryonic development will hatch and develop into an avian organism of the desired gender. Alternatively, in case of an embryo of the undesired gender, the fertilized egg laid by the transgenic avian female will contain a non-viable embryo, that its embryonic development has stopped at any embryonic stage. In most embodiments, such egg will stay an unhatched egg. As noted above, the methods of the invention may be applicable for fertilized unhatched eggs. The term “fertilized egg” refers hereinafter to an egg laid by a hen wherein the hen has been mated by a rooster within two weeks, allowing deposit of male sperm into the female infundibulum and fertilization event to occur upon release of the ovum from the ovary. “Unhatched egg” as used herein, relates to an egg containing an embryo (also referred to herein as a fertile egg) within a structurally integral (not broken) shell. It should be understood that unhatched egg, as used herein refer either to an egg containing a vital avian embryo of the desired gender, or alternatively a non-vital embryo of the undesired gender.

It should be understood that the invention further encompasses any viable avian subject of the desired gender that are progenies of the transgenic female and/or male avian subject, and in more specific embodiments, were hatched from an egg laid by the transgenic female that has been fertilized by the transgenic avian male of the invention. The invention further encompasses any eggs or offspring of such avian subjects of the desired gender and any uses thereof. Still further, it should be understood that the invention further pertains to any of the unhatched eggs that contain an embryo of the undesired gender and any use of such eggs and any parts or components thereof. More specifically, it should be noted that in some embodiments, in a fertilized egg laid by the transgenic avian female of the invention, an avian embryo of an undesired gender may comprise gRNAs that are directed against genes or products essential for embryonic development or survival, as well as the associated nuclease (e.g., Cas9), provided from the maternal and paternal transgenic avian subjects of the invention. In such case, destruction of essential genes may be lethal to the embryo or a progeny, and as such, may result in an egg containing an undeveloped non-vital avian embryo, or alternatively an embryo that die several days after hatching. It should be noted that such inviable embryo may be of any stage of embryonic development. It should be noted that “Embryonic development stage or step of avian embryo”, as used herein refers to the stage of day 1 wherein the germinal disc is at the blastodermal stage and the segmentation cavity takes on the shape of a dark ring; the stage of day 2 wherein the first groove appears at the center of the blastoderm and the vitelline membrane appears; the stage of day 3 wherein blood circulation starts, the head and trunk can be discerned, as well as the brain and the cardiac structures which begins to beat; the stage of day 4 wherein the amniotic cavity is developing to surround the embryo and the allantoic vesicle appears; the stage of day 5 wherein the embryo takes a C shape and limbs are extending; the stage of day 6 wherein fingers of the upper and lower limbs becomes distinct; the stage of day 7 wherein the neck clearly separates the head from the body, the beak is formed and the brain progressively enters the cephalic region; the stage of day 8 wherein eye pigmentation is readily visible, the wings and legs are differentiated and the external auditory canal is opening; the stage of day 9 wherein claws appears and the first feather follicles are budding; the stage of day 10 wherein the nostrils are present, eyelids grow and the egg-tooth appears; the stage of day 11 wherein the palpebral aperture has an elliptic shape and the embryo has the aspect of a chick; the stage of day 12 wherein feather follicles surround the external auditory meatus and cover the upper eyelid whereas the lower eyelid covers major part of the cornea; the stage of day 13 wherein the allantois becomes the chorioallantoic membrane while claws and leg scales becomes apparent; the stage of days 14 to 16 wherein the whole body grows rapidly, vitellus shrinking accelerates and the egg white progressively disappears; the stage of day 17 wherein the renal system produces urates, the beak points to the air cell and the egg white is fully resorbed; the stage of day 18 wherein the vitellus internalized and the amount of amniotic fluid is reduced; the stage of day 19 wherein vitellus resorption accelerates and the beak is ready to pierce the inner shell membrane; the stage of day 20 wherein the vitellus is fully resorbed, the umbilicus is closed, the chick pierces the inner shell membrane, breathes in the air cell and is ready to hatch; the stage of day 21 wherein the chick pierces the shell in a circular way by means of its egg-tooth, extricates itself from the shell in 12 to 18 hours and lets its down dry off.

Still further, in yet some further aspects thereof, the invention encompasses any egg derived, laid or fertilized by at least one of any of the transgenic avian subjects or animals of the invention, or by any progeny thereof, any component or any parts thereof or any product comprising said egg, components or parts thereof.

Still further, it should be noted that the present invention further encompasses any egg product or any product that contains or prepared using the eggs laid by the transgenic avian subjects of the invention or any components thereof (e.g., egg parts, specifically, egg shell, membrane, white and yolk, as well as any proteins, lipids or any substances comprised therein), or prepared by a process involving or using any of the eggs of the invention or any components thereof.

The term “egg products” refers to any product/s obtained from eggs, from their different components or blends, once the shell and membranes have been removed and that are destined for human consumption or any other use described herein. This term includes eggs that are removed from their shells for processing and convenience, for commercial, foodservice, and home use.

These products can be classified as refrigerated liquid, frozen, and dried products.

They can be partially complemented by other food products or additives and can be found solid, concentrated, liquid, dried, crystallized, frozen, deep-frozen or coagulated.

The possibilities in the use of egg products in accordance with the invention, are varied due to the techno-functional properties that they provide. Such properties may include foaming, emulsifying, and a unique color and flavor, which are important in several industrial products and processes, to name but a few, Confectionery, Bakery, Pastry, Dairy products, Ice creams, Drinks, Baby food, Creams and soups, Mayonnaise and sauces, Pasta, Ready cooked meals, Delicatessen, Pet food, Fish farming food, Cosmetic products, Glues (specifically, albumin), Tannery, pains, Pharmaceutical Industry. Still further, egg components and parts may also display useful properties and any uses thereof is also encompassed by the invention. More specifically, egg yolk and components thereof, may exhibit variety of properties such as, Flavouring, Coloring (by Xanthophyllis), Emulsifier capacity (by Lecithin, Lipoproteins LDL), Coagulant and binding substance (by Lipoproteins LDL and other proteins), Antioxidant (Phosvitin), Pharmaceutical uses (IgY, Cholesterol, Sialic acid). Egg white and its main protein, albumen may display Frother capacity, foam stabilizer (Lysozyme, Egg albumen), Anticrystallization (Egg mucin, Egg mucoid), Coagulant and binding substance (by Egg Albumin, Conalbumin), Preservatives (Lysozyme, Conalbumin), Rheological properties and Pharmaceutical properties.

In some embodiments, any of the eggs of the invention as disclosed herein or any component, element part or product thereof may be used for cosmetic applications. More specifically, egg white produced from the eggs of the invention may be used as a facial products, skin care, hair care and in lotions. Egg yolks produces from any of the eggs of the invention may be used in shampoos, conditioners and soaps. Cholesterol, lecithin and some of the egg's fatty acids may be used in skin care products, such as revitalizers, make-up foundations and lipstick.

In yet some further embodiments, the eggs of the invention may be used in animal feed. The excellent nutrition of eggs enhances various pet foods. Egg white may be used as a protein reference in feeding laboratory animals. Eggshells produced from the eggs of the invention may be dried, crushed and used to fed to laying hens as a rich calcium source and high-quality protein source (from egg white left inside the shells).

In yet some further embodiments, any of the eggs of the invention as disclosed herein or any component, element part or product thereof may be used for medical and pharmaceutical application. More specifically, fertile eggs provided by the invention may be used to manufacture vaccines (including influenza shots), as a source of purified protein and as an aid in the preservation of bull semen for artificial insemination.

Still further in some embodiments, any of the eggs of the invention as disclosed herein or any component, element part or product thereof may be used for nutraceutical application. More specifically, particular components purified and prepared from the eggs of the invention may be specifically applicable, in different products and processes. For example, lysozyme, an egg white protein, may be used as a food preservative and as an antimicrobial agent in pharmaceutical products. Avidin that is an egg white protein and biotin that is a vitamin found in egg white and, to a much greater extent, in egg yolk, may be prepared and purified from any of the eggs of the invention. Avidin-biotin technology in accordance with the invention may be used in various medical diagnostic applications such as immuno-assay, histopathology and gene probes. Sialic acid, an amido acid, that may be purified from any of the eggs of the invention, has been shown to inhibit certain stomach infections. Liposomes, fatty droplets found in eggs, are used as a controlled delivery mechanism for various drugs. Immunoglobulin yolk (IGY), a simple egg-yolk protein which has immunological properties, may be used as an anti-human-rotavirus (HRV) antibody in food products. Phosvitin, a phosphoprotein found in egg yolk, provides antioxidant benefits in food products. Choline, a B vitamin combined with lecithin in egg yolk, is important in brain development and is used to treat certain liver disorders. Eggs are one of the best food sources of choline. Ovolecithin, a phospholipid found in egg yolk, has a high proportion of phosphatidycholine and contains fatty acids—such as arachidonic acid (AA) and docosahexanoic acid (DHA), which have been shown to improve visual activity in infants and to improve fatty-acid status. Egg lecithin has both emulsifying and antioxidant properties and, beyond its usefulness in keeping the oil and vinegar of mayonnaise in suspension, it's used chiefly in medicine. Shell-membrane protein is being used experimentally to grow human skin fibroblasts (connective tissue cells) for severe-burn victims and in cosmetics.

In yet some further embodiments, the invention further provides the use of egg shells prepared from any of the eggs of the invention, as a dietary source of calcium for humans and other mammals. In further embodiments, these egg shells may be used as a powdered, purified product in fortification of breads and confectioneries, fruit drinks, crackers, condiments. Egg shell calcium in accordance with the invention may be also used as oral phosphate binder in low phosphate diets for e.g. patients suffering from renal failure.

Still further, in some embodiments thereof, the invention provides the use of any protein or substance separated and/or purified from any of the eggs of the invention or from any element or component thereof. More specifically, such separated proteins can be used in food and pharmaceutical industry as is or after enzymatic modifications. In some embodiments, ovotransferrin that may be separated from any of the eggs of the invention, may be used as a metal transporter, antimicrobial, or anticancer agent, whereas lysozyme may be mainly used as a food preservative, and ovalbumin may be used as a nutrient supplement. Ovomucoid may be used to as an anticancer agent and ovomucin as a tumor suppression agent. Hydrolyzed peptides from these proteins may be also used for anticancer, metal binding, and antioxidant activities. Therefore, separation of egg white proteins from any of the eggs of the invention and the productions of bioactive peptides from egg white proteins are all are encompassed by the present invention.

Still further, in the aquaculture field (comprising fish farms but also crustacean cultivation), several species exhibit different characteristics between the male and female gender which are of critical importance for the industry such as the size, the weight and the developing rate. Therefore, in some embodiments, the methods of the invention may be particularly useful for gender selection in fish. In yet some specific embodiments, the fish used by the method of the invention may be of the genus tilapia. In more specific embodiments, the methods of the invention uses as the transgenic heterogametic and homogametic organisms, tilapia fish that may be in some embodiments of the Oreochromis niloticus species.

Thus, in some specific embodiments (a), for selecting for males in tilapia fish of the Oreochromis niloticus species, the at least one nucleic acid sequence encoding at least one guide RNA directed against at least one target sequence may be integrated into the X chromosome of the heterogametic fish (male) provided by the method of the invention.

In yet some further embodiments (b), for selecting for females, at least one nucleic acid sequence encoding at least one guide RNA directed against at least one target sequence may be integrated into the Y chromosome of said heterogametic tilapia fish (male) provided by the method of the invention.

In more specific embodiments, the methods of the invention may be applicable for tilapia fish of any one of the species Oreochromis aureus, Oreochromis karongae or Pelmatolapia mariae.

In some embodiments (a), for selecting for males, the at least one nucleic acid sequence encoding at least one guide RNA directed against at least one target sequence may be integrated into the W chromosome of said heterogametic tilapia fish of any one of the species Oreochromis aureus, Oreochromis karongae or Pelmatolapia mariae (female) provided by the method of the invention. In yet some other embodiments (b), for selecting for females, the at least one nucleic acid sequence encoding at least one guide RNA directed against at least one target sequence may be integrated into the Z chromosome of the heterogametic tilapia fish of any one of the species Oreochromis aureus, Oreochromis karongae or Pelmatolapia mariae (female) provided by the method of the invention.

Still further, in some embodiments, the methods of the invention may be applicable for gender selection of crustaceans. In some specific embodiments, the crustaceans used by the method of the invention as the transgenic heterogametic and homogametic organisms may be shrimp. In more specific embodiments (a), for selecting for males, the at least one nucleic acid sequence encoding at least one guide RNA directed against at least one target sequence may be integrated into the W chromosome of the heterogametic shrimp (female) provided by the method of the invention; or

(b) for selecting for females, the at least one nucleic acid sequence encoding at least one guide RNA directed against at least one target sequence may be integrated into the Z chromosome of said heterogametic shrimp (female) provided by the method of the invention.

Methods for gender selection enables also pest control for example for controlling population of undesired insects. Thus, in some embodiments, the invention provides methods for gender selection in insects. The methods of the invention thus provide the use of transgenic homogametic and heterogametic insects. More specifically, methods of gender selection provided by the invention are of particular relevance in the case of mosquitoes that may transmit diseases.

Thus, in yet some further embodiments, the invention provides methods for gender selection in mosquitos, using as transgenic insects, transgenic homogametic and heterogametic mosquitoes. In some specific embodiments (a), for selecting for mosquitoes males, the guide RNA directed against at least one target sequence may be integrated into the X chromosome of heterogametic mosquito (male) provided by the invention. In yet some alternative embodiments (b), for selecting for female mosquitoes, the sequence encoding a guide RNA directed against at least one target sequence may be integrated into the Y chromosome of the heterogametic mosquito (male) provided by the method of the invention.

In some specific and non-limiting embodiments, the at least one target sequence may be a gene essential for embryogenesis, survival or development. More specifically, at least one gene essential for embryogenesis may be any one of Cyclin A, specifically comprising the nucleic acid sequence as denoted by SEQ ID NO: 16, IAP1 (Inhibitor of apoptosis 1), specifically comprising the nucleic acid sequence as denoted by SEQ ID NO: 17 and GPDH (Glycerol-3-phosphate dehydrogenase), specifically comprising the nucleic acid sequence as denoted by SEQ ID NO: 18. It should be noted that any of the essential genes disclosed herein above in connection with other aspects of the invention are also applicable for any of the methods of the invention.

In some specific embodiments, the mammalian organism may be a cattle, in more specific embodiments, such organism may be a cow.

In yet some further specific embodiments (a) for selecting for males, the at least one nucleic acid sequence encoding at least one guide RNA directed against at least one target sequence may be integrated into the X chromosome of the heterogametic cow (that may be in some embodiments, male). In yet some further embodiments (b), for selecting for females, the at least one nucleic acid sequence encoding at least one guide RNA directed against at least one target sequence may be integrated into the Y chromosome of the heterogametic cow (male).

In some specific and non-limiting embodiments, at least one target may be a gene essential for embryogenesis, survival or development. In yet some specific embodiments, a gene essential for embryogenesis may be any cow ortholog of any of the mouse essential genes disclosed herein before. In yet some further alternative or additional embodiments, the target sequence may be sequences appearing in non-coding regions of at least one chromosomes. Cleavage of such sequences by the targeted Cas9, leads to inability of repair and/or destruction of the chromosome, thereby leading to death of the cow embryo. In yet some further embodiments, such targeted cleavage leads to specific knock out of a target gene essential for embryogenesis, survival or viability of the embryo. In yet some further embodiments, where a non-active nuclease, specifically, catalytically dead Cas9 (dCas9) is used, or any fusion protein thereof with activator or repressor, activation or alternatively, repression of essential genes may result in defective or impaired development of an embryo of the undesired gender.

In the agricultural field, there is also a need for efficient methods of gender selection. Certain plants possess different characteristics whenever they are from the female or male gender, which may be of importance for a particular applications. Thus, in certain embodiments, the invention provides methods for gender selection in plants. In yet some further embodiments, the eukaryotic heterogametic organism and homogametic organism provided by the methods of the invention may be of the biological kingdom Plantae. In more specific embodiments, the organisms may be a dioecious plant.

In some more specific embodiments, the dioecious plant may be of the family Cannabaceae. In some more specific embodiments, the methods of the invention may be applicable for plants of the family Cannabaceae may be any one of Cannabis (hemp, marijuana) and Humulus (hops). In more particular embodiments, the invention provides methods for gender selection of Cannabis.

In some embodiments (a), for selecting for male Cannabis plants, the at least one nucleic acid sequence encoding at least one guide RNA directed against at least one target sequence may be integrated into the X chromosome of the heterogametic Cannabis plant (male) provided by the method of the invention. In yet some further embodiments (b), for selecting for female Cannabis plants, at least one nucleic acid sequence encoding at least one guide RNA directed against at least one target sequence may be integrated into the Y chromosome of the heterogametic Cannabis plant (male) provided by the method of the invention.

In yet some other particular embodiments, the invention provides methods for gender selection of Humulus. In some embodiments (a), for selecting for male Humulus plants, the at least one nucleic acid sequence encoding at least one guide RNA directed against at least one target sequence may be integrated into the X chromosome of the heterogametic Humulus plant (male) provided by the method of the invention. In yet some further embodiments (b), for selecting for female Humulus plants, at least one nucleic acid sequence encoding at least one guide RNA directed against at least one target sequence may be integrated into the Y chromosome of the heterogametic Humulus plant (male) provided by the method of the invention.

In addition, the invention provides methods for gender selection that also enable the modification of undesired traits in the selected progeny. Alternatively, the methods of the invention may provide gender selection that also enable the modification, and specifically, the enhancement of a desired traits in the selected progeny.

Thus, in a further aspect, the invention relates to a method for selecting a desired gender of an eukaryotic organism and for modifying at least one undesired trait and/or a desired trait in the selected organism. More specifically, the method comprising the steps of:

In a first step (a), providing a transgenic eukaryotic heterogametic organism comprising:

(i) at least one nucleic acid sequence encoding at least one guide RNA directed against at least one target sequence within at least one of coding and non-coding sequences or any product/s thereof, of at least one chromosome of the organism. It should be noted that the nucleic acid sequence may be integrated into one of the gender-chromosomes of said transgenic heterogametic organism.

The heterogametic organism further comprises (ii), at least one nucleic acid sequence encoding at least one guide RNA directed against at least one gene or any product/s thereof, encoding a product determining or a product essential for an undesired trait and/or alternatively, a product essential for a desired trait. It should be noted that in certain embodiments, these nucleic acid sequence may be integrated into the other gender-chromosome of the transgenic heterogametic organism.

In the next step (b), providing a transgenic eukaryotic homogametic organism comprising at least one nucleic acid sequence encoding at least one RNA guided DNA binding protein nuclease, or any fragments, domains, or subunits thereof or any non-active variant or mutant thereof and any fusion protein comprising the same. It should be noted that in certain embodiments, the nucleic acid sequence may be integrated into at least one allele of any chromosome of said transgenic homogametic organism.

The next step (c), involves breeding the transgenic heterogametic organism provided in step (a) with the transgenic homogametic organism provided in step (b), thereby obtaining a progeny predominantly composed of the one desired gender having at least one modified undesired trait.

In one embodiment the transgenic heterogametic organism provided in step (a) and said transgenic homogametic organism provided in step (b) of the method of the invention may be of the same species.

It should be noted that in some embodiments, the heterogametic organism provided by the invention will carry nucleic acid sequences encoding at least one gRNA encoding directed at sequences essential for viability, in the gender chromosome that is of the undesired gender. A non-limiting example, in the case of the XY gender chromosome system. The heterogametic organism, specifically the male (XY), carries sequences encoding or forming gRNAs directed genes essential for survival, at the Y gender chromosome, if the desired gender is female, and alternatively, in the X chromosome, if the desired gender is male. For further selecting for a desired gender that do not exhibit an undesired trait, or alternatively, exhibits a desired trait, nucleic acid sequences that encode or form at least one gRNA directed at a product essential for the desired or undesired trait, are incorporated in the other gender chromosome. Specifically, in case a female progeny is desired, the nucleic acid sequence encoding gRNA directed at genes essential for the trait are incorporated in the X chromosome. In case that the desired gender is male, these trait modulating sequences will be inserted to the Y chromosome of the transgenic male.

The transgenic female, that is the homogametic organism (XX), provides the nucleic acid modifier protein, specifically, nuclease, more specifically, Cas9, or any variant, derivative, fragments or fusion protein thereof.

In some embodiments, where selection of a progeny having a desired gender with modulated, specifically reduced undesired trait, gRNAs directed against nucleic acid sequences encoding a product essential for the trait are provided by the heterogenic male, and the nuclease is provided by the female, thereby leading to destruction of the essential sequences for the undesired trait in the progeny of the desired gender.

In case of enhancing a desired trait, gRNAs provided by the heterogametic parent may be directed at sequences that display negative control, specifically suppress the expression of a product that is essential for the desired trait.

In yet some further alternative embodiments, where the method is directed at the provision of a progeny of a desired gender that display a desired trait (e.g., improvement of body mass, milk production, resistance to pathogens and the like), the female parent (homogametic parent), will provide a first fragment of a nuclease or any derivative thereof, for example, dCas9 that lakes any nucleic activity. The mail parent (the heterogametic parent), provides sequences encoding gRNAs and fragments of the modifier protein. More specifically, the gender chromosome of the undesired gender (e.g., X in case males are desired or Y in case females are desired), comprises sequences encoding gRNAs directed at sequences essential for viability, and in addition, a fragment of the modified protein, for example, any repressor or fragment that together with the first fragment provided by the homogametic parent, form an active nuclease or repressor (e.g., methylase or transcription repressor). The other gender chromosome of the heterogametic organism (e.g., male), that is the gender chromosome of the desired gender (e.g., X in case females are desired or Y in case males are desired), contains nucleic acid sequences encoding gRNAs directed at nucleic acid sequences essential for a desired trait, and in addition, a fragment of the nucleic acid modifier protein, or in case dCas9 is provided by the other parent, a fragment of an enhancer (transcription factor, demethylase). Thus, an embryo of a desired gender, also contains gRNA and fragment of an enhancer that together with a fragment of the protein modifier provided by the homogametic parent, creates an active modifier (e.g., dCas9 provided by the homogametic parent and demethylase or transcription factor, provided by the heterogametic parent). Such active modifier, enhances and increase expression of sequences encoding product essential for the desired trait (e.g., body mass, for example myosin, milk production or resistance to pathogens).

It should be noted that in some specific embodiments, the nuclease encoded by the transgenic heterogametic or homogametic organisms provided by the methods of the invention may be at least one of: (i) a nuclease having a nucleolytic activity, specifically on DNA and/or RNA; (ii) a non-active nuclease and/or a fusion protein thereof, or alternatively (iii) any fragment, domain or subunit of the nuclease of (i) or the inactive nuclease of (ii) or of any fusion protein thereof.

As noted above, the nucleases (either active or inactive) used by the methods of the invention may be guided or non-guided nucleases.

In some further embodiments, the nuclease encoded by the transgenic organisms used by the methods of the invention may be at least one restriction enzyme. In yet some further embodiments, the target recognition element encoded by the other transgenic organisms used by the methods of the invention may be a restriction site of said enzyme. Such restriction site (either endogenous or exogenous) may reside within at least one of coding and non-coding sequences of at least one chromosomal or mitochondrial DNA of the organism.

In yet some other embodiments, restriction enzymes with long recognition sites (recognition site of at least 10 nucleotides) may also be suitable nucleases for the methods of the invention. In such embodiment, the recognition site represents the target recognition element may be incorporated into the chromosomal or mitochondrial DNA of either heterogametic/homogametic organism according to the methods of the invention.

In some specific embodiments, the methods of the invention may use guided nucleases. In yet some further specific embodiments, the guided nuclease may be any one of TALEN, ZFN, or any combinations thereof or any inactive mutants thereof and any fusion proteins comprising the same. In such case, the nucleic acid sequence encoding or forming the target recognition element may be the recognition sequence of these nucleases that may be inserted exogenously in the heterogametic or the homogametic organism used by the method of the invention as described above. However, it should be noted that the invention further encompasses the option of using the target recognition element for nuclease such as classical restriction enzyme (restriction site of up to 10 bp), a TALEN or a ZFN that have an endogenous recognition site that may be present in both the heterogametic and homogametic organism. In such case, the nuclease is split between the homogametic and heterogametic organism. More specifically, in some embodiments, the homogametic organism may comprise a nucleic acid sequence encoding a first fragment, domain or subunit of the nuclease whereas the heterogametic organism comprises a nucleic acid sequence encoding a second fragment, domain or subunit of the nuclease and vice versa. It should be noted that in some specific embodiments, PNAzymes that specifically cut RNAs or any artificial restriction systems such as argonautes with guides may serve as non-limiting examples for alternative nucleases applicable in the methods of the invention.

As noted above, in some embodiments, the target recognition element may be within a specific gene but also could be a repetitive coding/none coding region. In some embodiments, in the method of the invention may be used for gender and trait selection. In more specific embodiments (a), for selection towards the homogametic gender having at least one modified undesired trait, at least one nucleic acid sequence encoding at least one guide RNA directed against at least one target sequence may be integrated into the gender-chromosome specific for the heterogametic gender of the transgenic heterogametic organism, and at least one nucleic acid sequence encoding at least one guide RNA directed against at least one gene encoding a product determining (or essential for) an undesired trait may be integrated into the gender-chromosome specific for the homogametic gender of the transgenic heterogametic organism.

In yet some other embodiments (b), for selection towards the heterogametic gender having a modified at least one undesired trait, the at least one nucleic acid sequence encoding at least one guide RNA directed against at least one target sequence may be integrated into the gender-chromosome specific for the homogametic gender of the transgenic heterogametic organism, and at least one nucleic acid sequence encoding at least one guide RNA directed against at least one gene encoding a product determining (or essential for) an undesired trait may be integrated into a gender-chromosome specific for the heterogametic gender of said transgenic heterogametic organism.

In more specific embodiment, the undesired trait that may be modified by the method of the invention may be fertility. Fertility, as used herein, is the natural capability to produce offspring. Fertility differs from fecundity, which is defined as the potential for reproduction (influenced by fertilization and carrying a pregnancy to term). It should be understood that the invention encompasses both terms. A lack of fertility is infertility while a lack of fecundity would be called sterility. More specifically, the invention provides in some embodiments thereof methods for selecting organisms of a desired gender having modified fertility (for example, sterile subjects). In some particular embodiments, the obtained progeny may be a non-fertile (sterile) organism of a selected gender.

In yet some further embodiments, the RNA guided DNA binding protein nuclease used by the method of the invention may be any one of a CRISPR Class 2 or Class 1 system. In some specific embodiments, the RNA guided DNA binding protein nuclease may be of a CRISPR Class 2 system, specifically, nucleases of the CRISPR type II system may be used in the methods of the invention.

In some particular embodiments, the RNA guided DNA binding protein nuclease may be a Cas9. It should be appreciated that the methods of the invention further encompass the use of any fragments, domains, or subunits of Cas9 or any non-active variant or mutant thereof, for example, dCas9 and any fusion protein comprising the same (e.g., with repressor or activator as discussed before). Still further, in some embodiments, guided RNA nucleases that may be used by the invention may be CRISPR-Cas systems that target RNA. Non limiting examples for such nucleases may include CRISPR-Cas Type III and VI.

In some embodiments, the method for gender selection and trait modulation may be applicable for eukaryotic heterogametic organism and homogametic organism that may be of the biological kingdom Animalia.

In other embodiments, the eukaryotic heterogametic organism and homogametic organism of the methods of the invention may be any one of a vertebrate or an invertebrate. In some embodiments, the organism may be any one of a mammal, an avian, an insect, a fish, an amphibian, a reptile, a crustacean, a crab, a lobster, a snail, a clam, an octopus, a starfish, a sea-urchin, jellyfish, and worms.

In some particular embodiments, the method of the invention may be applicable for mammalian organisms. In yet some further specific embodiments, such mammalian organism may be a rodent. In some embodiments, the rodent may be a mouse and thus, in some embodiments, the method may be further applicable for selecting gender and modifying an undesired trait in mice.

Thus, in some embodiments (a), for selecting for males with a modulated undesired or desired trait, the at least one nucleic acid sequence encoding at least one guide RNA directed against at least one target sequence may be integrated into the X chromosome of the heterogametic mouse (male) and at least one nucleic acid sequence encoding at least one guide RNA directed against at least one gene encoding a product determining or a product essential for an undesired trait, may be integrated into the Y chromosome of the transgenic heterogametic mouse.

In yet some further embodiments (b), for selecting for females with a modulated undesired trait, or a desired trait, the at least one nucleic acid sequence or any product/s thereof, encoding at least one guide RNA directed against at least one target sequence may be integrated into the Y chromosome of said heterogametic mouse (male), and the at least one nucleic acid sequence encoding at least one guide RNA directed against at least one gene encoding a product determining or a product essential for an undesired trait, may be integrated into the X chromosome of the transgenic heterogametic mouse.

In certain embodiments, for selecting a specific gender, the target sequence may be a gene encoding a product essential for survival, embryogenesis or development of the embryo. In more specific and non-limiting embodiments, the at least one essential gene for embryogenesis may be any one of Atp5b (ATP synthase subunit beta, mitochondrial), Cdc20 (cell-division cycle protein 20), and Casp8 (Caspase-8).

In yet some further specific embodiments at least one nucleic acid sequence (spacer) encoding the guide RNAs directed against at least one gene or any product/s thereof, essential for embryogenesis may comprise the nucleic acid sequence as denoted by SEQ ID NO:1, SEQ ID NO:2 or SEQ ID NO:3.

In yet some further embodiments, the method of the invention may be applicable for avian organism. In some particular embodiments, such avian subject may be a domesticated and an undomesticated bird. In more specific embodiment, the avian organism may be any one of a poultry or a game bird. In some specific embodiments, the avian organism may be of the order Galliformes which comprise without limitation, chicken, quail, turkey, duck, Gallinacea sp, goose, pheasant and other fowl. In some embodiments, the method of the invention may be applicable for domesticated birds, specifically a chicken. Thus, according to some embodiments (a), for selecting for males with a modulated undesired or alternatively, a desired trait, at least one nucleic acid sequence encoding at least one guide RNA directed against at least one target sequence may be integrated into the W chromosome of said heterogametic chicken (female), and at least one nucleic acid sequence encoding at least one guide RNA directed against at least one gene encoding a product determining or a product essential for an undesired trait, may be integrated into the Z chromosome of the transgenic heterogametic chicken.

In some further embodiments (b), for selecting for females with a modulated undesired or desired trait, the at least one nucleic acid sequence encoding at least one guide RNA directed against at least one target sequence may be integrated into the Z chromosome of the heterogametic chicken (female), and at least one nucleic acid sequence encoding at least one guide RNA directed against at least one gene encoding a product determining or a product essential for an undesired trait, may be integrated into the W chromosome of the transgenic heterogametic organism.

In some embodiments, the method of the invention may be applicable for fish. In some particular embodiments, the method of the invention may be applicable for fish of the genus tilapia. In some embodiments, said tilapia fish may be of the Oreochromis niloticus species.

In some embodiments (a), for selecting for males with a modulated undesired or desired trait, the guide RNA directed against at least one target sequence may be integrated into the X chromosome of the heterogametic fish (male), and at least one nucleic acid sequence encoding at least one guide RNA directed against at least one gene encoding a product determining or a product essential for an undesired trait, may be integrated into the Y chromosome of the transgenic heterogametic organism. In yet some further embodiments (b), for selecting for females with a modulated undesired or desired trait, the guide RNA directed against at least one target sequence may be integrated into the Y chromosome of said heterogametic fish (male), and at least one nucleic acid sequence encoding at least one guide RNA directed against at least one gene encoding a product determining or a product essential for an undesired trait, may be integrated into the Z chromosome of the transgenic heterogametic organism.

In some embodiments, the method of the invention may be applicable for tilapia fish of the species Oreochromis aureus, Oreochromis karongae or Pelmatolapia mariae. Thus, in some embodiments (a), for selecting for males with a modulated undesired or desired trait, the at least one nucleic acid sequence encoding at least one guide RNA directed against at least one target sequence may be integrated into the W chromosome of the heterogametic fish (female), at least one nucleic acid sequence encoding at least one guide RNA directed against at least one gene encoding a product determining or a product essential for an undesired trait, may be integrated into the Z chromosome of the transgenic heterogametic organism. In yet some further embodiments (b), for selecting for females with a modulated undesired or desired trait, the at least one nucleic acid sequence encoding at least one guide RNA directed against at least one target sequence may be integrated into the Z chromosome of said heterogametic fish (female), at least one nucleic acid sequence encoding at least one guide RNA directed against at least one gene encoding a product determining or a product essential for an undesired trait, may be integrated into the W chromosome of the transgenic heterogametic organism.

In some embodiments, the methods of the invention may be applicable for crustaceans. In yet some specific embodiments, such crustaceans may be shrimp.

In some embodiments (a), for selecting for males with a modulated undesired or desired trait, the at least one nucleic acid sequence encoding at least one guide RNA directed against at least one target sequence may be integrated into the W chromosome of said heterogametic shrimp (female), at least one nucleic acid sequence encoding at least one guide RNA directed against at least one gene encoding a product determining or a product essential for an undesired or desired trait, may be integrated into the Z chromosome of the transgenic heterogametic organism; or

In yet some other embodiments (b), for selecting for females with a modulated undesired or desired trait, said at least one nucleic acid sequence encoding at least one guide RNA directed against at least one target sequence may be integrated into the Z chromosome of said heterogametic shrimp (female), at least one nucleic acid sequence encoding at least one guide RNA directed against at least one gene (or any product/s thereof) encoding a product determining or a product essential for an undesired or desired trait, may be integrated into the W chromosome of the transgenic heterogametic organism.

Still further, in some embodiments, the methods of the invention may be applicable for insects. Of particular interest are methods of the invention that may be applicable for mosquitoes. Thus, in some embodiments (a), for selecting for males with a modulated undesired or desired trait, said at least one nucleic acid sequence encoding at least one guide RNA directed against at least one target sequence may be integrated into the X chromosome of said heterogametic mosquito (male), at least one nucleic acid sequence encoding at least one guide RNA directed against at least one gene encoding a product determining or a product essential for an undesired trait, may be integrated into the Y chromosome of the transgenic heterogametic organism.

In yet some other embodiments (b), for selecting for females with a modulated undesired trait, said at least one nucleic acid sequence encoding at least one guide RNA directed against at least one target sequence may be integrated into the Y chromosome of the heterogametic mosquito (male), at least one nucleic acid sequence encoding at least one guide RNA directed against at least one gene encoding a product determining or a product essential for an undesired trait, may be integrated into the X chromosome of the transgenic heterogametic organism.

In some specific embodiments, the methods of the invention may be directed at selecting for sterile mosquito males. More specifically, such method may comprise the steps of providing a transgenic mosquito male that comprises: (i) a nucleic acid sequence encoding at least one guide RNA directed against at least one target sequence, integrated into the X chromosome; and (ii) a nucleic acid sequence encoding at least one guide RNA directed against a gene encoding a product essential for fertility, integrated into the Y chromosome of said transgenic male. Breeding of the transgenic mosquito male with a transgenic mosquito female that comprises at least one nucleic acid sequence encoding at least one RNA guided DNA binding protein nuclease integrated into at least one allele of any chromosomal or mitochondrial DNA of the female, results in sterile mosquito males. In some embodiments, mosquito genes essential for fertility may include but are not limited to at least one of the following genes with high ovary expression and tissue specificity were chosen from this analysis: AGAP005958 (ortholog of Drosophila yellow-g, a haplosufficient female-fertility gene expressed in somatic follicle cells); AGAP007280 (ortholog of Drosophila nudel, a haplosufficient female-fertility gene expressed in somatic follicle cells involved in dorsoventral patterning of the embryo); AGAP011377 (no apparent Drosophila ortholog but contains a probable chitin binding domain), as denoted by SEQ ID NO. 29, 30 and 31, respectively.

In some particular embodiments, the method of the invention may be applicable for mammalian organisms. In yet some further specific embodiments, such mammalian organism may be cattle. In some embodiments, the cattle may be a cow and thus, in some embodiments, the method may be further applicable for selecting gender and modifying an undesired or desired (e.g., milk production, body mass, resistance to pathogens) trait in cows.

Thus, in some embodiments (a), for selecting for males with a modulated undesired or desired trait, the at least one nucleic acid sequence encoding at least one guide RNA directed against at least one target sequence may be integrated into the X chromosome of the heterogametic cow (male) and at least one nucleic acid sequence encoding at least one guide RNA directed against at least one gene encoding a product determining or a product essential for an undesired trait, may be integrated into the Y chromosome of the transgenic heterogametic cow.

In yet some further embodiments (b), for selecting for females with a modulated undesired or desired trait, the at least one nucleic acid sequence or any product/s thereof, encoding at least one guide RNA directed against at least one target sequence may be integrated into the Y chromosome of said heterogametic cow (male), and the at least one nucleic acid sequence encoding at least one guide RNA directed against at least one gene encoding a product determining or a product essential for an undesired trait, may be integrated into the X chromosome of the transgenic heterogametic cow.

Still further, in some embodiments, the methods of the invention may be applicable for eukaryotic heterogametic organism and homogametic organism that may be of the biological kingdom Plantae. In some embodiments, such organisms may be a dioecious plant. In some embodiments, the dioecious plant may be of the family Cannabaceae. In some embodiments, method of the invention may be applicable for gender selection and modulation of an undesired trait in plants of the family Cannabaceae may be any one of Cannabis (hemp, marijuana) and Humulus (hops). In some embodiments (a), for selecting for male plants with a modulated undesired or desired trait, the at least one nucleic acid sequence encoding at least one guide RNA directed against at least one target sequence may be integrated into the X chromosome of the heterogametic plant (male) and at least one nucleic acid sequence encoding at least one guide RNA directed against at least one gene encoding a product determining or a product essential for an undesired trait, may be integrated into the Y chromosome of the transgenic heterogametic organism.

In yet some other embodiments (b), for selecting for female plants with a modulated undesired or desired trait, the at least one nucleic acid sequence encoding at least one guide RNA directed against at least one target sequence may be integrated into the Y chromosome of said heterogametic plant (male), at least one nucleic acid sequence encoding at least one guide RNA directed against at least one gene encoding a product determining or a product essential for an undesired or desired trait, may be integrated into the X chromosome of the transgenic heterogametic organism. Finally, the invention provides in further aspect thereof, a method for reducing the population of undesired organisms. Therefore, a forth aspect of the invention relates to a method for reducing the population of an eukaryotic species. In some embodiments the method may comprise the steps of:

In a first step (a), providing a transgenic heterogametic organism of the species comprising:

(i) at least one nucleic acid sequence encoding at least one guide RNA directed against at least one target sequence within at least one of coding and non-coding sequences of at least one chromosome of said organism. It should be noted that such nucleic acid sequence may be integrated into one of the gender-chromosomes of the transgenic heterogametic organism; and (ii) at least one nucleic acid sequence encoding at least one guide RNA directed against at least one gene encoding a product essential for fertility. In some embodiments, the nucleic acid sequence may be integrated into the other gender-chromosomes of the transgenic heterogametic organism.

In the next step (b), providing a transgenic eukaryotic homogametic organism comprising at least one nucleic acid sequence encoding at least one RNA guided DNA binding protein nuclease. It should be appreciated that the nucleic acid sequence may be integrated into at least one allele of any chromosome of the transgenic homogametic organism.

The next step (c), involves breeding the transgenic heterogametic organism provided in step (a) with the transgenic homogametic organism provided in step (b), thereby obtaining a sterile progeny predominantly composed of the one desired gender; and

The next step (d), involves releasing the sterile progeny obtained in step (c) into the wild.

In one embodiment, the transgenic heterogametic organism provided by the method of the invention in step (a) and the transgenic homogametic organism provided in step (b) may be of the same species.

In some embodiments, the invention provides methods for reducing the population of an eukaryotic species.

More specifically, in some embodiments (I), for obtaining a sterile progeny of a homogametic gender in step (c), at least one nucleic acid sequence encoding at least one guide RNA directed against at least one target sequence may be integrated into the gender-chromosome specific for the heterogametic gender of the transgenic heterogametic organism, and at least one nucleic acid sequence encoding at least one guide RNA directed against at least one gene essential for fertility may be integrated into the gender-chromosome specific for the homogametic gender of the transgenic heterogametic organism.

In yet some further embodiments (II), for obtaining a sterile progeny of a heterogametic gender in step (c), the at least one nucleic acid sequence encoding at least one guide RNA directed against at least one target sequence may be integrated into the gender-chromosome specific for the homogametic gender of the transgenic heterogametic organism, and at least one nucleic acid sequence encoding at least one guide RNA directed against at least one gene essential for fertility may be integrated into the gender-chromosome specific for the heterogametic gender of the transgenic heterogametic organism.

In some embodiments, the eukaryotic species of the methods of the invention may be an insect. As such, the invention provides methods for reducing the population of undesired insects.

In certain particular embodiments, the insect of the methods of the invention may be mosquito and the progeny obtained in step (c) may be a sterile male.

In yet another aspect thereof, the invention provides a transgenic eukaryotic heterogametic organism or any progeny, cell or product thereof. More specifically, the heterogametic organism of the invention may comprise one of the following two options. In one option (a), at least one nucleic acid sequence: (i) said sequence encoding or forming at least one target recognition element for at least one nucleic acids modifier protein; or (ii) said sequence encoding or forming said at least one target recognition element and a nucleic acid sequence encoding a first fragment, domain or subunit of at least one said nucleic acids modifier protein. In yet another option (b), the heterogametic organism may comprise at least one nucleic acid sequence encoding: (i) at least one nucleic acids modifier protein; or (ii) a second fragment, domain or subunit of said at least one nucleic acids modifier protein. It should be noted that said nucleic acid sequence is integrated into one of the gender-chromosomes of said transgenic heterogametic organism.

Still further, the invention provides a transgenic eukaryotic homogametic organism or any progeny, cell or product thereof. More specifically, the homogametic organism of the invention may comprise one of the following two options. In one option (a), at least one nucleic acid sequence encoding: either (i), at least one nucleic acids modifier protein; or (ii), a second fragment, domain or subunit of said at least one nucleic acids modifier protein. In yet another option (b), the homogametic organism of the invention may comprise at least one nucleic acid sequence that may be either (i) a sequence encoding or forming at least one target recognition element for at least one nucleic acids modifier protein; or (ii) a sequence encoding or forming said at least one target recognition element and a nucleic acid sequence encoding a first fragment, domain or subunit of at least one said nucleic acids modifier protein. It should be noted that the nucleic acid sequence is integrated into at least one allele of any chromosomal or mitochondrial DNA of said transgenic homogametic organism.

In some embodiments, the heterogametic organism of the invention may further comprise at least one nucleic acid sequence encoding at least one guide RNA directed against at least one gene, or any product/s thereof, said gene encoding a product determining an undesired trait or a desired trait of said organism. It should be noted that said nucleic acid sequence is integrated into the other gender-chromosomes of said transgenic heterogametic organism.

In some further embodiments, transgenic heterogametic organism and homogametic organism provided by the invention may be any organism of the biological kingdoms Animalia or Plantae as discussed herein above in connection with other aspects of the invention. More specifically, in case the transgenic organism of the invention is of biological kingdom Animalia, such organism may be any one of a mammal, an avian, an insect, a fish, an amphibian, a reptile, a crustacean, a crab, a lobster, a snail, a clam, an octopus, a starfish, a sea-urchin, jellyfish, and worms. In yet some further embodiments, the transgenic organism of the invention may be of the biological kingdom Plantae, specifically, a dioecious plant.

As noted above, the invention provides any of the transgenic organisms as discussed above and in connection with other aspects of the invention and in addition, further encompasses any progeny, cell or product of the transgenic organisms of the invention. In some embodiments, the transgenic organism is a rodent, such rodent may be for example a mouse. The invention also encompasses any progeny of the transgenic homogametic, the transgenic heterogametic mouse or of both. Such progeny may be in some embodiments a male or female mouse or any cell thereof. In some embodiments such male or female mouse may express one of: (i) at least one target recognition element for at least one nucleic acids modifier protein; or (ii) said nucleic acids modifier protein. In yet some further embodiments, the transgenic organism is an avian organism, for example, a chicken. In such embodiments, the invention further encompasses any progeny of said transgenic chicken, for example, an egg laying hen or a male or female broiler. The invention further encompasses any egg laid by the transgenic chickens of the invention, either comprising a vital embryo of a desired gender, or a non-vital embryo of an undesired gender, and any product of such eggs as specified herein before. In yet some further embodiments, the invention further encompasses any egg laid by the progenies of the transgenic chickens of the invention and any product or use thereof, as well as any cell thereof. It should be noted that in some embodiments the male or female chicken progenies of the transgenic chickens of the invention may express one of: (i) at least one target recognition element for at least one nucleic acids modifier protein; or (ii) said nucleic acids modifier protein.

Still further, in some embodiments, the transgenic organism is a mammal, in yet some further embodiments, the mammal may be a cattle. Thus, in some embodiments, the invention further encompasses any progeny of the transgenic cows of the invention (a progeny of at least one of the transgenic cows of the invention, and optionally of both). Such progeny as encompassed by the invention may be a dairy female cattle, a male or female beef cattle, or any cell or product thereof. In more specific embodiments, products encompassed by the invention may include any dairy or any meet products. In yet some further embodiments, the invention provide transgenic organism such as a fish, for example, a tilapia fish. In such case the invention further encompasses any progeny thereof, specifically, any a male or female fish, or any cell, fish roe or any product thereof. In yet some further embodiments, the transgenic organisms of the invention may be a crustacean, for example shrimp. The invention thus further pertains for any progeny of such shrimp, for example, a male or female shrimp, any cell or any product thereof. Still further, in some embodiments, the transgenic organism may be an insect, for example a mosquito or a bee. In yet some further embodiments, the invention provides any male or female mosquito or bee progenies of the transgenic insects of the invention, or any cell or product thereof (e.g., honey, beeswax). In yet some further embodiments, the transgenic organism may be a dioecious plant, for example, any one of Cannabis and Humulus. In such case, the invention further encompasses any male or female plant progenies of the transgenic Cannabis or Humulus plants of the invention, any cell thereof, and any product thereof. It should be understood that in some embodiments, the male or female progenies of the transgenic organisms of the invention (e.g., mice, cows, chicken, fish, shrimp, insects and plants) may express at least one copy of the transgene transferred by the parent transgenic organisms of the invention. Such transgene may be one of: (i) at least one target recognition element for at least one nucleic acids modifier protein; or (ii) at least one nucleic acids modifier protein (e.g., nuclease). In yet some further embodiments, it should be noted that any progeny or offspring as indicated herein may be either a vital or non-vital progeny. In yet some further embodiments, progeny is meant an offspring of at least one of the transgenic organisms of the invention or of any cell thereof (zygote, such as ovum or sperm). Still further, in some embodiments, the progenies according to the invention may be any progenies of breeding between the homogametic and the heterogametic transgenic organisms of the invention. In yet some further embodiments, such progeny (either vital or non-vital) may be a result of any of the methods of the invention as described herein.

It should be understood that in some embodiments of the systems, methods and transgenic organisms of the invention, the heterogametic organism may contain the nuclease or fragments thereof. In some embodiments, the nuclease or fragments thereof may be inserted in the gender chromosome of the undesired gender (for example in the Y chromosome, in case females are desired), and the gRNAs will be provided by the homogametic organism (incorporated to any chromosomes thereof, that may also include the gender chromosomes). Thus, the progeny of the desired gender will include only the gRNAs, where the non-vital progenies of the undesired gender will express the nuclease. Still further, in some alternative embodiments of the systems, methods and transgenic organisms of the invention, the heterogametic organism may contain the targeting elements (e.g., sequences encoding gRNA), as also exemplified in Example 1. In some embodiments, the targeting elements may be inserted in the gender chromosome of the undesired gender (for example in the Y chromosome, in case females are desired), and the nuclease will be provided by the homogametic organism (incorporated to any chromosomes thereof, that may also include the gender chromosomes).

The systems and methods of the invention relates all to nucleic acid sequences. Thus, for the preparation of two transgenic organism (one from each gender) used in the systems and methods of the invention, nucleic acid molecules should be provided. As used herein, “nucleic acids or nucleic acid molecules” is interchangeable with the term “polynucleotide(s)” and it generally refers to any polyribonucleotide or poly-deoxyribonucleotide, which may be unmodified RNA or DNA or modified RNA or DNA or any combination thereof. “Nucleic acids” include, without limitation, single- and double-stranded nucleic acids. As used herein, the term “nucleic acid(s)” also includes DNAs or RNAs as described above that contain one or more modified bases. As used herein, the term “oligonucleotide” is defined as a molecule comprised of two or more deoxyribonucleotides and/or ribonucleotides, and preferably more than three. Its exact size will depend upon many factors which in turn, depend upon the ultimate function and use of the oligonucleotide. The oligonucleotides (e.g., the target recognition elements), may be from about 8 to about 10,000 nucleotides long. More specifically, the oligonucleotide molecule/s used by the system of the invention may comprise any one of 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 200, 300, 400, 500, 600, 700, 800, 900, 1000, 2,000, 3,000, 4,000, 5,000, 6,000, 7,000, 8,000, 9,000, 10,000 or more bases in length.

Nucleic acid molecules can be composed of monomers that are naturally-occurring nucleotides (such as DNA and RNA), or analogs of naturally-occurring nucleotides (e.g., alpha-enantiomeric forms of naturally-occurring nucleotides), or modified nucleotides or any combination thereof. Herein this term also encompasses a cDNA, i.e. complementary or copy DNA produced from an RNA template by the action of reverse transcriptase (RNA-dependent DNA polymerase). In this connection an “isolated polynucleotide” is a nucleic acid molecule that is separated from the genome of an organism. For example, a DNA molecule that encodes the CRISPR-Cas9 or the gRNAs of the methods and systems of the invention, that has been separated from the genomic DNA of a cell is an isolated DNA molecule. Another example of an isolated nucleic acid molecule is a chemically-synthesized nucleic acid molecule that is not integrated in the genome of an organism. A nucleic acid molecule that has been isolated from a particular species is smaller than the complete DNA molecule of a chromosome from that species. In some embodiments, the nucleic acid sequences used by the methods and systems of the invention, specifically, nucleic acid sequences comprising sequences encoding the Cas9 or the gRNA of the invention, may be provided constructed within a vector. The invention thus further relates to recombinant DNA constructs comprising the polynucleotides of the invention, and optionally, further additional elements such as promoters, regulatory and control elements, translation, expression and other signals, operably linked to the nucleic acid sequence of the invention. The phrase “operatively-linked” is intended to mean attached in a manner which allows for transgene transcription. The term “encoding” is intended to mean that the subject nucleic acid may be transcribed and translated into either the desired polypeptide or the subject protein in an appropriate expression system, e.g., when the subject nucleic acid is linked to appropriate control sequences such as promoter and enhancer elements in a suitable vector (e.g., an expression vector) and when the vector is introduced into an appropriate system or cell. It should be appreciated that in some embodiments, at least one of the first and the second nucleic acid sequences provided and used by the methods and systems of the invention may be constructed and comprised within a vector. “Vectors” or “Vehicles”, as used herein, encompass vectors such as plasmids, phagemides, viruses, integratable DNA fragments, and other vehicles, which enable the integration of DNA fragments into the genome of the host, or alternatively, enable expression of genetic elements that are not integrated. Vectors are typically self-replicating DNA or RNA constructs containing the desired nucleic acid sequences, and operably linked genetic control elements that are recognized in a suitable host cell and effect the translation of the desired spacers. Generally, the genetic control elements can include a prokaryotic promoter system or a eukaryotic promoter expression control system. Such system typically includes a transcriptional promoter, transcription enhancers to elevate the level of RNA expression. Vectors usually contain an origin of replication that allows the vector to replicate independently of the host cell. In yet some alternative embodiments, the expression vectors used by the invention may comprise elements necessary for integration of the desired exogenous sequence of the invention into the genome.

In some specific embodiments, the exogenous sequence may be inserted and thereby integrated into at least one non-coding region of the target gender chromosome. Such approach avoids the disruption of genes that may be required for development and maturation of the embryo.

It should be noted that in some embodiment of the systems, methods and transgenic organisms of the invention, the modifier protein (e.g., nuclease), by modifying (e.g., cleavage, methylation, demethylation, transcription activation or repression) the nucleic acid sequence in a target (e.g., an essential gene or control elements affecting the expression, activity or stability of at least one product essential for survival and/or development of the organism), leads to modulation of the expression, activity or stability of the encoded product. Thus, in some embodiments “Modulation” as used herein means to decrease (e.g., inhibit, reduce, suppress, attenuate) or alternatively, increase (e.g., stimulate, activate, enhance, elevate) a level, activity or stability of the product by at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99% or 100%.

All scientific and technical terms used herein have meanings commonly used in the art unless otherwise specified. The definitions provided herein are to facilitate understanding of certain terms used frequently herein and are not meant to limit the scope of the present disclosure.

Before specific aspects and embodiments of the invention are described in detail, it is to be understood that this invention is not limited to particular methods, and experimental conditions described, as such methods and conditions may vary. It is also to be understood that the terminology used herein is for the purpose of describing particular embodiments only, and is not intended to be limiting, since the scope of the present invention will be limited only by the appended claims.

As used in this specification and the appended claims, the singular forms “a”, “an”, and “the” include plural references unless the context clearly dictates otherwise. Thus for example, references to “a method” includes one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. More specifically, the terms “comprises”, “comprising”, “includes”, “including”, “having” and their conjugates mean “including but not limited to”. This term encompasses the terms “consisting of” and “consisting essentially of”. The phrase “consisting essentially of” means that the composition or method may include additional ingredients and/or steps, but only if the additional ingredients and/or steps do not materially alter the basic and novel characteristics of the claimed composition or method.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. As used herein the term “about” refers to ±10%. It should be noted that various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between. The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

The examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

### EXAMPLES

Without further elaboration, it is believed that one skilled in the art can, using the preceding description, utilize the present invention to its fullest extent. The following preferred specific embodiments are, therefore, to be construed as merely illustrative, and not limitative of the claimed invention in any way.

### Reagents

### Animals:

B6J.129(Cg)-Gt(ROSA)26Sortm1.1(CAG-cas9*,-EGFP)Fezh/J mice, Jackson laboratories (Stock No: 026179; Rosa26-Cas9 knock-in on C57BL/6J) C57BL/6 mice

### Experimental Procedures

### Mouse Lines

All mice were bred under specific pathogen-free conditions in the animal facility at Tel Aviv University. Experiments were performed according to the guidelines of the Institute's Animal Ethics Committee.

### Mouse Line 1—Encoding Cas9

B6J.129(Cg)-Gt(ROSA)26Sortm1.1(CAG-cas9*,-EGFP)Fezh/J⁻CR/SPR-Cas9 knock-in mice Mice of the Cas9-line were purchased from Jackson laboratories (Stock No: 026179; Rosa26-Cas9 knockin on C57BL/6J) [Platt, R. J. et al. CRISPR-Cas9 knockin mice for genome editing and cancer modeling. Cell 159, 440-455 (2014)]. These mice constitutively express the SpCas9 endonuclease from a CAG promoter.

### Mouse Line 2—Encoding Guides on the Y Chromosome

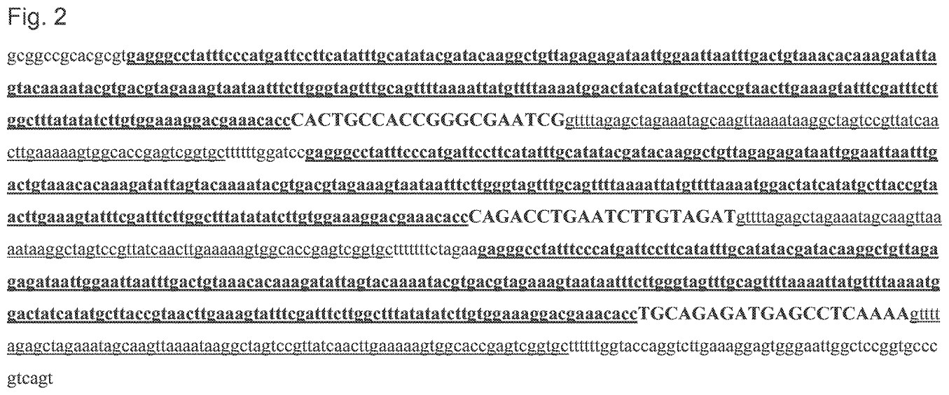

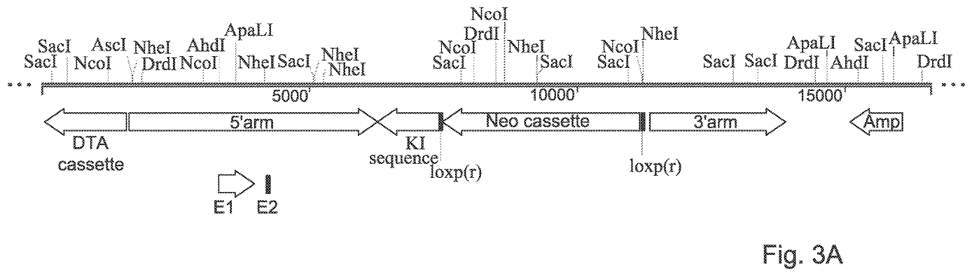

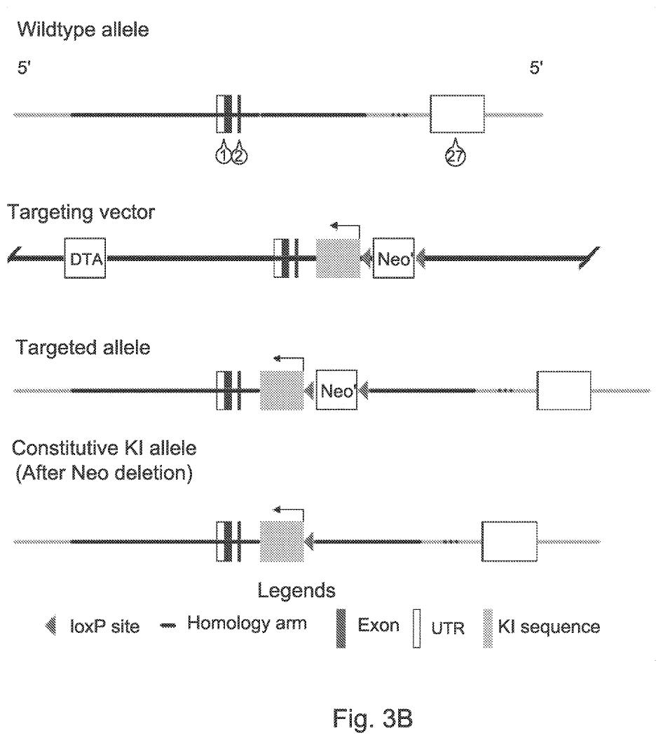

Mice of the Y-line were constructed by Cyagen Biosciences (California, USA). These C57BL/6N mice encode the following guide RNAs on their Y chromosome: 5′-CACTGCCACCGGGCGAATCG-3′; as denoted by SEQ ID NO. 1, 5′-CAGACCTGAATCTTGTAGAT-3′ as denoted by SEQ ID NO. 2; 5′-TGCAGAGATGAGCCTCAAAA-3′ as denoted by SEQ ID NO. 3, targeting the genes Atp5b, Cdc20, and Casp8, respectively. These guides were cloned into a vector targeting the reverse orientation of the 2^{nd} exon of the Y chromosome Uty gene, as presented by FIG. 2. More specifically, the Uty gene (NCBI Reference Sequence: NM_009484.3) is located on mouse chromosome Y. Twenty-seven exons have been identified, with the ATG start codon in exon 1 and the TAA stop codon in exon 27. In the targeting vector, the KI sequence was inserted into intron 2 of mouse Uty gene in the reverse orientation. In the targeting vector, the Neo cassette was flanked by loxP sites. DTA is used for negative selection. C57BL/6 ES cells were used for gene targeting. Mouse genomic fragments containing homology arms (HAs) were amplified from BAC clone by using high fidelity Taq DNA polymerase, and were sequentially assembled into a targeting vector together with recombination sites and selection markers shown in FIG. 3A. The targeting vector was digested by restriction enzymes (specifically, Ncol, Nhel, ApaLI/ScaL Ahdl/Drdl, SacI: and Ascl) for confirmation purposes. Sequence of the Final Targeting Vector is denoted by SEQ ID NO. 32. The sequence confirmed regions, the KI region (reversed orientation), 5′ and 3′ homology arms, loxp sites and exons 1 and 2, are also denoted by SEQ ID NO. 33, 34, 35, 36, 37, 38 and 39, respectively.

### Preparation of Transgenic Mice

The Uty gene (NCBI Reference Sequence: NM_009484.3) is located on mouse chromosome Y. Twenty-seven exons have been identified, with the ATG start codon in exon 1 and the TAA stop codon in exon 27.

Mouse genomic fragments containing homology arms to Uty gene were amplified from BAC clone by using high fidelity Taq DNA polymerase, and were sequentially assembled into a targeting vector together with recombination sites and selection markers shown in FIG. 3A. In the targeting vector, the KI sequence (FIG. 2) was inserted into intron 2 of mouse Uty gene in the reverse orientation, while the Neo cassette was flanked by loxP sites. DTA will be used for negative selection (FIG. 3B).

The Uty targeting construct was linearized by restriction digestion with Ascl, followed by phenol/chloroform extraction and ethanol precipitation. The linearized vector was transfected into C57BL/6 ES cells. The transfected ES cells were subject to G418 selection (200 μg/mL) 24 hours post electroporation. G418 resistant clones were picked and amplified in 96-well plates. Two copies of 96-well plates were made, one copy was frozen down and stored at −80° C. and the other copy of the 96-well plates was used for DNA isolation and subsequence PCR screening for homologous recombination. The positive clones from PCR screening were expanded and further characterized by Southern blot analysis. Genomic DNA was digested with either ScaI or AflII, and hybridized using a Neo probe. The Neo probe is expected to detect the following DNA fragment from targeted allele in the Southern analysis: ˜11.46 kb (with ScaI digestion) and ˜13.06 kb (with AflII digestion). All of the four expanded clones were confirmed to be correctly targeted. Targeted ES cell clone 2E6 was injected into C57BL/6 albino embryos, which were then re-implanted into CD-1 pseudo-pregnant females. Founder animals were identified by their coat color, their germline transmission was confirmed by breeding with C57BL/6 females and subsequent genotyping of the offspring. The Neo cassette is self-deleted in germ cells so the offspring were Neo cassette-free (FIG. 3B). Eight male heterozygous targeted mice were generated from clone 2E6 as final deliverables for this project (i.e. Y-line).

Experiments were performed in accordance with institutional guidelines and laws using protocols approved by local animal ethics committees and authorities (Regierungspraesidium Darmstadt).

### Crosses and Sex Determination

Males from the Y-line were crossed with females from the Cas9-line. As a control for progeny yield and for sex ratio, males of the Y-line were also crossed with wild-type C57BL/6J females. Sex was determined by observing the genitals at day 7 and was verified by PCR of the Y chromosome on DNA extracted from the animal's tissue. Tables 1 and 2, disclose oligonucleotides and PCR set-ups.

TABLE 1
Oligonucleotides used

| | | SEQ |
|---|---|---|
| | | ID |
| Oligonucleotides | 5′→3′ | NO. |
| Y forward | GGCAGTGGGTGTTTCGTCCTT | 19 |
| Y reverse | AACTGTTTCATTTCCCCTCTCCTC | 20 |
| Y-WT forward | GGTAAGGAGATAAAGAGTTTCCGTAC | 21 |
| Cas9-M forward | ACACCAGCACCAAAGAGGTG | 22 |
| Cas9-M reverse | GTAGGTCAGGGTGGTCACGA | 23 |
| Cas9-WT forward | AAGGGAGCTGCAGTGGAGTA | 24 |
| Cas9-WT reverse | CCGAAAATCTGTGGGAAGTC | 25 |

TABLE 2
PCR set-ups

| Reaction | Oligonucleotides used |
|---|---|
| Y-locus genotyping | Y forward + Y reverse |
| Sex determination | Y-WT forward + Y reverse |
| Cas9-locus genotyping | Cas9-M forward + Cas9-WT |
| | forward + Cas9-WT reverse |

### Preparation of Transgenic Chickens

### Chicken Lines

Chicken Line 1—encoding Cas9

### Constitutive CRISPR-Cas9 Knock-in Chicken

The first chicken line is generated using CRISPR-Cpf1-mediated gene targeting in chicken primordial germ cells (PGCs). The CRISPR-Cas9 are designed in silico and the sequences are introduced into a specific vector. The CRISPR-Cas9 knock-in chicken have constitutive expression of the SpyCas9 endonuclease directed by a CAG promoter. The construct contains a full-length attB site, the CMV enhancer/chicken beta-actin core promoter (CAGGS), a loxP-STOP (3×SV40 polyA)-loxP cassette (LSL), a 3×FLAG sequence, a nuclear localization signal (NLS), a human codon-optimized S. pyogenes cas9 gene (hSpCas9), a second NLS and the SV40 polyA signal. When used with single guide RNAs, the construct allows editing of single or multiple mouse genes in vivo or ex vivo.

Chicken Line 2—encoding Guides on the Z Chromosome

### Constitutive Knock-in Model in Chicken

The second mouse line is generated using CRISPR-Cpf1-mediated gene targeting in chicken primordial germ cells (PGCs). The Chicken line encodes guide RNAs on the Z chromosome (Z-strain). The guide RNAs on the Z chromosome are designed in silico and the sequences are introduced into a specific vector (dependent on the mediated gene targeting method).

Preparation of transgenic Chicken

CRISPR-Cas9 system (chicken line 1) and gRNAs (chicken line 2) vectors are introduced into White Leghorn (WL) chicken primordial germ cells (PGCs) using CRISPR-Cpf1-mediated gene targeting system similar to what was published using the CRISPR-Cas9 system (Oishi et al. (2016) Scientific Reports 6). Briefly, PGCs transfections for circular plasmids encoding CPF1, a gene encoding drug resistance, and the CRISPR-Cas9 system (chicken line 1) or gRNAs (chicken line 2) are carried out using Lipofectamine2000 according to the manufacturer's protocol, followed by transient antibiotic selection for insertion enrichment.

After 2-4 days of antibiotic the remaining PGCs are transferred to antibiotic-free medium and allowed to proliferate further for transplantation and analysis. Genomic DNA is extracted from PGCs and analyzed for CRISPR-Cas9 system (chicken line 1) and gRNAs (chicken line 2) insertion using PCR specific primer for the insertion and for the genomic location.

The White Leghorn (WL) chicken eggs are irradiated at 1 Gy/min and allow to develop to stage 13-15 HH before 1,000-2,000 PGCs, are injected into the bloodstream of embryos. The injected embryos hatch 2 weeks after injection. These are G0 birds (chimeric chickens). Immediately after hatch, the DNA is extracted from chick chorioallantoic membrane (CAM) samples of the hatched chicks and detection of the presence/absence of vector DNA is carried out by semi-quantitative PCR. Blood sample G0 chicks at 2-3 weeks of age and repeat PCR screen. G0 birds are raised to sexual maturity, 16-20 weeks for males. Cockerels are tested for semen production from approximately 16 weeks. Three roosters who carry >90% mutated cells in their semen are crossed with wild-type female Barred Plymouth Rock (BPR) (i/i) chickens by artificial insemination. The G1 chicks are hatch 3 weeks later and each individual chick wing banded and a CAM sample taken from the shell. Extract DNA from CAM samples and carry out PCR screen for presence of transgene, predicted to be single copy level. Repeat screen to confirm and sex chicks on DNA from blood sample 2-3 weeks later. Offspring that are derived from donor (BPR; i/i) and recipient (WL; I/I) PGCs are identified by their black (i/i) and white (I/i) feather color, respectively. For G2 production, a sexually mature G1 male and female are crossed.

### Preparation of Transgenic Plants—Agrobacterium-Mediated Transformation

Shoot apex explants are used for the transformation with Agrobacterium strain. On first day 1 (evening), a starter culture of Agrobacterium with a plasmid is initiated by inoculation of a single colony of Agrobacterium containing the vector into 3 ml yeast extract peptone (YEP) supplemented with 50 mg/L streptomycin, 35 mg/L chloramphenicol and 10 mg/L rifampicin. The cultures are incubated on a shaker for overnight at 28° C. and grown to OD600=0.8 to 1.0. On the second day, 100-200 μl of overnight grown starter culture is transferred to 30 ml of YEP (with all 3 antibiotics mentioned above for selection), 100 μM acetosyringone (AS) is added and the culture grown on the shaking incubator overnight to get an OD600 of 0.6-0.8. On the third day, the bacterial suspension is spun at 10,000 rpm (Eppendorf Centrifuge, Model-5810R, Rotor FA-45-6-30) at 4° C. for 10 min and the pellet is re-suspended in liquid SIM (MS+0.5 mg/L BAP) to obtain a final OD600=0.6-0.8. Then 100 μM acetosyringone (AS) is added. The bacterial suspension was transferred into a sterile 50 ml glass beaker.

Three days old shoot apex explants are excised and transferred into the 50 ml beaker containing bacterial suspension (40-50 explants/beaker). The beaker is incubated in orbital shaker at 28° C. for 10-15 min at 80 rpm. The explants are then transferred onto a sterile Whatman No. 1 filter paper to remove the excess moisture (around 5 min).

Infected callus is transferred onto the sterile Whatman No. 1 filter paper placed over the co-cultivation medium containing MS+0.5 mg/L BAP and 100 μM AS. The cultures are incubated at 28±2° C. for 3 days in dark.

After 3 days of co-cultivation, the explants are sub-cultured onto the selection medium (SIM containing 25 mg/l hygromycin and 250 mg/l cefotaxime) and incubated at 25±1° C. in light with light intensity of 50 μmol m-2 s-1 photosynthetic photon flux density (PPFD). The cultures are checked regularly for cell death, contamination (if any) and induction of shoots. Any dead explants were removed from the medium to prevent the release of phenolics into the medium. The explants are sub-cultured once every 2 weeks into the selection medium. The hygromycin resistant shoots are transferred onto the SEM containing MS basal salts and 25 mg/l hygromycin and 250 mg/l cefotaxime for the recovery of transformed plantlets. The rooted plants are separated, roots are washed with sterile water to remove the medium and transferred onto the paper cups containing sterile vermiculite and watered with 10× diluted MS basal medium, hardened and moved to the greenhouse and grown to maturity as mentioned in the tissue culture section above. The seeds (T1 progeny) from primary transformants (T0) are germinated on MS basal medium containing 25 mg/L hygromycin. The seeds which germinated and established are transplanted in to the green house for further assays. Transformation is confirmed by PCR analysis.

### Preparation of Transgenic Fish

Methods for the production of transgenic fish are reviewed in Tonelli F M P et al. (2017) Biotechnol Adv. 2017 Nov. 1; 35(6):832-844.

Among the new technologies applied to perform site-directed genomic integration of transgene, TALEN and CRISPR/Cas are efficiently used to generate knock-ins of specific genes in fish. These systems are commonly microinjected into newly fertilized eggs to achieve transgenesis. Other new systems have also been successfully employed to produce transgenic fish. For example, PhiC31 Streptomyces recombinase mRNA and a targeting vector DNA (containing sequences encoding fluorescent proteins) were microinjected into newly fertilized medakaeggs to generate fish with the target sequence integrated in a site-specific manner. PhiC31 recombines pseudo-attP sequences in genomic DNA with attB sites present in a DNA construction (containing the transgene for generating a knock-in). Similar to TALEN, zinc finger nucleases (ZFN) work as dimmers and can generate site-directed DSB in the target genome through recognizing 3-4 bp of DNA through domains attached to the FokI domain. Although knock-in fish have not been generated with the ZFN system, it was used to induce knockout zebrafish.

### Preparation of Transgenic Crustaceans

TALEN technology was recently applied to perform site-directed genomic integration of transgene to generate knock-ins of specific genes in crustaceans. This system is microinjected into newly fertilized eggs to achieve transgenesis.

Microinjection of Plasmids into Mosquito Eggs

The mosquito microinjection is performed as described previously Catteruccia F et al. (2000) Nature; 405:959-962.33). In brief, blood-fed mosquitoes are allowed to lay eggs on a wet filter sheet 72-84h after a blood meal. Eggs are laid and injected with plasmids within 90 min after oviposition. Injection was done by glass needles (Eppendorf, Hamburg, Germany) with a mixture of the pBac-plasmid (500 ng/μl) and piggyBac helper (300ng/μl) in injection buffer (5 mM KCl, 0.1 mM Na2HPO4, pH=6.8). After injection, the eggs were placed in water and observed for hatching.

### Example 1

### Gender Selection in a Mouse Model

Selecting a specific sex for offspring in mice is of great relevance for research. Many studies require only a single gender for research purposes. The other gender is often killed, thus resulting in unnecessary economic burden for the scientist as well as unnecessary killing of animals.

### Selection of Female Mice.

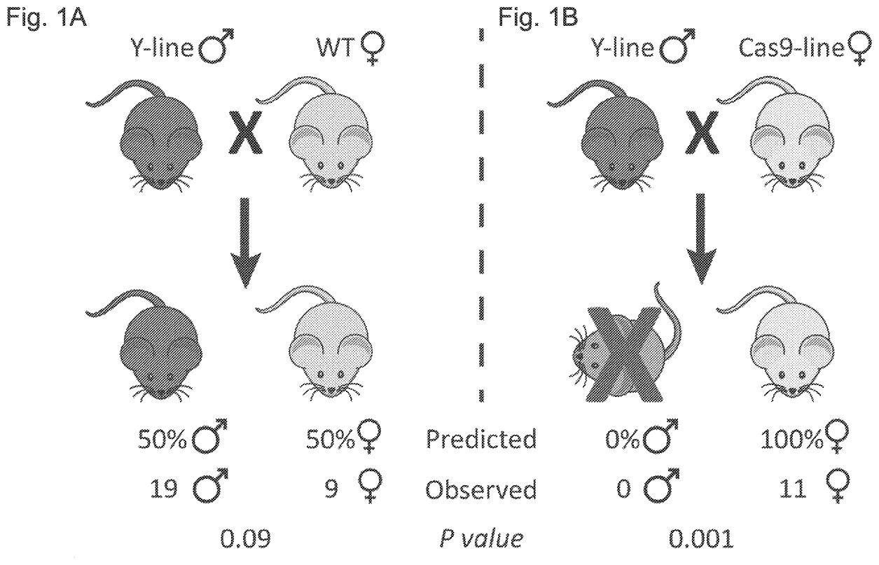

The inventors chose to provide a proof of concept for an approach that produces single-sex mouse progeny. For developing mice that produce only females, two self-sustained mouse lines were used, each producing males and females at an equal ratio. One of the lines, henceforth termed the “Cas9-line”, encoded the CRISPR-Cas9 enzyme from Streptococcus pyogenes, expressed from a CAG promoter [Platt, R. J. et al. CRISPR-Cas9 knockin mice for genome editing and cancer modeling. Cell 159, 440-455 (2014)]. The inventors generated the other line, as detailed in the experimental procedures, henceforth termed the “Y-line”, encoding on its Y chromosome three CRISPR guide RNAs (gRNAs) that target three genes. The mice used for breeding are presented in FIG. 1. The selected target genes, Atp5b, Cdc20, and Casp8, were all shown to be essential for mouse early development [Dickinson M E et al. (2016) Nature 537, 508-514; Varfolomeev EE. et al. (1998) Immunity 9, 267-276] (atp5b deficiency in mice results in embryonic lethality prior to organogenesis; cdc20 deficiency in mice results in metaphase arrest in two-cell stage embryos and consequently in early embryonic death [Li, M., York, J. P. & Zhang, P. Loss of Cdc20 causes a securin-dependent metaphase arrest in two-cell mouse embryos. Mol Cell Biol 27, 3481-3488 (2007)]; casp8 deficiency results in necroptosis and consequently in embryonic death [Kaiser, W. J. et al. RIP3 mediates the embryonic lethality of caspase-8-deficient mice. Nature 471, 368-372 (2011)]. These guides were cloned in a cassette comprising the knocking in (KI) sequence (as denoted by SEQ ID NO: 6) illustrated in FIG. 2, to allow functional expression in cells harboring the Y chromosome. This construct also encodes homologies to sequences on the Y chromosome to which it is integrated.

The construct enabling proper knock-in of the KI cassette into the chromosome Y was designed. The targeting strategy is schematically represented in FIG. 3B.

The targeted gene is the Uty gene (NCBI Reference Sequence: NM_009484.3, also denoted by SEQ ID NO: 7) which is located on mouse chromosome Y. Twenty-seven exons were identified, with the ATG start codon in exon 1 and the TAA stop codon in exon 27.

In the targeting vector, the KI sequence was inserted into intron 2 of mouse Uty gene in the reverse orientation. To engineer the targeting vector, homology arms are generated by PCR using BAC clone RP24-208N6 from the C57BL/6 library as template. In the targeting vector, the Neo cassette is flanked by loxP sites. DTA is used for negative selection. C57BL/6 Embryonic Stem cells are used for gene targeting.

The inventors selected targeting three different genes to reduce the probability of simultaneous non-targeting of the three genes, or simultaneous in-frame corrections of these three genes, or such combinations that may result in viable males. The gRNAs have been validated to efficiently cleave their targets in mouse models [Sanjana, N. E., Shalem, 0. & Zhang, F. Improved vectors and genome-wide libraries for CRISPR screening. Nat Methods 11, 783-784 (2014)]. Without being bound by any theory, the inventors hypothesized that crossing these two lines would result in a progeny consisting of female-only mice, since the resulting male mice, encoding both the Cas9 and the Y chromosome gRNAs, cannot develop normally. The inventors further hypothesized that the litter size would be half the normal size, since half of the progeny do not develop properly. To test these hypotheses, the Y-line males were crossed with the Cas9-line females, and as a control, the Y-line males were crossed with C57BL/6J females (FIG. 1A). The control crosses between the Y-line males and the C57BL/6J females produced 32 pups with an average of 6.4 pups per litter. The cross between Y-line males and the Cas9-line females produced 18 pups with an average of 4.5 pups per litter. Physical examination of the sex of the pups at day 7 revealed a ratio of 19:9 live males to females in the cross of the Y-line males with C57BL/6J females, compared to 11 live female pups with 0 males in the cross between the Y-line males and Cas9-line females (FIG. 1B). These results demonstrate, for the first time, the production of a single-sex progeny in mammals. Furthermore, they indicate that a system can be established whereby a reservoir of parents of both sexes can be stably maintained for producing single sex progeny.

From the crosses between the Y-line males with the WT females 4 pups were either killed by their mothers or died prematurely within seven days. From the crosses between the Y-line males with the Cas9-line females, 7 pups were either killed by their mothers or died prematurely within seven days. Remains of only five out of these seven pups could be analyzed. The inventors carried out PCR amplifying the Y chromosome to determine the sex of these pups. The PCR results indicated the presence of two males and three females among these pups. One of these males was most likely born dead or died immediately after birth. This male lacked developed limbs, and its body was deformed (FIG. 4A, 4B). The other male appeared paler and smaller compared to its siblings, but not deformed (FIG. 4C). Sanger DNA sequencing of PCR fragments of the target genes in the deformed male showed that at least one of its target genes, Cdc20, contains indels in the protospacer sequence in at least some cells, indicating that the gene was targeted, and corrected perhaps with residual functionality that enabled partial development of this male. In the other male, both the Casp8 and Cdc20 targeted protospacers were mutated in at least some cells. The Cdc20 contained a mismatch mutation that probably maintained the gene functionality while evading the Cas9 cleavage. The Casp8 gene contained indels in at least some cells of this male. Interestingly, in both males, the Atp5b protospacer was intact, at least in the majority of the cells, indicating that it escaped targeting or that the guide is not functional in these settings. The inventors speculate, in light of these analyses, that addition of more gRNAs to target more essential genes, or alternatively using gRNAs having multiple targets in the genome will totally eliminate the appearance of these sporadic male mice. They therefore suggest to design multiple gRNAs accordingly in future transgenic animals. The statistically significant distortion of the sex ratio is valid despite the sporadic birth of these males.

The females obtained using this approach are genetically modified organisms (GMO) because they retain the Cas9 enzyme in their genome. The system can also be modified to obtain females that instead encode the gRNAs in their genome, if in that case Cas9 is encoded on the paternal Y chromosome. This may be beneficial, as a non-enzymatic transgene, such as short gRNAs, may be considered more amenable for regulatory purposes than the Cas9 nuclease is. In this respect, it is noteworthy that a GMO salmon, which grows faster than its parental non-GMO strain, owing to a transgene regulating a growth hormone, has been approved by the US Food and Drug Administration for the food industry. Thus, GMOs may, in principle, be approved for food production.

### Selection of Male Mice

Based on similar principles, one can also establish lines producing only male progeny. The paternal line should be engineered to encode the gRNAs on its X chromosome, and should be crossed with the maternal Cas9-line, resulting in male-only progeny. Thus, two genetically-engineered mouse lines are used for breeding. Female expressing functional CRISPR-Cas9 system, lacking guide RNAs (Cas9-strain) are crossed with male encoding guide RNAs on the X chromosome. Upon fertilization, the guide RNAs on the X chromosome target genes that are crucial for embryonic development, in the presence of the CRISPR-Cas9 provided in the bred mouse line. This breeding consequently self-destructs the female embryo in utero, whereas the male embryos, lacking the guide RNAs on the Y chromosome develop normally.

Reciprocally, in order to select for male mice, female expressing guide RNAs are crossed with male encoding functional CRISPR-Cas9 system, lacking guide RNAs (Cas9-strain) on the X chromosome. Upon fertilization, the Cas9 enzyme on the X chromosome target genes that are crucial for embryonic development, in the presence of the guide RNAs provided in the bred mouse line. This breeding consequently self-destructs the female embryo in utero, whereas the male embryos, lacking the Cas9 enzyme on the Y chromosome develop normally.

### Example 2

### Gender Selection in Chicken

The sex of the offspring in chicken is selected by manipulating the heterochromosomes in Female (ZW) to include guide RNAs and crossing them with Male (ZZ) with constitutive expression of CRISPR-Cas9. In farms that produce eggs, males are unwanted, and chicks of an unwanted sex are killed almost immediately to reduce costs to the breeder.

In order to select only for female offspring in chicken for egg production, the guide RNAs are introduced into the Z chromosome of females. The next step involves breeding of these transgenic gRNA-Z female with a transgenic male that expresses CRISPR-Cas9, results in death of all male offspring (ZZ) and obtaining 100% female offspring.

Reciprocally, in order to select only for female offspring in chicken for egg production, the CRISPR-Cas9 is introduced into the Z chromosome of females. The next step involves breeding of these transgenic CRISPR-Cas9 female with a transgenic male that expresses gRNAs, results in death of all male offspring (ZZ) and obtaining 100% female offspring.

Alternatively, when male chicken are desired, sequences encoding guide RNAs directed at genes essential for embryogenesis are introduced into the W chromosome of females. The next step involves breeding this transgenic gRNA-W female with a transgenic male expressing CRISPR-Cas9. In this configuration, all female offspring (ZW) die and a progeny of 100% of male is obtained (ZZ). Reciprocally, when male chicken are desired, sequences encoding CRISPR-Cas9 are introduced into the W chromosome of females. The next step involves breeding this transgenic CRISPR-Cas9 female with a transgenic male expressing guide RNAs directed at genes essential for embryogenesis. In this configuration, all female offspring (ZW) die and a progeny of 100% of male is obtained (ZZ).

Design of guide RNAs

The sequences of the guides were designed in silico and are as follows:

| | |
|---|---|
| gRNA1. | ATACAAAGACTGCACTGCAG |
| | (as denoted by SEQ ID NO: 8). |
| gRNA2. | CTCGGGCCCCGTTAAAGCGGAGG |
| | (as denoted by SEQ ID NO: 9). |
| gRNA3. | GATGGTCGCTACCTGGCCAG |
| | (as denoted by SEQ ID NO: 10). |

These guides respectively target Casp8, Atp5b and Cdc20 (as denoted by SEQ ID NO. 28, 26 and 27, respectivelly). It should be noted that the target Indian hedgehog (IHH) gene was suggested to be essential for embryonic chick development, as its deficiency was only found in the whole-genome sequencing of lethal embryos and Creeper chickens (Jin, S. et al. (2016). Sci. Rep. 6, 30172), and may therefore also used as a target herein. In addition, the other target genes were shown to be essential for embryonic development in other organisms (Dickinson M E et al. (2016) Nature 537, 508-514; Varfolomeev E E. et al. (1998) Immunity 9, 267-276). All of these guides were generated to efficiently cleave their targets in chicken models. These guides are cloned in a cassette comprising the knocking in (KI) sequence, to allow functional expression in cells harboring the Z chromosome. This construct also encodes homologies to sequences on the Z chromosome to which it is integrated.

### Determining Progeny Yield and Female:Male Ratio in Chickens

A number of 10 wild-type rosters lacking CRISPR are bred with hens from line 2 (i.e. the hens encoding guide RNAs on the Z chromosome) to control that the observed phenotype is CRISPR-dependent. In addition, 10 rosters from line 1 (expressing Cas9) are bred with hens from line 2 (guides on Z chromosome). In the latter breeding, significantly less progeny in total is expected and rosters in particular, as the guide RNAs of the Z strain bred with the Cas9 strain self-destructs the male embryos. Number of progeny and their gender are documented and statistically analyzed.

### Example 3

### Gender Selection in Plants Gender Selection in Cannabis

Female plants of cannabis are selected by creating a transgenic male plant by manipulating the Y chromosome in the male plant to include guide RNAs directed against gene essential for embryogenesis and crossing them with Female plants with constitutive expression of CRISPR-Cas9. Upon fertilization, the guide RNAs on the Y carrying plants, together with the CRISPR-Cas9 provided by the transgenic female plant, target genes that are crucial for development. This breeding consequently self-destructs the male plants, whereas the female plants, lacking the guide RNAs from the Y chromosome develop normally.

### Gender Selection in Hops

Female plants of Humulus lupulus are selected by manipulating the Y chromosome in male plants to create transgenic male plants comprising nucleic acid sequences that encode guide RNAs directed at essential developmental genes. The next step involves crossing these transgenic male plants with transgenic female plants that constitutively express CRISPR-Cas9. Upon fertilization, the guide RNAs on the Y carrying plants target genes that are crucial for development, using the CRISPR-Cas9 provided by the female plants. This breeding consequently self-destructs the male plants, whereas the female plants, lacking the guide RNAs from the Y chromosome develop normally.

### Example 4

### Gender Selection in Fish

Female salmonid fish are selected by manipulating the Y chromosome to create transgenic males that comprise nucleic acid sequences encoding guide RNAs and crossing these transgenic males with transgenic females displaying a constitutive expression of CRISPR-Cas9. Upon fertilization, the guide RNAs on the Y strain target genes that are crucial for embryonic development, using the CRISPR-Cas9 provided by females. Male embryo are self-destructed prior to death whereas the female embryos develop normally.

Males from the Nile tilapia O. niloticus are selected by manipulating the X chromosome to create males that comprise nucleic acid sequences encoding the guide RNAs and crossing them with transgenic females that display constitutive expression of CRISPR-Cas9. Upon fertilization, the guide RNAs on the X chromosome target genes that are crucial for embryonic development, using the CRISPR-Cas9 provided by the females. Female embryo are self-destructed in utero, whereas the male embryos develop normally.

In yet another alternative embodiment, males from the tilapia species O. aureus, O. karongae and Tilapia mariae, are selected by manipulating the W chromosome in females to include the nucleic acid sequences encoding guide RNAs and crossing them with transgenic males that display constitutive expression of CRISPR-Cas9. Upon fertilization, the guide RNAs on the W chromosome of females target genes that are crucial for embryonic development, using the CRISPR-Cas9 provided constitutively by males. Female embryo are self-destructed in utero, whereas the male embryos develop normally.

### Example 5

### Gender Selection in Shrimp or Lobster

The sex of the offspring in shrimps and lobsters is selected by manipulating the heterochromosomes in Female (ZW) to include guide RNAs and crossing them with Male (ZZ) with constitutive expression of CRISPR-Cas9. In order to select only for female offspring, the guide RNAs are introduced into the Z chromosome of females. All male offspring (ZZ) die and a progeny of 100% female is obtained.

### Example 6

### Reducing Overall Mosquito Population

In mosquito, sex is determined by heterogamety, males being XY and females being XX. In order to reduce mosquito populations, two strategies are employed: elimination of females and sterilization of males. For the elimination of females, the X chromosome of males is manipulated to include the guide RNAs while female transgenic mosquitos are engineered to have constitutive expression of CRISPR-Cas9. Upon fertilization, the guide RNAs on the X chromosome target genes that are crucial for embryonic development, using the CRISPR-Cas9 provided by females. Female embryo are self-destructed in utero, whereas the male embryos develop normally. In addition, the Y chromosome of male mosquitos is also manipulated to include guides RNAs targeting male fertility genes (viable). crossbreeding of these transgenic males with a CRISPR-Cas9 encoding female results in only sterile males. These mosquitos can be released to the wild to compete with natural males and contribute to reduce the overall mosquito population. These guides target for example the Cyclin A, IAP1 and GDPH genes which were shown to be essential for embryonic development.

1-46. (canceled)

47. A system comprising:
(A) a transgenic eukaryotic heterogametic organism comprising one of:
(a) at least one nucleic acid sequence:
(i) said sequence encoding or forming at least one target recognition element for at least one nucleic acids modifier protein; or
(ii) said sequence encoding or forming said at least one target recognition element and a nucleic acid sequence encoding a first fragment, domain or subunit of at least one said nucleic acids modifier protein; or
(b) at least one nucleic acid sequence encoding:
(i) at least one nucleic acids modifier protein; or
(ii) a second fragment, domain or subunit of said at least one nucleic acids modifier protein;
wherein said nucleic acid sequence is integrated into one of the gender-chromosomes of said transgenic heterogametic organism; and
(B) a transgenic eukaryotic homogametic organism comprising one of:
(a) at least one nucleic acid sequence encoding:
(i) at least one nucleic acids modifier protein; or
(ii) a second fragment, domain or subunit of said at least one nucleic acids modifier protein; or
(b) at least one nucleic acid sequence:
(i) said sequence encoding or forming at least one target recognition element for at least one nucleic acids modifier protein; or
(ii) said sequence encoding or forming said at least one target recognition element and a nucleic acid sequence encoding a first fragment, domain or subunit of at least one said nucleic acids modifier protein;
wherein said nucleic acid sequence is integrated into at least one allele of any chromosomal or mitochondrial DNA of said transgenic homogametic organism.

48. The system according to claim 47, wherein said nucleic acids modifier protein is a nuclease, said nuclease is at least one of: (i) a nuclease having a nucleolytic activity; (ii) a non-active nuclease and/or a fusion protein thereof; (iii) any fragment, domain or subunit of the nuclease of (i) or the inactive nuclease of (ii) or of any fusion protein thereof.

49. The system according to claim 48, wherein the nuclease is an RNA guided nuclease, and wherein said at least one target recognition element is at least one ribonucleic acid guide (guide RNA) directed against at least one target sequence within at least one of coding and non-coding sequences, or any product/s thereof, of at least one chromosomal or mitochondrial DNA of said organism and wherein said nuclease is at least one RNA guided DNA binding protein nuclease, optionally, said system comprising:
(a) a transgenic eukaryotic heterogametic organism comprising at least one nucleic acid sequence encoding at least one guide RNA directed against at least one target sequence within at least one of coding and non-coding sequences, or any product/s thereof, of at least one chromosome of said organism; wherein said nucleic acid sequence is integrated into one of the gender-chromosomes of said transgenic heterogametic organism; and
(b) a transgenic eukaryotic homogametic organism comprising at least one nucleic acid sequence encoding at least one RNA guided DNA binding protein nuclease, wherein said nucleic acid sequence is integrated into at least one allele of any chromosome of said transgenic homogametic organism.

50. The system according to claim 47, wherein said transgenic heterogametic organism further comprises at least one nucleic acid sequence encoding at least one guide RNA directed against at least one gene, or any product/s thereof, encoding a product determining an undesired trait or a desired trait; wherein said nucleic acid sequence is integrated into the other gender-chromosomes of said transgenic heterogametic organism.

51. The system according to claim 49, wherein said RNA guided DNA binding protein nuclease is any one of a clustered regularly interspaced short palindromic repeat (CRISPR) Class 2 or Class 1 system, optionally, said RNA guided DNA binding protein nuclease is a CRISPR-associated endonuclease 9 (Cas9) system.

52. The system according to claim 47, wherein said eukaryotic heterogametic organism and homogametic organisms are of the biological kingdom Animalia, optionally, said organism is any one of a mammal, an avian, an insect, a fish, an amphibian, a reptile, a crustacean, a crab, a lobster, a snail, a clam, an octopus, a starfish, a sea-urchin, jellyfish, and worms.

53. The system according to claim 52, wherein said mammal is rodent, optionally, wherein said rodent is a mouse and wherein:
(a) said at least one nucleic acid sequence encoding at least one guide RNA directed against at least one target sequence is integrated into the X chromosome of said heterogametic mouse; or
(b) said at least one nucleic acid sequence encoding at least one guide RNA directed against at least one target sequence is integrated into the Y chromosome of said mouse.

54. The system according to claim 52, wherein said avian organism is any one of a domesticated and an undomesticated bird, optionally, wherein said domesticated bird is a chicken and wherein:
(a) said at least one nucleic acid sequence encoding at least one guide RNA directed against at least one target sequence is integrated into the W chromosome of said chicken; or
(b) said at least one nucleic acid sequence encoding at least one guide RNA directed against at least one target sequence is integrated into the Z chromosome of said heterogametic chicken.

55. The system according to claim 52, wherein said fish is of the genus tilapia and wherein (A) or (B):
(A) wherein said tilapia fish is of the Oreochromis niloticus species, and wherein:
(a) said at least one nucleic acid sequence encoding at least one guide RNA directed against at least one target sequence is integrated into the X chromosome of said heterogametic fish; or
(b) said at least one nucleic acid sequence encoding at least one guide RNA directed against at least one target sequence is integrated into the Y chromosome of said fish; or
(B) wherein said tilapia fish is of any one of the species Oreochromis aureus, Oreochromis karongae or Pelmatolapia mariae, and wherein:
(a) said at least one nucleic acid sequence encoding at least one guide RNA directed against at least one target sequence is integrated into the W chromosome of said fish; or
(b) said at least one nucleic acid sequence encoding at least one guide RNA directed against at least one target sequence is integrated into the Z chromosome of said heterogametic fish.

56. The system according to claim 52, wherein said organism of the biological kingdom Animalia is any one of crustaceans or insects, and wherein:
(I) said organism is a crustaceans, wherein said crustaceans are shrimp, and wherein:
(a) said at least one nucleic acid sequence encoding at least one guide RNA directed against at least one target sequence is integrated into the W chromosome of said shrimp; or
(b) said at least one nucleic acid sequence encoding at least one guide RNA directed against at least one target sequence is integrated into the Z chromosome of said heterogametic shrimp; or
(II) said organism is an insect, wherein said insects are mosquitoes, and wherein:
(a) said at least one nucleic acid sequence encoding at least one guide RNA directed against at least one target sequence is integrated into the X chromosome of said heterogametic mosquito; or
(b) said at least one nucleic acid sequence encoding at least one guide RNA directed against at least one target sequence is integrated into the Y chromosome of said mosquito.

57. The system according to claim 47, wherein said eukaryotic heterogametic organism and homogametic organisms are of the biological kingdom Plantae, wherein said organisms is a dioecious plant, optionally, wherein said dioecious plant is of the family Cannabaceae, and wherein:
(a) said at least one nucleic acid sequence encoding at least one guide RNA directed against at least one target sequence is integrated into the X chromosome of said heterogametic plant; or
(b) said at least one nucleic acid sequence encoding at least one guide RNA directed against at least one target sequence is integrated into the Y chromosome of said plant.

58. A method for selecting a desired gender of an eukaryotic organism, the method comprising the steps of:
(A) providing a transgenic eukaryotic heterogametic organism comprising one of:
(a) at least one nucleic acid sequence:
(i) said sequence encoding or forming at least one target recognition element for at least one nucleic acids modifier protein; or
(ii) said sequence encoding or forming said at least one target recognition element and a nucleic acid sequence encoding a first fragment, domain or subunit of at least one said nucleic acids modifier protein; or
(b) at least one nucleic acid sequence encoding:
(i) at least one nucleic acids modifier protein; or
(ii) a second fragment, domain or subunit of said at least one nucleic acids modifier protein;
wherein said nucleic acid sequence is integrated into one of the gender-chromosomes of said transgenic heterogametic organism;
(B) providing a transgenic eukaryotic homogametic organism comprising one of:
(a) at least one nucleic acid sequence encoding:
(i) at least one nucleic acids modifier protein; or
(ii) a first fragment, domain or subunit of said at least one nucleic acids modifier protein; or
(b) at least one nucleic acid sequence:
(i) said sequence encoding or forming at least one target recognition element for at least one nucleic acids modifier protein; or
(ii) said sequence encoding or forming said at least one target recognition element and a nucleic acid sequence encoding a first fragment, domain or subunit of at least one said nucleic acids modifier protein;
wherein said nucleic acid sequence is integrated into at least one allele of any chromosomal or mitochondrial DNA of said transgenic homogametic organism; and
(C) breeding said transgenic heterogametic organism provided in step (A) with said transgenic homogametic organism provided in step (B), thereby obtaining a progeny predominantly composed of said one desired gender.

59. The method according to claim 58, wherein said nucleic acids modifier protein is a nuclease, said nuclease is at least one of: (i) a nuclease having a nucleolytic activity; (ii) a non-active nuclease and/or a fusion protein thereof; (iii) any fragment, domain or subunit of the nuclease of (i) or the inactive nuclease of (ii) or of any fusion protein thereof, optionally, wherein the nuclease is an RNA guided nuclease, wherein said at least one target recognition element is at least one guide RNA directed against at least one target sequence within at least one of coding and non-coding sequences, or any product/s thereof, of at least one chromosome of said organism, wherein said nuclease is at least one RNA guided DNA binding protein nuclease, and wherein said method comprising the steps of:
(a) providing a transgenic eukaryotic heterogametic organism comprising at least one nucleic acid sequence encoding at least one guide RNA directed against at least one target sequence; wherein said nucleic acid sequence is integrated into one of the gender-chromosomes of said transgenic heterogametic organism;
(b) providing a transgenic eukaryotic homogametic organism comprising at least one nucleic acid sequence encoding at least one RNA guided DNA binding protein nuclease, wherein said nucleic acid sequence is integrated into at least one allele of any chromosomal or mitochondrial DNA of said transgenic homogametic organism; and
(c) breeding said transgenic heterogametic organism provided in step (a) with said transgenic homogametic organism provided in step (b), thereby obtaining a progeny predominantly composed of said one desired gender, optionally, said RNA guided DNA binding protein nuclease is Cas9.

60. The method according to claim 59, wherein:
(a) for selection towards the homogametic gender, at least one nucleic acid sequence encoding at least one guide RNA directed against at least one target sequence is integrated into the gender-chromosome specific for the heterogametic gender of said transgenic heterogametic organism; and
(b) for selection towards the heterogametic gender, said at least one nucleic acid sequence encoding at least one guide RNA directed against at least one target sequence is integrated into the gender-chromosome specific for the homogametic gender of said transgenic heterogametic organism.

61. The method according to claim 59, wherein said transgenic heterogametic organism provided in step (a), further comprises a nucleic acid sequence encoding at least one guide RNA directed against at least one gene, or any product/s thereof, encoding a product determining at least one of an undesired trait and a desired trait; wherein said nucleic acid sequence is integrated into the other gender-chromosomes of said transgenic heterogametic organism.

62. The method according to claim 58, wherein said eukaryotic heterogametic organism and homogametic organism are of the biological kingdom Animalia or of the biological kingdom Plantae, optionally, wherein said organism of the kingdom Animalia is any one of a mammal, an avian, an insect, a fish, an amphibian, a reptile, a crustacean, a crab, a lobster, a snail, a clam, an octopus, a starfish, a sea-urchin, jellyfish, and worms, and wherein said organism of the kingdom Plantae is a dioecious plant of the family Cannabaceae.

63. The method according to claim 62, wherein said organism is any one of:
(I) an organism of the kingdom Animalia, said organism is any one of:
(i) a mammal, optionally, said mammal is a rodent, and wherein said rodent is a mouse and wherein:
(a) for selecting for males, said at least one nucleic acid sequence encoding at least one guide RNA directed against at least one target sequence is integrated into the X chromosome of said heterogametic mouse; or
(b) for selecting for females, said at least one nucleic acid sequence encoding at least one guide RNA directed against at least one target sequence is integrated into the Y chromosome of said heterogametic mouse;
(ii) an avian organism, optionally, said avian organism is any one of a domesticated and an undomesticated bird, wherein said domesticated bird is a chicken and wherein:
(a) for selecting for males, said at least one nucleic acid sequence encoding at least one guide RNA directed against at least one target sequence is integrated into the W chromosome of said heterogametic chicken; or
(b) for selecting for females, said at least one nucleic acid sequence encoding at least one guide RNA directed against at least one target sequence is integrated into the Z chromosome of said heterogametic chicken;
(iii) a fish, optionally, said fish is of the genus tilapia and wherein (A) or (B):
(A) said tilapia fish is of the Oreochromis niloticus species, and wherein:
(a) for selecting for males, said at least one nucleic acid sequence encoding at least one guide RNA directed against at least one target sequence is integrated into the X chromosome of said heterogametic fish; or
(b) for selecting for females, said at least one nucleic acid sequence encoding at least one guide RNA directed against at least one target sequence is integrated into the Y chromosome of said heterogametic fish; or
(B) said tilapia fish is of any one of the species Oreochromis aureus, Oreochromis karongae or Pelmatolapia mariae, and wherein:
(a) for selecting for males, said at least one nucleic acid sequence encoding at least one guide RNA directed against at least one target sequence is integrated into the W chromosome of said heterogametic fish; or
(b) for selecting for females, said at least one nucleic acid sequence encoding at least one guide RNA directed against at least one target sequence is integrated into the Z chromosome of said heterogametic fish;
(iv) crustaceans, optionally, said crustaceans are shrimp, and wherein:
(a) for selecting for males, said at least one nucleic acid sequence encoding at least one guide RNA directed against at least one target sequence is integrated into the W chromosome of said heterogametic shrimp; or
(b) for selecting for females, said at least one nucleic acid sequence encoding at least one guide RNA directed against at least one target sequence is integrated into the Z chromosome of said heterogametic shrimp; or
(v) an insect, optionally, said insects are mosquitoes, and wherein:
(a) for selecting for males, said guide RNA directed against at least one target sequence is integrated into the X chromosome of said heterogametic mosquito; or
(b) for selecting for females, said guide RNA directed against at least one target sequence is integrated into the Y chromosome of said heterogametic mosquito;
(II) an organism of the biological kingdom Plantae, wherein said organism is a dioecious plant, optionally, said dioecious plant is of the family Cannabaceae, wherein:
(a) said at least one nucleic acid sequence encoding at least one guide RNA directed against at least one target sequence is integrated into the X chromosome of said heterogametic plant; or
(b) said at least one nucleic acid sequence encoding at least one guide RNA directed against at least one target sequence is integrated into the Y chromosome of said plant.

64. The method according to claim 58, for selecting a desired gender of an eukaryotic organism and for modifying at least one of at least one undesired trait and at least one desired trait in said selected organism, the method comprising the steps of:
(a) providing a transgenic eukaryotic heterogametic organism comprising:
(i) at least one nucleic acid sequence encoding at least one guide RNA directed against at least one target sequence within at least one of coding and non-coding sequences, or any product/s thereof, of at least one chromosome of said organism; wherein said nucleic acid sequence is integrated into one of the gender-chromosomes of said transgenic heterogametic organism; and
(ii) at least one nucleic acid sequence encoding at least one guide RNA directed against at least one gene encoding a product determining an undesired trait or a desired trait; wherein said nucleic acid sequence, or any product/s thereof, is integrated into the other gender-chromosome of said transgenic heterogametic organism;
(b) providing a transgenic eukaryotic homogametic organism comprising at least one nucleic acid sequence encoding at least one RNA guided DNA binding protein nuclease, wherein said nucleic acid sequence is integrated into at least one allele of any chromosome of said transgenic homogametic organism; and
(c) breeding said transgenic heterogametic organism provided in step (a) with said transgenic homogametic organism provided in step (b), thereby obtaining a progeny predominantly composed of said one desired gender having at least one modified undesired trait, optionally, said undesired trait is fertility, and wherein the obtained progeny is a non-fertile organism of a selected gender.

65. A method for reducing the population of an eukaryotic species comprising the steps of:
(a) providing a transgenic heterogametic organism of said species comprising:
(i) at least one nucleic acid sequence encoding at least one guide RNA directed against at least one target sequence within at least one of coding and non-coding sequences, or any product/s thereof, of at least one chromosome of said organism; wherein said nucleic acid sequence is integrated into one of the gender-chromosomes of said transgenic heterogametic organism; and
(ii) at least one nucleic acid sequence encoding at least one guide RNA directed against at least one gene, or any product/s thereof, encoding a product essential for fertility; wherein said nucleic acid sequence is integrated into the other gender-chromosomes of said transgenic heterogametic organism;
(b) providing a transgenic eukaryotic homogametic organism comprising at least one nucleic acid sequence encoding at least one RNA guided DNA binding protein nuclease, wherein said nucleic acid sequence is integrated into at least one allele of any chromosome of said transgenic homogametic organism;
(c) breeding said transgenic heterogametic organism provided in step (a) with said transgenic homogametic organism provided in step (b), thereby obtaining a sterile progeny predominantly composed of said one desired gender; and
(d) releasing said sterile progeny obtained in step (c) into the wild; optionally, wherein said eukaryotic species is an insect, and wherein said insect is a mosquito and the progeny obtained in step (c) is a sterile male.

66. A transgenic eukaryotic heterogametic organism or any progeny, or a transgenic eukaryotic homogametic organism, or any cell or product thereof, wherein:
(I) said heterogametic organism comprises one of:
(a) at least one nucleic acid sequence:
(i) said sequence encoding or forming at least one target recognition element for at least one nucleic acids modifier protein; or
(ii) said sequence encoding or forming said at least one target recognition element and a nucleic acid sequence encoding a first fragment, domain or subunit of at least one said nucleic acids modifier protein; or
(b) at least one nucleic acid sequence encoding:
(i) at least one nucleic acids modifier protein; or
(ii) a second fragment, domain or subunit of said at least one nucleic acids modifier protein;
wherein said nucleic acid sequence is integrated into one of the gender-chromosomes of said transgenic heterogametic organism; or
(II) said homogametic organism comprises one of:
(a) at least one nucleic acid sequence encoding:
(i) at least one nucleic acids modifier protein; or
(ii) a second fragment, domain or subunit of said at least one nucleic acids modifier protein; or
(b) at least one nucleic acid sequence:
(i) said sequence encoding or forming at least one target recognition element for at least one nucleic acids modifier protein; or
(ii) said sequence encoding or forming said at least one target recognition element and a nucleic acid sequence encoding a first fragment, domain or subunit of at least one said nucleic acids modifier protein;
wherein said nucleic acid sequence is integrated into at least one allele of any chromosomal or mitochondrial DNA of said transgenic homogametic organism.


SEQUENCE LISTING (43 sequences)

SEQ ID NO: 1 - DNA, 20 bp, Artificial Sequence
  gRNA1 for mouse Atp5b
  misc_feature: Synthetic
cactgccaccgggcgaatcg

SEQ ID NO: 2 - DNA, 20 bp, Artificial Sequence
  gRNA2 for mouse cdc20
  misc_feature: Synthetic
cagacctgaatcttgtagat

SEQ ID NO: 3 - DNA, 20 bp, Artificial Sequence
  gRNA3 for mouse caspase 8
  misc_feature: Synthetic
tgcagagatgagcctcaaaa

SEQ ID NO: 4 - DNA, 76 bp, Artificial Sequence
  sgRNA core for mouse
  misc_feature: Synthetic
gttttagagctagaaatagcaagttaaaataaggctagtccgttatcaacttgaaaaagtggcaccgagtcggtgc

SEQ ID NO: 5 - DNA, 249 bp, Artificial Sequence
  U6 promoter
  misc_feature: Synthetic
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacc

SEQ ID NO: 6 - DNA, 1128 bp, Artificial Sequence
  Knocking in (KI) sequence
  misc_feature: Synthetic
gcggccgcacgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacccactgccaccgggcgaatcggttttagagctagaaatagcaagttaaaataaggctagtccgttatcaacttgaaaaagtggcaccgagtcggtgcttttttggatccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacccagacctgaatcttgtagatgttttagagctagaaatagcaagttaaaataaggctagtccgttatcaacttgaaaaagtggcaccgagtcggtgctttttttctagaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacctgcagagatgagcctcaaaagttttagagctagaaatagcaagttaaaataaggctagtccgttatcaacttgaaaaagtggcaccgagtcggtgcttttttggtaccaggtcttgaaaggagtgggaattggctccggtgcccgtcagt

SEQ ID NO: 7 - DNA, 5270 bp, Mus musculus
  misc_feature: mouse Uty gene Accession number NM_009484.3)
attggaaagcctggctttagggagaggttatggacgttacgtgcggttcacactgacggtattataacaactttatgcggatgctagcgaagtttgatattctaagaacttcgcttaggttctgcaacaatgagttcttcttgcgttcaccatctgcgtggtaatgtgaccagatagcctctgccgctttctcctttgccagtttccatgaaatcttacggattatctctcactactgctgccctaggtaatgaggaaaagaaaatggcggcggaaaaggctagaggcgagggcgaagaggggtccttcagtctcacagtcgaagagaagaaggcgctttgtggattagatagcagcttcttcgggttcttgacccgatgcaaagatggcgccaagatgaagacgctgttgaacaaggcaattcacttctatgagtctttaatagtaaaagctgaaggaaaagtggaatcagatttcttttgtcaattaggtcacttcaatctcttactggaagattattcaaaagcattgtcatcttaccagagatactacagtttacagactgactactggaagaatgctgcctttttatatggtcttggtttggtctatttctactacaatgcatttcagtgggcaattagagcttttcaggaagtcctttatgttgaccctaatttttgcagagccaaagaaatccatttacgacttggttttatgttcaaaatgaatacagattatgaatctagtttaaagcattttcagctggccctgatcgactgtaatgtctgtactttgtccagtgtcgaaattcagtttcatattgctcacttatatgaaacccagaggaaataccattctgcaaaggcagcctatgaacaacttttacagattgaaagcttgccatcacaagtcaaagcaactgtattgcaacagttaggttggatgcaccataatatggatctaattggagacaataccaccaaagagagatatgctattcagtatctccaaaagtcattggaggaagatcctaattctggtcaatcttggtatttccttggaaggtgttactcatgtattgggaaggttcaggatgcctttgtatcttacaggcaatccattgataaatcagaagcaagtgcagacacatggtgttcaataggtgtgctgtatcaacagcaaaatcaacctatggatgccttgcaagcctatatatgtgctgtacagttggatcatggacatgcagcagcctggatggacttaggtattctttatgaatcttgcaaccaaccccaggatgccattaaatgctacttaaatgcagctcggagcaaatcttgtaataacacctctgcacttacatcaagaattaagtttttacaggctcagttgtgtaaccttccacaaagtagtctacagaataaaactaaattacttcctagtattgaggaggcatggagcctaccaatacccgcagagcttacctccaggcagggtgccatgaacacagcacagcagagtgtttctgatacctggaatagtgtccagacagcttcacatcattcagtacagcagaaagtttatacacagtgcttcacagcacagaaattacagagttttggtaaagatcaacaacctccatttcaaactgggtccacacgatatctccaagcagctagcactaatgatcagaatcagaatggaaatcatactctgcctcagaattcaaaaggggatgctcaaaatcacttcttacgtattcctacctcagaagaacaaaagatcattaactttaccaaagagagcaaggattcaagaagtaaatccttgacttctaaaacaagtaggaaagatagagacacatctaatatctgtgttaatgcaaagaagcattctaatcatatttatcagatctcatcagttccgatttccagccttaataataaagaatctgtgtcaccagatttaataattgtagacaatcctcagctttctgtcttagttggagaaactatcgacaatgtggaccatgacattggaacttgtgacaaagtcaataatgttcatctagccattcacaaaaagcctgataatctttctgcttcttcaccctcttcagccatttccacagaaacactttctctaaagctcactgaacagacacacattgttaccagctttatcagtcctcacagtggattacacacaattaatggagaagggcatgagaacttagagagttctgccagtgttaatgttggcctcagacctagatctcaaatcataccatcaatgtctgtgtccatatactccagttcaacagaagttctgaaagcgtgcaggagtctaggtaaaaatggtttgtctaatggtcatattctcttggatatatgcccacctccaagaccaccaacttcgccatatcctcctttaccaaaggagaagttgaatccacccactcctagtatttatttggaaaataagcgagatgccttttttcctccattacatcaattttgcatcaatccaaaaaaccctgttacagtaattcgcggtcttgctggagctcttaagttagatcttggacttttctctaccaaaactttggtagaggctaacaatgaacatatcgtagaagtgaggacccaattgttacagccagcagatgaaaactgggatccctctggaacaaagaaaatctggcgatatgaaaataaaagttctcatactacaattgctaaatatgcacagtaccaggcctgctccttccaggaatcattgagagaagaaaatgaaagaagaacacaagttaaagactattcagataatgaatcaacatgttcagataattctggaaggagacagaaagcaccctttaaaaccataaaatgtgggattaacattgatctgtctgataacaaaaagtggaagttgcagttacatgagctgacaaagcttcctgcttttgtacgtgttgtatcagcagggaatcttctcagtcatgttggctatactatattggggatgaattcagttcaactatgtatgaaagttccagggagtagaataccaggtcaccaagaaaataacaatttctgttctgttaacataaatattggtccaggtgattgtgaatggtttgttgtacctgaagattattggggtgttctgaatgacttctgtgaaaaaaataatttgaatttcctgatgagttcttggtggccgaaccttgaagatctttatgaagcaaatgttcctgtttataggtttattcaacgacctggtgatttggtttggataaatgcaggcactgttcactgggttcaggctattgggtggtgtaacaacatcacctggaatgttggtccactcacagccttccaatataaattggcagttgaacgatatgaatggaataaattgcaaagtgtcaagtcagtagtacccatggttcatctttcatggaatatggcacgaaatatcaaagtctcagatccaaagctttttgaaatgattaaatattgtctcctcaagattctaaagcattgtcagactttgagagaagctcttgttgcagcagggaaagaggttttgtggcatgggaggataaatgatgaaccagctccttactgtagcatctgtgaggtggaagtttttaatttactttttgtcactaatgaaagcaattctcaaaagacatatatagtacattgtcagaactgtgcacgaaaaacaagtggaaatttggaaaattttgtagtgctagaacaatacaaaatggaggacctgattcaagtctatgaccaatttacattagctccttcgttatcttctgcatcttaatattgttccagaaatactgaggcgtttgttcttcttgaaatatactacttattttcttgttattctcataagactgctatctcaaagctaaatttatgcaacctttcaagagtagagaatcaggagactaaacagagcattacttctattccaagatttacaaagttgacccaaatgtatttcataattaaaagttaatagtaatagtcacatgatttgtatatatctgccagaaataacattttgacacaaacaaatacctggaaaacaactttttatatctccatttcctggatgacagatttctttatgcttgtaagttaccccaaattttttgtttagaatcaaatagtatcttatttggctacttctttttaagataactaacaaattgaaaaaaaattttttcttttaccaattttttgctaaagtcaaaataatatgaaaaaggtttatattgctctgtcactattgttttgaagtcttgatttcatgtgattacaaaataaaaatattaacagactggtaaaaagattataggttgcatataaataatttcaaagtgatggttttttgttttgttttgaatcttgacacaaactcaaactattcataaatacatatatatatgtgtatatatgtatgtatacatatatgtacatatatatatatatacacatacatacatatatacatatatatgtatattgctggaaaatccttgtgaattgaatgttatttttttctagatttacttgccattaaatactaaggagttgtataattttaaatgccacaattaatttttatatgtgtaaataaagctacttagtattgtacataaactcttattaaaattatttagtgtataaagaatttttcaatgtttgagttttgaagattatatagttccaatctttgttccttttcaaagttaatgaatatatgtatttaagtctgccaacacatgtatgtatgtagagatctcagtatttcactcaacaagaaaatcacaatttttcaattttttatggctgaaagtttttgaaggtatttgcttacaaaataggtatgcttatttttaaagaccatcagtgtcttttagtctgattaggttacaccttatgaaatgacaagtagttgctgtcagaaatattttttaagggtagaaaataaaggtaaattaagatttttttataagaaacatagatttgacaagtgcttatcatttagagttttgctttaatggaaaagttcattgcaatgatgatctctattttagagtttaatattcctaggaacttttaaaacagataattttgaaattccatgtattcctgcttgttcaatctggtgtgtgttgggagacttacgcattaaaatttattattagtaaatcttaaaaaaaaaaaaaaaaaaaaaaa

SEQ ID NO: 8 - DNA, 20 bp, Artificial Sequence
  gRNA1 for chicken Caspase 8
  misc_feature: Synthetic
atacaaagactgcactgcag

SEQ ID NO: 9 - DNA, 20 bp, Artificial Sequence
  gRNA2 for chicken Atp5b
  misc_feature: Synthetic
ctcgggccccgttaaagcgg

SEQ ID NO: 10 - DNA, 20 bp, Artificial Sequence
  gRNA3 for chickenCdc20
  misc_feature: Synthetic
gatggtcgctacctggccag

SEQ ID NO: 11 - DNA, 4107 bp, Streptococcus pyogenes M1 GAS
  misc_feature: Cas9  Accession number AAK33936.1
atggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactga

SEQ ID NO: 12 - protein, 1368 aa, Streptococcus pyogenes M1 GAS [sequence omitted]

SEQ ID NO: 13 - DNA, 7119 bp, Mus musculus
  misc_feature: mouse gene Atp5b (ATP synthase, H+ transporting mitochondrial F1 complex, beta subunit)
gcaggaccggcggagaggaccgcttcggcgctcagtctccacccggattccgccatgttgagtcttgtggggcgtgtggcctcggcctcggcctccggggccttgcggggactcagcccttcggcggctctgccacaggcgcagcttctactgcgagctgctcccgccggggttcatcctggtaagcgccttcctggaggagctagttcggttcttctccccgtgaccttgagcttgggcctatcggatccgttctccataaggattcccgggcagcggactactgtccagccgaggagtgcaaggacagtcgcccgtgggagcgttgatataaccagcagcgtcccattttgtgaggctagagtggccggcagctgcctcgaccttgaggttttgagggggataccaaactcgagaataatggcggttctgtgcacgaggtcgggaaaacagtgcgggtgggttcccgattgcagcgtgcccttctccatgcctcctcctatcttgcaggagacttagtctgggctcagctaaccttccctcgggggataaatctgggattcgggtgaccttaagctgtcctaacaccaacgccttccaatacctttttgtctgctagccagagactatgcggcgcaggcgtctgcggccccgaaggcaggcactgccaccgggcgaatcgtggcagtcatcggcgctgtggtggacgtccagttcgatgagggattaccacccatcctaaatgccctggaagtgcaaggcagggacagcagactggttttggaggtggcccagcatttgggtaagtaggacgttttagaggtgggaaagagcttgttaggcttaaataacacgagatcgtgccatctccattctgtgatgactttaccaaatgactttcgttcttctgagtttgctgaagccacactcaggtgctgagaggggaggcctgtttgcttttgatcacccttcattacatactgttaggttttggtgcttataacgtgttaatgctgattgacaggggagagcacggtcagaactattgctatggatggcactgaaggcttggttagaggccagaaagtactggattcaggggcaccaatcaaaattcctgttggtcctgagaccttgggcagaatcatgaatgtcattggagaacctattgatgagagaggtcctatcaaaaccaaacagtaagtttctctcactgcatttgtgaacatgggaaccttttcttgcagttgtacacatggggtaataactggtagtaaagcaacattttcctacagtactacttccatttctgttgtattcagttattgaaaatgaagatacagaagtttggattgttgaaaacttgctacatagaaccccctgttactttttttaaagctgtgttacattatggattctctaaaatttacaggtttttattttcttattgttatataaagaaactttgttatattaagttcagtaaaaggaaaatatcaaaaaagaggattatttgttcttgagactttttttttttggtttttcgagacagggtttctctgtatagccctggctgtcctggagctcactctgtagaccaggctggcctcgaactcagaaatctgcccgcctctgcctcccaagtgctgggatcaaaggcgtgcgccaccacgcccggcgagacaatattttttattgcagagagatgatgttctgtgacagcttaacttgaacaagattagacttgactgtttgtggtggccagtggcttttgagcatggtgcctggttctgaccctttcctccctgaaccttacagatttgctcctattcatgctgaggctcctgagttcatagagatgagtgttgagcaggagattctggtgactgggataaaggttgtggatctgctggccccatacgccaagggtgggaaaatcggtacgttggtttttggtccgtgttttccaaatacagtaaagcatggagacccttttttcttttgagagtgcttgagccttcagtgatgattagcttctgactttcgttcttctgaatttgctgaagccagatgccgttcctgagaaggggaaaatggacagaactgaacatttttgaaggctgtttaatttgggaatgcagagatctgaaatatttcttcttaaactaggactctttggaggtgctggcgttggaaagacagtactgatcatggagctaatcaacaatgtcgccaaagcccatggtggttactctgtatttgctggtgttggtgagaggacccgtgagggcaatgatttataccatgaaatgattgaatctggtgttatcaatctaaaagatgccacttccaaggtgagctccattcagaaaactaatgtgttatgtttgatgtgatgagccccgggttctgtaaacaaaatagttgtcttcccaccaccattttccagaataccagtgtacacttctctggtgtgagatatttctagactttttgatgagtaagtaatccaatttgaaagctgatttaaaaggtgtagaatggaagccactcacatatctcagtcttacttttagtttagaaattatttccagtgttctcaatttcatgaaggttccaaacagaaacattggcatccagtggaagatcccattagtggtgtctctctctctcttcccctctcttctctctgattgggaatcttggatttctctgtggcttccttactactctgctgtcttttctgacttgtgtatgttttacaggtagcgttggtatatggacagatgaacgaaccacctggcgctcgagcccgggtagctctgactggtttgaccgttgctgaatacttcagagaccaggagggccaagatgtcctgctgtttattgacaacatcttccgctttacccaggctggctcagaggtaggggggaagcctcgaggcctggtttgttgagccagttgggcatattagaagaaagatgtagacacctaagaccattattctttacttctgtcttttaggtgtctgccttattgggcagaatcccttctgctgtaggctaccagcccaccctagccaccgacatgggcacaatgcaggaaaggatcaccaccaccaagaagggatcgatcacctcggtgcaggtaatgaaaaaaaccaacctagggagataataacaccatattatacttggggctagagagataattcacagttaagaatgcttttggctcttccggttgcccagtgtccatcaggtggcttaccatctgtaactccagcttaggagacatggcgccctcttttggcttccatgggtacctgcatatacagtaaacatggtctggggcgcggagatgtacaaaaatcatgtgctaggtgaaatggcacttacctttaatcccaacacttagaaggcaggggcaggtggatctctgagattgaggccaacctgctctatgtagagttccaggacagacagtgttgcttcttgaggtcctgagtttgattcccagcaactacatggtggctcaaagccatctgttatgggatcttctggcatgaaggtatatatacagattgagcactcatgtataaaataaaaaataaatttaaaaagattatgtatttgtatgctagattgtaaaggttaagttagaaacaaatgctcattagctgggcgtggtattacatacatatacctgggatcctagcacatgggaaacaggcagaaggatcaggaactcaatgctatttccacctacatagtgaattcctgcctaacttcactatatgacactgtctcacaccagaaaataggacattttttttttttttaaagatttatttattgattatatgtaagtacactgtagctgtcttcagacactcaagagggagtcagatcttgttacggatggttgtgagccaccatgtggttgctgggatttgaactcctgacctttggaagagcagtcgggtgctcttacccactgagccatctcaccagcccccaataggacatttttttaagtaattctaaggtactggggctgaagagatggctcagtagttaaaagtactttctgtaggcaggtggtggtagcccagaggaagcagaggccggcagatctccaagtgtgaggtcagagtggtctaggaccaattccaagataaccagtgctacatagagaaaccctgtctttttttttttaaagatttattttattattatatctaagtacactgtagctgtcttcagacacaccagaagagggcatcaggtcttattaaggatggttgtgagccaccatgtggttgctgggatttgaactcatgacctttggaagagcagtcagtgctcttaccctctgagccatctctccagcccccgaaaccctgtcttaaagaaagaaaaagaaaaagaaaaaaagccatactttttgggattgtattcccagttggagaggatcagtccgactcctcgggtacattctgcaggctcagtcgtgcatatgatacacaatgaaaatattgttgtatggtttttgacattagatctacttgtgtagcttcagctggcctggtactcactttctgttcagctctgtttggcagctcatagcagtcctcctgcctcagtgtcctaagagttgggattatagacaggagctgctacatctagctttcattgttatcttgattgtgtgctgtgtattccgaggcacataataagcaagcaacaagcaacacataagcaagataatgatgataacagaccttaccatgcaccctcgtggtcatatttacttagtattgcagtgcaggaacatcttttaagagcacaacataaataaaagttggtaccatcacaataaagtgttggcatagtatgcagacccagataaagggagtccatagcttaatccagtcccagagatgccaagaaagaaaaaaaaagaccttgattttaagagggaaatgaaaagagcaggcttaagcaggagtggggttactgctgatctcctgggttcttgactctaggctatctatgtgcctgctgatgacctgactgaccctgcccctgcaaccacctttgcccatttggatgctaccactgtgttgtcccgggctattgctgagttgggcatctatccagctgtggatccactggactccacctctcgaattatggatcccaacattgttggcaatgagcattatgacgtcgcccgaggagtgcagaaaatcctgcaggtgagtatctttatcctcagagtcaatggccatcaaattgtaaggaatttgagacctctataaggactagatttttttttccccctagtcatttgtgtgaacaaggaaagttgagaatggattgatactgctgatgaggtgtccccagtttcagggtactctacttcacccttagaatccagttgtcagactggcagtagtggtgcacatctttaatcccagcactcaggaggcagaggccagcctggtctacaaggatagccaaggctacacaaagaaactctttctcaaaaaaataatcagttgttttctaacctagttgaataggcagaataagacactaaccttggttgatggcttcctggaccttctgaaagtgtaggcacttaactctgtccttcctttcatcttgcttgcttttaggactacaaatctctccaggacatcattgccatcttgggtatggatgaactttctgaggaagataaattgactgtgtcccgggcaagaaagatacagcgcttcttgtctcagccattccaagttgctgaggtcttcacgggtcacatggggaagctggtgcccttgaaggagaccattaaaggattccagcagattttagcaggtgaggttttttctttttatcaagcctagccataaaagtgagagaaaacaagcctatcttctcattctaacatgatcaaatgaaatgatacatacctttcttttctttcttaaagatctattatgtgtatgtacactgtagttttcttcagacactccagagacagcgtcagatcccattacagatggttgtgagccaccatgtggttgctgggaattgaactcaggacctttggaagaacagtctatgctcttaacctctgagccatctctagcccccttgtctatacttttcttaaagtataccattaaaggccattaccctgccttccattaactgtcccatgtcactgagttgaaagtgagaattttaactcaggtgctccagattacgggtttttaagtgcttgtgtgtctgagtgttgtatgtgatgtgagtacacgatcccatgaggagttagatcctcccagaacagtcagtggaggttaggagtggtgtctggtcactggaattagaggtgataaagcagtagtcagtgttaaaccaccactcaagccccaaacatattcttgatctactacatcttgtcttaaattacacactttacactttaaaaacaattatacttggaagggggtggatatggtgcacacctttagtgctgggactcctaaatagatctaggggcttgagttccaggtcagccaggaacacacccccaccccccaaaagtaaatttcacgtggaagttgaaaagtttccttgcatgataaggtttcacagtgggctcaagtggtgaggtgtacatgctgcaggaagctgatataactcagcttcttactgtgttttcccctatgtaggtgaatatgaccatctcccagaacaagccttctacatggtgggacccattgaagaagccgtggcaaaggctgataagctggcagaagagcacgggtcgtgagggactccagccaaaggcagcactgcaactgatctctccatatcaagcgagagctcaggtttccttccatgcaggccacacaagagccttgattgaagatgtgatgttctctctgaagagtatttaaagttttcaataaagtatatacccctcatttatgtctgtttatgttgcttctgaaacagcttataattgagctcacagtggtgggccacatttaggaaattgttagaaacaggattattattaaaaagatcaatttattaatgaga

SEQ ID NO: 14 - DNA, 4452 bp, Mus musculus
  misc_feature: mouse gene Cdc20 (cell division cycle 20)
gccaatgaacgctagcagcggagagtttaagaacaacagtccggcgtgtccttagcttgtctggaaaagaacgcgttcggcaggcgcactgacgcgctgcgaggagctgggggtttgtgttcgggagagctgagtacggagagggaccgggctgctgcgagctgtggcaggcggagcccaggagcccggcgaggatcgcgcttggtcgcctttcgccccggtagcggtgccatggcgcagttcgtgttcgagagcgatttgcactcactgcttcaactggacgcacccatccccaatgccccggttgcgcgctggcagcgcaaagcaaaggaagccacaggcccagccccctcgcccatgcgggccgccaacagatcacacagcgccgggcggaccccgggccgaactcctggtgagtgggtggagggagctggaatcgctgagagtccgtcttcatgctccctccgggagcctcgtcaggctgtctgacttctctctccctgcccccaggcaaatctagttccaaggttcagaccacccctagcaaacctggaggtgaccgctttatcccccaacgcagtgcttctcaaatggaggtggccagcttcctcttaagcaaggaaaaccagccggaagacagggggacgcccaccaaaaaggtatgattccacaggggtcatgagacttgaggtgtctgtcccctggctgataccggtcttcctcccctcacttgccgtgtatttcaggagcatcagaaagcctggtctctcaacctgaacggttttgatgtggaggaagccaagatcctcaggctcagcggcaaacctcagaatgccccagaaggtaaggagtgagtgacatcatcgaggctggtgtcagtccttcctaaggggagacatgtggctgcttttccacatgtggatgggtatcagtttccaagtctagacccactctcttgccacaggctaccagaacagactgaaagtactgtacagtcagaaagccacgcctggctccagcaggaagacttgcagatacatcccttctctgccagacaggatccttgatgcccccgaaatccggaatgactactgtgagtgcactgttgtctttgcggatggagaaagatggcctaggaatggaccagtccaacaaaggacctcatgttttcttccttcccctggcagacctgaatcttgtagattggagctctggaaatgtattggctgtggcactggacaacagtgtgtacttatggaacgctggttctggtgacatcctgcagttgttgcaaatggagcagcctggagactacatatcctcggtggcctggatcaaggagggcaactacctagctgtaggcaccagcaatgctgaggtccaggtgagcctggttgcctgtattgtggctctgtggtcactgggctcaggggggagcttgcccaccttacatctgtgaccctgtggtcattcctctgcagctctgggatgtgcagcagcagaaacgacttcgaaacatgaccagtcactccgctcgagtaagctccctcagttggaacagctatatcctatccaggtcggtgtttttcccagtctatagcagaacctttccggtttctgccatccggaactaaatctcttcttctctcagtggttcacggtctggccacatccaccaccatgatgttcgggtagcagaacaccatgtggccacactgagtggccatagccaggaagtatgtgggcttcgctgggccccagatggacgacatctggcaagtggtggcaatgataacattgtcaacgtgtggcctagtggtcctggagaaagtggatgggctcccctgcaaacattcactcaacatcaaggcgctgtcaaggtgagaaagcatttgagctgaggtctttacagacactcagctataaacgagggactactaagaggagaggacaaggcggggttgagtttaattgtgacacttagagttgagagatggttttgtccactgagtattcataatagatgatgactactgtgcccagagagctgggtgccttccagcatcgtcacgattattgaagcagctcctgtcttggagttgtttgaaaattgttatcacctctgattggctaataaagagctgatcaacctgtagctggggaggagagtttaagatgggtgggaaaagaggtgaggtcaccaggagggagtctccaggagatggtggaggaggagcagccagggcgagacggagatgtcaggttacagggcaggtgctgggtgttaggcatcagagctgggttagaatagatgagaaccctgcccagctatagtgcaagaagctctttaatacatattccaggtttctgtgccatttcaagggaaaggagggcatggaaaagctcagttcttttacacgtagggctgcttctgcctctctgttgacctgacccagcctttatccattccacccaggctgtggcatggtgtccctggcagtccaatatcctggcaacaggaggaggaaccagtgaccggcacattcgcatttggaacgtctgctcaggggcctgtctgagtgctgtggatgtgcattcccaggtagttttgttgtgattgctactggtggtgattcatggtttaacctatcccaggcagaggtgtgttcctctatgtgacatttctgaacagccacttctactccaactctctacctgatcatttctaaatttctgactcagccctcactggcattcccatttcctagtttggcttctctccaaccctaggtcctcaagaatcctcctcctccgtatgtcctaattaagcttgtcagttcaccttgccttcctgaaatgtactgtgttccacttgtactgttttagatgcagaacctgcttagaaatctgaaaagccacctagtctttcatccttctcaggatatttcagtgttagcttgaaataccattcacatccgtcccttcctcagcactctcattcattcattcactcattcattcattcattcattcattgaggcagggtcttgtgagacagagtcttgcctttgctggcctcagtcctgcttttaacatcccaagtgttgagattaccagcatgtaactccatgcccaggctcccacacattttctctctcttccagttccctttatacaatccgagtggtatcttactgtccttaaacctgtgatgactcccacaacctacagcataagatccaaatacattagacccgggctgtgttgtgcctttaatcccagcacttgggaggcagaggcaggcggatttctgagttcgaggccagcctggtctacaaagtgagttccaggacagccagggctatacagaaaaagaaaccctgtatcggaaaaaaaaacaaaaaaccaaatacattagaacatttgaggctcttgctgctgatatgccttgctgtaaatactccagttttccctatctgctcccctcactcggccttcactgcaggtgtgggctctgaaggcagatggttgaagttgccaagggagtcgcttatgggatgactttgggtaacatgaaacttcccttctggtggattcatcttcatcaccatagatgtgtgtctgggggcttcagtgaggactgtgaccattaagttaaatctgtagtgcctgacagggtgtctaatactagaatggactgactcggttctgagtaaccctgcctgactcatgtccgtgatgaccttctccacaggtgtgctccatcctctggtctccccactataaggagctgatctcaggtcacggctttgcccagaaccagctggttatttggaagtatccaaccatggccaaggtggctgagctcaaaggtaggtggcaataggaagccaggcagaaaggccacatagtgtatgtttccatccatatgaaatattgagaataaacagactgatatgacttgcctggggatttggcaagggtggtgggagggtatgattgcacgagaatgggtaaatgcagagttaacaggcttagatttagaatgtcgtggtactggttataacaatgtacaacctaaccttgtaaatacagtaacaccactgaattataaaagggtgagtgtatggcattcaaattgcatttcaaggtgactgtcaaagttagagggaggcctgtgtgtatactcaaatgcactgctctcgccctaggacacacagcacgggtcctgggtcttacaatgagtccagatggggccacagtggcatcagcagcagctgatgagactctacggttgtggcgctgctttgagatggaccctgcccttcggcgtgagcgggaaaaagccagtgtagctaaaagtagcctcatccaccaaggcatccgttgaacaaacgcatttcctttcttgtttttattttattttttctaataaagttcgtatcttcctta

SEQ ID NO: 15 - DNA, 52130 bp, Mus musculus
  misc_feature: mouse gene Casp8 (caspase 8)
ggaataaggaagtgtcctccagaactggcgggagctcttaaggcgggcagaaagcgaagcagcctatggcctctagggctggcaccaggatgccacctctaatgattaactcaaacgcctctaaacattcggaggcatttctgtcccctatgccctagttctctcagttgtctttcattctgacttcggtgcttaaaagtccagcttctcggaatcggtagcaaacctctgtgagccggcgtggaacaggaagtgagtacagttctggggagcgccggcccgggctggaggctcggaaagcccaagccagcggaggcctcgcccgagctggagttgtgaccggcgcggcaggtactcggccacaggtgagtgctgcagagcagtcgggttcctttcccaccagcgggccgagatcccaccggtgggtgtcgatctcgcgtggcgctccccgaaatgcagccatcggggtgccgtcgccgcctctccagaaaactttattggacttcttaggcttactttctttgtagtttggctcaggacctggagctgagttaatcctccggggagtcctctagcttgagctgcgtttcctccggcgattttcgtgctcttcccaactcagacgtccatcaactctaatcccaaattctctcttcctttctccttctcctccaacatctcccctccctttttctctccctcttcttctctttgccttctcctctccctccctctcctcttcctctctccctccctccctctgtccctctttggtttcatgatttcctgcagaagctcaggcttcttgaacttacgaccttgcttccacctcctgagtgctggcagtacagggggtggggggtcgtagggcactagctctgcacttaaaacaccaaccccccaccccctcaccccaacccccacagagaggggatgagagaggagggagggagggggctacaggtgacctgaggccggtaattcctctacgtgctaagcagttgtaagggtgactggcttgttaagtacctgtaaggaattatcatacagtcatatttctgtttcttctttctttctttctttctttctttctttctttctttctttctttctttctttctttctttctttctctctctctctctctctctctctctctctctctttctttctctttctttctttccttccttccttcctttcttttcttttcttttcttttcttttcttttcttttttctttcttttttttctttttcttttttgtctttttaaagggagtcctctaagtgccttctgcttggctaggtctcagtttcattctttaaaaaattatttatttctattttaatttcatgagtatgagtattttgcctgcatgtatgtatgtgcacctggtactgggcggggggggggggggtggcggcacagattccttgaactggatttacagacagtttcagccattatatgggtgctggaaactgaacccaggtggtctgcaagagcattgagtgctcttaaactcctgagttgtctcatcgacaaatctttctttttttaaataaatatggcattgattgaattagcatgaggacgattagtgctcccgtttcttacaaacctatttaaagaagactcaagaactcgtatgtggacacagaggagttgctggtcatggctgtgtgtctcccatgtctctttcccaggagggagaaaggtgaccccatagcaggccatttagctgttatttgattttgcccagtaatttctgtttgtgggtaggttcactccaggaactaatatgatttctccagcagcctgcatagcctgaccacctcaccctatcaggttgctcttttttctttttcttttcttttctttctttctttttttttaatcttccaacttaaaagaaattctaataagaaagtgggcatcttcttgctttatttgacattcctcggatttactgacccatgtgatgtgtcatcctatgctctcatgggcttagtgcctataggggagctccatgtgttttttccagcaagaccacacccccattgcccctgtacagaggttcattgcctctttacacagtatcttctcattgatgattttcaggctgttgttatgtggttcaagtattaccatgagagcttcagttgctgcactcacaggaagttcgtggtctctgacaggaaattctcagaaaacagtgagccagaactggggtagaggttatagaggatgctgggaaaactatagtaactcctggagtggactgggccacagacaggatttgattttgctgcttccatggtgcccaggttcctgggcagaatcaaatgccagctaaaggtcctggtacgatggtgtcacctttgcccagacatcgagggtggacatatgcttttgggcgcctcattcagttccctcagctgtgaagttggcgctctttactctgaaagtttacattctattttaggtagcagaaaacacagtttcaaagtccttttctttttcttctctttaacaccagaaaatattttaactaattcccagcaaggagcttatgtgtttgtacacaggtttgctcacacaatgaaggacccagggtttgcctctttggggagtttgctgtaggaaagccctgattttgactttaggcttattttttgtttgtttgtttgtttggttggttggttggttggttttttttgtttttttgtttgtttgtttgtttgttttgttttgttttgttctgttttagctgtaagtagaaactatcgaactaggagcaaggagaacatccctgtgtaacagtaagacttagctgtggaagtcagcaaaccctcaaagcccaggcaggactctaggcccatcagaggtgaaacacttctggggcctatttttgctagttaataaaattttagaaattaattcttttattttttttaagattctaaaagcaggtagaatgaaagtttttcaggtatattcatatgatttttctaaatttcattgatattagttgtaatgtcttccttgttgcttctaactttgttaatgtgggttttctctcttttgcttggtttagtttgtttgtttgttgtttttaatcttttcttcatatcaactctgcttctttaattctttgtgaaattttttgttttgttttggtttgtttcattaatttctgccctgatcttaattatttttcccatatgtcttaatttttctgttgctatgaggaaacaccatgaccaaggcaacttttagaagaaagcatttaattgggagcttgcttccagtttcagaaggttagttcattatcatcattgtggggcgcatggggacaggcaggcaggaaaagtgctggctggaccagtagctgagctcacctgatccacaagttacaggccaatggagagtgagaaactgggcctggcatgggcttttgaaacggtgaagcccacccccagtgatatgtctttttccagcaagaccacacctcctaatccttctcaaagagttccagtaagggctgttctcattcagaccaccacagcatctgttgctttggggtttagcttgctctcattttgccacagtcttaaggtgttttcattgagttactaatttgaaacctctctgacttttttcccatttgtttagtgtgtgtgtgtgtgtttagctatgtgcatgccaccgtgctcttgtttcaccctgtgagttccaggctttagaagcaagcacctttatctgctgagccatcttgccagtccctctcgaagtttgtgtgtcgaaggtttctttttattattatttttaatcatttaaaattatgcgtatatgggtgtgtatatgcatgtgagtgccatgcccaagaagccagaagagagtgttgggccccttggagttggagttacagatggttatgagctgcccaatgctgggaattgaactctggtcctctgtaagtgcagcaagtgctcttaaccactgagccacctctccagtccctctctctgctttgtaatgtaggtgcttatagctataaatttgcctcttagggcagctttcattgtatcccacaggttttagtgtgttgtgttttcattttcaattttgcctagggattttttttttaatttccttcttgacttcatcagtaaactggtcatcattcactagcgtgttatttacttagtgttaattagtgtgttaatgagtttgtatatttttttaagcattcttttgctgttaatttctagctttacttcacggttgtcagacagagcgtgagatgttctttccactttcctgtatttgctaaggcttgctttgtgtcctcttacattatccgagagctctgaggaccaccagaagacacgcactcggcagtgtttgatggaaaccttctgtagatcgctgttaactccatttgatctggtatctcatgtaactcagatgattctctacttagagatgggaatgaagtggtgaagttacccacttactgtgctgggtcagttcgtgactttagcctgagtagaatttgttctatgtatacatcaatgtttagaagttcaatgtcctcttgatggattgttcccttaatccatatgaagtaaacttctgtaccttctgactagttttgggtagaagtctctcttgtcaagtatttgaacaatgatgcttacttgtctcctagatgcattccttgcgtccatgataccgtacccttctaccccttcaccttcaaggcagtgttcctttatctttgatgtcaaagggttttttttttcccctcaatgacaataggtggatcctgtttccttgttttgtttttgtttttaaatgcaatctgcagttcgtgacttttgattgtgatcccaagaccactaatacccagaattacttttgaaaggcattgctcaagaagtaagtccaacagttgccaaatgagacctcatgaaactaaaaagctctttgtatgtcgaaggaactattaattgggcaaagagacagcattcagaatgggagggaaatctttgcaaactatacatctgacaaaatattagtctagactatgcaaagaactaaaaaagtaaacagtaagaaatcaaacaatccaacaaatgaacaagtggctcacaaaaattgaactaaagatccagggggttggctgaggggcgaaggtgctgggtcaccgagctggacaacctgagttctatcccagagcatacacgacagacggatgaggatggacggacggacgaggacagactcccaaaggctgtctctgacttccacaagtggcaagaatgcctctcactctcggttgaataaataaacaaaaaaagtaattgattaaataaatggtaagaaaacaatgaaaacgttctccgctccactggccatcagagaagtgtgagttaaagacctttgagtttgcatctcatccctgtcagaatagcagtcattaaaataacaaataatggggcctagtgagatggctcagtgggtaagagcaccctactgctcttccaaaggtccgaagttcaaatcccagcaaccacatggtggctcacaaccatctgtaatgagatctgactccctcttctggagtgtttgaagacagctacagtgtacttacatataataaaaataaaaataaattaaaaaacaaataacatttattctgatggagttaggaattttgctgtatctctttggaatttatttagctgcaagagagctaacgaaactgaatttattagttagtaattgcaattcctgtgcccccatactttgtagtcacatttttaaaaaattagatattttctttatttacatttcaagtgctatcccgaaaattcctactttaatgatgacatagaaaagccaaaaattactaaacactaatactccactaatactctagatggtaattgttaaaattatcatcttttatttacctagtcacttgaaaacatgagtatctaaaacctggcagaagctatgttatacaacaagaataaaaataaagttatacaatgaacaataaaatcaacaaaatgaatccagcaagattggaagacaaaagatcaatatatgaaagatttattgatcggtttgggcaatgtatacctgtgaccctccgaaggcaggacttaagctttaagagaatcatgagtttgagaccaacctatgtattcataaaaaaaattaaatatattcatttgaaaaaaaagataataaataacaacaaacgctggcccttatacactcctgatgggaacgtgagctgggccagcctctaggaagtcaaaatggaggttgttaaaaaaaaaaaaaaaaaaaaaaaagctcttccatggacccagctgtatttatgcaaaggactctaagtcagcatatcatggagtcacttgcacatccatacttattcataatagttaagttatggaaccaaccgaccattaaaaacagaggaatggatgaggaaattgtggcattttatatatatatacacacacacacacacactcactcacacactctcactctctcacacacactcactcacactcatactcatgcacacacttacacattcactctctcacactcacatacatactcacgcacacactcacacacacatacacactcacacaaatttcagccataaagaacggagtgacagcccagcgatggtggcgcacacctttaatcccagcacttggaaggcagaggcaggtggatccctgagtttgaggctggtctcgtctacaaagccagtttcaggacagccagggtcaaacaaaaccaaagccaaaccaaaaaacaaaacaaaatgggaaatgtttgacagaaaatagacaaaaaactgagataatcattttaagtgaactgaaccaatctcagaaggacagatctcatttgttttttcatttgtggttcctagattttattaagacccgtaaattcaaacgtacgctgcgaaagcagaaacgaaactgggagaccaagggactaacaggaaatgggggatgggcaaatggggagactatggctaatgtagtgtatacatgtatagaactttgcacagccttagccagtgtcattctactactcaggccagcctgcttgaggctgcctcctcctatgtcctgtctcaggaaagcaggaagtgtgagaggctctaccctccagctctctgttgccccctgcctgccacagaccccagacagagaaggggcttgggagcaaggtgcctcctgaggatcctcgggacttgtttctgataaggtcagctgggcttttaagcagggatggatcttgattctaaagctttctaggtttcggagtgcgggatctggagtccacaaggccatagagggtttcagggtttgaatactgaaagggaagccgggagctttctgtgtgagtagctgccttcttccctcttcgagggttctcccccccctccctcctgggtgttcctggacagccatctattgtccccctccttgaaaagaagccaaaattcagatctcaattgatttggctgaactgtctcctcaccgtctcacccaccctgaaactacacatctgatactcacacacgcacaccccaacacgcatactcccaaacacagacacaatctagtgattttatctatttatgtttttaaatccgcagttccgggaattgaattcagggccttacctgctaggcaagggctctatcaccaaactccctcctcagcctatcgtctctatttttttgagaaattaaaaagaaacaagacagcccagagaaggaaacaataggaagcacagtttaagagcccagaaatccgttccagcgtcaggttacaagttatattcagttagaaaacgacggggttggttgttcattttattgacgtttgattgcctttaatgaggtggatccaacacctgcctgctgcggcttgttgatcgtgctcctgtgtgatgtgtggagttctttggggtttaggggagcccaattctgggagcagccttgcctagaaggcagaggcagagctcaaggttaggatttggtctgatggagcctctcagattctctcaggctttggatgacgtattgacttttatccctggaaaggtttttatccctggaaaggtttttatccctggaaagttctagcaagtttcattttatgtctccccaccacctccatctcggagtcggttatctaaatctcaggaggggcagaatgtaccttattatttatggaataatgattagaaacagtaacctggcacaagagggtgtggtggagcttggatcatagatcgggcctccggtctaagtagactagcacttcctcaacagtgggcaggccacacactccattttatgtgcagttttttccgtttttttcttttttttttaattctttaacttttaatctttggccattgtacccatttgaaaatacgactattagtaattctctattgaatttcctcattgtcttctaatagaataaaatagcatatctctttctgtatatgtgtgcgtgttgtggtatgctcactcactgttctgtgtgcattcttcaggtgctatcttgggtcttttgctggcctgaaactctccaggtagctcaggcagcctggctggtgttttaaaaactccacattttaatgtgtgtgcttttggaactactgagtgactggtgagtctgagcctatctctggctgactagctgatttaagttatgatttagtgcaataggaaaatgaagactgtagtaaccatttcttcagaggcttctagcaaaatgagataagaaaatgatttatgtagttatgggtacttctgaatgcttctaagtcataatgtaaacattgatttacatttaaatattttaagttattcatatataaaatatgaaatgttctatatatcataaagtggaaacatatttagattatttgacagcaataataaaatatataaataatatgataaactcaaatacttctcaaatttatttatacatatctatttttgtagagagatgtgaataaatttcaggacacaatgccaaataggaccagagagttactttagaatgataagatttaaatgattttcttttcttttcttttcttttcttttcttttcttttcttttcttttcttttcttttcttttcttttcttttctcttctcttcttttcttttcttttcttttcttttcttttcttttcttttcttacttttccacactttctctgtgaggtgttttacacttagtgtcttcagtaacaaaaacaacagcaagcatttcaaaaatattaacagtgttaatttcccacaagttcaagtcaccagtttaggtcccataaaatgaggccacagtgtggtccccgttcttatagagtgagtgactaaactcacccatggtctcacaaattccatgaaactgattgctttttgcggggctatgagtagagaaatgtgtgacccaaacaggaacaagcaagcaagatggaacgtcaggtaccatggagaggcctgggaagctaagaggccttaccaactccttcagaagcagtcacctcatagaggtgcagggaagagtgagctgggatgggcacaggggccagatgcccatcacaccatgtccagactcactgtcacactgacctcaggtcataacgtgggggaaggtgttgttctgtgggaaagggacttgtaaggaggaaggagggtgtggggtgggaggaggagaagagatggtgggatcgtcaagggtaaacagaaagggtgatagacataaatgaaagaaatggtcaaagaacaagcctagccaattaaaacaattaaaagttttaaacagctctctttaagctctcacctttcttactctctagccctccttctctctctcttccctcctctctccacgtggccatagccggcatctctctctttctaccttctccctttctccctgccttcctacaataaagctgtttaaaaaaaaagttttaaacaaaagaaatgctgttgaatttgtgatgcctgaaaaggcttattaactaaattccttgaccgtataatattataggaaagtattctaaggaaataggattcagagatttacattttaaaattatcctgagtttaataatgactggttatgtgaattacgtacttatggtagatgccttatggatactaaaatctgtgttatagagaaaaaagacatcagaacgtattttcagtattattaatgggaaaaaagcatgattaaaatatttggcagattaagcagtaaaatattactacatatttatctttggttggtggaattacaggtagttttatttatttttaaaatattattttattatttaatgtatatttaatgtatatgtcttcagacacaccagaatagggcatcagatcccattacagatggttgtgagccaccatgtggttgttgggaattgaactcaggacctctggaagagcagtcagtgctcttaaaccattgagctgtctctccagcccagaagtcttgtttctttttcttgtggattttttttccctattaaatgggattttaacagattaaactgctttaaaattttaagggtgtttggcctaatgttgatcaacacactggccctaagttaaggtacttttgtgattacatcatggatttgggaaggacgcatgttctctcctgggagagattcacagctagaatatgccccctgatctgtctgttcactttgggtttctctgaaagatgtgtgagctgggtttttttcccatatgaagttgagtattgttctttcaaaatatataaaaacattgtattggaatattgatggggatagcattgaatctggagattgcttttgataaaatggccatttttactttgttaatcctactgatctttgagcatgggctatctttccatcttctgataccttcttcagtttctttcttcaaagacttgaatcttttgtcatgcaggcctttcagttgcttggttagagttaccccaagatattttatagtatctgtggctattgtgaaggatgctgttctcttctcttctcttctcttctcttctcttctcttctcttctcttctcttctcttctcttctcttctcttctcttctcttctcccctcccctcccctcccctcccctcccctcccctcccctcccctcccctcccctcttctcttctcttctcttctcttctctcttgttttactgagtcctgcctctctggggtacccaggcacctgggaggggacagggcaaagtccgaatctttgggggaaggcaagtttccagttctctaagaacttggttctaatgctaagttttcttaagagcatttgtgctgacgcccagactgagaggcacttcctgccctgccttggacttcctggaagtcaacttcctgtcttctacaccatacagtctttaacacgggaacacacccagcatttcttttcattctcttattttctcttcctctccaagtctaacaatttatttaagaaaggtgcttatttttcaaagctggcaacctggctcaatccccaaaacccacacaaatgtggaaggcaggagctcttcccagaggtcctggtacacttgctgtggcacacacatgcctatacccaacacacacacacacacacacacacacacacacacaaataatgaattcagtaaaagggaaataagtcagaacttatagaagatgagagaaatgtctttgtgtggtgtcattccaacaattaatatgtgaatgtactgggcatggcactcatcttcaattccagctcttgagaggccatggcaggaagacagtaccggtgcacactgaattccagaacttttttccttttttccttttttccttttccttttcctcccctcctctcctctccctcccctttttccttttctccttttcttttctttttcttctcttttctttcttttctttctcgtcgtactgagtcctgcctctctggggtacccaggaacctgggaggggtcagggcaaagtccgaatcagggagtccacaatgattcctggtcttctctctggcgtaccatcccagggagtcaggaactggtctgcggtctttgcttctcctcagggcatctttgtgacaagcaggagaggggagatggagccccacatgagcagaattcagtctgattaggagtgaggggctggagaggaggcagccttcttctggagtcttaggcatggctccttagtcaaaggccttcttcttgggtgatccttaatggagcagctttctgagacaatccttgcttgttttttattgctttatttgatggtggatgtaaatgcgtacactttattcgggatgaccaaggggttatataccttttgggggcaggatatccaggaagggaagctcattggctgaaggctcaaggctatctagacacctcattaacatgaagaagaattccaagtgttatgctatgtgactgactgtctggtcttctcagtggggtgtagtggttacatgttgtagagcaggtacagagacaggtagggaatggctccattcctgccaccaaatctctgagattcctggtttgagctcattcagccttaaattcttatgcctgtctggtagtgcattcccacatgtcctacattctaacgtttttcctcctttcttgtggttcttctatttctctccctacctgggacccctggctgggatcagaagtccagccttcctgtctcccctgcccagtcataggctgatcatcattttattaacctatcagagataattggaggatattctttacataacattgacacaggagatagttcaacaatcaatacaaagctagtgttcatactgtaaccagatctctgggcacagaaatcagccaacacacaattagcccaccaccatttgttggagatgttttctttctttttttttttttttcaattgtatttttctggcttctttatcaaaaccaggtgttcataggtgtggggatttacttctgtgtcttcaatttgattccactgatcatatacctgtctgtttttatgctaatacaatgtgaatttattactacagctctatgacagagcttaaaaccaagggtcgtgcgggacgtggtggcgcacgcctttaatcccagcactcaggaggcagaggcaggcagatttctgagttctaggtcagcttggtctacagagtgagttccaggacatccagggctacacagagaaaccctgtctcgaaaaaacaaacaaacaaacaaacaaacaaccccaaaatacaaaaacaaaaaaacaaaccccaagggtcgtgatacaagttccttactgtacaggatggtttaatctatcctggggttttttttcccatatgaggttgagtattgttctttcaaagcatataaaaacattgtattgaaatattgatagggatagcattgaatctggagattgcttttgataaagtggccatttatttatttattttgttttttcaagacaaggtttctctgtgtagtcctggctgtcctggaactcactttgtagaccaggctggtctcgtactctgaaatctgcctgcctctgcctcccaagtgctgggattacaggcatgtgccaccactgcctggcctaaaatggccatttttactttgttaatcctactgatctatgagcatgggagatctttccatcttctgataaacttcttcattttctttcttcaaagactttaatcttttgtcatgcaagtctttcagttgcttggttagagttaccccaagatattttatagtatctgtgactattgtgaaggatgctgttcccctgattttttttctcagtgatttattatttatgtatagaagggctagtgacttttttgtttgttcgtctgtttgtttgttttgttaatcttgtatccagccacttcaccaaaagtgtttatcagctgtaggagttccctggtagaatttttgaggtcacatatgtatactatcatatcatctgcaaatagcaatactttgacttcttccattacaatttgtgtcttcttgatctccttttgtcttactgactagctggaacttccagcactatgctgaatagatacagagaaagaggacagccttgacttgctcctgattttagtggaattgcttttgagtttctctccatttaatttgatattggctatacgcttgctgtaaattgtttactttactatgctttctttttttttaattatatttttctgggttccttatcatgtttaggtatgtccattgtatcccaaatctcttccagacttttaaaatgaatgggtgttggattgtgtcaaaggctttttccacatctaatgagatgacaatgtagggttttgtctttcagttcctttatatgatctattacattgatagattttcatatgttgcgtcatccctgcatctctaggatgaagcctacttgatcaaggtagatgatctttttggtgtgttcttggatttggtttgtgagtattttattgaacatttttgcatcggtgttcatgggggaattgatctgtaattctctttctttgttgaatctttatgtggtttcagtatcagggtgaatggcctcataaaacaattttggcaatgtttcttctgtttctgttttgtggaataacttgaggagaattggcattagttctttctttgaaagtctggtagaaatctgtgctaaaatcttctggatctgtttttatgtttttggttttggttgttgttgttgttgttgtttggtttttgttcatttgtttttgagttgggagacttttaatgactacttccatttccttgggggttataggtcttttaaaaatgtttatctgatattgatttaactttggagagtggtatctatcaaaatgtccatttgtcttagatatttcaattttgtggggtacagggttttgaagtatgactgaatgattctttccatttccttagtgttgttttaaatcccccttttcgtttctaattttgttcatttggatattctctcttctttttagttaatctagataaaagtttatcttgttggctctcaaagaactaactctttatttcattgattgctttgtattattctcatttctattttagtaatttcagccctcagtttgattatttcctaccatatactcctcctgagtgtgtttgcttcttttttgttctagagctttcaggtgtgctgttaacttgctagtatgaaatcttctttatgaagccacttagtgctatgaactttcctcttagcattgctttcattctgttccataagtctgggtatgtctggcattaattttcattgaattctaggaagtctctgatttctttctttatttttgccttgacccagtagtcatgcagtagagagatggtcaattactacaaactgtagactttctgttgcttctgttgttgttgaaatcgagctttaatctgtggtagtctgataaaagagcttatttcaattttcttgtatctgttgataattgctttgtgactgaacacgtggtcaattttggaaaaagctgacaaagtgctaagaagaaggtacattctgttgtgtttgggtgaaatgttgtatagatagctgttaggtccatttggttcataacttctgttagctccactatttctctacttagttttgcctggatgccctgtccattaatgacaatggggtatttaagtcttccactaatcatgtgtgaggttcaatgtgtaatttaagcctttgcaatgttccttttatgaatgtgggtgctcttatctttggggtagagatgttaaggattttttgtttgatgagtatggtatccttccccatctcttttgattaattttggtttgaactctatcttgttggatatttcaaaagatataacagctttcttcctctgtccatttacttggaaaatggtttttctatcttttactctgaggtaatgtctctttgttggtgaggtgtatttcttttatgctgcataaggaaggatcatgttttcatagccattcttgtagccggtgttttttttattggggaattgagtctactgatattgagagatattaatgaccaaagattattaattcctgttattttgttgttgttagtgatggtggtgtgtgtgtgtgtgtgtgtgtgtgtgttattagaagcgttctcacgcccggccaggaagaacaccacagaccagaatcttctgcggcaaaactttattgcttacatcttcaggagcaagagtgtaagaagcaagagagagagaaaacgaaaccccgtccctattaaggagaattatccttcgcctaggacgtgtcactccctgattggctgcagcccatcggccgagttgacgtcacggggaaggcagagcacatggagtggagaaccaccctcggcacatgcgcagattatttgtttaccacttagaacacagctgtcagcgccatcttgtaaaggcgaatgtgggcgcggctcccaacatctccccctttccttttaataagagcaaataggccacccatattaatgagagtggagatagaggtcaaatccccagtgtgtaggtaaaggagccatgtacaggattagctcttaggctcacaggcttttacccagagcaaccctgacctgctcccgtgtcgttttgcctggagagaaggacacttggacactcaaccttcttgaaagatgacatgtctccctagaataggctcatatatgccgcagagcctttctactgcagtgcttagccgtgcaactctctcgggctgctgaagcacactcactctatcccgtgcaatgagactagcctcatgggatataagagctgagtggccagcgacctattgcctaagcatagatatatcaggggaagctccatgttctagtcctgcaagcgcctgggcaataaccaccttgtctctcctagtttgggccttaagcttacagaccaatcaaagaagcaacactaatccacagcaaagtgtatctccaaataatattaatcccacccattttttaaagaaggaaaatgctgaggagatccaattgggtaatcctttggtcagcgacaggtccaagcgcgtggagttgacctgaagtctcaattcccgaagggatctgttcaaattcagccgtccaattctgtaacatatactgaaaaagactttttgacaaattagctgtcctagtaaatttctcatactgaatggaagtaacacacaatctcggaaacttttgttcacatctcagctgagctatttgccataatacatctagttgtttctggacaagatctatgagttgattaaccagcatgagacctctctgtatctagacattagttgaggcctgtttatctatgactgtagtcactgaggctgacaaagtgttaatggtgtcagtcgtctggacctgtccagacagagccaaggctgtctgaatcaaggctaaacccagttcctagttagtggtaaaaaagcaggagaatacttgagcattatacatcaccgtcattgggagtggaaatatcgacattctcccccaacgctgctctctttttattattgacgccctggacatcaccaagacgagggacatcagtattcccttggtcagtctggatttttcgggtgagtctttctggtatccaaaatgggttgtcttcattctgtgggaaaacacagatagctcccctggatcttatcaaaataggatccgggccataccatttattatcaaggacatttttccatttaaccatctcattgggcctatctggctctgaacaatgacgttcagccgcagtatggccatgagcatcaatatttaaaaaattgagtgtaaagagtgccaaagacacagacactcttggtgctcggggtaaaatctcttcaattcccctcttctgttttataagataggttttgagggtgcgatgcgcacgctcaacaataccctgtccttgagggttgtatggaagtccagtcaggtgggtcacgtccatctgacggcagaactgttggaatttttgagacgtataagctggtccattatcagtcttaaggagtctgggtttcccccaagcactccatgcctcaagacaatgttgaatcacatgtgaggctttttctccggttaacggagaagcaaacatgatgccagaacatgtgtcaatggacacatggagatattgaagttttccaaaggaagaaacatgtgtaacatccatttgccagacctgtagaggtcgaataccgcgtgggttaattcccacatgaggaactggcaagaactcacagcagctttgacattgagtaacaatgtcacgggcttcttttcttgtcagggagaaacgactgcgtaatgtttcagccgtcacatgaaaattgttatgaaaatttcttgcagcctctaccggggatgatagggcagcagccatcactttagtggccttatctgccaaatcatttcccagagccatggggccaggtaggcctgaatgggctctaacatgagtaatataaacaggaaatcttctagataacaaaactaattgtatctgctgaaaaatattggctactctactggaaggcttaatcactccagccacttctaaaagatttactgcattaaccacataacaggaatctgacacaatattaaggggttctaaaaaggtttttaaaacttctaagaccactaaacattctaccacttgaggtgaaatttcattatactgtttggataccactttaccattagccacataggcacctatgccagtttttgatccatcagtatataccacaatcccatttttaagtgggtttcttactgttatttgtggaaacacaacagattgattttggacaaactgtaaaagtggatgttttggataatggttatctatttgtcctgaaaaggaggtaactaaaactgcccaatcattagatgtggctgccaaggtttgaacctgtgcagcggtataaggtacaattaaaagatatggacttcgcccaaagtgggtgattgctgcttttagacctttaagggcaagctgtgcaattgcatcaggataccaatctattattttagctggggatacgtttggatggatccacaacaatggcccattctgccacaaaactgcagttggcaattgtgctgtcttaaagacacacaaactgaaaggctgcgaatcctcaatacattgtaattgtgcattctgtaaggcattttccactttttgtaaggcctggttagcagctagagtaagagtcctaggggaggagatatgaggatctccttctaaaataccaaacaaaggccttaactcagcggaaggaatctttaaaaaaggtctgagccaattaatatctcccaacagcttttgaaaatcatttaaggtatggaggtgatctcttcttatctctaccttttggggcacaatcttatctggggacaccacagagcccaagaattgtcctgtatcagaaatttggaccttttctgtggctatctgtaaaccccactgacttaaagttttaagtagaaaaggatatgccttttgtagcatggtaaggtctttatggcacaggaggatgtcatccatgtaaaggagcaaaattaaagaggggaattgttccctcactggcaaaagagcttcttgcacataaagttggcacattgtaggactattggacattccctgtggtaagaccttccattgatacctcttatcaggttccatgtgattaatagaggggatggtaaaggcaaatctgggcctatcccttggacacaaaggtatagaaaagaaacaatctttaatatctataataattaaattccagccacgtggtaaggcggaaagtacagggagacccctctgtactgggccaaataagttcatttgctcattaatggctctgaggtcatggagcagtctccactttcctgactttttcttaattacaaaaattggagtattccaaggtgaggtagatggctcaatatggcctagttttaattgttcctctaccagttgaatcacagcttctagtttttcagaggataggtgccattgaggaacccacactgggtcccctgttttccatggtatgggtcgtgctgccccaatggccgctatggaaaacccagaccctgtctgtcttggtttccattaggtaagataggctctatccttccctgttcttgatgtcctaacccttttccttctttataacccatctttgccatgatattttttgctttagttgaataccctcccgatggggcgttttcattggacaaaataaggcccaaatgctgcataatatcccttccccagaggttaaccgggagtgggagcacataaggtatgaatttcccttgctgtccttcagaggattcccacgtcaaggcaacggagcttatagtgggacatgattgatatcctaggccctgtaatgaatgagatgactctgtggtgggccacgctttgggccaccaatgtgtagagattatacttttatctgctccggtatcaaggatgccttcaaactcttttccgttaatcttaaggcggagcttaggtctatcatttaaagatacaaccaaataggcagaatcatttcctgaggagcccattttctttatctcaggtcctgcagatttctccctggtattatcagggaggagcagcagctgagctatcctatctcctttactaatagaaaaaacgcccttagggcttgagcacaggacctgtatttcaggggaatgttgacaatccataactccagggtggactactaagccctgcaaggtgagtgaaccccggccgagaataaggcccatggttcccgggggcaaggatggtataggctccactggcaccggctgaatactcatttgaggcattaataggaagtcggaggcggcacgcaggtccacccttgtgggtcttcctgggtcgcctctctgactgcttcctgggtcctgacaaaccggttcccatatctttgagggccctgggaccgagggcccgatgacccgttttttggcacataagctgattgactatcaggtgggggaagaattctgccctttatatccctcacagagcgacactggtcggctctatgataacccttgccacacttagagcaaagagtgagagtccctccctgtttatctggagctctgcaatctttcttaaaatgcccaggctttccgcaattaaaacatgtcctctgatcatttctgcccatggagcggttctgagattgaaggatggcggccgctaagcctgcattggtgagaggtcccccaagctctcgacagaccctgagccagtcttgtaagcctttgttctttcttggggctatggccgctcggcactcctttgtggcttgctcatagattagctgttctaccagaggcgcggcttgctctgaatctccaaaaatacgctctgctgcctctgtcattctggccacaaaatctgagaaggattcctgaggtccctggacaacttttgttaattgaccagtggtttcacctgctcgggagagcgccttccaggccctaatagccgtggaggaaacttgggcataagctccccaatggtagtttgtttgatcagcagaataagctccctgacccgttaacaagtcaaaagtccaatctctctgctctggagtcaaagcagctgcgtttgctcgggcctgcgcttgtgcagtttcatgccaaagagctctccattccatatatttgcccatactagggagagcggcttttacaaccgtttgccagtcggcaggagttagtgccatgccggcaagcctgtctaactgcaccaaggtaaaattagcattggttccgtatttacggaccgactcggcaagttctttaatttgtaagtattctaccggagcgtggacacgcccaccctcggctccttcaaagactggaaatgcctgttgtattttcctttgttcctctctgggaatgaatgagtctgcgcactgcctctctgcgcactgcctctctgcgcactgcctctctgcgcatctctctgcgcattgcctctctgcgcatctctctgtgcattgcctctctgcgcatctctctgcgcattgcctctctgcgcatctctgacgcactacgcagggcggggactccgcatagggcggaccttgaaaccgactgccctgaggccaatcagcaaactggccttcgccagccgcttttggcttccttaactgattagctagcactttacctggctggtacccttttttctcataatgagctgcttcttcctcccagtctgtttcttcagaggagaattcatcatctgattcagagctaccaagagctggctccccgagcccatccagcgatgagcacaggctccttttcctagagacctccgctaattgatctttcttcttttccctttttcctctttttcttctaatctctctccaggtattcctacctaaccttaacttttcctcgggttcaagacccttggaaaggcctgtatacttattttgtgtaccatatttcctctttgttcctactctctctccccactttacttctgatagcttgtcctgaatttcctctagaattttcagccctatcttaatcacttgataacatgtgaaaaggaacaaaagggctcctaacaccagaaaaaattcaaggccaaacattttccactttacttctgatagactgtcttgaatttccttagaaagttcaaggccaggcttacctcgtaaagctatactcactggtactctcgttccccagctgaaaagttctgaattcatgcagttgaatccttcttaacagtctgctttacgggaacctttattaccgcgacccgcagttctggttctggaatgagggatcttccttgcgccagtcccgagttttttctcgtcccggaattcggcaccaattgttattagaagcgttctcacgcccggccaggaagaacaccacagaccagaatcttctgcggcaaaactttattgcttacatcttcaggagcaagagtgtaagaagcaagagagagagaaaacgaaaccccgtccctattaaggagaattatccttcgcctaggacgtgtcactccctgattggctgcagcccatcggccgagttgacgtcacggggaaggcagagcacatggagtggagaaccaccctcggcacatgcgcagattatttgtttaccacttagaacacagctgtcagcgccatcttgtaaaggcgaatgtgggcgcggctcccaacatgtgtgtgtgtgtgtttcccttctctgggttttgctagtgtgagattatttatttcatgttttcatagttatagttaatcttcttgggttggagctttccttctaataccttgtgtagggctagatttgtggatagatattatttaaatttgactttgtaatggagtatcttgttttctccatctataataattgaaattttgctgggtatagtagtctgggctgttatctgtgatctcataagagtctgcaagacatctgttcaggcctttctgtcttttagagtttccattgaggaatcagatgtaattctgagaggtatccctttatatgttacttggccttggtttctgttttgcttttggtcttttggtctttgtggtctgtatttctggtgactgttgtgacctccctctctatttttctgtctatacagggtctctctatgtagccctggctaccctggaactcacactgtagaccaggctggtcttgaactcagatatccacttgtttctgcttcctgagtactgggagtaaaggtgtgtgccaccactggttggtaaaggacctgtttcaaaaaatgaaaacagctgggcttggtggcacaagcctttaatctcagcacttgggaggcagattcaggtggatttctgagttcaaggccagcctggtttacagagtgagctccaggacaaccaggactatacagagaaaccctgtctcaaaaaatcaaaagaaaaaaaaaaagaagaagaagaaaaaaaatgaaaacacagagcaaaagcaagaacaaacaaacaaacaaaaaacccaaccctgttgcattagttttcaacccaatataagaaacacacaatctggttattgtatttataagctgtatgcctagattgggcagatctagtgctatactagtttctcctggctccatcttcgtttcctcctcttcctctctcttctcccttccttcttctctttctcctcctaactgcccccactctccaagacctcccatcccacctttcccttccactgcccaatcacagcctttagtctttatttgaccagttcaaatggggaaaagattcacatgaggtctcatgagtatgtgatcagctgattgacagggagcagcctgtcttgaggaagcagaattaacatcagaatacaaacagcatcagggaacccacagcatttccctcttttgttcaataaaaaaggatcttttctttcaggtataaattgagcacaaccataacaattatgtaaattgtaaagcaggatatacacttaacatctagtctatcatatttgtcagttagataaagtactctaccacctaccctaatttaaagagttaataagtctatgtctaaattatgttgattttaacttgtattcccatctgaaatccatgcttacaaatctagataatctttcttaatgttaaaccatttaagtttgattatgagactataactagtcttcaatcccatcagaggtctgagaaagaattaaatattacctgaatatgtaggaggcacaatgacgtagcttccaagattatacaatttgtagagacagctgactgccctaatttcttataatgttggatctgtctttagccttctggcccagaacaatctgacagacctaagtaaagcaggaactttgaaggactagcttaccctgttttgacagagcttaacaattgactatcccacatctgtttgtccttttttggacagtatttttgtctgcagatgaattagggcacttcttgcccaatggctaccttgccacgatcaaagcaactccccctggaggtaatgatgctcaatagcctccttgaagtggaatgtgatactgtcatgaacacatctgtctcatagtcatatgatctttgatgatgaaataatattaaatgccatattctgtgaatctctgatgcttttgaagactatctaaacatataccataacagaattgtcaaacatccattactcttagctatttactattaaaataatcttgaaaacacttgtaattaactaaattagaatctaataaagccatgaatttcagtatctgacttgcatcacttaatcatcctaaacagtttgtaacagcagctattaaaaggactggatctaagccttgtattcttaaatgagttacataggcataatgcctatctaagagtaacaatattaattttaaattttgtactaatatacaaagatttataccaatgaaaaccttaaacctgtatcaatatatataaatagtgtaacaatgtaagaattaaaacttcaattttgtatcagatataaaggtttctaccaatgtaagattatggtgatttgatgctttgtttaagtgtaaatttaatatcctatctctatctgtccatttgactatatttccccttccctttccatcttcctctctttacctaagaaggaaggatagagaagaggaaacgaaaggtagaaatccctgagtctaggctctctagtttcctccctgtccaaagtcacacttgtaagttatccactaagatgacaatatatctataacttataaaatagccagaactatccactccaacctaagggactgggacaacaatcttcttttgtttgcttctagctaaattggggtgaggaattcccttctgggggcccaaaggggaataggaaaattggtcttgtcaaaggcagactagctggcatcatttgcctttcggtctctatgtgctgagaaagtttatggcttagctgaagtcctgactgtaatagtctgaaaggctagatgagtgaattagctgttctgaaactgtcctggaagcaagtctttagaggaaacagctccgatgtagttgaggtagaatcaagcaaggagctggaatggaaggacctggaatctcagcgtccccgagtgtgaatcttgtcagggttgtctctctcctctttcattcttcagtatacaaaccttacaagcaactcgtgggctctgtaaagacattcatgtggaacgtgcagggtgcacagattaattaaggaagatcaataaagaaagacaactgcctttcttcagaggttagtttgatcacataatcaaaatacaatataaatagctattcctgccatcttaaaggatggtagctaataaaaagttatgaaaattttgtgctcaacaagtgttattggctgtatatagacagttatatctcagcaggtcccagagctgtctctgtgtacagagtcatatatacatcaccacatatacatcacttatcttgttgatcttgtcaatctccttttgtctgtagccaaggtctttagggggttctccccttgtcatgtctgatttttatcaacttggaaggaactcatagcttttcttttcctgtggaaatataggcaaaacctctcccccagagttagaagatctttaaagtaaacaggctggtctagctcagcgggttttccccacaatccaatgtctttcagcagctatatttttttgttcattaccattttaaaaatttaaggttaataaaacactatgaaatctatttctgagagtctttgttacccccttttgtctgataagcatctcttttagagtacaattagatctttccacaattgcttgtcctgtaggattatgtgatacgcttgaaacatgttttatataataatatgttaaaaacttttttcatcttattagacacatatgtcggaccattttctgttttaatttgtatgagtattcccatcatggccacaacgtctaacaaatgtgtaattacagaatctgccttttctgaagctaaagcagttgtccattgaaatcctgaatatgtgtctattgtatgatgcacatatcttagtttaccaaactctataaaatggaacacatccatttgtcaaatttcatttctttgggtacctttagggtacttcctggaggttaatggagtttggttatgtaatgagcaagtaggacattgcctcacaatttctttggcttgttgtcaagtgatagagaattctttctttaaacctttgttgttaacatggtgatttttatgcaatttcgaagcctctagtaggcaggtctgtggtgtattttcttggtcacgattgatgtggaaggccctgctcactgtgggcagtgccactgatggtcttgggtgccacaggaaggcaggtggagcgagttacgaggaacaaggcagtaagcattgctcctccattctctgcatccgtttctgtctccaggttcctgccctgacttcccgtgggtgatggactctaaagctataacagtaaatgaactttttcttctccaggttgcttttggtcatgatgtttttaccataacaatagcaacaaactgagacaagctccatgaccaggggtttctctttcttggaagttggtgtggctttgtttgttagtagctgaatgttacttgatgttaccccagattcacagcttcaggagaaaggtagacttctacccttcatttaacacacaaagacatgtaaaatactctgaggaagcagcttcccgaggcagagtggccagtgatacagccaggtttcaatctaggaggagcccgtgttcccagccaggactcttactatcttcctgcagagtcatcaacaatctagtctagcctctttggggttgttctactgaactctggtgtctgtccaatttgtctccgcattttgtatgcacttttcctttaagagccgcttttcttgcctcctccccaaaggtgtcttgccctattcccaagactgttaaggcacctaggaggtgactatctgaccagtggctttctgtatattcacctctctaaacaaaagcttccgagaatcaggaacttcctgttttgtttcctgttattattctccattttttttccttctaaagtgtctggaatatggtagatgcagttagtttttgacaggaaacaagctggtagctgacacttaacttcctcacttgatcattagcatcttgtgttgacccaggttacagctcttctacctcttgataagaatggatttccagagttgtctttatgctattgctgaagaactgggcagtgaagacctggctgccctcaagttcctgtgcttggactacatcccacacaagaagcaggagaccatcgaggatgcccagaagctatttctgaggctgcgggaaaaggggatgttggaggaaggcaatctgtctttcctgaaagagctgcttttccacatcagtcggtgggacctgctggtcaacttcctagactgcaaccgagaggagatggtgagagagctgcgggatccagacaatgcccagatttctccctacaggtgggtagaaaggctgtggtggggggactgggaagtgtgggctgaagggggcatgcccctgtgacatgtgtgaagtgacttgtgagactgcgggctaaatgtaggggctaaatacataattcagagaaacatgctaaagccataaaataatgtatctttcatacagcatggtacagaaagggacttatgaggaggaaaagggatttggctacaggagtcagagagagaccagtgtaggggaatatgattctgtgtggagtagttactttgctgttgctgtggaaaaataccacgaccaaaagcaatttatagaagagtttatgttggattacagttccagagtgctaatgccggagataagatggtaactagcaggcagggcagcaggggcagggagctgagagttcacatctgccatgcacacgggaaacagaaagtgtgacccgaaagctgggtgaggtcaaacccctcaaagtctgtgcctggtgatcatgatgtgcttcctgctgcaaggctctgcgttctaaaagctccataacctctccagacagcctgggaccaagtgttcaaatacatacgcctaggaaggaccctgttcattcaaaccaccacaatgtataatttaaatttttcatttttcaaagtgaaacattaccttctagtatgctgatagtgagatttttttaaagatttttttaatttttattttacgacataggtattttgtttaccgatgtgtctgtacagcatttgcatgtaggcttcctgcagaggccagaagagggcattaaatcacctggacatggagttatagacagttgtgagctgccatgtaggtgttgggaattggacctgtgtgcttggaagagccatcagaactttgtttgtttgcttgtttcactatttatttatttttatttattttacatcctgatcaaagccccctcccttttctcctcccagtcctgccctcccaccgcccttctccctcccccaccccaccacctgccctggcacagtaagtcacagcaggagactaagtgcatcctctctcactgaggccagacaaagcaacccaggtagaggaaaggaatccaaaggcaagcaacagagtcataggcagacacctccctcacctctgtgcctgctccagttattggagaacccacatgaagaccaagctgcacatctgctacatatgtgggggggcgggggctaggtccagccccatatgctctttgattggttgttcagcctctgtgagcccccatgggtgcaggttagttgactctgtaggtcttcgagtggtccccttgacccctcaggctctttcaatcctatcccccactcttccacaagactccccgagctctgcctgatgtttggctgtgggtctctgaatctgtcagttgctggatgaagcgtctccgaagacagtcatgtgaggctcctgtctgcaagcagagtttcaaataagtattttagctcttcctcttctttcttgttaaagaaagttaagatgacagaggtttaaaatagtctggggacactaaggagaacaaaccaggattccagcaggctatgtggtagctgcttccaccagccaagaaggactgggctctggagaaacaacaaggcattgttgccctttcccccacgtaacctgagtaactttgtaagtagtcccacatttatacatgttctgatgtagaaattgtaatatctgatgtaacaaattgtaacagacaggattgtgggtaatgatgacttctaagagagtctctgtctatcagggaaaggaccacccagcctgcccctggactgggcaaggggagggaaaatggagaagtctagaggtgtgctcactttcagggatgttttctcacctcctgaaataagctggaaatactttgcattacacacgctgcagggtgaattctcaggaacttaaaaaccgagactgcaggcctggatcaataagaaggcagattcacacatcggtgacacacatgtatcttggtggctcacatttaaagacacagaagggttccatatgcatacacaagcaggtgggaagggttgtcactgtgggaagcagcaagaccctaaccatgaacaaacacatcttgcccagcctggaagtggccaccttctgtaaccaggcaggacttccagtggagggatagagacgccagcccacccacagaattttgtcctgcctacaagatgtgtttgtagtctggggcccctgcactcaaacattacacctcacaaatctcaggttaatgaaaagtgaaaaaggcatgggccaggctgttaggggacttggcagtttataaacaggctgttgccatatgtgagttctttcaggggcgggggtggggggtgaatgaagatttagtgagagaacagagccccgaggactccctggcaactaatgagattgcaggtgggcagtggatgcatgcaaggagacacagggaatgtatggtggggagagcacacggcagctggctgtgttgagagccttgggtgttggtaagagtggaaagaggtgtgtgtgtgtgtgtgtgtgtgtgttcagagtcccttttattctagaaccaaatattcagcctcctcctgaaggaaattgctgagagaacatggaccaatgcttagaagggagtcagctctgttgtgcaccatggagaatataattcccccaaatcctcgcatctggatcttgatttttatatcgacataggagcagggagattcttttagttgaagttagttggttacaaaggtggtggcacttcgcatgggtgggttggttggacctgactctatagacttccaccagtcaggaatagttgactattttaaggaagccctgggagtgagccctgctgtgactgtgtagtggtgggaaatgatctagaacgtgtttctaaagtgcctctgtttcctgccacagggtcatgctctttaagctctcagaagaagtgagcgagttggaattgagatcttttaagttccttttgaacaatgagatccccaaatgtaagctggaagatgacttggtaagacctaatctcctgaagatgggtcacctctggggtttctgagaccagagagccctgctaaacataaatagtattcaaggttgtgatggtgttgttgttgttttgaaacatggtatcctatagcccaggctaaatccaaacttgctatttagctgtcttcattagagttttcctgctgtgaatagacaccatgaccaaggcaactcttataaggacagcatttaattagggctggcttacaggttcaggtccattatcgtcaaggctggaacgtggcagcgttcaggcagatatggtacaggaggagctgagagttctacatcttcatctgaaggttgctagcagaatactggctcccaggcagcaagaacaagggtattaaagcccacacttacaaggccacacctactccaacaaggccacacctcccaacagtgccacacctcccaacagtgccactccctgggccaaccatgtacaaaccatcactgtagccaaagttgaccctaaacttctgatcttcctgcctttatctcctgttaatgtggttccaggatgtggaactcagcactttctgcaccgtaagcaagcactctgtccactgagcagcccctgtcttctcactagaagaaagtagttagataggaacaggtaggtatggctctagtgcctctgcacagtgaagtgttactcggccacagggccgagccagaagccggagagagcaagttgtgcagttcaacaatacatgggacccagcagggccaaggagaaaaggtggcccagggtcacatttccctacttctgctgcccttgaaacacggcgtgttcgccccctctcactccatcccggtgtgtattatgtgtctttatgagtaggttgtatgatgtgtctgtgtctacatgttgtgtatattgtgggtgtttatgtgtggtgtaggtgcctgggtatacctctgtgtggcttctctgttggtctgagtatatatgtgtgtgatacctgtgtgttggcgggggttgggggttctctcactctgtaaatcagtgtgtctctgtgtgctctgtggtagatggcctaaatttgatgtatgctttgtccagatgtatctctgtgcgtttgcttagggtacccgtgaacacaagtgtgtggcatctttctgtgtgttcggtgcatatgactctgtgtgtattaactgaggtttagagaaagctaaggtaccaggggcaatactggggaataattcagtctctaggattggcccatccacattcctcaggctggtccatagctgaaggcattctcatactggttgagaacaagacctggggactgggtgacacctgacatggcttattcatctttccatcttttaaaatactttcaggccttcctgagtactgtcacctgtgagagatcggcctgtgcttggaggtgggagggggatctatgtgggaaataagactgggccctgagtttggaagaagtagagactaaacgaagccccaaatctccaagcctgtgctctcaagccttttgtgcttcactctcctccccagagcctgcttgaaatttttgtagaaatggagaagaggaccatgctggcagaaaataacttggaaaccctaaaatcaatctgtgaccaggtcaacaagagcctgctggggaagatcgaggattatgaaagatcaagcacaggtagccagctagtcaacagctgcgagttgggaaagccctcagagaaacagctctttctgatcaaatttttctttttcaattcagagagaagaatgagccttgaaggaagggaagagttgccaccttcagttttggatggtaattcgcacagatacattccttagattctgaggtgcttgatttccgacttcctcttgccttctacccaccatcttctgcctcatcctgattaagatgaaagttcagtggcctcttgtcacctctgcagctctctgaaagccccatggaaacattctcacagttactctgtaagaggctcttaactctgctctggcctttgcttttgaagagagttgtggggagctagacacaaatcttttcctggacatccttttgttttgttttgttttgttttgttttgttttgttttgttttgttttgttttgttttgttttgttgagacagggtctttctacctcatcctggttgtcttggaactctcaatgtagaccaggctggccttgaaatcatagaaatctatctgcctctgtctcaagtgttaggatcaacacctgactttttccagaatgttgttaaccaagtttcttcaagtttttctgctctgtgagaggcccctcagccaagtgtctcctgttaagcccaggcacccaggtccacacacacctgagttcctccattgtgtgcctgctccatcctgggatactcaagccaagttgcctagctcatcccagaatggaatttgtccagtcctctgtttccttacctaggaaactggatagaacaaacagaaggattccttcaacgtagggtgtctcagttagggttctattactatgaagagacaccatgacctaggcaactcttataaaggaaaacatttaactgaggctggcttacagtttcagaggttcagtccattattgccatggtaataaaacggcagggcaggcagatgtggcactggagatagagctgagagttctacatcttgatcctaaggcagcaaggagactgtgccactgggcatggcttgggcatatgagagatctcaaaccccacctccacagtgacatacttcctttaacaaggccacatctcctaatagtgccactctccaggggccacacattcaaacacacgagtctatgagggccatacctattcaaacctattctccctatgtagctagctagcctggaatttgctatgtagaacaggctggcctccaactcatagagatccatctgcctctctctgccttccaagtgctggggttaaaggtgtgtgctacaacacctggctctttcattttttaaaaaatttttatttatttttcaactatgtgtatgtgtgaaaacatgagtacatgagtgtaggtacccatgaagtctggaagagggcatcagacctcctgaaactgaagttacttacagatggttgtgagccaccatattgatgctgggaacctaatctgcacactctgcaagagcagccagagctcttaactactgagcatctctctagtcccctccaaatttttgattaatctaatattctgattataaaaattttctgtgctcttttgggggcagtttttaaattcagagaaaattaaaacaccatcactactctgtaaatactaatattgataatatctcttctgctctcactattgtttttgctgtgtaatatattttggatcactctttattttaaatttatgtgtaaatgtgtgtggctgagtaagtttatgcatgctgcatttgtgtaggagcctgaatacgtcagaagatagcatcagatcccctggaactggagttacagatggttgtgagccaccatgtgggtgttgggaatctctggtcctctgcaaaaatgttaaggtctcaacagctgagccatctctttagccgtgaggataatcctcctttaaagactgtgcctgagtaagagagccagttctttcttccatccacgcacacctaagcctagctctttccaactaccttcaggatgtttgactcagtgactcatcctgggctcacgcttttaatcagtaaccgcctagcaaatgaggcaatgtgctcttggttttcaaggggtgctaagtacccttcctacaagattgatatatctgaaatatgttcaaggcagtttcaatgtttatttttaaaaaatacctaagaggccagtgaaatgccttagtaggtaaaactgtctgccatacagggcaggtgatctgagtttgatctctggaacacatgtaggagggaggaagggagccaatctgcagagtcctctgtgatctccacacacacatgccatagcatgagagctccctacatcaacacacacacacacacactctccacacacacatgccatagcatgagagctccctacatcaacacacacacacacacacacacacacacacacacacacacacacgcatgcacacacgtgcaagaacgcacacattcacgtacatacgcgtgcatgcacttgcatcctgaaaatgttttaaggtccttaggttgtcctaagtgtgacgtttttttggttgcttgcagagatgagcctcaaaatggcggaactgtgtgactcgccaagagaacaagacagtgagtcacgggtaggtgtgtctcctacctctctctttgcattggtgttcctgtttcctttggttggttccttttcagctgccagataagccaataaggggtagagccttttggaaactctggagagaaaaacagaaaaaacaaacaaaacaaaacaaaacaaacaaaacaaaacaaaacaaaaaaaccaaacaaacctacaaaagaaccagttaagatagcctctccagttttctttgtgttacattatcactgttttgtttgtcgtttgagacagtgtctcctatagctcagattggctttgaactcattatgtagctgaggctaaccctgaactcctgattcccctgcctttacttcccaagtgctaggattataggcataggcctataataattacggtcacactgggccataattgtattataaggtcaagggcagaggtaaggggttattgaaggggagagactgtgtgtggcttcccccctctagagcagcggtgttagctaaaaatagcacatgggggatggagtggaagcttcaggactttctctggcaggttttctcttttagccagacaccacacagagttttctcctttgtgttaggcaacaaataggatctcagcatgtcttaggtgttgtctccacgaagcacatagagagatttccttcctggacatagcttccgtttataaccttcagtagctcaccaaatatggctcagccatgaggctccatgtggcaggaggctcaggaaatcctcagcactatgaatagggtctgtactagggagggctcctgagcagatcctcaggcctgtgttggtcaggaagtcaaagatcatggactccatgggctgctctgaagcatagtggttttctacagggtcaccctctccaaacagaggatgccatgtcactaacttagtagctgttagcattacagcatctgaatacattaatacaagttggaaacgtaggataataacaaatactgaatattctttgtgcttaataaagtactacatgtcatagaaaacatattgaaaggcaaaggacttttacagtatgaagggacactcaaatgtgttacaaaatatatgaagatactaggagattaggtgagctgcccagattcccatggtttcaagccatagaagctggcatttgtcttctagcctggggtccttttcttcaggagacagaagaggcttccacccaaatgctaaaaatatcagcccgaagaaaataaccacaacgttattttatgtatactacatattttatgtataccacactattgctgtcttcagacacaccagaaaagggcaatgattccattacagatggttgtgagccaccatgtggttgctgggaattgaactcaggacccctggaaaagcaatcagtgcttttaactgctgagccatctctccagcccctgtcatctggtttttactgtcaggatttggtaaacacaacttgacgagctccttgctctccaccaccatggagttcctttgcattgggttaaggcatcagcaataaccacccagagactgcactgtatcctgtgtgaagggcaggtcctcaactgttagccaagaaacacagatgagctgagaaggccaccaaaagattcccaaggtgatagagagacatcctaagacatgacccttgagtgaactgagagtcataaaactctagtctccacgatggcacagatggcatctgccctgaagctcatctgtatggtcagggacactgataggccgtgatgacagccatgatgtacacagagggtattcagcaggtccaggcctactgaccatgcagctcatgaggagccaagcaatggaaggagcctatgcttgcctgaggggctgagcagcaatccagcttcttcctgagcacttggacatagccagtgtctgagctgcagcatcaacccctggattgggcttgtgttttccagacttcagacaaagtttaccaaatgaagaacaaacctcggggatactgtctgatcatcaacaatcatgatttcagcaaggcccgggaagacataacccaactccgaaaaatgaaggacagaaaaggaacagactgtgataaaggtatggtgggatccaaaggagaagcatgagaaaattttattcttatggacctttctttaaccaaaaaggagagagcagtagttatacttaaagggatatatatatatatatatatatatatatatataaattaattaaagcatttattatgttgaaaatttttaaacaatttttattagatattttcttcatttacatttcaaatgctatccagaaaatccccaatacccttcccccgccctgctccccaacccacccattcccacttcctgcccctggcattaccctatactggggaatataatctttgcaagaccaggggcctctcctcccaatgatggccgactaggccatcttctgatacatatgcagctagagacatgagctcagggtgtactggttaattcatattattgttcctcctatagggttgcagaccccttcagcaacttgggtactttctctagctcttccattggggtccctgtgttccatccaatagctgactgtgagcatccacttctgtgtttgccaggcaccggcatagcctcataagagacagctatatcagggtcctttccgcaaaatcttgctggcatatacaatagtgtctgcgtttggtggctgattatgggatggatcctcaggtggggcagtctctggacggtctttccttccatctcagctccaaattttgtctctgtaactccttccatggatgttttgttccccattctaaggatggacaaagtatccacactttggtcttccttcttcttgagtttcatgtgttttccaagttgtatctttggtattctaagttttggggctaatatccacttatcagtgagtgcatatcaagtgacttcttttgtgattgggttacctcacttaggatgatatcctccagaaccatccatttgcctaagaatttcataaattcatttttttgatagctgagtagtactccattgtgtaaatgtaccacattttccgtatccattcctctattgagggacatctgggttctttccagcttctggctattataaataaggctaccatgaacataatggagcatatgtttttattaccagttggaacatcttctgggtatatgcccaggagaggtattgcttggattctcctgtagtactatgttcaattttctgaagaaccgccagactgatttccagagtggttgtacaagcttgcaatcccaccagcaatggaggagtgttcctctttctccacatcctcgccagcatctgctgtcacctgaatttttgatcttagccattctgactggtgtgaggtggaatctcagggttgttttgatttccatttccctgataattaaggatgttgaacattttttctggtgcttctcagccattcgtattcctcagttgagaattctttgtttagctctgtgtcccattttttaatggggttattggattttctggagtacagcttcttgagttctttgtatatattggatattagtcccctattggatgtaggattgggaaaaatcctttcccaatctgttggcggcctttttgtcttattgacaggttcttttgccttacagaagctttgcaattttatgaggtcccatttgtcgattctcaatcttacagcagactccatttctgttctgttcaggaattttttcccctgtgcccatatctttgaggctttcccccactttctccactataagttttagtatctctggttttatgtggagttccttgaaccacttagacttgagctttgtacaaggagataagaatggatcaatttgcattcttctatataataaccgctagttaagccagcaccatttgttgaaaatgctgtcattttttccactggatggttttagctcctttgtcaaaagatcaagtgaccataggtgtgtgggttcatttctgggtcttcaattctattctattgatctacctgtctgtagctgtaccagtaccatgcagtttttaccacaattgctttatagtacagcttgaggtcaggcatggtgattccaccagaggttcttttattgttgagaagagtttttgctattctaggttttttgttattctagatgaatttgcaaattgccctttctaactcagtgaagaattgagttggaattttgatggggattgcattgaatctgtagattgcatttggcaaaataaccatttttactatattaatcctgccaatccatgagcatgggagatctttccatcttctgagatcttcttggatttctttcttcagagacttgaatttcttatcatacagatctttgacttccttagttagagtcacaccaaggcattttatattatttgtgactattgtgaagggtgttgtttccctaatttctttctcagcctgtttatcctctgtgtagagaaaggccactgatttgttttgagttaattttatatccagctactgcactgaagctgtttatcaggtttaggagttctctggtggaatttttagggttacttatatatactatcatatcatctgcaaatagtgatattttgacttcttcctttccaatgtgtattcccttgatctccttttgttgtctaattgctctggctagaacttcaagtgctatattgaataggtagggaaaaagagtgcaggcttgtctagtccctgattttagtgggattgcttcaagtttctctccatttagtttgatgttggctactggtttactgtatattgcttttattatgtttaggtatgggccttgaattcctgatctttccaagacttttatcatgaacggacgttggattttgtcaaatgctttctcagcatctaatgagatgatcatgtggtttttgtctttgagtttgtttatatagtgaattacgttgacgggtttccatatattaaaccattcctgcatccctgggatgaaacctacttgatcatgatggatgatcgtattgatgtgttcttggattcggtttgtgagaattttattgagtaattttgcatggatattcataaggaaaattggtctgaagttctctttctttgttggatctttatgtggtttaggtatcagagtaattgtggcttcatagaatgaattgggtagagtgccttctgtttctattttgtggaatagtttgaggagaattggaattaggtcttctttgaaggtctggtagaactctgcactaaacccatctggtcctggggtttttgttgttgggagactattaatgacggcttttatttctttacgggggatatgggactgtttagatcgttaatctgatcctgatttaactttggtacctggtacctgtctagaaaattgtccatttcatccaggctttccagttttgttgagtatagccttttgttgtaggatctgatgatattttggatttcctcaggttctgttgtcatgtctcctttttcgtttctgattttgttaattaggatactgtccctgtgccctctagttagtctggctaagggtttatctatcttgttgtttttctcaaagaaccaactcctggtttggttgattctttgtatagttcttttcatttccacttggttgatttcagccctaagtttgattatttcctgccgtctactcctcttgggtgaatttgcttcctttttttctagagcttttaggtgtgttgtcaagctgctagtgtgtgctctctctagtttctttttggaggccctcagggctatgagttttcctcttaagactgctttcattatgtcccataagtttgagtatgttgtggcttcattttcattaaactctttgatttcattctttatttcttccttgaccaaggtatcattgagtggagtgttcttcagcttccacgtgaatgttggctttctattatttaattgaagatgatatttcattttttaagacagggtttctctgtgtagccctggctgtcctgaaactcactctgtagaccaggctggcctcgaactcagaaatctgcctgcctctgcctcccaagtgctgggattaaaggtgtgcgccactactgcctggctaacttgtctgtttttataccaatactgaggtcagagatgatgatacctccagaagttcttttattgttcaggattgttttggctattctgaggtttgtttgttttttccacatgaagttgatgattgctctttcaaagtctataaataattgtgatggaattttgatgaggaatgcactgaatctgtacattgcttttagtaagatggccatttttactatgttaatcctatcaatccataagcttgagtgatctttccatcatctaatatcatcttcaattcttcttttattcagagacttgatgttcttatcacacaggtctttcactggcttgcttagagttacaccaagatattttatattgtttgtggctattgtgaaggatgttgtttccctactttttttctcagcccctttattgtttgtgtaaaagagggctagtgattttttttttttgagttaatttggtatccaagatttgaggtgtttatcagctgtaggagttctctgacagatttggggggagggtcacttatgtatacgatcatatcatctgtgtgtagctttactttgacttttttagttgtcttattgctctagctagaactttactattgatagatatggagagagtagatagccctgttttgtccttgattttagtggaattactttaagtttctcttaatttaatttaaatttgggttgactatcagcttgctgtaaattgcctttattatgcttaggtacgtaccttgtatcccttttatcatgaaggtatgttggattttgtcaaggctttttcagcatctaatgaaatgatcatgtggttttagttttaattttaagtttgtttatggtagattccattgatggatttccaaatgttgatccctgcatccctgagatgaagtatacttgaccatagtggataatctttatgatgtgttcttagatttggtttgtgagtattttattgaatatttttgcatcaatgttcataagggaaattggtctgaaattctgtttctttgttgagtctttgtgtggtttctgtgttaaagtgactgggacttcataaaatgaatttgacaatgtttcttctgtttctattttgtggaataatttgaggagtgctacctcaaattattacctcttctttgaaagtcaagtagaattctgtgctaaaacaatctggctctgggctttctttggttgggaaacttttaatgaccgcttctatttccttaggagttataggactatttaaattgtttacctgatcttgattcaactttggttaagtagaatctatcaagaaaatcatccatgtcccttaaattttccaattttgtggagtacaggctttagattctttacatttccccagtgtctgttgttatgtcccccttttcatttctgagtctgttaatttgggtactgtttctctgccttttagtcagtttagttaagggttttttctatcttgctgatgttcttaagaaaccagctcttggttttgttgattctttgtatagttctctttgtttctgatttatcgattttagccccaagtttaatttttcctaccattaactcctcttgggtgtgtttgctcctttttgttctagaacttttaggtgtgcttttgaattgctagactgagatctctccaatttctttatcaagacacttagcactatgaaagttcctcttagcattgctttcatggttccataagtttgggtatgttgtgtcatcattttcattgaattctagaaagtctttatttttttccctgatccagttgtcattgagtagagggagttgttcggtttccaggagtttgtagtctttctgctgtttctgtagttgaagttctgctttaatctatggtggtctgataagaaacaaagcattgtttcaattttgttgtatctgttttggcttggtttgtgaccaatatacggtcaattttggagaaggttccatgaggtgctgaggaggaggtaatcttttgtgtttgtgtgaaatgttctatagatctgtaacacctatttgatttataatgtctgttagttttactatttctctgttttgtttctgtctctatgatctgtccatggtgacagtgaggtgttgaagtctcccactattatgtggggtttggtgtgtgatttaagctttagtaaagtttcttttatgaatgtgggttttcttgaatttggggcatagatgttcagaattgaggcatcatcttggtacatttttcctttgaggaacataaagtgtccttccccatctcttttgattatttttggttgaaagtctattctattagatattagaatggctaccccaggttgcttcttaggtctgtttacttggaaagtctttgtccagccctttactctgagataatgtcaacctttgttgttgaggtgtgtttcttctgtgcagaataatgatggatcctattttttcatccattcagttagcctgaatcttttttattagggaattgagtccattgatgttgagagatattaatgaccaatgattgttaattcctgttattttgatggtggtggtagtagtgtgtgtgtgtgtatgtgtgtgtgtagtatttcccttcttttggttttgctgatgtgatatttatttcatgttttcatgggtgtagttaatcttcttgggttggagttttccttctactagggataaatttgtggacagatatggttttgtcatagaatattttgttttctccattggtggtgattgacggttttgctgggtatagtagtctgggctgacatctgttgtctcttaggatctgcaagatatcttcccaggcccttctggcttttgagaagtcatgtgtattgcctttgtatgcaacatggccttttccccttgcagtttttaatgttctttctttgttccaatatttagtgtcttcattattatgtggaggaaggattttcttttctgatccaatttattttgtattctgtaggcttcttgtttgtttatagctatgtctttctctaggttgggaaagttttcctgtatgattttattgaaaatattttctgggacttggagttgggaatcttctccttctagtcctgttatttttggtttgatctttttctaattttccagatttcttggatattttgtgtcaggaactttttagatttagcattttatttggttgatatatccatttcctcaatagtatcttccatgactgagattctgtcttccatttcatattctgtcagctatagttcctcttctcttccatctccaagattccctgtttgtcttttctatattgctactatttccattttcatgtcttgtacagttttttttcatttctttcacctgtttgattgtactttgttttgattgtattttcctatatttcttaatttcctgtttaaaggcctctatcatctttattagattgaatttaaggtcatcttcctgtgctttagctgtgctaggatatttagggcttggtgtggtccaatagctgggctctggtggtgccatattgccctggctcttgctggttgtattcgtatctggcctttagcacatggttgtccctggtgtttgctggatgttcctgtcactgacagtactcctcagggaggcaggcagagccttgggtctgacaagagagctcaggaaacagcaagctgctcacctctgctgactgtacctcaggtggccctaagtgttcctggagtctagcaggcctccttctagggaagggaggcagagccatgggcttgccaatgtcacagagcaaagcttgtctgtagttgtgccccaggtggaatcagcagtcagaggataggtgaggtagggtctaatctgggtgttcctggggacctacatgcctggatgttcctgaggctgggcaggtctcctggagaggaaaggccatggtgctgggggcaggggaataggaggagtggacacagcatgcagctcacctctctatcaagcatttcttattcattttatttatttatatttcttacatatctattttctgctgtttcaaaaaataggttttaaaatcaaagtaatatattcatcatgataaaatgatagctttctttttttgtttttgttttttattttacttgtaattcatttttttacactccatattccattcccggcctctccccatccaccctctgactgctccacatcacacacctcctccccaccccaccccatctccacattccccagctcatctgacctctatactctctggggcatctagtctcttgagggttagatgcatcatctgtgaatgaacacagacctgaaagtcctctactctatgtgtgttgagggcctcatatcagctggtgtatgctgtctgattgctggtccaatgtttgaaagatctcatgggtccagattaattaaggctgctggtcctcctacagaattgcccttctcctcagcttcttttagccttccctaattcaacaacaggggccaactgcttctgcccattgattgactgcaaatatctgcatctgactctttcagctgcttgttgggtctttcagagggcagtcacgataggtccctttttgtgagtattctatagccttagtaatagggtcaggccttgggacctccatttgagctgaaaatgatagccttctaacatttataatgaagagcatcaatgaactttaaaatttttttaaattttatttcatatatatatatatatatatatatatatttctatgtatgcccctttgtgtgtgtgcatatgtgtgtgtgtatacttgtgtgtgtgtgtgtgtgtgtgtgtgtgtatgtttgtgtgtgcgagggcctttggatccagaacaggacaagttctatgcaagagcagcaaggactcttaaccactgagcaatctctctagccctgagctttacatttaactcaactaaatgaaaaatatttctggagaatgaattagtcaaaaccaattgctatggtatcctacttactctgggtagtttgctcatgaaagtttctcaattgcagcaggagttgtgagctcccagcaagatgctaaactagtgagacctgccttggggcagcctacttatgtttatactgagagctgacaagttaaatggaacaatattcatttataagtatacaatatacctataacaagtataagaatacagaaatgggtgccgggcatggtggcgcacacctttaatcccagcactcgggaggcagaggcaggtagatttctgagttcgaggccagcctggtctacaaagtgagttccaggacagccagggctacacagagaaaccctgtcttgaaaaaaaaaaaaaagatacagaaatggggctcaaagatggctcaagagcactgactgctcttccagaggtcctgagttcaattcccaggaaccacatggtggcttacaaccatctgtaatgagatcctgtgccttcttcccgtatgtctgaagacagctactgtgttctcatatacataaaataattcctaccaattcttaccaagaattctttctcctctcaatgtcccaccttctctttcctgcctaagccataggccataggctttttaattgacaggtagtgcatccatacaatgcacataagatgctctctctgcaggtgactcagtagttaaaagcactgactgcttgttctgaggcttcagattcaattcccagcacccacatggcagctctcatgctgcctgtatctccagttccagaggagtcactctcttcttctggcacgcacatacatgcgataaaagcactcgtacacacaaaatgataatgtaaagcatttaaaatacatggacaacaataaacatatgcaaggttaagactcaggaagtcagaccaaaagaaaacactgatggaaaatgggaacaataggagttgaaatggagaatactggtcacagatgccagatctgtgaccatgagagccataagacaaagacaaggaaatggacctgtgcaagaagtgagtgtacgttctccagtctgaaagcgcttgttgcatctttgagattgagagggaggggtatggacaagtcagatacgtagagcagtcacagatcaggatatcactgaatttcataatagcagatctggaagctaaaagatgaaaattcagaggaaaggggattttggaacgaatgctatatctagctagactgtcaaaaagaatgagagtagaaatgaatcctcatggtgcaagaactcagcaaagttccagctggcagtacacagcactttcagcaggcaacaataaacataaggagcctgtacaatccgattggtggtgatgcagaaggaccgctcaggttctggttcttgcctacaggctgtgttctgggaatgattgtaattcatggtttcccatcttacttttcagaggctctgagtaagacctttaaggagcttcattttgagatagtatcttacgacgactgcactgcaaatgaaatccacgagattctagaaggctaccaaagcgcagaccacaagaacaaagactgcttcatctgctgtatcctatcccacggtgacaagggtgtcgtctatggaacggatgggaaggaggcctccatctatgacctgacatcttacttcactggttcaaagtgcccttccctgtctgggaaacccaagatctttttcattcaggcttgccaaggaagtaacttccagaaaggagtgcctgatgaggcaggcttcgagcaacagaaccacactttagaagtggattcatcatctcacaagaactatattccggatgaggcagactttctgctgggaatggctacggtgaagaactgcgtttcctaccgagatcctgtgaatggaacctggtatattcagtcactttgccagagcctgagggaaagatgtcctcagtaagtttggcctcctgggcccctctcagggttatgcttccttactcatttctgtggttagagcccattagaaggtgctttatggccgagataatatatactaagcagagaagtagccattggcatgttccatctctgaatgtctagctgtagctgacacagttgtatttcccaggtcagtggcaaagtgggagagatggcacgtattgttctccttagttcatagatgggaaaagaatatgtttactgaagagtttcctttatttagcaaccgaggtaccacagagccagagaggttgctaataccacacagtccagctctgtctttgggtagtaactgactaaggcaactcaacccctttggggagtcttcccagatggaagcaaaccactatgtttaacatacctcacctttgtcagaagccatgttttagagctggcaagatggctcactgggtaaaggcacttggttataagtcttataacatgaattagatccccagaggcctatatggtggagaagaccaactccttcaaattgtcctatgaccaccaaatgtgaaccatgtcatgtgcctgtgtgaataagtaaatgtaatttttaaaagttaagaaagaaatcctctccaattgctatctagccttgaatcttcttagcaaactataaccgtactgaagagcaggggacctctatatctctactaatttgacctgctaggaatccacaagtgtgggatgatagccctgtggcatatggttcatctccagttaaattagggctccatacttcaggttccctcccacttacacctggctgatagctgaacaatgaggagcccttctacgggctccttttacatctctgtcttatcgtctggtgtctgaagagtgagaatttcccatggcatgtggagagtagacaggaacaccatcgcagtgctttcattttgtaacacagaccctgagtcctacaggatccatgtcagctatgcagattgatgaagaggttgctccacaagattgctcagactgctggtgtcagagatggggaggtagctacgggaaatgcttagcctttacttgcctacattttagtagaaatctgtggatgatcctgtaggagcaatgctgatttaggcagagggtactgggggaagagagggaggcatttgtgagatccaaatggcaacatgggaaacatggaggaacacataggttaacagagaaggaaagcaaatctgataggaactctggtttcttgtaaccagcagagattccaagtacacaaaagtaactctgtgtgctctgagaaaactgagttgccagaacagcggtgagaggtcttgattcagtgactttcaactgcaaatgtccgatgctctctctgagagacagaactagagaacctacctcaggcacagccagtgaggtggtgggcacagccagtgagacagtgggcactgccagtgagatgtgggtggcacagccagtgagacagtgagcacagccagtgagagggtgggcacagccagtgagacagtgggcactgccaatgagacgtgggtggcacagccagtgagacagtgagcacagccagagagagggtggacacagccagtgagacagtgggcactaccagtgagacagtggatatagccagtgagacagtgagcacagccagtgatacggtgggcactgccagtgagacggtgggcacagccagtgagacggtggacactgccttgtcccatatgcttacgctgctttctttttcagaggagatgacattcttagcatcctgactggcgtgaactatgacgtgagcaataaagacgacaggaggaacaagggaaagcagatgccacagcccaccttcacactacggaagaagctcttcttccctccctaatgatgtgtgctctccacagttcacatggcttatctgtgcacttttgtgtggatgagtctaatttattttttagaatttcttttgcttttgaatttacatttacataattttcccttttcttccctttaaacccttctttgttatgttccaatttcaaatacatggcctcttttctcattaactgttgtacacacacatacatacacacacacacacacacacacacatttctaaatataacctgtatactatcacttgtatgtatgttttgttttgctttttttaaaaaatggagcgacctttggtaggccaatcacactccaaggcaggtccctctcccaagaatagttgggcaatacaaattggcttccatgggttcacctttaatctcagcactctggagacagaggcaggaggatctctgagttcaaagccaacctgctctacatagcaagttctgagatagccagggctacacagagaaagcttgtcttgaaaaaaacaaaaaagaaaacttgaagatgcatgagtaagaaagcaagactggagcatgaatctggttaacagaatgggtgaatgacgggagcagggacctggggcaagttaacagagggggtgaatgacgggagcagggacctggggcaagttaactgctgaaggtgagtcagcagaatggggtgaatatcatcagcattctctgcatgaaattctcagaggtaataataatattaaacttttttctgtgcagt

SEQ ID NO: 16 - DNA, 1053 bp, Anopheles gambiae
  misc_feature: mosquito gene Cyclin A (AGAP012413)
cgtagcgctcctttgcaagagctaaaacatgcagaactgctcgaaactcccatgtcagtgggagaaaacttttctccaatgtcaatagacaagtcgactagcataattgtcgaagaaggacccatccctaggaacgatcgcgagcgtttcttcgaggtagaggagtaccaggaagacatactactttatcttaaagaagccgaaaagcgcaatcgtccgaagccgggatatatgcttaaacagactgacataacccattctatgcgcacaattctggtcgattggttggtggaggtttccgaagaatacaaactgcagggagaaacacttgcccttgccgtatcttacatcgatcgattccttagcttcatgtcggtagttcgtgccaagcttcagttggtaggcacagccgccatgtttatcgcagccaagtacgaagaaatctatccaccggacgtgagcgagtttgtttacataacagatgatacgtacaccaagacccaagtgttgcgaatggagcagctcatactgaaggttctttcatttgatttgacggtaccaacttcgttggtatttactaatacctactgtgtgatgaacgacgtacccgataaagttaaatatttaacaatgtatctttgcgagttgtcattgctggaagcagatccattcctaacgtatatgccatccaaaattgcagccggtgctttggcattagcaagacgcactctcgatttaccgatgtggtcgaagatgttagagaataatactggttacaaattggtcgacatgagagacataattttagatctaaacaaaactcatgttgatgcagtaacaatgcaacaacaagcaatacaggaaaaatacaaatccaaaacataccatgaagtcgcatccttacctgctactgagataacaaaagaatcttttgataagatatgcgctacattatgcaattcttctgctgcaacactgctactttctaacaccgacagtgagttgttgaaggaaatggggcgagaaaatccatcaaacttacttttcgtttag

SEQ ID NO: 17 - DNA, 1110 bp, Anopheles gambiae
  misc_feature: mosquito gene inhibitor of apoptosis 1 (AGAP007294-PA)
atggcccatgcactatcgctacccgccccaccgatcgacctgcccgacaacaagcacaaggatgacgatctcgcctgcatgtcaccggagtacttccacatcgaggagaaccggttacgcagcttcacctcccgctggccggtcacgttcatcagcccgaacgtgctggcccggtacgggttttactacgtcggcaccgatgacacggtcaagtgttacttctgccgggtggagatcgggctgtgggaaccgcaggacgacgtgatccaggagcatctgcgctggtcgccgtactgtccgctgctgaagaagcgccccaccaacaacgtgccgctgaacgcgaactatctcgatgccgtgccggagccgagcttcgacacctgcggcatcagcgtgcggcacaattcgtacgcggagaatgccgacgatcgggtgcggatagatttggaccggctgagcggcgattcgtggagcggggcgagcgatatcagtctgagcagtgccgctggtgcagcggctgcttccgccggggagagcgagccgatgccgtcggtcgggagtggatacccgaactacgcgatcgaggcggaccggctgaagtcgtacgaggactggccgacgtcgctgaagcagaaaccgcagcagctgagcgacgctggcttcttctacacgggcatgagcgaccgcgtcaagtgcttcagctgtggcggtgggctgaaggactgggagcaggaggacgacccgtggcagcagcacgcgatctggtacagcaactgccactacctgcagctgatgaagggccgcgagttcattcagaagtgcaacgagctgaaggaggcggcttccgcctcctccgctgccagcacatcttccgcgatgtcttccgcctcgtcccagccgtccactgacgagggcgaggacgatgcgggtggcgatcggcgcgtaccgtccgacggtaagatctgcaagatctgcttcgtcaacgagtacaacaccgcgttcatgccgtgcgggcacgtcgtcgcctgcgccaagtgcgcatcgtccgtcagcaagtgtccgctctgtcagcaaccgttcatcaacgtgctacggctgtacctgtcgtaa

SEQ ID NO: 18 - DNA, 2193 bp, Anopheles gambiae
  misc_feature: mosquito gene Glycerol-3-phosphate dehydrogenase (AGAP004437-PD)
atggcatcgagacttcgaaagttcggcgtgacagcagccggtatcgcgatcggtgccgccctgtccacctatgccctgcagcacaaagatacgccccagtatcaggttcaaatggaggaaatgcaacgtattcgacgtaagcgaacgctgccttcacgatcggaacagatcaaggcgctgcagagcgacgaagagtacgatgtgctgatcatcggtggtggagccactggggccggttgtgcactggattcggtcacgcgcggacttaagaccgccctggtcgaagcggacgactttgccagcggtacctcgtcacgatcaaccaagctgatccatggtggtgtgcgatatctgcagaaagccattctcggtttggacattgaacagtaccggatggtaaaagaagctctgcacgagcgcgcctcaatgctgcgatcggcgccacatctgaccagaccactgccgatcatgcttcccgtttacacctggtggcaaattccgtacttctgggtcggcattaaggcgtacgatttcgtcgctggcgatcgtaacgtgaaaagctcgtactatctgtcacgcgccgatgcgctggaactgtttcccatgttgcgcggcgataaactgtgcggagctatcgtgtactacgacggacagcaggatgacgcacgtatgaatctggccatcgcactgaccgcggcacgccatggcgctgccatcacgaatcacgtcgaggtgttggagctgctgaagaaaaagggcgacgatggtaaggatgtgctgtgtggcgccaaggtccgtgataacattagcaaaaaggagtggacgatcaaggcaaagtgtatcatcaacgcgaccggtccgtttacggactcgatccgcaaaatggataacccgacggtgaaggaaatctgctgcccgagctcgggcgtgcatattgtgctgccgggatactacagcccacagcagatgggtctgctcgatccggacacttcggacggtcgcgttatcttcttcctgccctggctgaacggcacgatcgccggtaccacggactcgccgtgcgatgtaacgcgcacccccacgcccaccgaggacgagatccagtttattctgagcgaaatcaagaactatctcaacaaggacgttgatgttcgccgtggtgatgtgctgtcggcgtggagtggcatccgaccgctggtgtccgatccgaacaaggaggacacgcaatcgctggctcgcaaccacatcgtgcacgtgagcgactcgaagctgatcacaattgccggtggcaagtggacgacgttccgagcgatggccgagcacacgatcgatgctgccatcaaggcgtgcaacctgaagcccgagcgcggttgtgtcaccgatgggctgtggattgagggcgcccagggatggacaccgacgatgtacatccgcttggtgcaggatttgggtctggaggtagaggtggccaaacatctggccatatcgtacggcgatcgtgcgtttgccgtggccaagctggctacgctgactggcaagcgttggcccatcatcggcaagaagctgcatccggagttcccgtacattgatgcggaggtgcgctacggcattcgcgagtatgcctgcacttgcgtggacatgatttcgcgccgtttgcgactgtcgttcctgaatgtgcaggccgccatcgaggcgctccccatgattgcagacatcatggcggaggagctaaagtggtcaaaggacgaaaaggagcgccagataaagcagtgcgaacacttcctgcagacgcagatgggccaccaagctaaccgcacactgaaggagaaggtaccgatcaatctctccaagcaggaggtcgacatgtacgtgaagcgcttcgaaacgatcgacaaggagaagaagggctacgtgtcgatcaccgacattaagcgcgcaatgaagtcgttcggcgatgcggaggtcagtggcgaagagctgcacgacattctcaaggaaatcgacaccaacatgaacggacaggtcgagctggaggagtacctacagatgatgtcggcaatcaagtccggatttgtatcgcactctcggtttgctgcggtggcggaacaggaagaaatccgcaaggagcaggaacgtctcaagaagcagatcaccatcgagcgatctggagccactcaacgaaagtcaaactaa

SEQ ID NO: 19 - DNA, 21 bp, Artificial Sequence
  Y forward primer
  misc_feature: Synthetic
ggcagtgggtgtttcgtcctt

SEQ ID NO: 20 - DNA, 24 bp, Artificial Sequence
  Y reverse primer
  misc_feature: Synthetic
aactgtttcatttcccctctcctc

SEQ ID NO: 21 - DNA, 26 bp, Artificial Sequence
  Y-WT forward primer
  misc_feature: Synthetic
ggtaaggagataaagagtttccgtac

SEQ ID NO: 22 - DNA, 20 bp, Artificial Sequence
  Cas9-M forward primer
  misc_feature: Synthetic
acaccagcaccaaagaggtg

SEQ ID NO: 23 - DNA, 20 bp, Artificial Sequence
  Cas9-M reverse primer
  misc_feature: Synthetic
gtaggtcagggtggtcacga

SEQ ID NO: 24 - DNA, 20 bp, Artificial Sequence
  Cas9-WT forward primer
  misc_feature: Synthetic
aagggagctgcagtggagta

SEQ ID NO: 25 - DNA, 20 bp, Artificial Sequence
  Cas9-WT reverse primer
  misc_feature: Synthetic
ccgaaaatctgtgggaagtc

SEQ ID NO: 26 - DNA, 2973 bp, Gallus gallus
  misc_feature: ATP5F1BACCESSION   NC_008465
gggcggagcgcggggctcagtcggcgccgccatgttggggctcgcgggtcgctgctccgccgccgccgcctccgccgcccgcccggcgctgcgccgcgctgccgggccgtctcatggcttcctgccgctgttactcagccgcggggccggcccggccgccgccgtcggcgcgcgtgagtgtggagcggccgcggggtcaccttggggcctacgcgcgcggggggcgtcgcggcgagcgccattctcgggccccgttaaagcggaggggccctcagcgcccgccctgactcgctctctccttcaggccgggaccacgcggctcaggcggcccccgccgccaaggccggctccgccaccgggcgcatcgtggccgtgatcggcgcggtggtggacgtgcagttcgatgaggggctgccgcccatcctgaacgccctggaggtgcagggccgggagacgcgcctggtgctggaggtggcccagcatttgggtgagtggggaggggggggcctgggggggtcctccggtgatgatggatatcggtgactttcgttcttctgagctcgctgaagccacacgcgaaaatctgaggaggaggaggagggcaatgggtgctggggggctgcgcgttgtggggggctgcggcccacagtggctgtgcttggggctgctatttccctgcaccgttttgtccttgggggcctctcccagcaccgcatccccttggcaggggtgggaggaagcgtgcagctgtccccagggtcagcccgagggcactgcggtaactctgccctcggcaggggagaacacggtgcgcaccattgccatggatggcacggaagggctggtgaggggacagaaggtgttggactccggcgctcccatccgcatccccgtcggccccgagaccttgggaaggatcatgaatgtcatcggggaacccatcgacgagaggggccccatcacaacgaaacagtgagtgatgctgcaggccgggccgtggtgccgtgtggggtttggaggctcggggtctggcagcagttgctgtccgtaagggatccactttggtcgctcatttgcttccctctggtgctgtaggtttgctgctatccacgctgaagcccctgagtttgtagagatgagcgtcgagcaggagatcctggtgacgggcatcaaggtggtggacctgctggccccctatgccaagggtggcaagattggtacgtagccctcagagggagggcggagcagaggcactgtgcccaccacgaggctcagagcccatccccacacagcagcttccttcgctgatgagtaaccttttgactttcgttctactgagcttgctgaagcaacgttcattacctgaggaggaaaggggtgaagggagaccccaggggggctggattggctccgggttgagtgggtcacccttttcccccaggtttgtttggaggcgctggcgtcggcaagacggtgctgatcatggaactgatcaacaacgtggcgaaagctcacggcggttattcggtgttcgctggtgtgggggagcgaacccgcgagggcaacgatttgtaccacgagatgattgaatccggggtcatcaacctgaaagatgccacctccaaggtgcgtgaggcccgggctgggctgggggcccgctggcacggcagctgtgctgatgttgggtctctccttgtgccaggtcgctctggtctacggacagatgaatgagcccccgggtgcccgtgccagggtggctctgactgggctgacggtggcagagtacttcagggaccaggaaggccaggacgtgctgctcttcatcgacaacattttccgcttcacccaggctggctcagaggttggtgctgaccaccgggcagccagctgtcccagcatcaacagcccttcatcaggcccatccccataacaatgctgctcttcatgtgggctctgctggttgtctcaccgtctctgttccctcctaggtgtcagccttgctgggcagaatcccctccgccgtgggctaccagcccacactggccactgacatgggcaccatgcaggagcggatcaccaccacacgcaaaggctccatcacttcagtgcaggtagtgccagtgctgtgcctgctgggctggggacccccacagcgcccgcctctgtcccatggtgcccacccttcctgcaggccatctacgtgccagcagatgatctaacagaccctgcccctgccaccaccttcgcccatttggatgccaccacggtgctgtcccgtgccatcgctgagctgggtatctaccctgccgtggaccctctggattccacctctcgaatcatggaccccaacatcgtggggcccgagcactacgacgtggcccgaggtgtgcagaagatccttcaggtgaggtggggatggggcagcgacccctggggatgccatggggtgcacgaagccctgactgctcaccgcctgctttgtccccccccccgccaggactacaagtccctgcaggacatcatcgccatcctgggcatggatgagctgtctgaggaggacaagctgaccgtggcccgggcccgcaagatccagcgctttttgtcacagcccttccaggtggccgaggtcttcaccggccacatgggcaagctggtgcccctgaaggagaccatcaagggcttcaagcagatcctggcaggtgaggagggggcacggggagctgggggaccccacgccgggcctggcaccacctccccctaccgctggctgtgtttcctcccctcaggtgaatacgaccacctcccagagcaggccttctacatggtggggcccatcgaggaggcggtggccaaggcagagaagctggctgaggagcacgcgtgagcagggctgccttcacccctcccggctccgtgtggggccggacccaaagtatttaaggtttccaataaaatgttggtgaaaacagcccg

SEQ ID NO: 27 - DNA, 5358 bp, Gallus gallus
  misc_feature: cdc20 (cell division cycle 20)Accession number NC_006095
gtgaggctgagcggggacgcggaggtgagaggggctgcggtggggctccgggtcgggggggtgttgaggcctgaaggccccgtcgaggagggggggcccgaggctcggcctaacgggaggggggcggccgtgcccggcggtgccccgctgacccggcgggccgcccgcagcgccatggcgcacttcgtgttcgaggcggacctgcacgggctgctgaagctggacacgccgatccccaacgccccgcccgcgcggtggcagcgcaaggccaaggagagcgcctgcccgggccccggccccagccccgccgccagcatgtcgcccatgaagccggccaaccgctcgtacagcggcagcaagacgccgtccaagactcccggtgagcggaggcgcccagtggcggggcggggtgggagggcgagcggggctgtgccgggccgccctggccgggtgggacggagggcgtccctcgcttctgggtgcgcggagctccgcgctggggctgggagaaccgccgcccccaacccctcggccgcccgcgtgatgcggctctgccgggacggcgtgggagggctgcgagcctcgtcagcttcgtgcgctgtaacagtgggaaatggccctcccttctttgtgagccccggcgctgtcgttgcggcagaacctgggagttatctgtttggccgttcccgatctgagctcggcgcattcgatgcacggaggtacgggggaggccgggaatagctgcgagctgcggctcgagcggcctgtgtgctcccaggcctgctgcggtcatggagcttgtctgtgctgggggctgaagtggtaaaatgcttcttgggaacggtgctagttttgctttccaagcttgttgaattcagactcactcagtatctctaaaacacactttatatatgtgaacgcaggcaaatctggatccaaagtccagagcaccccaacaaaagctggaggggaccgctacattcccaaccgcagcactatgcagatggaaatggccaatttcctcctaaccaaagaaaacgaccctgctgaaaattcccctaccaagaaggtgagctttatctcttactgagattaatctctcactaagattagtatgacgatcgttgccaaatacctaatccttcatgacctgtcagagaagcagactttaaaagcaggcatggggatgagggctcctcccaactccacttccagtgctgggccttcagaagcctgcaagtggaactggcgcacagcctgcacacgtgggcctgagcacagctgctctgcagaggtatatccttgagcacaggcttttactttggcctgtgcagcttgtgatagcagaacttgggctactgagactttgaaatagagaaggttacggagaccagagctgtgagcctgagcgctgtgaaactgctggcagtagctgttctaggacaatgtctgcatggggagcaattctggaatgcgcacacaatgctctgaaatgtgtttatgaagcatttggtcccttgtggctccagctttctgcgctggtgatgagacgctgaatttgcctcttctctgcaggaaaatagcaccatgtcctgctgaatcccacagcgcaaccgaactatcttctaccagctttgtttaagggtggtatttcaaatgagtcttgatgagggtggtttgttttcctaggagcaacagaaatcctgggcaatgaatcttaatggctttgatgtagaagaggctaagattctccgccttagtggaaagccacagaatgctccagaaggtatgatgcagggtcagggtggtgcgtgcagggtctgtttgagggacttgggtgcagagatctgcgtggggaactgcaacacacatggccctatttctggccatgggagagatcagctggctgcatctgagttctttgcacactgaaatctctctcaggtttcctgaagttcaggggttggctaaaagctggcatgttgtttgtgtgaggtttctcattgtgctgttttatgagagttggattatgactgccaattgcgaacttgtactgcagataatgtatttttctgaagtctgcctatgaaagcttccaactccctaagcaaggaagtgggtagtagcaacagtcagttaaacgtgttctggtgaactggaacccactctgattcagcaatactgccagtgatgctgtaacacagtgctcttctgcccagatctcttttccagggctttgttctaactggaaggggttttctagtgatatcctgcttcctgaccattcactcaaagctgtttctctgtgtgcaggctatcagaacaacctgaaagtgctctacagccagaagactacacctgcttccagtaggaagcatggtagatacattccctcaatgccagacaggatcctggatgcaccagagattcgcaatgactattgtaagtaggctgcggtgttgggatcattttgcagcggggtgaaatacttaggcctgtgtggtctgatgaactgaaactcttaattgggctgtttcagttctcatgcacccagactggcactgaccacttctggagtgtctctgtgctgcctaatagtccaaggtaccatgcgggtgacttgaactggggctacttgactgggcatggtgaatcccaggaggctgccccttacctgcatgcgcacatggctggtgcaggtgggaggtgtggcatcatcacacagcactgcagtgaaatattttcagtaatgctgaagatgtgggtgaggctccgcatgtaagcactgtgtgagctgcactgctctttcaaattgtagatctgaatctcattgactggagctctcagaacttcctggcagtggctctggacaactgtgtttatctgtggaatcactctactggggaaatcattcagctgctgcagatagagaatccagatgattatgtatcctctgtgtcatggattaaagaaggaaactaccttgctgttggtacaagaaatgctgaggtccaggtgagtgcagtaagatgagaggtgttgtccaaggattgctggagctgtggggccttggaacaaagtcttggctagcacaggaaagatacagtgttgtatctttcatacattctgacttgtcttaagcacaggggagaggagtgttctaggacttgttatagtccagtgaattgctgcccactgtgggagcagaagagtgaagtggatgacttgtgacagaggcctgtagggaacttgtcactgctgtgcttctctgtcagagagcaagattgtctgaaggtgtccttatgctcacatcatcataaggtagcacaaatcctaagtagtttgtaatcacagtaatccttgctgtggtcttgtgactattagtagttacagtctctgtcttgtgatgctgagggtgctaagactagcataagtggaagtgtccccatccagctgtggtcagtgacaaactgcagtagtgaggcagactttagggattttctcatttctggaataactttgttctgtatgtgagaaattaactgctgctcccatactgaaaaggtcaaatgccagagcctatgtagtaaggtttcctggcaaagcaacaatgctgttggtccttaccctctggagaacataatactcttgcctctctagctatgggatgtacagcagcagaagcgtcttcgaagtatgaccagccattcttctcgtgtggggtccctcagctggaacagctacatcctctccaggtaaagctgggggtgaagcagtgtccagctggacacagtcagttgtgaagaggttctgtggctgctctgctcatgtcaacaggccagtgatggtgatcaacactggttgaaacccagtggcaaaaaaggcagccttgcaatagcacaaaaatggcttttggtgaaaatgagctagtctgacactgtgaacctcctctctgcagtgggtcacgaactggccacatccaccaccatgatgtcagagtggctgagcaccatgtggccactcttactggccacacacaagaggtgtgcggactcaagtggtctctagatggtcgctacctggccagtggtggcaatgacaacctggtgaacatctggccctctgtccagggagacagtggagacttcactcctgtacagaccttcactcagcaccagggtgctgtcaaggtgagtgcaggtctgctgtgtgtagtgtgggagcatgtctgtgtgaaaggcaggtgtgtagcacttgctgtaaccctgccttgttggagggagaatgccctgaagctgctgtggcctgagctctggaagatgcaataggggacaggttgccttttgtactacttaaaatgaactgtttcttcctcaggctgtggcatggtgtccatggcagtcaaatgttctagccactggaggtggaaccagtgacagacacatccgcatctggaatgtctgttccggcacctgtctcagtgctgttgatgcccactcccaggtgagacataaacatcctcgtgataaattctttctggcacacatgatggtgcctgggccctgggaaaggtcagggagccagcaggggctcatccttgctgatgagtatcagcacaagtagcttgtgtcttcatggtatttctgcctctttgcaggtctgttctatcctgtggtcaacaacctacaaagagttcatttcaggccatggctttgcgcagaatcagctagtgatatggaagtatccaacaatggccaaggtcactgaacttcgaggtaagctgagttctttgtctgaggaagagagggctgaaggcagattcaagcctcagtttgagtagagactgctgcttgtctaacctgaagcaccaataataaaaagatagtgtccagggccatcctccagaatacttaagtgctccaagagtaggccaaggatgtgacctcatgaggcttcagcatgttacagggacaatgatgcaggctgttgtggtttatgagggacatacaagtgtaaaaggtggggggaaccatggttgtgtttagaatcagcacagccctgactgcatgcttccttaggtcacactgcccgagtcctgaacctgaccatgagcccagatggtgtaacagtagcatcggcagctgctgatgaaacgctacgactttggcgctgttttgagatggatcccataaagaagaaggaaaaagagaaggcaaacagtgccaaaagcagcatcattcaccagggcatccgataagtgcacagagttggtgttgtgggaagctgggacatgggcttacacactgtacagtattttaagtgttaataaacagctttaatttgcctttgccttttaaa

SEQ ID NO: 28 - DNA, 12369 bp, Gallus gallus
  misc_feature: Casp8 (caspase 8) ACCESSION   NC_006094
aataacaatgtcctcgattaggaatagcttctggaatggacttccagaaggaatttaagagaacttgcaagagaatttgtctgggtctgtgtagttcgttgctgtcctgcaaggagacatctgacctaaatttttcaaatgtgtaatgtgtaatgagtcttctcaaacttgaattggtaggtcttctaaggcaatggaggagtaaagacttgctttcctgtcctgctgaagcaatgaaatctattcagccctggtcttaagtgtgaggaagcacctccaaccagtatgatttttaactgttatattacactggaattatcatttctatctgttgagagagtaagtccttgaaatctgaagaaaagaacaggaagatccagagtgccagatcccttcccagggaacttgtgtaatagatttacacaggactgagtgactgggtatctctggaaggtgtgtagtttttcacagaaaataatgtctctgtctagaaggacaaaagaaaaatgattgttatatatctggttcttttccttcacatttttttgtttgctttatgttattaatttgcatggttgggcagcattgctggctaggatttttaataataagttccaattgtgctctttctcagagtgctttggaagcattatcttttgaaaggctgttggccactggaagtaaggtcccactccatgagactctcaattctgagatgctgtcacctcctggcaactggctgtagcttctaccggctcctaatctctattgtaataaacactgttggttgtcacatcacccttgtttgtggtgttttcctctgaggagctgactgagggtcagtcagcacaacaaacctcctttgcttcatctgaagaagttacttttgctttcactgaagtattctatggcagacaccctgagggaggggtggcgtgttgatattctttcattagcagatggagagatggtagaaaagagtaagcttaaagttaggacattcttgataatgggagctgattccagtgaagctgttcttaccttgtatcttcagcgatacacatgtagtatctgggagccatctggaattctaatggctatatccattttgagctttataacaagacaaaggatcaatgtgcaacactttataacttctgccttttttaacttggattttttttttttttttttttttttttttgagatctgtgtctagctgtaatgcaggggttctttattcagtcctgtttgtcttcctgaagtgaggcttttttactcctaattcctcacagtatgttgacagttgctctgcttaccattttcactttccaagtaaaagagagaatgtctgttcttagaatcacagctctgtgattctaaaaacccaaaaagtcagaaggatcgtgaggccccgctttcacggtctgtatttgtactgaaaatcaagatcaagtgagctttcacccttctgcatacaggtttcgtaaataatacattgatattatgagcactatataaatgagaatgctgtttgtggcagatattagcagaaggtgttaggagttatgatgcatttctatagctgatgtttggttaactgttgaggaaacatgacaattcacgtttttacagcattttcataagtgcgaccttgctataaatgtagtagttaatttttttttttttaaattttatggaaatatagtttgaatgaaattgctgcttcctctgggagaagcttgctggaaggtggcagctgctccagtagaattaagagaaaatattactggtattagtgaagtgatgaatctacagcacttccgtttctttctatgtctccgtgtcagaattctgtcctctcagactaggttcttttatatactccacatgaggagaatttaatgtctaatatcaattttacttgtttggtgttacttgggtttttttatatagtaataataaacataatattataaaataggaggcatgacttttggagaagcccttgtacttagaagtaatttcaggaagaagtcatactgtgagttgatacgtggctcaatgaaggagaaaatgcactttgattttttgtggttctgagcaaaataaacactcacagccccatggctgagtgactgcgatgtttctgaccatatttgttgccctctaccagagctgagagttatgaaggaagcttgaaggctgtacaatttcctgactgttagcttctaacagtctgtgactgactgactgtgcttctctgtagcacaacccggtgaaaggggaggggtaactgcctctctgttcagaaggctatagttagtcatctagttattcagcctgcctttcccttttcttcaactcatgtctgggaggcagaactttttgcactgaaacaatgctggcacttaagtctgttctcagccaatggaagcgttaccagcgggaagtgaatcacaagcttggcaggaaggcaacgctaagtcctgcagaggtaacctagaaagcaggcaaagtgacgctgtttgggaaagaaaatgctaagtgagggaggggcattatctgcttaggcaggaacagtgttcattcagaggcacgaatgtctgccctttgatttgcatcttcttttcctatttgttcagttgaatgtcagtttaacgtcagtgtcccacagacatgttgatgccagccttccctgggaaatttatcattctctcatttttagggtaacctggtaatctgctgctgccggggtgggttattggcaagttttaggagcctgcctgctcactgctgctttctctcattgcatgggctgctgtaagtatgtgcttttccactacctctgtaagggttcactcttttcaaagtttttaaagtgctgaaaagtttgattacaggggatttttttatttaattacttacttatttatatttaactgttggctgtgtctgtcgtctacaacatctcacacaggaaatcgtggaaactcatgggaagtggaaacaaattccacatcctttagaagatccagttgtgcagcatggagtccagagctagaacgttctcaggaatttttcagtgttgagtccttgatggccatcctttttattttgacccctaacttacatgggcacatgttctctggaattattcagatctacctttttctgctaaagaggtgtaaacttacagagaacatgtttactttacaacggtttctgtgcagtgtactaaaatatagtttagatctttgggaatttccaagtactcatttgtgtttcctttctgaagctgtgcctttgcttactgctgtatttgttctatttttgacagccatgggaattccccagtcacataatgtgactagcttttgcagttctcaactgtaatacattcctagctaggtacaaagttatggcctgggtcttttatatcagattttcctgtagctactttgtttttaggctgctttgtccagacatgtcagtgcaaaaagaataatccatacctcctgcagtctgttcactaggagtttgttttaaaaggacttgaggggatattaactcataagctgttaaaaaaaaaaaaaaaaatgtctcactacattcagattacttccacctttgacccaatctatatgcaggcagtaaccaaatatcttgcttagggctttgaacattggtgacttttatgataataatgtaaatatttctgcttaattgctgtgttaaagggtatatatccaagttggcagattcttttgaataccaataatgtgaataacacagctctgaactatgaaatacccctacagagatttctgtgtttcccagggaagcccgtggtgaaactgagtgtgaaaatcttccccagtaggtatccctgtgggggcgggaagagaggctcagcccattgactggttccagaaatcagcagtgtcctcctgacttgtctgcgatagaatctttcccaaaaattcctcctccttttttttttttttttttaatactattttcttactttcaagtggagcttgagtgactaatattacctgtctttgtcgcgattggtgcaacagaaaactgaaaaactgctatctcagtttgattgctatgcagtttatcagttccacagtaccaccaagttcccatccctgtgcttagggagctgtcatggaatacactccatgcatatttactgcattccaccctggaggtttctacaacacttttcttcttttgagaggagaatatccctataagcaacttccggtaatcattccacataatcgatcccatgactacagttgctcaagactcacaatacttagaaggaactggggagggcatgtgcagcctttttattaggattagagttcaaggggctctgaggctgttcactatttatagggtaagatgattggcacagtcatagtatgtagcagaactgtgttatcagccctgaaaacagccttgggtgtaacagccaggccaaaggaggagattgggggtggggggaagcacactctcagattactgtctaaaaaaatgttggtttttttttttttttttgaaagagagaagtagtaggaagaggttccgttttaaaaccagtgctgaggctgagcgcagatctcctgccatggtgtaaatgttcggttttagaaccatgaaactccaacaagtaaaaacatctccctccttcctcctgttgttatgaaataccatctgtgtgttagaatttataacagaggcgatgcaccatcccaaggtctcaccccatcgtgccacttcctgtgggcttgctcctcctgctgaaagcctccagcaaatcccgagggctgggaagggagttggttccgtgacttgtgcccgttaatagggaaaagggtcaaatacctgtactgagcgtgctgtgcttcctgtcggcagcctgggggcagcttgtgaggaggaagtggtgttctgagattctttcattagtattaaaatgatgatgaattgttagtgccctcagactgtggaggtgtgactgcagacagagcccaggcagagggagagctcctgacctgcaaaaataagttcagttttctcaagactccttttcatgccagaaatatccttgggatgcatgtgaagataatggcgcgatttgagctcaaactcttattggagaactctgaagtcctcttgtttactatgcttaaccactgaagtgccactctggcaattgattttcacgtgtctctgtgtgtgtttcttctgaagtacttcagcacagaggcaaagctcttcctttggcttcccccacgtttccacttactggttttgctgagtgggtgaatgaatcttatgaagcaaaagggaagaattcaaaaaagcagtgggcagcgtttatgtaaaacagcacagaatgctgtgttttgcatgtaatacaagatcagccacgtacgtaacattcacttcttcctgtcagtagtatgtgtgtttgaagtgatatgaggaatctctcctccaggaaaagagcaccgatctgctattgtttttgtggggtttttttttcttcttttttcttttttctttctttctttcttcttcttttttttgtttttttaacagtaaagagagatgtatcggtatttcacggtaagagaagaaaaggaaatatgaagtacagtgccgcccaggctgaaggaaagagctcagcatttggacagcagtgacgcctgtggagataggcgtaggggaggtgggcgctggtgagcgctgaccggcccccgtgctctcccccgggccgtgccaagcgcacagcagggattttctacttcttcagtctctattaaaaaaaaaaaaaaatagcgctccgtaactgtgaacggagacggcctcagctgtgtcagaaagctcccgtctggcaggcaagcagtttctgagttgaagtgggagttttcaagtcaggaatccagacggaactacagctcccaggagcctgggcgctgcctcgcctgtccggaggcgggcggaagcgcagaggcgcgggtgcacgaggaagcggcgcccatcccggcccggggctggagctgctgcggggcgatggagttctcgcagctgctcttcgtgatcagcgaggcgctggacaggactgagctggcgtctctgaagtttctcagcctggagcacgtcactgtaaggaagcgggaagatattgaggagcccaaggccttcttccaagcattacaggagaaagggatgatcgaggtgggggacctgttcttcttgaaggagctgctctatcggatcaatcgaatagaccttctagcttcctacctgggctccagccgagaggagatggagagagagctccaggttccaggcagggcacgggtgtctccgttcaggtgagcggctgacagtgtgggttcaccagggtcctggaggcgaaagcctagcagagcctgaccctggagttcctctctggggatcagttatctttcttgggtggggaggtgcaagatttttacagatgagttaacaccagggtaagatttggcaaaacacaagcatctccatctttccaaggaaaagtgggtataagcagtagagaaagcttctgtccagtaaaggagagaagaccaagctttcttctctgtaagagccatgaggaactgtaatggactgtccagagaggtggtttagtcactgtgcctagaggtgttcaagaaacttgtggatgtggtactaagggacatgatttagtgggaaactattggcagtaggtgggcagttagactacatgatcttagagctcttttccaaccttggcgattctatgattctgagataacacccgcctgggtgttgtgggctgcagtcagtgactgggtgtgactgtctttgccctggtgtcccagtgcagaaactctctgtgcaattctgattttgggaaaacagatatttttagttgcagctgaaaatggtttgacttctgtctgttctgggactgatgtcctcaattaaagagaaaaaaaaagctgccctgggaaggcagctgaggggatggttcaatggtacagtgtaaaactgtgcgaaggagaatgctagagaacataatgaagctgtcctccactgttccttctaggtatctgctctttcagctgtcagaaaacatcaccaaagatgacatgaagtgtttcaagtttcttctggggaaagaattaccaaaatgcaagctaagtcctgaaactgtaagaattgttttgttctgttttatttttcttttgttttcttttacatcattgctataatacatagcaacatttaggctgcattgaggggaatgagtgtcgcttttccataaggaaaagggaggatctggaatggtgcatagaggacatgttgcatgccagtgttacgtgtagctccagcagtactgctccagcccagaaagcacaggccagccctggaccaggcatgacgcctgcagacccaagctgtgtatagggttgactggctttcttcagtaggaggctgagcactgtcactcatcttttccagctggttttcttttggctgctttctaagtaagtaattagcaaatgaagaatcacccactagggctattcctgtaatgttttggttctgtttatttccaaacgtagaagaaaaccccaaacagatctaggcagagatccttactagagctagggcccaagagcttgaagttaacttacatcattgcaagagatccagctgctctttacactatgtcaacctgcccttttgactgctaggtactttgttctgcctaatgaaatctcttttaaaatcttccctctgttcttagaggataaactgtgcctgttcagtggtggctcttctagtaaatccatgggtgaccactgtgctctgcgactctctttggcaaaagcttctgtcccttgggttctgtctttcttcagagaactctttataccatatttgcatggttatacccatttttgctacagtgatgcttgtgtttcagtcaagtgtacaaagttgccactgcaagtcagataaatgataggagtcattaaaaatgacattatttgttcctgagtgattaccattagttctcctgggttagatcttactagctctcagcatctctgagcagtgggagggaaataagcttgttttgggaaaatatttctagtcattgtatttctagcttttgtcaacctacatttaattgaatcagcttgaggtatcagctgagacagtgagactgacagtggttcacttgtgttgtcaatacattaaaaccagctcggttgctcctaaacatgctgcagcttccttctcagtagcagcatggatctgccctaagagcaaactacctttctgattgctcagacaatgccggacgttttcattgagatggagaagaaggggattttgaaagaagacaacttaactgtcctgaagactatttgtgggaaagtggacaagagcctgttgaagaaaattgaagactatgaattaaacttacttggtaaaacaaaacaaggcctctttcccacactttttatccttgaagtgatgtataaggtgtcttacaggactaataatgaatgctttccaggtgaaggagagatgcttgtaacagaggggcaaagaagcagcacaggagcccctgaaggtaactgaaactccttcgaaaatgaacgtttagatcttttgcttttttccagcttttggactctctacactacttgcatcttgcctctttaaacatgtgcctatctgagaagtgatcaggtatccttttgcttactaactgatcacagcactttaaactcccttttaattgtttacttatacatatgttttgtgtgtgtattcagacagtgccatttggctggcatcatctgtggcacccgattctctgggcaactgtgatcagtcttcacaggtaggtcagtttgtgtcttagtaatgtcacaattttgtctcagaagagaccattcatttatgaagtacacatgaagtattgtaagcacagctgttaaaggcacggtctggaaaagcaaagatgcactactctgtaatgtcctttattttgaaactatgtaccaggcagaagctgcagcagcattaaaacttaagaaatgtttttaagggtagaagatacccctaacttttaataccaggagacaggcagataaataggcacagagaagcgggtgaatgcaacaccccttgtgggtggagtgtgatggtggaggctgcaagaggtgggttcttctgctgtgaggcagcacgtggccaagtcctgggagcagagctgtacggggagagctgcccagatagaaagctggctggctttttaccctctgtgccactacaaaggctgagaagcagtcacaagaagtctttctttccaacagcttgaagtttataaaatgaccagccgaccccgtggagtctgcctgatcctgaataatcacaattttgcaaaagccagggaagcagtgccagaactcagaaggatgaaagatcggaatgggacacatgtggatgcaggtacagtgaatgttacagatgagtttacatgtattcagggagagtggaccgtacataaaaacaggaagcattttgtgttgtgtttcagactaggagtctgaagttcaagcttccatttcaaggctatgtttcttgtagtggtagacttgcatactactaaaatacacagaatcagaaacagatttgctggagaaccagaattttactttatatctagcgtaagagtgggtggggacctgacttcaggcagtattacaaagggccctcactttagcaggctgtcctgcagaagagcagcacagctcatggaaggtctgaggggctgtgaaatgggtttatttgcactacattgaccaggagagaaaaatccatcatcagagtacaggttatcttcttgagacgcaggtactacctgaatatattgacagaaatatttatttatataaataaatatagaatcgttatataatatatattttaattaatatatataatataattatatattataataatataaaaataaaataaaattatattatataaataataaaattatattacatataatacataccataataatatatattatatattatttatattatatataatttataaattatagatattatatattttatattatatgtaatatacaatataaatatatgtgatatatattataatatacatatatacatataaatataatataaatatatgtaatatatattatatatgtagcattatacatatattatatataatatataaaatatatgatcaaaacatggaggtaaatgtcagtatttttagtttgtcatattttaatcgatgtactaactttgtttttattgaatgcgtgatcaagctgtcctaggggaggtttagatagatgttaggaagaatttcataagggtgtgggtggtcaagcactggaatgggctatccagggaggtggtagagtatctacccctggaggtatttaagagctgtgtggacatggccctaaggtgcagaatttagtgatgggatgtggtaggtctggttgatagttgaattctatgatcttgaaggtcttttccaacctcgatgattctaggagaaatcactcccacccatctagagttgaaacaactgttaacaaatagcaaatgccctccactcttggggaatggagtggcactgtttgacatgtgttgcaagatgcagagaacagctgtaaagttctgtgagagctaacagctaagtctgcacttttcccttgcagacaatactgaacagcaagatcacaaggaattcttttaatgagagacaagggaatgtaatacaaatatgactagtgatcagacaactagattattttcagagaaagaatgtaaatacctggatagtagggtggcgggattaaaaaaaaaaaaaaaaaaaaaaaaaggtttatttgtaagaagtgtccaagttttagaaaactgctttaatccccccttggtttactttccagatgctctgagaaaagtctttagcaaccttcattttacagtagcagaatacaaagactgcactgcagaggaaatccgtaacatagtgaacaactatcggtgcatggaccacaacaacaaagactgttttgtttgctgtattctctctcatggaaaaaaaggcattatatatggtgttgatgggcaggaagtacctatccaagaactgaccacttctttcactgggcaaaattgccaatcacttgctggaaaaccaaaagtcttctttgttcaggcctgccaaggtgatgcttatcagaaaggtgtaaccattgaaacagattctggggagcaagattattctctagaaacagatgcaagatttcagctggactgcatcccctcagaggcagacttcctcttgggcatggctaccctgcaagattatgtctcctacagaagcccaagccaggggacctggtacatacagtcactgtgccaacacttggagagcagctgtcctaggtaattatacttcctaagatactttttcgcaacacctttatcttccttctgaaggacacaagtttgatttggatgcagaggtagataacctgcagggggagtgatcactcagtgaaaagagcaatgtagaatagcaacactgattgtttgaagcagttcacccaactgctttaaagacggtttagaggagccattagcactcgcacaggtcaagatgatatataagaatgcagtaagttttctgatgttacatacaaatactctatacaaaaccgcttgtattttttgcagaggagaagatattctcaccatactgacagcagtgaatcaagaggtgagcagcaagattgacaagcagaatgcagggaagcaaatgccacagcccagtttcacactgaggaaaaaactcatatttcctgtaaactaaatgggagcagaggcagagagcactgcagtaacctggaaaccactcatctcagctaaaaattcagtaagacatcagtctgctcttagcacatatgcaaatgtttttattggtaacacataaggaataaaggctttacgaatataagagttaatctgtgtgacgctttttatacttggaagtatctcagagctggtctccagtttgcaaccttacaatatatggagggctcttccagatgtactctatgctgttgttgttgttttaagatacttttcaaatacaaactcatataaagccttgtgtattttatgtttaacttttcataaaataaaatgcaggggactgcaaattttatagaacgca

SEQ ID NO: 29 - DNA, 1364 bp, Anopheles gambiae
  misc_feature: Yellow-g (AGAP005958)
cgcgtttgttcttcgttgtggatcacgggtttgtgttggcgctggggataaacgaacgcacagaacacgatgagttgccgcgtgacggggctaccgcttgccctgctgtttgtgctggtgtccgcgctcgctagcacggacgatctcgagtactgcaacatcgtgtaccggatcaatggcaattcgatcgactttccctgcctttcgaccaagaacatctacatctccgcacatcgctaccagccacgcaatgtgatgccggtgcggtgccagtacgacaatcaacgcatgctgatggcactgccaagattgcgctcgggcgttcctaccacgcttgggcagatcgatctgaacaaaccgaactgctacgcgcacattaagccgtacccttgctgggcctatcaggaggagggcaattgcaactcgctccaatccgtcatcgatctgtacgtggatgtgaaggtacgttgttgtgttaccgtggccaccgtgcttttgcggtaggatgaaaacatgctaacgtgtcgtctctttccttgcagcgcatcgcgtgggtgctggactcgggcatcaccaacttcctggagcaaccgatcaagcgctgcccgccgaaggtgtatgccttcaacttgagcaacgacaagacggtcaagacgatcgacctgtcggagattgtgaaatccagctcgcggctccagtatctcgtcactgattacaacgagctcggccatccgtttgtgtaagtattgtgttgggtaaagcatgcaaagtaaaatcacgtgcgtgatgctaacgatctcccaacttgcagctacatttccgatggcgatggtgcaatcattgtgctggacgtgaacaacaacaagagcttccgcgtggtgctaccgcgtgccatctcggctggctgcggcgattcggacgtgctctacctgctgctcatcccgaaaccgaagggccagagcctggtgatcttcagctatctgtgcggccagaaggtttactcgatcaagtcggaacatctgcgcagcggcaagggttcgagcgcgatcgtcgagctcggcaccaagcgcaaacactccgtcctgctcggcacggacggtggcaagggggtggtgctgcggtaccgtggcgaggccgagctgtacctctgggacagcgatcagccgtacagggagtgtaactttgagctgattcagaacgcggacgagtgccggctgtcgacgcacgtggtcgtgggcaagaacgagcatctactgtcgctgtcgtcgaacttgagcgattttctcaacaacacgtgcggcgttggtggcgcctcgtcccggttgaagtacgtttgcaaggagtgtgacgatgattgttactaa

SEQ ID NO: 30 - DNA, 8612 bp, Anopheles gambiae
  misc_feature: AGAP007280
atggcggatcacgatcaaactgtgctagatggtaagtgccaacgacgcaatgcgtgtcctacatatggctgcatgtttttaccaacgtttcgaaaacaaactgccaaagcaattgatttaaaaaaacacactcacacacataaagctttcttgtcctcaattgctcaggagacggtccacacgcataagcacaaaaaaatgcataagaaatctatacattttccctcccctttgaacgatttactttgaaactttcaacatacccccaaaatgtgtacaagtaaacaaacggactgcggtccgtccatccaccaggtgatatgaatgaaaagggagagagagagggcaacgggtggggaggtttgggcaggctcggctcaaaagtttgtcaacagtgcgtccaattactcgtacgtaagcggaactatcatcggacccgcgcctgtcccggtgtataaacaattattgggctttttaaaccgattgacataacataacctagttcgggtggaggattggaggtcgtgcacgaaacggggtcttaccttcttggctttccttcctttccaacacggcagaagagttggcaacgatcaactcgacgagaatgcagcaattggtgggtgctggcaggacgaactggctctgcctgatgatgacggcgatcggtggagtgatcgtgatggctgcgatcgcgttcggtttctacctaaccatacccgcatcaggtacgtgatagtagatcaagaggtgagcgactcgtatgtcatttttaatacaccttcctcctgttagctctctcccaacaaccatctaacctgattgagctagaacctatcgaagtacaaagccccgaagagctggaaaccatccgcgaaacgtacttcaaagcggaagcacatgacatcctgatgggatcactggaattgctggaactgttaaacgatcttgatccactgctccaacgtcaccgctcgcgccgagaagcctttggagcagaacccgtcccgaaccgtatctcccgcggagacgatcgttccggcgagattacgtacggtgtgcgaaagcgcgtacagcgtgagttcgatctcggtacagtaccgatcgactaccggggtatactggtcggttctgagtctccccgtgcgccacgtaagcgacgccacaccgacatctcgctcgacgagctggagcgtgcaaagcgcagtgcacagaaagactaccagcggctggagaaggagtacaaccggtgtcggaaggaagcaccgaacgggaagctgtgcgatcagatctacgaaaagctgcaacgtctgtcggaggaagtgaatgcacgctttctcgagatggcgaacctgctgcaggggttcagcgagatgggtccagccaaagagaagacgacgccacagtatgaggagatgtactatagtagcaccgagcgaagtacaagttatcgaccggagatgcacgctccgaaaaacgatcccaagataacgactgttgagcagcttgtgaatcagatgaacattacacaggccatccccaaggagaagctaaggacgagtgcggaggatgagatggtcaccaccactcccatgctaccggtgtttgatccgagcctgcataaaccgatcgagaatagttctatacggagtttggatgatctgttcgatcatatgagactgagcggagaggacgcttccaccaccgagtggactacagagcggcgtgagctgtttgagttcgaaatcgactccacttccacgtcgcctgtcaccgtgaagccatcaacccaaccgacgagtaagcgacgaacaacaaccaaaagcccactgcaagcggctcgcaatctccacgatgttgtccatcaactgcaactagcacagatgcttcagccacaccccttccacgaggatcttgtgatgagtccaccggaaaacattaacgagtttgtcatgtcacgatcgcgcaatcgggaggaaatgcatcacgacacgacccccagtccgatgcgtgccgtcgtccatcaacaacatcaacatctgcagcatcagactcaacctcaatcacctcaccaaccgttggtcgttggtccaagtgctccctttctgaatctgtgcgatcaacttcgatcggcagcaggaaatggccacctgaagccaacacaccctggggcaacattccaacactcaccaggcattccgatcacgggtgaagcaacgaaagcctcctcccagatcattgtcaactcggcgtttggggctgggtatgtgccgaatacggtttgcttctaccagaacgcaccaccagtaggggcacaaccaacggcgtacggctttcgtcctgccggttccggtgctccttacgtgtatccgaacgcgccagggtatccaccgtaccagccacaccagcaacagccttatcatcaacaacaccaccatcatcatccgggtgccggaggtaagcttccgttgaatgatcccgcgatcgatgcactcattcaaccgcgcattcccgaaggtgattagtgtgacatccacactctgaagagagagagacagagagttcttactgtatttttgtgtctttttcctttcccctccagctatgatcaacatgcccgttagcatacagaaccttcatcaagccggtgctcgtacctcggagctgccggccgatcagaacggtcccatcctcctctgctcaatgatgcagaacgtggagcaaccgacgcgacatcacgccgggaatgaaacgcacccagaggacgccgaagaagagctggacgaaaatctagacacgatgtttgcggcgggacgctcactggcagcacgggccaagggacgccaccactgccgacggggctcggtaccgtgctttagctcgcatcagtgcgtcaaacgcagcagctggtgtgactccaagacggactgtatggatggtagtgacgagagtgcgtgctcctgcgtttcgcgcctccccaagcgcaagctctgcgatggttatgccgactgtccgctcggtatggacgagatgggatgctttgggtgtgaaaagttctccttctcctgcttccacacggaggcggagtaccgttcggctcaccggtctgggtcgatgtgctacagtctgctcgaaaagtgcgacggtttcgataactgtctgaacaggaaggatgaacaggactgtacgatgttggtgcgtgatctggggcactatttggcgtactcggtcccgcactctactggggtgttgcatcggaactatcgcgggaagtggtacccggtgtgtcataatcctacgcagtgggcaagggaagcttgcgagacggagctcgggccactcgatcgtgagccactgttgagccacggccatggcagtcttcccggaccgtacattagccagcgagcgaacagtgccgtaccgcagcccgagtttagtgaagcttgtaatggggtttacgtacacgtgaagtgtccggcggtgcgatgtggtacgagtcggatgcacgaacagcacgccgcccgaatcaatgtgcggacaaggcgtgaggcgaacgagagtgaaattgtggagagtgttcgcatcgttggaggatcacatgccgatccggaagcgtacccgttcatcgtcggtatcttccgggatgggaagtatcactgtggtggcagcatttacaacgaacattgggtaagaggagcaagagcctatcattttgcattggctcttacactatgtgtacttcttttagatcatttctgccgctcattgctgtgacaacttcgaccagcactacttcgaggtacggtcgggcatgctgcgcaagcggagctttgcgccgcaggttcaaattacacgcgtcacgcacatgatcgttcatcacgcgtacagctcgtcgttgatggcgaacgatattgcgctgatgcgggtagagcatccgttccactacaatcgatgggtgcgtccgatctgtatgccggagcgccatcgtaccacggacgatcgggattggatctggggaccaaaggctggcactgtctgcaccgctattggatggggtgcactgcgggagcgtggtggagcacgtatgtatcattggcttctcggaggacgcacacatcttctctaatccagcgcaatccttctctttagccgatcatctgatgcaggtttcggtgccgatcttaggctactgcaagcacaaatccgatcgtgacagtctgcagatctgtgcggcagaagaagacggcggtcatgatgcgtgccagggtgattccggtggaccgtttgtgtgtcagagcaagtcgaacccgttcgagtggtatctggccggtgtggtaagtcacggggagggatgtgcgcgtgcacacgaacccggtgtctacacgcgagtggcactgtttatcgattggattgcggagaaggtaaatgcgccactgccagcacgtactgcgcgggctgactgtccggggatgcgatgtatctggggtggagggatctgtctaccaccgggcaagaagtgtaacggatacgttaactgtcttggtggtgaggatgagtcgggctgtgggatggatcagatgctacgatcgatgtcgcagcgaggtgaggaagaaagtgaggaggagggtggtagtgccacacagagagcttcggatgtggatacaacggaagctgagactaccgtattgttcacatcggaagaaacgacgacgttaagcgtgccggaagaagcgagtgtagaagcgcaggagagtatgatcatgacgaacacagcacagacggaaccgatcactactactacaagtacagttgccactaccacacctcagcttgagctatctactgctgaagaaagcacaacggaggacagctctacctcagcaagtacctcagaagatgcacctacggaagcttctacctcgccggagtcaaccacattggaggtcacaacagacctcagtacagagccatcttccactacgaatgaaccaacaacaaccttcacatcaacaaccgactctgcgatcatcttccccatacacgatcacgaacaggatcgtcctaagcaagactttgaattcattgcaccgatcacctcctccacaacggaagtcatttggaaggcgttggaaaaggcagtcaaagaggtacgtggacaggtacgcactgggaatggcacttcagtggtgaacgccacaacggaaccaatggaagtgttctctacggaactgccactggaagccacgtccgaagatccgctggaagattcttccgaccatccattcttggtggagttggacgtgatgcagcagcagaaacacaagcgagtggcccaattccgcatgacggtgcacaatgtgcacacgaacacgaaggtgcagacgatgctgccgaatgatacggcacagtacaggcagttttcctgcaggaagtaagacttacggtggagttagctgttgaaaaaagactgtctaaattatctttaacctgtttttctagtatcacccagaagatcaatgtggcacaccggtgcgatcggatcgtagactgtgaagatggcagtgatgagctgaactgtacctgtcgcgacttcctgaaggataagtttgatttcctgatctgcgacggcaagacggactgtctcgatcaaacggatgagctgggttgtagtaagtgatcttctacggtgaggtagttttgatcaaaacaagctaaaacaagctcttctgtttcctcagtgaactgtcaggctggtcagtatgcgtgccggatcagccaggtttgcattcccggggtgcaggtctgcaacggtcatccagactgtccgatgcatgaagatgagctggattgctgtaagtattgctgtggacacattgttctgtgaagtcatactaacctttggtgctttagtggctctaacggatggacatcgtgtgtacttcgatgcaaacaatctcaccctcttccacaacacgggcattgtgacgaaaaacacgaacggaacttgggagatactgtgtggagcggttctcacggcaaagacggaacatgctgtggagaagatctgttccttcttgggatttgcgtaagtcctttatcaccgcgcgagcttcagaaacacctcatcatacacctttcgcttctttttagtggtcatcgaaactactcactgctctcactcgcaccgaccgatctgcctgttgggctgcttgcaaatccaaacaatctacactacgtcaaccttacgctccatgctccctgtcaagcgttgcgtgtctcctgcgtccagcacatcaacgctaccgagcacgacattgcacactttgagcacaaccacaagcaagacccggtacaggttaacattcgcccactgaacccgatcaaccggccccatcacatgccccagatcgtgttccaggagaacgctcacattgagctggtggaaaactttggcgatgattacgattggccctggaatacgaacatctatctcgatggagtaatgatctgcagtgggctgatcatcgatgcgagctggatcattgtgtcggggagctgtacacggttggtgaacctgcggcatcagtatctggcggtggtggcgggcggtgccaagtcgtacctacacattgaagggccgtacgagcaggtggtgcgggtcgattgctaccactacataccggaggcggaaacggtgatgctgcatctggcgacgaagctaagctttagccggcacgtgctgccgacgtttgtgccggagaagtaagttgtgatgcgaaatctgtctctcaccattggataaaacattattaatggattggggtttatttgtttgtagtgaaaacctaacggacagtgagtgtcttgcggtgggacaggataagtacggaagaacgaaaacgctgcgggtgcatctcaacaccaccaactgcccaggcgaacgggtgcgatgctaccataaggatctgaagcagccctactatcatcatgaatcttgttacacaccaggtaaggagaacgagagagattacttcaagaactcctactgattactctcatcttaattcttcctcgcagaagcgacacgatcaggtgtgatcgtgtgcaaaacaagtcgctccggttggtatccagtcggattctaccagcacaagcgtggtctgtgcggattcaacgaagtggtgcgtgttacgagcctggtagaatcgtaccacaggatccagaacgtgctacacaaggaacagtgcggtgatcagctgtacgaggaacctcattgcggaggaaagcgctgtcgctatggaaagtgtgtcggtgagaagttgctgtgcgatcgcaaaccggactgttccgatggatcggatgaggaaccggcaatgtgtgcctcacgcaatcaaactggaagtgagtagatggtgtcagtgtgttgtatctgatcattatacgaactcttttatgtcatttttttaagattgtctgccacatcaactacgttgcgcgaatgaacggtgtattgacaagtctagtttctgcgatcgcaagaacgattgtggagactcgaccgatgaaccgcacgattgctcctgctacacctatcttaagtaagatcactgtaagatcaggtcgcaatacagggtttcccacgatttattggttggatcccatcaatttttggttggttcccatatttttttagtgcgttcccacgattttttggccgtttcccagatttttttagtccaattgtattgatatccattcggaaaataccaataaattatgggaacgaactaaaaatccatgcgatacgaccaaaaattcgtgggaattcaccaaaaaatcatgggaactgaccaataaatcgtgggaaaccctgtaaggaggtgttttactatttttctctccttctctctctctctctctctctcgttgtcttagaattaccgacccaggcaagatctgcgacggagtgcgcaactgttgggacaaatcggacgagaatccacgcgtctgtcggtgccactcgacgagcttccggtgtggcgagtccgacatctgcgtgccgtatgattttgtgtgcgacaaggaacgggactgtccgaatggggaggatgagctgtactgttacgcactgcagcagaactcttatgaagcgtaagaatctctactacatttctacgtcttgtggtatgtcacgtttggagttaacatttccctttttactctttcatcattcccctagtgggtacggtgagctgatggagcaaagctacggcatctggcactcaaaatgcttccccacgacgacccagttcgacgacgagtacatgaagcgcatctgcgagcagttgggctacagtcaggtgcgcaaaatctttggccgagcgatcgtgcaaggtgctcggttgcgtacggccaaccagacggaaacgcccgttgataagctgcgtcgttcggcgaccaaagcgatcgtgcagaacaagttctccaaggtcgttattaaccagaaccatacgttttacatgaaaccgagtcggcccatgtttaaggtgatcaactggaactatgaggacgagcaaaactgccatcggttggagttgctgtgtgccccgtga

SEQ ID NO: 31 - DNA, 859 bp, Anopheles gambiae
  misc_feature: AGAP011377
tgtccctcaattgctctataccgtgtgtaatggcccgcaggatgaggtacggtgcagtggtgctgctagtgctactggtggctggcagtctcgccatgtacgccaccaacaatgagcgctgtacgtgcgcccccggaccaccagactatctctgcccgtcggcaccactgctgcaggtgtacctgccgcacgagctgtactgtacccgctactacaagtgtaccgatgggcgtgcgatcgagttccagtgtccttacggattgtacttcgatacgcagaacaatacctgtacgtgtgactttacgcagtgctaccggacagatccctgcatcgtgttcgttgatagctgccgttgctgcaacagacagttcgaaaacacgggcctggcgccggaaaatttctacgtctgctacaacgacggatatgcggtagcgacgacctgcccggtagcgctcgatccctgcactggctcgcaggtgcagctacagtttgtaaatcaaaagtgtgaggtgccaccgccgtgtaagtttaagtggtgttctttgcagagcggctctggattgaaaatccatcttggaatttcgtttcagctactaccacggtggctaccacgaccacgacagcggcctcaactacgacaacaacaaccgaggctacaacaacgaccaccacggaagctacaacaacgacgaccacggaggctacaaccacgacaaccacggaggctactacaacgacgacggcggccccggtagcataggagttccaccgaactgtagccaaccagttcatatttatttggttttggatctttccgttctactgccgcgcgcagcagaataactccggacaaggaagc

SEQ ID NO: 32 - DNA, 16603 bp, Artificial Sequence
  Sequence of the Final Targeting Vector
  misc_feature: Synthetic
cgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattggagctccaccgcccgggctggttctttccgcctcagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctaggatctgcattccaccactgctcccattcatcagttccataggttggaatctaaaatacacaaacaattagaatcagtagtttaacacattatacacttaaaaattttatatttaccttagagctttaaatctctgtaggtagtttgtccaattatgtcacaccacagaagtaaggttccttcacaaagagatcgcctgacacgatttcctgcacaggcttgagccatatactcatacatcgcatcttggccacgttttccacgggtttcaaaattaatctcaagttctacgcttaacgctttcgcctgttcccagttattaatatattcaacgctagaactcccctcagcgaagggaaggctgagcactacacgcgaagcaccatcaccgaaccttttgataaactcttccgttccgacttgctccatcaacggttcagtgagacttaaacctaactctttcttaatagtttcggcattatccacttttagtgcgagaaccttcgtcagtcctggatacgtcactttgaccacgcctccagcttttccagagagcgggttttcattatctacagagtatcccgcagcgtcgtatttattgtcggtactataaaaccctttccaatcatcgtcataatttccttgtgtaccagattttggcttttgtatacctttttgaatggaatctacataaccaggtttagtcccgtggtacgaagaaaagttttccatcacaaaagatttagaagaatcaacaacatcatcaggatccatggcacgcgcttctacaaggcgctggccgaagaggtgcgggagtttcacgccaccaagatctgcggcacgctgttgacgctgttaagcgggtcgctgcagggtcgctcggtgttcgaggccacacgcgtcaccttaatatgcgaagtggacctgggaccgcgccgccccgactgcatctgcgtgttcgaattcgccaatgacaagacgctgggcggggtttgctcgacattgggtggaaacattccaggcctgggtggagaggctttttgcttcctcttgcaaaaccacactgctcgacattgggtggaaacattccaggcctgggtggagaggctttttgcttcctcttgaaaaccacactgctcgatttgttagcagcctcgaatcaacccgggcgatcctaggcgatgagatctagctgtcgcgaagagtggcgcgccaccaccgcccagctagctttcttaaattaagcagtttctgtcagtgttagggatttttacctgcaattccatcatatttgaatactctgtgactgagactgttagggtccagtaatagccatacaaacccctcagaagccaatctcagtcatgtatcaatggtttattgaacacattcctagactgattagtcaggagtggggatcgaaaacttcaataaggcattcctcagaaccaatttttatatgggaaaaacaaggacttggtacatttgcaatgtttataacatttgcaacatttatgctatctagacaaagcagtgctgtctggcctttaagctgataggctatgtatagagacaaggagacaaggagacctccaaagttcacaaggctcagtgaaaaagtataacaactttttttcagggcaagaaacaataggtcaagttaaatttatgagctacaaaatgaagattgagggaatatggcagaattttaatatttaacagagaaatgtccttaaacccccaaagagtaatttaaaagggtttttattatcaaacacctttttctgttttaagattctctatgtagagtaggttgcattggacaattatctttctggctttacttactgagttctgaaatttgaaaggattcaccattatttctttgtggcaaatatttgtcaaccccagaactcagcagatacaatagtggaagaaataagtcagctcttgctccctcccttacctctattctctagctcttttgctcccctgctccccctctctccccatccccttccttgctctctctatgtggtcatggctgccttctgcttctctactatctcacactctttgcctttctacaataaatgccttaaaaccaaaaaataaaaaataagaaaaaagtcagcagaggagaatgaaaggatggactactactacttcagaactgtttaaacctgagaattttgaggagaaagattgtttagtttcaacccttacaagggactgaggtacatattttacatattaaagcagtcctggcctccagattctcctagcatccctcagtcccaacctggcattctgtctccaaacctaaacttttcaggtcagaggctgggctacctttcccccagaagcttctccctatataatccagacatttggacaatgactcccccccccatgtccccctccttttccttttcctcctcttcttcctcttcctccccaccttgtcttatcctctttctctctctctgtctatctctctctctctcttctcccttctctgtgtacatgagctttctctctccatggtaacttccctggcctcagttattggggccagtgaacttgcccaagagcagcttcccaataagcctgcctatatataaaatattaaagacctgttccacaatatccaactaaagcaagctttatttaacactgactaggaagttcaacactgactacattcatgccaggattccaagagaaatggctttgaattaaaatcagtcatgtgatcatgcaaagaaacctctacaaatctatgaactttccatatttgtccaattaggggtaagcatacatcctcactgacttcctgtctgcatatttctcacctgtgtgtgatcaagcatatcctgagcacttggggcaacaaaccttgctattgaactaaaacacaacctccagtattctgcagttatttgtccttaggtaagtgtagcatacacattaactttaaagaattcccagagctgattttttgtttgtttgtttgtttgtttgtttgtttgtttttgagagaaagtttttctgtgtagccctggctgtcctggaactcactctgtagaccaggcttcctcaaactcagaaatccacctgcctctgcctcccaagtgctgggattaaaggcgtgcaccaccacggctggctctgactttttttttttttaagagttactttatttatatgagtacactgttactgttttcagacaccccagaagagggcattggatcccattacagatggttgtgaaccaccatgttggtggtgggaattgaactcaggacctctggaagagcagtcagtgctcttaaccataccatgcagttagtatctgagagtaaagtagcagaagctatacaccgtctgagaatcaattcttgtagtgtaggcttctggagtcccagaaactgcttttcaagttaaattgggtgcagggagggagggggagaataggtctaattgtttctgaaaccttttggcagaagggtaaaagataagtcagattgcctgttttttgcatcttatggcttccaggtctgcaaagtgttttagaagcttcctagaattttgttatggaagcttttgaaaagacttagacatgttttgtggacctggtttgggggtagggaatttacttgaagagggattaggtagtgtttgctagccagtcttcaatacaattcaagtttaaggtcatcttagatttataaatatttgtgttggagacttgtgatactggtttctgagatggggaactaggtttccttgtgaaggtccaggcccagtaagtaatggtaatacaagtcaattcaaaactcctgagaacagcaaaattggaacttgtcttattatattttttattattgtattatatataaatgaatgctcctcttacaaagtgttggatcatatttacaaattttagctacaacaaccaaaaggtcaaaagaaatggtattgttctcaaatggttactgcatcttcaaaatgcaatgagggattcccccgtccttcccccccccccgccccctcacgtcactccttgcccccgcagggataacctttgtgagggactgttcattcactatgatctgttgtttaaggacaaagtcatgtgacttttataaagtttttttggtttgatgtggttgagtggatgggtaattaatgagtttagctatggtggtgacaaaaaaacacgacccagttactcattgaggatgatttcaattttagctgaatacattttgttttcccaacgtgtgtgtcattacttcaagatcttaattttaaagtttgctagaattagtattaaggagcaccgattctgggaacttaacagaaggtgttctgtaaggcaagaggaaaatttttttttttaactatttggtctctaattggctttcatgtgccctaaaaaccgatgtttgatgtattttttcttagtgctctttgttccttgtgagatcagtatagcaattaaatgaaagggtctggatatctagtgtggaataagaaagagttacaaagcatacctaaaactaatttcactgtggtcggtaggtagagctcttaggctgacatcacgagctagcgaagtagcaagtttagctgcactggtgatgacgcaagtcaacaaaagtcacgtgggataaaacggttgcttccattggaaagcctggctttagggagaggttatggacgttacgtgcggttcacactgacggtattataacaactttatgcggatgctagcgaagtttgatattctaagaacttcgcttaggttctgcaacaatgagttcttcttgcgttcaccatctgcgtggtaatgtgaccagatagcctctgccgctttctcctttgccagtttccatgaaatcttacggattatctctcactactgctgccctaggtaatgaggaaaagaaaatggcggcggaaaaggctagaggcgagggcgaagaggggtccttcagtctcacagtcgaagagaagaaggcgctttgtggattagataggtacgcactgctaatacttacaggttgtctttctctctccgggttcaaaagactctccaggaggcctgactgcatcttgcttgcggtgacgactcgctttcgcctagcactgttaacaatgttttgggagattttttactgatgtctttctgtgatcattaacaagagccatgtatttcttccacagcagcttcttcgggttcttgacccgatgcaaagatggcgccaagatgaagacgctgttgaacaaggtcagcaactgtttaaacataattacttatatttttctcctaagcttttcactgttccaagcctggagcctctccctactctgaaagtgctcattatagcttttagggatctttgaaactttttttttccttccccgtttctcctttcttggcttactttcctgatagtttgaggccctgcctcagcatacagcccagagcactgcttattttgcagtttttctgaggtcagctatatcctccagttactcatctcaagttatgagaagacgcttggttgttgcagcctcttgtgatatacacacgttttattattttcccataagggacaagttataggtaaggagataaagagtttccgtacttaaaaagaattttttacgatgcagtagtaatactatgcagaaataatactgaattccagtttggaatgttaatttttttttttttttacttccttgtagtttattttccagagagtgagagagcacacggcatttcctttgtgaaactttgcattgtcatttagttattggctgctggtatcaatcaagatggtggcttaagactgacgggcaccggagccaattcccactcctttcaagacctggtaccaaaaaagcaccgactcggtgccactttttcaagttgataacggactagccttattttaacttgctatttctagctctaaaacttttgaggctcatctctgcaggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcttctagaaaaaaagcaccgactcggtgccactttttcaagttgataacggactagccttattttaacttgctatttctagctctaaaacatctacaagattcaggtctgggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcggatccaaaaaagcaccgactcggtgccactttttcaagttgataacggactagccttattttaacttgctatttctagctctaaaaccgattcgcccggtggcagtgggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcacgcgtgcggccgcctcgagggacctaataacttcgtatagcatacattatacgaagttatattaagggttccgcaagctctagtcgagccccagctggttctttccgcctcagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctagagaattgatcccctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcatcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatggccgatcccatggtttagttcctcaccttgtcgtattatactatgccgatatactatgccgatgattaattgtcaacaggctgcaggtcgaaaggcccggagatgaggaagaggagaacagcgcggcagacgtgcgcttttgaagcgtgcagaatgccgggcctccggaggaccttcgggcgcccgccccgcccctgagcccgcccctgagcccgcccccggacccaccccttcccagcctctgagcccagaaagcgaaggagcaaagctgctattggccgctgccccaaaggcctacccgcttccattgctcagcggtgctgtccatctgcacgagactagtgagacgtgctacttccatttgtcacgtcctgcacgacgcgagctgcggggcgggggggaacttcctgactaggggaggagtagaaggtggcgcgaaggggccaccaaagaacggagccggttggcgcctaccggtggatgtggaatgtgtgcgaggccagaggccacttgtgtagcgccaagtgcccagcggggctgctaaagcgcatgctccagactgccttgggaaaagcgcctcccctacccggtagaatttcgacgacctgcagccaagctagcttcgcgagctcgaccgaacaaacgacccaacacccgtgcgttttattctgtctttttattgccgctcagctttacagtgacaatgacggctggcgactgaatattagtgcttacagacagcactacatattttccgtcgatgttgaaatcctttctcatatgtcaccataaatatcaaataattatagcaatcatttacgcgttaatggctaatcgccatcttccagcaggcgcaccattgcccctgtttcactatccaggttacggatatagttcatgacaatatttacattggtccagccaccagcttgcatgatctccggtattgaaactccagcgcgggccatatctcgcgcggctccgacacgggcactgtgtccagaccaggccaggtatctctgaccagagtcatccttagcgccgtaaatcaatcgatgagttgcttcaaaaatcccttccagggcgcgagttgatagctggctggtggcagatggcgcggcaacaccattttttctgacccggcaaaacaggtagttattcggatcatcagctacaccagagacggaaatccatcgctcgaccagtttagttacccccaggctaagtgccttctctacacctgcggtgctaaccagcgttttcgttctgccaatatggattaacattctcccaccgtcagtacgtgagatatctttaaccctgatcctggcaatttcggctatacgtaacagggtgttataagcaatccccagaaatgccagattacgtatatcctggcagcgatcgctattttccatgagtgaacgaacctggtcgaaatcagtgcgttcgaacgctagagcctgttttgcacgttcaccggcatcaacgttttcttttcggatccgccgcataaccagtgaaacagcattgctgtcacttggtcgtggcagcccggaccgacgatgaagcatgtttagctggcccaaatgttgctggatagtttttactgccagaccgcgcgcctgaagatatagaagataatcgcgaacatcttcaggttctgcgggaaaccatttccggttattcaacttgcaccatgccgcccacgaccggcaaacggacagaagcattttccaggtatgctcagaaaacgcctggcgatccctgaacatgtccatcaggttcttgcgaacctcatcactcgttgcatcgaccggtaatgcaggcaaattttggtgtacggtcagtaaattggacaccttcctcttcttcttgggcatggccgcaggaaagcagagccctgaagctcccatcaccggccaataagagccaagcctgcagtgtgacctcatagagcaatgtgccagccagcctgaccccaagggccctcaggcttgggcacactgtctctaggaccctgagagaaagacatacccatttctgcttagggccctgaggatgagcccaggggtggcttggcactgaagcaaaggacactggggctcagctggcagcaaagtgaccaggatgctgaggctttgacccagaagccagaggccagaggccaggacttctcttggtcccagtccaccctcactcagagctttaccaatgccctctggatagttgtcgggtaacggtggacgccactgattctctggccagcctaggacttcgccattccgctgattctgctcttccagccactggctgaccggttggaagtactccagcagtgccttggcatccagggcatctgagcctaccaggtccttcagtacctcctgccagggcctggagcagccagcctgcaacacctgcctgccaagcagagtgaccactgtgggcacaggggacacagggtggggcccacaacagcaccattgtccacttgtccctcactagtaaaagaactctagggttgcggggggtgggggaggtctctgtgaggctggtaagggatatttgcctggcccatggagctagcttggctggacgtaaactcctcttcagacctaataacttcgtatagcatacattatacgaagttatattaagggttattgaatatgatcggaattgggctgcaggaattcgatagcttggctgcaggtcgacgtacgtagcaagcttgatgggccctggtaccgaaatagagggaggagaggggaaatgaaacagttttagaaaggaaacagggtagttttaccaagaaaatattaaacctagcaagtacaggttaatgaaaagtattccccattacttaaaaggagaattatcaagtctttttaaacctttaaaaaaagattcttagccgggcggggtggcacatgcctttaatcccagcacttgggaggcagaggcaggcagatttctgagtttgaggccagtctggtctacagagtgagttccaggacagccagggctacacagagataccctgtctcaaaaaaaaaaaaaaaattcttaaaggtaacatatttagatacttgtaaaccatagcaaaaaaagcacagatctgtgacatacatacttgttaccattatatagtttccgttctaatgaaaatcagtgattataaacttgataatttcataccaccaatgaagtattaatagtattcttttttggtttttggagacagggtttctcagtatagctctggttgctatacagttaatagtgttcttaacatatatgtgtatatataaatttatgtatgtatgtatatatatatatacatatatatatatgcatattatgcacacataatttttggtttggtttggttgtgagcctagcctttaaggactgagtccaagatttaaacactcttcaggtgatatgtatagaaacatatttctgtttactagtaggttgctaggtttctggtgtacagtgggtaattttaaatttgatgttaacctccacactcttttaagatttcatggtaaaagcttaaagaaatgattttgtctagtttttatttttattttttaattttttttatttttttggtttttggagacagggtttctctgtatagctctggctgtcctggaactcactttgtagaccaggctggcctcaaactcagaaatctgcctgtctctccctcctgagtgctgggattaaaggcatgcaccaccacgcctgacgtggtttttaaagatataattatttaactatatcttagcatttcttttttttttttagagtagtaattcttatattataaaaaagacttggtgttgaatgtatttttggagtgactacaacaactatgtgtgatcctgtttgtcctagaactcacagacagaaatatcactttctctgctttctcattgctcattgctaagattaaagccctgttttcctaaaactggcttcttttaaatttaataattttgtatatattcatgtctggaagttataggacaaattgtggaagtcagttttcttcttctatcatgtcagggctggaggcaagtccttttactagctaagccacttccttgaactttcagtttatgtgtatagtgtttctactgcttgtaggtctattccacttgagtgtctggttctactaaggagtttagaagagacagacctatggaactggaattagtgaaggttgtgagcttcactaatcaaaccagtgttctcttgcagagctctttagtttatttaagtagcatatatttttcaggtgctttgtaatatcaatttatatgttagtaaactgttttcaaatattgaaataacaggtattttttaactaattgcaacagcattttaaaaatgagaagttaaaaacaaaatatgagaaactatcaagtgccactgttcttagaacatttatgagttgtgcctttattaaacattgagtaatatgtatttttgttttagatcagtctcagcctttctaatgcagtttctcatgttgtgatgacccccaaccataaaattatttttgttgttattcataactaattttggtgctgttatgaatcattatgtaaatatctgaatagtttattagtgaccaaaaaggtattttgacccataggttgagaaccattattttagacactgtcttagtctatattttgttgcccagaatagctttaaatttgaagaagtactcatctctcagcctccaagtattgagatttcaggcatgagccactactctttaacagttattaaaatgtattacaattataatttaaaatgacaagtattaaaaactgaaatatagggctggtgagatggctcagtgggtaagagcacccgactgctcttccgaaggtccagagttcaaatcccagcaaccacatggtggctcacaaccatccataacgagatctgactccctcttctggagtgtctgaagacagctacagtgtacttacatataataaataaatctttaaaaaaaaaaaaacaaaaaaaaacctgaaatatataaagggcaattgatggatggtggtggaatatccctttatcctagcactgtgcagaggcagttgggtctcttataagtttgaggccagcctgatctacatagcaagtttcaggacagcaagattagacagagaaacctgcctcaaaaaaggacaatttgtttttaagcctgacagtggtggcatacacctttaaacccagcacttgggggacataggcagatagatctttggcggccgcgtaccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaa

SEQ ID NO: 33 - DNA, 6594 bp, Artificial Sequence
  Sequence confirmed regions of the Final Targeting Vector
  misc_feature: Synthetic
ggaccgcgccgccccgactgcatctgcgtgttcgaattcgccaatgacaagacgctgggcggggtttgctcgacattgggtggaaacattccaggcctgggtggagaggctttttgcttcctcttgcaaaaccacactgctcgacattgggtggaaacattccaggcctgggtggagaggctttttgcttcctcttgaaaaccacactgctcgatttgttagcagcctcgaatcaacccgggcgatcctaggcgatgagatctagctgtcgcgaagagtggcgcgccaccaccgcccagctagctttcttaaattaagcagtttctgtcagtgttagggatttttacctgcaattccatcatatttgaatactctgtgactgagactgttagggtccagtaatagccatacaaacccctcagaagccaatctcagtcatgtatcaatggtttattgaacacattcctagactgattagtcaggagtggggatcgaaaacttcaataaggcattcctcagaaccaatttttatatgggaaaaacaaggacttggtacatttgcaatgtttataacatttgcaacatttatgctatctagacaaagcagtgctgtctggcctttaagctgataggctatgtatagagacaaggagacaaggagacctccaaagttcacaaggctcagtgaaaaagtataacaactttttttcagggcaagaaacaataggtcaagttaaatttatgagctacaaaatgaagattgagggaatatggcagaattttaatatttaacagagaaatgtccttaaacccccaaagagtaatttaaaagggtttttattatcaaacacctttttctgttttaagattctctatgtagagtaggttgcattggacaattatctttctggctttacttactgagttctgaaatttgaaaggattcaccattatttctttgtggcaaatatttgtcaaccccagaactcagcagatacaatagtggaagaaataagtcagctcttgctccctcccttacctctattctctagctcttttgctcccctgctccccctctctccccatccccttccttgctctctctatgtggtcatggctgccttctgcttctctactatctcacactctttgcctttctacaataaatgccttaaaaccaaaaaataaaaaataagaaaaaagtcagcagaggagaatgaaaggatggactactactacttcagaactgtttaaacctgagaattttgaggagaaagattgtttagtttcaacccttacaagggactgaggtacatattttacatattaaagcagtcctggcctccagattctcctagcatccctcagtcccaacctggcattctgtctccaaacctaaacttttcaggtcagaggctgggctacctttcccccagaagcttctccctatataatccagacatttggacaatgactcccccccccatgtccccctccttttccttttcctcctcttcttcctcttcctccccaccttgtcttatcctctttctctctctctgtctatctctctctctctcttctcccttctctgtgtacatgagctttctctctccatggtaacttccctggcctcagttattggggccagtgaacttgcccaagagcagcttcccaataagcctgcctatatataaaatattaaagacctgttccacaatatccaactaaagcaagctttatttaacactgactaggaagttcaacactgactacattcatgccaggattccaagagaaatggctttgaattaaaatcagtcatgtgatcatgcaaagaaacctctacaaatctatgaactttccatatttgtccaattaggggtaagcatacatcctcactgacttcctgtctgcatatttctcacctgtgtgtgatcaagcatatcctgagcacttggggcaacaaaccttgctattgaactaaaacacaacctccagtattctgcagttatttgtccttaggtaagtgtagcatacacattaactttaaagaattcccagagctgattttttgtttgtttgtttgtttgtttgtttgtttgtttttgagagaaagtttttctgtgtagccctggctgtcctggaactcactctgtagaccaggcttcctcaaactcagaaatccacctgcctctgcctcccaagtgctgggattaaaggcgtgcaccaccacggctggctctgactttttttttttttaagagttactttatttatatgagtacactgttactgttttcagacaccccagaagagggcattggatcccattacagatggttgtgaaccaccatgttggtggtgggaattgaactcaggacctctggaagagcagtcagtgctcttaaccataccatgcagttagtatctgagagtaaagtagcagaagctatacaccgtctgagaatcaattcttgtagtgtaggcttctggagtcccagaaactgcttttcaagttaaattgggtgcagggagggagggggagaataggtctaattgtttctgaaaccttttggcagaagggtaaaagataagtcagattgcctgttttttgcatcttatggcttccaggtctgcaaagtgttttagaagcttcctagaattttgttatggaagcttttgaaaagacttagacatgttttgtggacctggtttgggggtagggaatttacttgaagagggattaggtagtgtttgctagccagtcttcaatacaattcaagtttaaggtcatcttagatttataaatatttgtgttggagacttgtgatactggtttctgagatggggaactaggtttccttgtgaaggtccaggcccagtaagtaatggtaatacaagtcaattcaaaactcctgagaacagcaaaattggaacttgtcttattatattttttattattgtattatatataaatgaatgctcctcttacaaagtgttggatcatatttacaaattttagctacaacaaccaaaaggtcaaaagaaatggtattgttctcaaatggttactgcatcttcaaaatgcaatgagggattcccccgtccttcccccccccccgccccctcacgtcactccttgcccccgcagggataacctttgtgagggactgttcattcactatgatctgttgtttaaggacaaagtcatgtgacttttataaagtttttttggtttgatgtggttgagtggatgggtaattaatgagtttagctatggtggtgacaaaaaaacacgacccagttactcattgaggatgatttcaattttagctgaatacattttgttttcccaacgtgtgtgtcattacttcaagatcttaattttaaagtttgctagaattagtattaaggagcaccgattctgggaacttaacagaaggtgttctgtaaggcaagaggaaaatttttttttttaactatttggtctctaattggctttcatgtgccctaaaaaccgatgtttgatgtattttttcttagtgctctttgttccttgtgagatcagtatagcaattaaatgaaagggtctggatatctagtgtggaataagaaagagttacaaagcatacctaaaactaatttcactgtggtcggtaggtagagctcttaggctgacatcacgagctagcgaagtagcaagtttagctgcactggtgatgacgcaagtcaacaaaagtcacgtgggataaaacggttgcttccattggaaagcctggctttagggagaggttatggacgttacgtgcggttcacactgacggtattataacaactttatgcggatgctagcgaagtttgatattctaagaacttcgcttaggttctgcaacaatgagttcttcttgcgttcaccatctgcgtggtaatgtgaccagatagcctctgccgctttctcctttgccagtttccatgaaatcttacggattatctctcactactgctgccctaggtaatgaggaaaagaaaatggcggcggaaaaggctagaggcgagggcgaagaggggtccttcagtctcacagtcgaagagaagaaggcgctttgtggattagataggtacgcactgctaatacttacaggttgtctttctctctccgggttcaaaagactctccaggaggcctgactgcatcttgcttgcggtgacgactcgctttcgcctagcactgttaacaatgttttgggagattttttactgatgtctttctgtgatcattaacaagagccatgtatttcttccacagcagcttcttcgggttcttgacccgatgcaaagatggcgccaagatgaagacgctgttgaacaaggtcagcaactgtttaaacataattacttatatttttctcctaagcttttcactgttccaagcctggagcctctccctactctgaaagtgctcattatagcttttagggatctttgaaactttttttttccttccccgtttctcctttcttggcttactttcctgatagtttgaggccctgcctcagcatacagcccagagcactgcttattttgcagtttttctgaggtcagctatatcctccagttactcatctcaagttatgagaagacgcttggttgttgcagcctcttgtgatatacacacgttttattattttcccataagggacaagttataggtaaggagataaagagtttccgtacttaaaaagaattttttacgatgcagtagtaatactatgcagaaataatactgaattccagtttggaatgttaatttttttttttttttacttccttgtagtttattttccagagagtgagagagcacacggcatttcctttgtgaaactttgcattgtcatttagttattggctgctggtatcaatcaagatggtggcttaagactgacgggcaccggagccaattcccactcctttcaagacctggtaccaaaaaagcaccgactcggtgccactttttcaagttgataacggactagccttattttaacttgctatttctagctctaaaacttttgaggctcatctctgcaggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcttctagaaaaaaagcaccgactcggtgccactttttcaagttgataacggactagccttattttaacttgctatttctagctctaaaacatctacaagattcaggtctgggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcggatccaaaaaagcaccgactcggtgccactttttcaagttgataacggactagccttattttaacttgctatttctagctctaaaaccgattcgcccggtggcagtgggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcacgcgtgcggccgcctcgagggacctaataacttcgtatagcatacattatacgaagttatattaagggttccgcaagctctagtcgagccccagctggttctttccgcctcagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctagagaattgatcccctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccag

SEQ ID NO: 34 - DNA, 1128 bp, Artificial Sequence
  KI region
  misc_feature: Synthetic
actgacgggcaccggagccaattcccactcctttcaagacctggtaccaaaaaagcaccgactcggtgccactttttcaagttgataacggactagccttattttaacttgctatttctagctctaaaacttttgaggctcatctctgcaggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcttctagaaaaaaagcaccgactcggtgccactttttcaagttgataacggactagccttattttaacttgctatttctagctctaaaacatctacaagattcaggtctgggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcggatccaaaaaagcaccgactcggtgccactttttcaagttgataacggactagccttattttaacttgctatttctagctctaaaaccgattcgcccggtggcagtgggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcacgcgtgcggccgc

SEQ ID NO: 35 - DNA, 4646 bp, Artificial Sequence
  5′ Homology arm
  misc_feature: Synthetic
accaccgcccagctagctttcttaaattaagcagtttctgtcagtgttagggatttttacctgcaattccatcatatttgaatactctgtgactgagactgttagggtccagtaatagccatacaaacccctcagaagccaatctcagtcatgtatcaatggtttattgaacacattcctagactgattagtcaggagtggggatcgaaaacttcaataaggcattcctcagaaccaatttttatatgggaaaaacaaggacttggtacatttgcaatgtttataacatttgcaacatttatgctatctagacaaagcagtgctgtctggcctttaagctgataggctatgtatagagacaaggagacaaggagacctccaaagttcacaaggctcagtgaaaaagtataacaactttttttcagggcaagaaacaataggtcaagttaaatttatgagctacaaaatgaagattgagggaatatggcagaattttaatatttaacagagaaatgtccttaaacccccaaagagtaatttaaaagggtttttattatcaaacacctttttctgttttaagattctctatgtagagtaggttgcattggacaattatctttctggctttacttactgagttctgaaatttgaaaggattcaccattatttctttgtggcaaatatttgtcaaccccagaactcagcagatacaatagtggaagaaataagtcagctcttgctccctcccttacctctattctctagctcttttgctcccctgctccccctctctccccatccccttccttgctctctctatgtggtcatggctgccttctgcttctctactatctcacactctttgcctttctacaataaatgccttaaaaccaaaaaataaaaaataagaaaaaagtcagcagaggagaatgaaaggatggactactactacttcagaactgtttaaacctgagaattttgaggagaaagattgtttagtttcaacccttacaagggactgaggtacatattttacatattaaagcagtcctggcctccagattctcctagcatccctcagtcccaacctggcattctgtctccaaacctaaacttttcaggtcagaggctgggctacctttcccccagaagcttctccctatataatccagacatttggacaatgactcccccccccatgtccccctccttttccttttcctcctcttcttcctcttcctccccaccttgtcttatcctctttctctctctctgtctatctctctctctctcttctcccttctctgtgtacatgagctttctctctccatggtaacttccctggcctcagttattggggccagtgaacttgcccaagagcagcttcccaataagcctgcctatatataaaatattaaagacctgttccacaatatccaactaaagcaagctttatttaacactgactaggaagttcaacactgactacattcatgccaggattccaagagaaatggctttgaattaaaatcagtcatgtgatcatgcaaagaaacctctacaaatctatgaactttccatatttgtccaattaggggtaagcatacatcctcactgacttcctgtctgcatatttctcacctgtgtgtgatcaagcatatcctgagcacttggggcaacaaaccttgctattgaactaaaacacaacctccagtattctgcagttatttgtccttaggtaagtgtagcatacacattaactttaaagaattcccagagctgattttttgtttgtttgtttgtttgtttgtttgtttgtttttgagagaaagtttttctgtgtagccctggctgtcctggaactcactctgtagaccaggcttcctcaaactcagaaatccacctgcctctgcctcccaagtgctgggattaaaggcgtgcaccaccacggctggctctgactttttttttttttaagagttactttatttatatgagtacactgttactgttttcagacaccccagaagagggcattggatcccattacagatggttgtgaaccaccatgttggtggtgggaattgaactcaggacctctggaagagcagtcagtgctcttaaccataccatgcagttagtatctgagagtaaagtagcagaagctatacaccgtctgagaatcaattcttgtagtgtaggcttctggagtcccagaaactgcttttcaagttaaattgggtgcagggagggagggggagaataggtctaattgtttctgaaaccttttggcagaagggtaaaagataagtcagattgcctgttttttgcatcttatggcttccaggtctgcaaagtgttttagaagcttcctagaattttgttatggaagcttttgaaaagacttagacatgttttgtggacctggtttgggggtagggaatttacttgaagagggattaggtagtgtttgctagccagtcttcaatacaattcaagtttaaggtcatcttagatttataaatatttgtgttggagacttgtgatactggtttctgagatggggaactaggtttccttgtgaaggtccaggcccagtaagtaatggtaatacaagtcaattcaaaactcctgagaacagcaaaattggaacttgtcttattatattttttattattgtattatatataaatgaatgctcctcttacaaagtgttggatcatatttacaaattttagctacaacaaccaaaaggtcaaaagaaatggtattgttctcaaatggttactgcatcttcaaaatgcaatgagggattcccccgtccttcccccccccccgccccctcacgtcactccttgcccccgcagggataacctttgtgagggactgttcattcactatgatctgttgtttaaggacaaagtcatgtgacttttataaagtttttttggtttgatgtggttgagtggatgggtaattaatgagtttagctatggtggtgacaaaaaaacacgacccagttactcattgaggatgatttcaattttagctgaatacattttgttttcccaacgtgtgtgtcattacttcaagatcttaattttaaagtttgctagaattagtattaaggagcaccgattctgggaacttaacagaaggtgttctgtaaggcaagaggaaaatttttttttttaactatttggtctctaattggctttcatgtgccctaaaaaccgatgtttgatgtattttttcttagtgctctttgttccttgtgagatcagtatagcaattaaatgaaagggtctggatatctagtgtggaataagaaagagttacaaagcatacctaaaactaatttcactgtggtcggtaggtagagctcttaggctgacatcacgagctagcgaagtagcaagtttagctgcactggtgatgacgcaagtcaacaaaagtcacgtgggataaaacggttgcttccattggaaagcctggctttagggagaggttatggacgttacgtgcggttcacactgacggtattataacaactttatgcggatgctagcgaagtttgatattctaagaacttcgcttaggttctgcaacaatgagttcttcttgcgttcaccatctgcgtggtaatgtgaccagatagcctctgccgctttctcctttgccagtttccatgaaatcttacggattatctctcactactgctgccctaggtaatgaggaaaagaaaatggcggcggaaaaggctagaggcgagggcgaagaggggtccttcagtctcacagtcgaagagaagaaggcgctttgtggattagataggtacgcactgctaatacttacaggttgtctttctctctccgggttcaaaagactctccaggaggcctgactgcatcttgcttgcggtgacgactcgctttcgcctagcactgttaacaatgttttgggagattttttactgatgtctttctgtgatcattaacaagagccatgtatttcttccacagcagcttcttcgggttcttgacccgatgcaaagatggcgccaagatgaagacgctgttgaacaaggtcagcaactgtttaaacataattacttatatttttctcctaagcttttcactgttccaagcctggagcctctccctactctgaaagtgctcattatagcttttagggatctttgaaactttttttttccttccccgtttctcctttcttggcttactttcctgatagtttgaggccctgcctcagcatacagcccagagcactgcttattttgcagtttttctgaggtcagctatatcctccagttactcatctcaagttatgagaagacgcttggttgttgcagcctcttgtgatatacacacgttttattattttcccataagggacaagttataggtaaggagataaagagtttccgtacttaaaaagaattttttacgatgcagtagtaatactatgcagaaataatactgaattccagtttggaatgttaatttttttttttttttacttccttgtagtttattttccagagagtgagagagcacacggcatttcctttgtgaaactttgcattgtcatttagttattggctgctggtatcaatcaagatggtgg

SEQ ID NO: 36 - DNA, 2550 bp, Artificial Sequence
  3′ Homology arm
  misc_feature: Synthetic
gaaatagagggaggagaggggaaatgaaacagttttagaaaggaaacagggtagttttaccaagaaaatattaaacctagcaagtacaggttaatgaaaagtattccccattacttaaaaggagaattatcaagtctttttaaacctttaaaaaaagattcttagccgggcggggtggcacatgcctttaatcccagcacttgggaggcagaggcaggcagatttctgagtttgaggccagtctggtctacagagtgagttccaggacagccagggctacacagagataccctgtctcaaaaaaaaaaaaaaaattcttaaaggtaacatatttagatacttgtaaaccatagcaaaaaaagcacagatctgtgacatacatacttgttaccattatatagtttccgttctaatgaaaatcagtgattataaacttgataatttcataccaccaatgaagtattaatagtattcttttttggtttttggagacagggtttctcagtatagctctggttgctatacagttaatagtgttcttaacatatatgtgtatatataaatttatgtatgtatgtatatatatatatacatatatatatatgcatattatgcacacataatttttggtttggtttggttgtgagcctagcctttaaggactgagtccaagatttaaacactcttcaggtgatatgtatagaaacatatttctgtttactagtaggttgctaggtttctggtgtacagtgggtaattttaaatttgatgttaacctccacactcttttaagatttcatggtaaaagcttaaagaaatgattttgtctagtttttatttttattttttaattttttttatttttttggtttttggagacagggtttctctgtatagctctggctgtcctggaactcactttgtagaccaggctggcctcaaactcagaaatctgcctgtctctccctcctgagtgctgggattaaaggcatgcaccaccacgcctgacgtggtttttaaagatataattatttaactatatcttagcatttcttttttttttttagagtagtaattcttatattataaaaaagacttggtgttgaatgtatttttggagtgactacaacaactatgtgtgatcctgtttgtcctagaactcacagacagaaatatcactttctctgctttctcattgctcattgctaagattaaagccctgttttcctaaaactggcttcttttaaatttaataattttgtatatattcatgtctggaagttataggacaaattgtggaagtcagttttcttcttctatcatgtcagggctggaggcaagtccttttactagctaagccacttccttgaactttcagtttatgtgtatagtgtttctactgcttgtaggtctattccacttgagtgtctggttctactaaggagtttagaagagacagacctatggaactggaattagtgaaggttgtgagcttcactaatcaaaccagtgttctcttgcagagctctttagtttatttaagtagcatatatttttcaggtgctttgtaatatcaatttatatgttagtaaactgttttcaaatattgaaataacaggtattttttaactaattgcaacagcattttaaaaatgagaagttaaaaacaaaatatgagaaactatcaagtgccactgttcttagaacatttatgagttgtgcctttattaaacattgagtaatatgtatttttgttttagatcagtctcagcctttctaatgcagtttctcatgttgtgatgacccccaaccataaaattatttttgttgttattcataactaattttggtgctgttatgaatcattatgtaaatatctgaatagtttattagtgaccaaaaaggtattttgacccataggttgagaaccattattttagacactgtcttagtctatattttgttgcccagaatagctttaaatttgaagaagtactcatctctcagcctccaagtattgagatttcaggcatgagccactactctttaacagttattaaaatgtattacaattataatttaaaatgacaagtattaaaaactgaaatatagggctggtgagatggctcagtgggtaagagcacccgactgctcttccgaaggtccagagttcaaatcccagcaaccacatggtggctcacaaccatccataacgagatctgactccctcttctggagtgtctgaagacagctacagtgtacttacatataataaataaatctttaaaaaaaaaaaaacaaaaaaaaacctgaaatatataaagggcaattgatggatggtggtggaatatccctttatcctagcactgtgcagaggcagttgggtctcttataagtttgaggccagcctgatctacatagcaagtttcaggacagcaagattagacagagaaacctgcctcaaaaaaggacaatttgtttttaagcctgacagtggtggcatacacctttaaacccagcacttgggggacataggcagatagatctttg

SEQ ID NO: 37 - DNA, 34 bp, Artificial Sequence
  loxP sites
ataacttcgtatagcatacattatacgaagttat

SEQ ID NO: 38 - DNA, 353 bp, Artificial Sequence
  Exon 1
  misc_feature: Synthetic
attggaaagcctggctttagggagaggttatggacgttacgtgcggttcacactgacggtattataacaactttatgcggatgctagcgaagtttgatattctaagaacttcgcttaggttctgcaacaatgagttcttcttgcgttcaccatctgcgtggtaatgtgaccagatagcctctgccgctttctcctttgccagtttccatgaaatcttacggattatctctcactactgctgccctaggtaatgaggaaaagaaaatggcggcggaaaaggctagaggcgagggcgaagaggggtccttcagtctcacagtcgaagagaagaaggcgctttgtggattagatag

SEQ ID NO: 39 - DNA, 64 bp, Artificial Sequence
  Exon 2
  misc_feature: Synthetic
cagcttcttcgggttcttgacccgatgcaaagatggcgccaagatgaagacgctgttgaacaag

SEQ ID NO: 40 - DNA, 4335 bp, Artificial Sequence
  Cas9 adapted for mammalian expression
  misc_feature: Synthetic
atggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagggaagcggagccactaacttctccctgttgaaacaagcaggggatgtcgaagagaatcccgggcca

SEQ ID NO: 41 - protein, 1445 aa, Artificial Sequence
  Cas9 adapted for mammalian expression
  misc_feature: Synthetic
MDYKDHDGDYKDHDIDYKDDDDKMAPKKKRKVGIHGVPAADKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKKGSGATNFSLLKQAGDVEENPGP

SEQ ID NO: 42 - DNA, 5052 bp, Artificial Sequence
  Cas9-2A peptide-GFP cassette
  misc_feature: Synthetic
atggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagggaagcggagccactaacttctccctgttgaaacaagcaggggatgtcgaagagaatcccgggccagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 43 - protein, 1683 aa, Artificial Sequence
  Cas9-2A peptide-GFP
  misc_feature: Synthetic
MDYKDHDGDYKDHDIDYKDDDDKMAPKKKRKVGIHGVPAADKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKKGSGATNFSLLKQAGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK